(19) United States
(12) Patent Application Publication
(10) Pub. No.: US 2007/0031853 A1
(43) Pub. Date: Feb. 8, 2007

(54) Gene sequence variations with utility in determining the treatment of neurological or psychiatric disease

(75) Inventors: Vincent P. Stanton JR., Belmont, MA (US); Martin Zillmann, Shrewsbury, MA (US)

Correspondence Address: FISH & RICHARDSON PC, P.O. BOX 1022, MINNEAPOLIS, MN, 55440-1022, US

(73) Assignee: Variagenics, Inc., a Delaware corporation

(21) Appl. No.: 11/120,291

(22) Filed: May 2, 2005

Related U.S. Application Data
(63) Continuation of application No. 10/227,555, filed on Aug. 23, 2002, now abandoned.
Application No. 10/227,555 is a continuation-in-part of application No. 10/131,272, filed on Apr. 23, 2002, now abandoned.
Application No. 10/131,272 is a continuation-in-part of application No. 10/043,735, filed on Jan. 11, 2002, now abandoned.
Application No. 10/043,735 is a continuation-in-part of application No. 09/954,696, filed on Sep. 17, 2001, now abandoned.
Application No. 09/954,696 is a continuation-in-part of application No. 09/883,480, filed on Jun. 18, 2001, now abandoned.
Application No. 09/883,480 is a continuation-in-part of application No. 09/833,240, filed on Apr. 11, 2001, now abandoned.
Application No. 09/833,240 is a continuation-in-part of application No. 09/813,526, filed on Mar. 21, 2001, now abandoned.
Application No. 09/813,526 is a continuation-in-part of application No. 09/780,397, filed on Feb. 9, 2001, now abandoned.
Application No. 09/780,397 is a continuation-in-part of application No. 09/733,654, filed on Dec. 7, 2000, now abandoned.
Application No. 09/733,654 is a continuation-in-part of application No. 09/709,205, filed on Nov. 8, 2000, now abandoned.
Application No. 09/709,205 is a continuation-in-part of application No. 09/695,804, filed on Oct. 24, 2000, now abandoned.
Application No. 09/695,804 is a continuation-in-part of application No. 09/677,418, filed on Oct. 6, 2000, now abandoned.
Application No. 09/677,418 is a continuation-in-part of application No. 09/641,371, filed on Aug. 18, 2000, now abandoned.
Application No. 09/641,371 is a continuation-in-part of application No. 09/590,752, filed on Jun. 8, 2000, now abandoned.
Application No. 09/590,752 is a continuation-in-part of application No. 09/494,313, filed on Feb. 1, 2000, now abandoned.
Application No. 09/494,313 is a continuation-in-part of application No. 09/489,379, filed on Jan. 21, 2000, now abandoned.
(63) Continuation-in-part of application No. 09/451,252, filed on Nov. 29, 1999, now abandoned.
(63) Continuation-in-part of application No. 09/427,835, filed on Oct. 26, 1999, now abandoned.
(63) Continuation-in-part of application No. 09/414,330, filed on Oct. 6, 1999, now abandoned.
(63) Continuation-in-part of application No. 09/389,993, filed on Sep. 3, 1999, now abandoned.
(63) Continuation-in-part of application No. 09/370,841, filed on Aug. 9, 1999, now abandoned.
(63) Continuation-in-part of application No. 09/300,747, filed on Apr. 26, 1999, now abandoned.
(60) Provisional application No. 60/121,047, filed on Feb. 22, 1999.
(60) Provisional application No. 60/131,334, filed on Apr. 26, 1999.
(60) Provisional application No. 60/131,191, filed on Apr. 26, 1999.

(51) Int. Cl. C12Q 1/68

(52) U.S. Cl. 435/6

(57) ABSTRACT

The present disclosure describes the use of genetic variance information for genes involved in neurologic and phychiatric diseases and in the selection of effective methods of treatment of such disease or condition. The variance information is indicative of the expected response of a patient to a method of treatment. Methods of determining relevant variance information and additional methods of using such variance information are also described.

### RELATED APPLICATIONS

This application is a continuation-in-part of U.S. application Ser. No. 10/131,272, filed Apr. 23, 2002, which is a continuation-in-part of U.S. application Ser. No. 10/043,735, filed Jan. 11, 2002, which is a continuation-in-part of 09/954,696, filed Sep. 17, 2001, which is a continuation-in-part of 09/883,480, filed Jun. 18, 2001, which is a continuation-in-part of U.S. application Ser. No. 09/833,240, filed on Apr. 11, 2001, which is a continuation-in-part of U.S. application Ser. No. 09/813,526, filed Mar. 21, 2001, which is a continuation-in-part of U.S. application Ser. No. 09/780,397, filed Feb. 9, 2001, which is a continuation-in-part of U.S. application Ser. No. 09/733,654, Dec. 7, 2000, which is a continuation-in-part of U.S. application Ser. No. 09/709,205, filed Nov. 8, 2000, which is a continuation-in-part of U.S. application Ser. No. 09/695,804, filed Oct. 24, 2000, which is a continuation-in-part of U.S. application Ser. No. 09/677,418, filed Oct. 6, 2000, which is a continuation-in-part of U.S. application Ser. No. 09/641,371, filed Aug. 18, 2000, which is a continuation-in-part of U.S. application Ser. No. 09/590,752, filed Jun. 8, 2000, which is a continuation-in-part of U.S. application Ser. No. 09/494,313 filed Feb. 1, 2000, which is a continuation-in-part of U.S. application Ser. No. 09/489,379, filed Jan. 21, 2000, and claims the benefit of 60/121,047, filed Feb. 22, 1999. This application is also a continuation-in-part of U.S. application Ser. No. 09/451,252, filed Nov. 29, 1999, and a continuation-in-part of U.S. application Ser. No. 09/427,835, filed Oct. 26, 1999, and a continuation-in-part of U.S. application Ser. No. 09/414,330, filed Oct. 6, 1999, and a continuation-in-part of U.S. application Ser. No. 09/389,993, filed Sep. 3, 1999, and a continuation-in-part of U.S. application Ser. No. 09/370,841, filed Aug. 9, 1999, and Stanton and Adams, U.S. application Ser. No. 09/300,747, filed Apr. 26, 1999, and claims the benefit of U.S. Provisional Patent Application Ser. No. 60/131,334, filed Apr. 26, 1999, and U.S. Provisional Application Ser. No. 60/131,191, filed Apr. 26, 1999, all of which are hereby incorporated by reference in their entireties, including drawings and tables.

### SUBMISSIONS ON COMPACT DISC

Tables 3, 4, and 19 of the present application are being submitted on CD-ROM concurrently with the present application as permitted under 37 C.F.R. §1.52(e). Two duplicate copies (copy 1 and copy 2) of each of 2 CDs, either containing Table 3 and Table 4 (CD1 of 2) or Table 19 (CD2 of 2) of the present application, are being submitted. The contents of Copy I and Copy 2 of “CD1 of 2” are identical. The contents of Copy 1 and Copy 2 of “CD2 of 2” are identical. “CD1 of 2” contains the following files:

Table_{—}3.txt; 0.97 MB in size; created on Jul. 29, 2002

Table_{—}4.txt; 126 KB in size, created on Jul. 29, 2002 “CD2 of 2” contains the following file:

CNS.FA, referred to herein as Table 19; 3.92 MB in size, created on Jul. 30, 2002

The contents of the above-identified CDs are incorporated herein by reference in their entirety.

### BACKGROUND OF THE INVENTION

This application concerns the field of mammalian therapeutics and the selection of therapeutic regimens utilizing host genetic information, including gene sequence variances within the human genome in human populations.

The information provided below is not admitted to be prior art to the present invention, but is provided solely to assist the understanding of the reader.

Many drugs or other treatments are known to have highly variable safety and efficacy in different individuals. A consequence of such variability is that a given drug or other treatment may be effective in one individual, and ineffective or not well-tolerated in another individual. Thus, administration of such a drug to an individual in whom the drug would be ineffective would result in wasted cost and time during which the patient's condition may significantly worsen. Also, administration of a drug to an individual in whom the drug would not be tolerated could result in a direct worsening of the patient's condition and could even result in the patient's death.

For some drugs, over 90% of the measurable variation in selected pharmacokinetic parameters has been shown to be heritable. For a limited number of drugs, DNA sequence variances have been identified in specific genes that are involved in drug action or metabolism, and these variances have been shown to account for the variable efficacy or safety of the drugs in different individuals. As the sequence of the human genome is completed, and as additional human gene sequence variances are identified, the power of genetic methods for predicting drug response will further increase.

In this application, we address the difficulties that arise in treating neurological and psychiatric disease. Diseases of the central nervous system (CNS) present unique medical challenges to clinicians, patients, and caregivers. These diseases often progress to severely debilitating conditions. Further, the efficacy of available treatments is limited and there are serious, mostly unpredictable, side effects associated with some drugs. The progressive nature of neurological and psychiatric disease makes the passage of time a crucial issue in the treatment process. Specifically, selection of optimal treatment for neurological and psychiatric diseases is complicated by the fact that it often takes weeks or months to determine if a given therapy is symptomology producing a measurable benefit. Thus the current empirical approach to prescribing pharmacotherapy, in which each course of treatment for a given patient is a small experiment, is unsatisfactory from both a medical and economic perspective. Even when an effective treatment is ultimately identified, it often follows a period of ineffective or suboptimal treatment. Accordingly, a method that would help caregivers predict which patients will exhibit beneficial therapeutic responses to a specific medication would provide both medical and economic benefits. As healthcare becomes increasingly costly, the ability to rationally allocate healthcare expenditures, and in particular pharmacy resources, also becomes increasingly important.

### SUMMARY OF THE INVENTION

The present invention is concerned generally with the field of identifying an appropriate treatment regimen for a neurological or psychiatric disease based upon genotype in mammals, particularly in humans. It is further concerned with the genetic basis of inter-patient variation in response to therapy, including drug therapy. Specifically, this invention describes the identification of gene sequence variances useful in the field of therapeutics for optimizing efficacy and safety of drug therapy. These variances may be useful either during the drug development process or in guiding the optimal use of already approved compounds. DNA sequence variances in candidate genes (i.e. genes that may plausibly affect the action of a drug) are tested in clinical trials, leading to the establishment of diagnostic tests useful for improving the development of new pharmaceutical products and/or the more effective use of existing pharmaceutical products. Methods for identifying genetic variances and determining their utility in the selection of optimal therapy for specific patients are also described. In general, the invention relates to methods for identifying patient population subsets that respond to drug therapy with either therapeutic benefit or side effects (i.e. symptomatology prompting concern about safety or other unwanted signs or symptoms).

Limited Selectivity of CNS Drugs

It is known that compounds that may have initially been designed to mimic one endogenous neurotransmitter often interact with multiple neurotransmitter receptor classes (as either agonist or antagonist). For example, an adrenergic compound at pharmacologically active concentrations may interact with both alpha and beta adrenergic receptors and, at higher doses, dopamine receptors and possibly other catecholamine receptors. Also, a compound may interact with multiple receptor types at the same concentration. For example, atypical neuroleptic agents (e.g. clozapine) interact with dopaminergic, serotonergic, histaminergic, adrenergic, and muscarinic (cholinergic) receptor types within the central nervous system (CNS). This broad range of pharmacological interactions has implications for the design of experiments to identify genetic determinants of drug response. In particular, because of the broad pharmacological interactions of compounds being developed as CNS drugs, it may be beneficial to determine the effect of DNA sequence variances in a number of different sets of genes (belonging to different biochemical pathways) in order to identify a variance or variances responsible for interpatient variation in drug response.

Multiple Receptor Subtypes

In addition to the phenomenon of compounds that act simultaneously on different receptor classes, one should also consider that most CNS receptor classes have many members, each with its own anatomical distribution, expression levels, ligand binding properties and signal transduction pathway. For example, there are 5 dopamine receptors, 14 serotonin receptors and 12 cholinergic receptors (four muscarinic, eight nicotinic). The cholinergic receptors are made up of multiple subunits. The expression of these receptor families in different neuronal populations is highly overlapping. Thus, a single neuron may express several receptor types, even within the same receptor class. The specific pattern of expression of receptors in a given patient—the anatomical distribution of neurons bearing receptors of each type and the level of expression of receptors in each neuron—are expected to be at least partially under genetic control, and are likely to account for some fraction of interpatient variation in drug response. In providing lists of genes that may account for variable responses to different drugs we have taken into account these different subtypes.

Neuromodulation

In addition to the complexity of overlapping signaling systems in virtually all higher order functions of the CNS, there is also the phenomenon of integration of disparate signals by individual neurons. A post synaptic terminal may receive multiple presynaptic inputs, some excitatory and some inhibitory. These presynaptic signals may be integrated by a neuron and result in release of a mix of excitatory and inhibitory neurotransmitters. Similar complexity and redundancy exists at the level of intracellular events (which are also frequently targeted by CNS drugs).

This broad range of pharmacological interactions with receptors, transporters, enzymes and other proteins which are differentially expressed in different populations of cells in the CNS has implications for the design of experiments to identify genetic determinants of drug response. In particular, because of the broad pharmacological interactions of compounds being developed as CNS drugs it may be necessary to study the effect of DNA sequence variances in a number of different sets of genes (belonging to different biochemical pathways) in order to identify a sequence variance or set of variances responsible for interpatient variation in drug response. Methods are described herein for identifying relevant DNA sequence variances and associating them with drug response phenotypes.

While the complexity of CNS physiology creates challenges for pharmacogenetic studies, it is also the case that the pharmacological treatment of CNS diseases provides broad scope for the methods of this invention, because (i) the hereditary component of many CNS diseases is well-established, indicating a major role of genetic (as opposed to environmental) factors in disease etiology, (ii) the molecular pharmacology of CNS drugs is generally well understood, providing a rational basis for selecting genes for pharmacogenetic investigation (iii) the heterogeneous responses of patients to CNS drugs suggests that the factors governing response extend beyond presently understood mechanisms; genetic variation can affect virtually all aspects of pharmacology, and is, for the reasons cited above, likely to account for much of the heterogeneity in drug response. In this application we describe methods for improving the treatment of neurological and psychiatric diseases, movement disorders, neurodegenerative diseases, disorders of sensation, and cerebrovascular diseases. In specific examples, we address the treatment of migraine, pain, epilepsy, schizophrenia, stroke, depression, anxiety, spasticity, Parkinson's disease, dementia, demyelinating disease, amyotrophic lateral sclerosis, and Huntington's disease. Described in the Examples and Tables are genes and gene sequence variances useful in the genetic analysis of treatment response for each of these diseases, and exemplary compounds being developed to treat each of these diseases.

The inventors have determined that the identification of gene sequence variances in genes that may be involved in drug action are useful for determining whether genetic variances account for variable drug efficacy and safety and for determining whether a given drug or other therapy may be safe and effective in an individual patient. Provided in this invention are identifications of genes and sequence variances which can be useful in connection with predicting differences in response to treatment and selection of appropriate treatment of a disease or condition. A target gene and variances are useful, for example, in pharmacogenetic association studies and diagnostic tests to improve the use of certain drugs or other therapies including, but not limited to, the drug classes and specific drugs identified in the 1999 Physicians' Desk Reference (53rd edition), Medical Economics Data, 1998, the 1995 United States Pharmacopeia XXIII National Formulary XVIII, Interpharm Press, 1994, Examples 5-18 or other sources as described below.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests which may include, among others, laboratory tests to determine the presence of DNA sequence variances or variant forms of certain genes in a patient. Symptoms are subjective evidence of disease or a patients condition, i.e. the patients perception of an abnormal condition that differs from normal function, sensation, or appearance, which may include, without limitations, physical disabilities, morbidity, pain, and other changes from the normal condition experienced by an individual. Various diseases or conditions include, but are not limited to; those categorized in standard textbooks of medicine including, without limitation, textbooks of nutrition, allopathic, homeopathic, and osteopathic medicine. In certain aspects of this invention, the disease or condition is selected from the group consisting of the diseases or conditions identified herein and the types of diseases listed in standard texts such as Harrison's Principles of Internal Medicine (14th Ed) by Anthony S. Fauci, Eugene Braunwald, Kurt J. Isselbacher, et al. (Editors), McGraw Hill, 1997, or Robbins Pathologic Basis of Disease (6th edition) by Ramzi S. Cotran, Vinay Kumar, Tucker Collins & Stanley L. Robbins, W B Saunders Co., 1998, or the Diagnostic and Statistical Manual of Mental Disorders: DSM-IV (4^{th} edition), American Psychiatric Press, 1994, or other texts described below.

In connection with the methods of this invention, unless otherwise indicated, the term “suffering from a disease or condition” means that a person is either presently subject to the signs and symptoms, or is more likely to develop such signs and symptoms than a normal person in the population. Thus, for example, a person suffering from a condition can include a developing fetus, a person subject to a treatment or environmental condition which enhances the likelihood of developing the signs or symptoms of a condition, or a person who is being given or will be given a treatment which increase the likelihood of the person developing a particular condition. For example, tardive dyskinesia is associated with long-term use of anti-psychotics; dyskinesias, paranoid ideation, psychotic episodes and depression have been associated with use of L-dopa in Parkinson's disease; (and dizziness, diplopia, ataxia, sedation, impaired mentation, weight gain, and other undesired effects have been described for various anticonvulsant therapies. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “therapy” refers to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such therapy can involve, for example, nutritional modifications, administration of radiation, administration of a drug, behavioral modifications, and combinations of these, among others.

The term “drug” as used herein refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The term “drug” may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”. Most preferably the drug is approved by a government agency for treatment of a specific disease or condition.

A “low molecular weight compound” has a molecular weight <5,000 Da, more preferably <2500 Da, still more preferably <1000 Da, and most preferably <700 Da.

Those familiar with drug use in medical practice will recognize that regulatory approval for drug use is commonly limited to approved indications, such as to those patients afflicted with a disease or condition for which the drug has been shown to be likely to produce a beneficial effect in a controlled clinical trial. Unfortunately, it has generally not been possible with current knowledge to predict which patients will have a beneficial response, with the exception of certain diseases such as bacterial infections where suitable laboratory methods have been developed. Likewise, it has generally not been possible to determine in advance whether a drug will be safe in a given patient. Regulatory approval for the use of most drugs is limited to the treatment of selected diseases and conditions. The descriptions of approved drug usage, including the suggested diagnostic studies or monitoring studies, and the allowable parameters of such studies, are commonly described in the “label” or “insert” which is distributed with the drug. Such labels or inserts are preferably required by government agencies as a condition for marketing the drug and are listed in common references such as the Physicians Desk Reference (PDR). These and other limitations or considerations on the use of a drug are also found in medical journals, publications such as pharmacology, pharmacy or medical textbooks including, without limitation, textbooks of nutrition, allopathic, homeopathic, and osteopathic medicine.

Many widely used drugs are effective in a minority of patients receiving the drug, particularly when one controls for the placebo effect. For example, the PDR shows that about 45% of patients receiving Cognex (tacrine hydrochloride) for Alzheimer's disease show no change or minimal worsening of their disease, as do about 68% of controls (including about 5% of controls who were much worse). About 58% of Alzheimer's patients receiving Cognex were minimally improved, compared to about 33% of controls, while about 2% of patients receiving Cognex were much improved compared to about 1% of controls. Thus a tiny fraction of patients had a significant benefit. Response to treatments for amyotrophic lateral sclerosis are likewise minimal.

Thus, in a first aspect, the invention provides a method for selecting a treatment for a patient suffering from a disease or condition by determining whether or not a gene or genes in cells of the patient (in some cases including both normal and disease cells, such as cancer cells) contain at least one sequence variance which is indicative of the effectiveness of the treatment of the disease or condition. The gene or genes are specified herein, in Tables 1, 3, and 4. Preferably the at least one variance includes a plurality of variances. Preferably the at least one variance, or plurality of variances provides or constitutes a haplotype or haplotypes. (In each of the aspects of this invention, at least one variance or a plurality of variances preferably provides one or more haplotypes.) Preferably the joint presence of the plurality of variances is indicative of the potential effectiveness or safety of the treatment in a patient having such plurality of variances. The plurality of variances may each be indicative of the potential effectiveness of the treatment, and the effects of the individual variances may be independent or additive, or the plurality of variances may be indicative of the potential effectiveness if at least 2, 3, 4, or more appear jointly. The plurality of variances may also be combinations of these relationships. The plurality of variances may include variances from one, two, three or more gene loci.

In preferred embodiments of aspects of the invention involving genes relating to psychiatric or neurological disease or related conditions, or to pharmacological responses to compounds used to treat such diseases or conditions, the gene product is involved in a function as described in the Background of the Invention or otherwise described herein.

In some cases, the selection of a method of treatment, i.e., a therapeutic regimen, may incorporate selection of one or more from a plurality of medical therapies. Thus, the selection may be the selection of a method or methods which is/are more effective or less effective than certain other therapeutic regimens (with either having varying safety parameters). Likewise or in combination with the preceding selection, the selection may be the selection of a method or methods, which is safer than certain other methods of treatment in the patient.

The selection may involve either positive selection or negative selection or both, meaning that the selection can involve a choice that a particular method would be an appropriate method to use and/or a choice that a particular method would be an inappropriate method to use. Thus, in certain embodiments, the presence of the at least one variance is indicative that the treatment will be effective or otherwise beneficial (or more likely to be beneficial) in the patient. Stating that the treatment will be effective means that the probability of beneficial therapeutic effect is greater than in a person not having the appropriate presence or absence of particular variances. In other embodiments, the presence of the at least one variance is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient with a particular variance or variances than in a patient who has a different variance or variances. Also in preferred embodiments, the presence of the at least one variance is indicative that the treatment is both effective and unlikely to result in undesirable effects or outcomes, or vice versa (is likely to have undesirable side effects but unlikely to produce desired therapeutic effects).

In reference to response to a treatment, the term “tolerance” refers to the ability of a patient to accept a treatment, based, e.g., on deleterious effects and/or effects on lifestyle. Frequently, the term principally concerns the patients perceived magnitude of deleterious effects such as nausea, weakness, dizziness, and diarrhea, among others. Such experienced effects can, for example, be due to general or cell-specific toxicity, activity on non-target cells, cross-reactivity on non-target cellular constituents (non-mechanism based), and/or side effects of activity on the target cellular substituents (mechanism based), or the cause of toxicity may not be understood. In any of these circumstances one may identify an association between the undesirable effects and variances in specific genes.

Adverse responses to drugs constitute a major medical problem, as shown in two recent meta-analyses (Lazarou, J. et al, Incidence of adverse drug reactions in hospitalized patients: a meta-analysis of prospective studies, JAMA 279:1200-1205, 1998; Bonn, Adverse drug reactions remain a major cause of death, Lancet 351:1183, 1998). An estimated 2.2 million hospitalized patients in the United Stated had serious adverse drug reactions in 1994, with an estimated 106,000 deaths (Lazarou et al.). To the extent that some of these adverse events are due to genetically encoded biochemical diversity among patients in pathways that effect drug action, the identification of variances that are predictive of such effects will allow for more effective and safer drug use.

In embodiments of this invention, the variance or variant form or forms of a gene is/are associated with a specific response to a drug. The frequency of a specific variance or variant form of the gene may correspond to the frequency of an efficacious response to administration of a drug. Alternatively, the frequency of a specific variance or variant form of the gene may correspond to the frequency of an adverse event resulting from administration of a drug. Alternatively the frequency of a specific variance or variant form of a gene may not correspond closely with the frequency of a beneficial or adverse response, yet the variance may still be useful for identifying a patient subset with high response or toxicity incidence because the variance may account for only a fraction of the patients with high response or toxicity. In such a case the preferred course of action is identification of a second or third or additional variances that permit identification of the patient groups not usefully identified by the first variance. Preferably, the drug will be effective in more than 20% of individuals with one or more specific variances or variant forms of the gene, more preferably in 40% and most preferably in >60%. In other embodiments, the drug will be toxic or create clinically unacceptable side effects in more than 10% of individuals with one or more variances or variant forms of the gene, more preferably in >30%, more preferably in >50%, and most preferably in >70% or in more than 90%.

Also in other embodiments, the method of selecting a treatment includes excluding or eliminating a treatment, where the presence or absence of the at least one variance is indicative that the treatment will be ineffective or contra-indicated, e.g., would result in excessive weight gain. In other preferred embodiments, in cases in which undesirable side-effects may occur or are expected to occur from a particular therapeutic treatment, the selection of a method of treatment can include identifying both a first and second treatment, where the first treatment is effective to treat the disease or condition, and the second treatment reduces a deleterious effect of the first treatment.

The phrase “eliminating a treatment” or “excluding a treatment” refers to removing a possible treatment from consideration, e.g., for use with a particular patient based on the presence or absence of a particular variance(s) in one or more genes in cells of that patient, or to stopping the administration of a treatment.

Usually, the treatment will involve the administration of a compound preferentially active or safe in patients with a form or forms of a gene, where the gene is one identified herein. The administration may involve a combination of compounds. Thus, in preferred embodiments, the method involves identifying such an active compound or combination of compounds, where the compound is less active or is less safe or both when administered to a patient having a different form of the gene.

Also in preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum.

Also in this context, a “suitable dosage level” refers to a dosage level that provides a therapeutically reasonable balance between pharmacological effectiveness and deleterious effects. Often this dosage level is related to the peak or average serum levels resulting from administration of a drug at the particular dosage level.

Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects (and preferably while still being able to have reasonable patient compliance for self-administered drugs). Thus, it is desirable to maintain the serum level of the drug within a therapeutic window of concentrations for the greatest percentage of time possible without such deleterious effects as would cause a prudent physician to reduce the frequency of administration for a particular dosage level.

A particular gene or genes can be relevant to the treatment of more than one disease or condition, for example, the gene or genes can have a role in the initiation, development, course, treatment, treatment outcomes, or health-related quality of life outcomes of a number of different diseases, disorders, or conditions. Thus, in preferred embodiments, the disease or condition or treatment of the disease or condition is any which involves a gene from the gene list described herein as Tables 1, 3 and 4.

Determining the presence of a particular variance or plurality of variances in a particular gene in a patient can be performed in a variety of ways. In preferred embodiments, the detection of the presence or absence of at least one variance involves amplifying a segment of nucleic acid including at least one of the at least one variances. Preferably a segment of nucleic acid to be amplified is 500 nucleotides or less in length, more preferably 100 nucleotides or less, and most preferably 45 nucleotides or less. Also, preferably the amplified segment or segments includes a plurality of variances, or a plurality of segments of a gene or of a plurality of genes. In other embodiments, e.g., where a haplotype is to be determined, the segment of nucleic acid is at least 500 nucleotides in length, or at least 2 kb in length, or at least 5 kb in length.

In preferred embodiments, determining the presence of a set of variances in a specific gene related to treatment of neurological disease or other related genes, or genes listed in Tables 1, 3, and 4, includes a haplotyping test that involves allele specific amplification of a large DNA segment of no greater than 25,000 nucleotides, preferably no greater than 10,000 nucleotides and most preferably no greater than 5,000 nucleotides. Alternatively one allele may be enriched by methods other than amplification prior to determining genotypes at specific variant positions on the enriched allele as a way of determining haplotypes. Preferably the determination of the presence or absence of a haplotype involves determining the sequence of the variant sites by methods such as chain terminating DNA sequencing or minisequencing, or by oligonucleotide hybridization or by mass spectrometry.

The term “genotype” in the context of this invention refers to the alleles present in DNA from a subject or patient, where an allele can be defined by the particular nucleotide(s) present in a nucleic acid sequence at a particular site(s). Often a genotype is the nucleotide(s) present at a single polymorphic site known to vary in the human population.

In preferred embodiments, the detection of the presence or absence of the at least one variance involves contacting a nucleic acid sequence corresponding to one of the genes identified above or a product of such a gene with a probe. The probe is able to distinguish a particular form of the gene or gene product or the presence or a particular variance or variances, e.g., by differential binding or hybridization. Thus, exemplary probes include nucleic acid hybridization probes, peptide nucleic acid probes, nucleotide-containing probes which also contain at least one nucleotide analog, and antibodies, e.g., monoclonal antibodies, and other probes as discussed herein. Those skilled in the art are familiar with the preparation of probes with particular specificities. Those skilled in the art will recognize that a variety of variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. (See Current Protocols in Molecular Biology by F. M. Ausubel, R. Brent, R. E. Kngston, D. D. Moore, J. D. Seidman, K. Struhl, and V. B. Chanda (editors, John Wiley & Sons.)

In other preferred embodiments, determining the presence or absence of the at least one variance involves sequencing at least one nucleic acid sample. The sequencing involves sequencing of a portion or portions of a gene and/or portions of a plurality of genes which includes at least one variance site, and may include a plurality of such sites. Preferably, the portion is 500 nucleotides or less in length, more preferably 200 or 100 nucleotides or less, and most preferably 45 nucleotides or less in length. Such sequencing can be carried out by various methods recognized by those skilled in the art, including use of dideoxy termination methods (e.g., using dye-labeled dideoxy nucleotides) and the use of mass spectrometric methods. In addition, mass spectrometric methods may be used to determine the nucleotide present at a variance site. In preferred embodiments in which a plurality of variances is determined, the plurality of variances can constitute a haplotype or collection of haplotypes. Preferably the methods for determining genotypes or haplotypes are designed to be sensitive to all the common genotypes or haplotypes present in the population being studied (for example, a clinical trial population).

The terms “variant form of a gene”, “form of a gene”, or “allele” refer to one specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants”. The term “alternative form” refers to an allele that can be distinguished from other alleles by having distinct variances at least one, and frequently more than one, variant sites within the gene sequence. Other terms known in the art to be equivalent include mutation and polymorphism, although mutation is often used to refer to an allele associated with a deleterious phenotype. In preferred aspects of this invention, the variances are selected from the group consisting of the variances listed in the variance tables herein or in a patent or patent application referenced and incorporated by reference in this disclosure. In the methods utilizing variance presence or absence, reference to the presence of a variance or variances means particular variances, i.e., particular nucleotides at particular polymorphic sites, rather than just the presence of any variance in the gene.

Variances occur in the human genome at approximately one in every 500-1,000 bases within the human genome when two alleles are compared. When multiple alleles from unrelated individuals are compared the density of variant sites increases as different individuals, when compared to a reference sequence, will often have sequence variances at different sites. At most variant sites there are only two alternative nucleotides involving the substitution of one base for another or the insertion/deletion of one or more nucleotides. Within a gene there may be several variant sites. Variant forms of the gene or alternative alleles can be distinguished by the presence of alternative variances at a single variant site, or a combination of several different variances at different sites (haplotypes).

It is estimated that there are 3,300,000,000 bases in the sequence of a single haploid human genome. All human cells except germ cells are normally diploid. Each gene in the genome may span 100-10,000,000 bases of DNA sequence or 100-20,000 bases of mRNA. It is estimated that there are between 60,000 and 150,000 genes in the human genome. The “identification” of genetic variances or variant forms of a gene involves the discovery of variances that are present in a population. The identification of variances is required for development of a diagnostic test to determine whether a patient has a variant form of a gene that is known to be associated with a disease, condition, or predisposition or with the efficacy or safety of the drug. Identification of previously undiscovered genetic variances is distinct from the process of “determining” the status of known variances by a diagnostic test (often referred to as genotyping). The present invention provides exemplary variances in genes listed in the gene tables, as well as methods for discovering additional variances in those genes and a comprehensive written description of such additional possible variances. Also described are methods for DNA diagnostic tests to determine the DNA sequence at a particular variant site or sites.

The process of “identifying” or discovering new variances involves comparing the sequence of at least two alleles of a gene, more preferably at least 10 alleles and most preferably at least 50 alleles (keeping in mind that each somatic cell has two alleles). The analysis of large numbers of individuals to discover variances in the gene sequence between individuals in a population will result in detection of a greater fraction of all the variances in the population. Preferably the process of identifying reveals whether there is a variance within the gene; more preferably identifying reveals the location of the variance within the gene; more preferably identifying provides knowledge of the sequence of the nucleic acid sequence of the variance, and most preferably identifying provides knowledge of the combination of different variances that comprise specific variant forms of the gene (referred to as alleles). In identifying new variances it is often useful to screen different population groups based on racial, ethnic, gender, and/or geographic origin because particular variances may differ in frequency between such groups. It may also be useful to screen DNA from individuals with a particular disease or condition of interest because they may have a higher frequency of certain variances than the general population.

The process of genotyping involves using diagnostic tests for specific variances that have already been identified. It will be apparent that such diagnostic tests can only be performed after variances and variant forms of the gene have been identified. Identification of new variances can be accomplished by a variety of methods, alone or in combination, including, for example, DNA sequencing, SSCP, heteroduplex analysis, denaturing gradient gel electrophoresis (DGGE), heteroduplex cleavage (either enzymatic as with T4 Endonuclease 7, or chemical as with osmium tetroxide and hydroxylamine), computational methods (described herein), and other methods described herein as well as others known to those skilled in the art. (See, for example: Cotton, R. G. H., Slowly but surely towards better scanning for mutations, Trends in Genetics 13(2): 43-6, 1997 or Current Protocols in Human Genetics by N. C. Dracoli, J. L. Haines, B. R. Korf, D. T. Moir, C. C. Morton, C. E. Seidman, D. R. Smith, and A. Boyle (editors), John Wiley & Sons.)

In the context of this invention, the term “analyzing a sequence” refers to determining at least some sequence information about the sequence, e.g., determining the nucleotides present at a particular site or sites in the sequence, particularly sites that are known to vary in a population, or determining the base sequence of all or of a portion of the particular sequence.

In the context of this invention, the term “haplotype” refers to a cis arrangement of two or more polymorphic nucleotides, i.e., variances, on a particular chromosome, e.g., in a particular gene. The haplotype preserves information about the phase of the polymorphic nucleotides—that is, which set of variances were inherited from one parent, and which from the other. A genotyping test does not provide information about phase. For example, an individual heterozygous at nucleotide 25 of a gene (both A and C are present) and also at nucleotide 100 (both G and T are present) could have haplotypes 25A-100G and 25C-100T, or alternatively 25A-100T and 25C-100G. Only a haplotyping test can discriminate these two cases definitively.

The terms “variances”, “variants” and “polymorphisms”, as used herein, may also refer to a set of variances, haplotypes or a mixture of the two, unless otherwise indicated. Further, the term variance, variant or polymorphism (singular), as used herein, also encompasses a haplotype unless otherwise indicated. This usage is intended to minimize the need for cumbersome phrases such as: “ . . . measure correlation between drug response and a variance, variances, haplotype, haplotypes or a combination of variances and haplotypes . . . ”, throughout the application. Instead, the italicized text in the foregoing sentence can be represented by the word “variance”, “variant” or “polymorphism”. Similarly, the term “genotype”, as used herein, means a procedure for determining the status of one or more variances in a gene, including a set of variances comprising a haplotype. Thus phrases such as “ . . . genotype a patient . . . ” refer to determining the status of one or more variances, including a set of variances for which phase is known (i.e. a haplotype).

In preferred embodiments of this invention, the frequency of the variance or variant form of the gene in a population is known. Measures of frequency known in the art include “allele frequency”, namely the fraction of genes in a population that have one specific variance or set of variances. The allele frequencies for any gene should sum to 1. Another measure of frequency known in the art is the “heterozygote frequency” namely, the fraction of individuals in a population who carry two alleles, or two forms of a particular variance or variant form of a gene, one inherited from each parent. Alternatively, the number of individuals who are homozygous for a particular form of a gene may be a useful measure. The relationship between allele frequency, heterozygote frequency, and homozygote frequency is described for many genes by the Hardy-Weinberg equation, which provides the relationship between allele frequency, heterozygote frequency and homozygote frequency in a freely breeding population at equilibrium. Most human variances are substantially in Hardy-Weinberg equilibrium. In a preferred aspect of this invention, the allele frequency, heterozygote frequency, and homozygote frequencies are determined experimentally. Preferably a variance has an allele frequency of at least 0.01, more preferably at least 0.05, still more preferably at least 0.10. However, the allele may have a frequency as low as 0.001 if the associated phenotype is, for example, a rare form of toxic reaction to a treatment or drug. Beneficial responses may also be rare.

In this regard, “population” refers to a defined group of individuals or a group of individuals with a particular disease or condition or individuals that may be treated with a specific drug identified by, but not limited to geographic, ethnic, race, gender, and/or cultural indices. In most cases a population will preferably encompass at least ten thousand, one hundred thousand, one million, ten million, or more individuals, with the larger numbers being more preferable. In preferred embodiments of this invention, the population refers to individuals with a specific disease or condition that may be treated with a specific drug. In embodiments of this invention, the allele frequency, heterozygote frequency, or homozygote frequency of a specific variance or variant form of a gene is known. In preferred embodiments of this invention, the frequency of one or more variances that may predict response to a treatment is determined in one or more populations using a diagnostic test.

It should be emphasized that it is currently not generally practical to study an entire population to establish the association between a specific disease or condition or response to a treatment and a specific variance or variant form of a gene. Such studies are preferably performed in controlled clinical trials using a limited number of patients that are considered to be representative of the population with the disease. Since drug development programs are generally targeted at the largest possible population, the study population will generally consist of men and women, as well as members of various racial and ethnic groups, depending on where the clinical trial is being performed. This is important to establish the efficacy of the treatment in all segments of the population.

In the context of this invention, the term “probe” refers to a molecule that detectably distinguishes between target molecules differing in structure. Detection can be accomplished in a variety of different ways depending on the type of probe used and the type of target molecule. Thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and nucleic acid hybridization probes. Thus, in preferred embodiments, the detection of the presence or absence of the at least one variance involves contacting a nucleic acid sequence which includes a variance site with a probe, preferably a nucleic acid probe, where the probe preferentially hybridizes with a form of the nucleic acid sequence containing a complementary base at the variance site as compared to hybridization to a form of the nucleic acid sequence having a non-complementary base at the variance site, where the hybridization is carried out under selective hybridization conditions. Such a nucleic acid hybridization probe may span two or more variance sites. Unless otherwise specified, a nucleic acid probe can include one or more nucleic acid analogs, labels or other substituents or moieties so long as the base-pairing function is retained.

As is generally understood, administration of a particular treatment, e.g., administration of a therapeutic compound or combination of compounds, is chosen depending on the disease or condition that is to be treated. Thus, in certain preferred embodiments, the disease or condition is one for which administration of a treatment is expected to provide a therapeutic benefit; in certain embodiments, the compound is a compound identified herein, e.g., in a drug table (Tables 5-14 and 16-18).

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the presence of one or more sequence variances or alleles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Effectiveness is measured in a particular population. In conventional drug development the population is generally every subject who meets the enrollment criteria (i.e. has the particular form of the disease or condition being treated). It is an aspect of the present invention that segmentation of a study population by genetic criteria can provide the basis for identifying a subpopulation in which a drug is effective against the disease or condition being treated.

The term “deleterious effects” refers to physical effects in a patient caused by administration of a treatment which are regarded as medically undesirable. Thus, for example, deleterious effects can include a wide spectrum of toxic effects injurious to health such as death of normally functioning cells when only death of diseased cells is desired, nausea, fever, inability to retain food, dehydration, damage to critical organs such as arrythmias, renal tubular necrosis, fatty liver, or pulmonary fibrosis leading to coronary, renal, hepatic, or pulmonary insufficiency among many others. In this regard, the term “contra-indicated” means that a treatment results in deleterious effects such that a prudent medical doctor treating such a patient would regard the treatment as unsuitable for administration. Major factors in such a determination can include, for example, availability and relative advantages of alternative treatments, consequences of non-treatment, and permanency of deleterious effects of the treatment.

It is recognized that many treatment methods, e.g., administration of certain compounds or combinations of compounds, may produce side-effects or other deleterious effects in patients. Such effects can limit or even preclude use of the treatment method in particular patients, or may even result in irreversible injury, dysfunction, or death of the patient. Thus, in certain embodiments, the variance information is used to select both a first method of treatment and a second method of treatment. Usually the first treatment is a primary treatment that provides a physiological effect directed against the disease or condition or its symptoms. The second method is directed to reducing or eliminating one or more deleterious effects of the first treatment, e.g., to reduce a general toxicity or to reduce a side effect of the primary treatment. Thus, for example, the second method can be used to allow use of a greater dose or duration of the first treatment, or to allow use of the first treatment in patients for whom the first treatment would not be tolerated or would be contra-indicated in the absence of a second method to reduce deleterious effects or to potentiate the effectiveness of the first treatment.

In a related aspect, the invention concerns a method for providing a correlation or other statistical test of relationship between a patient genotype and effectiveness of a treatment, by determining the presence or absence of a particular known variance or variances in cells of a patient for a gene from Tables 1, 3, and 4, or other gene related to neurological disease, and providing a result indicating the expected effectiveness of a treatment for a disease or condition. The result may be formulated by comparing the genotype of the patient with a list of variances indicative of the effectiveness of a treatment, e.g., administration of a drug described herein. The determination may be by methods as described herein or other methods known to those skilled in the art.

In a related aspect, the invention provides a method for selecting a method of treatment for a patient suffering from a disease or condition by comparing at least one variance in at least one gene in the patient, with a list of variances in the gene from Tables 1, 3, and 4, or other gene related to neurological disease, which are indicative of the effectiveness of at least one method of treatment. Preferably the comparison involves a plurality of variances or a haplotype indicative of the effectiveness of at least one method of treatment. Also, preferably the list of variances includes a plurality of variances.

Similar to the above aspect, in preferred embodiments the at least one method of treatment involves the administration of a compound effective in at least some patients with a disease or condition; the presence or absence of the at least one variance is indicative that the treatment will be effective in the patient; and/or the presence or absence of the at least one variance is indicative that the treatment will be ineffective or contra-indicated in the patient; and/or the treatment is a first treatment and the presence or absence of the at least one variance is indicative that a second treatment will be beneficial to reduce a deleterious effect of or potentiate the effectiveness of the first treatment; and/or the at least one treatment is a plurality of methods of treatment. For a plurality of treatments, preferably the selecting involves determining whether any of the methods of treatment will be more effective than at least one other of the plurality of methods of treatment. Yet other embodiments are provided as described for the preceding aspect in connection with methods of treatment using administration of a compound; treatment of various diseases, and variances in particular genes.

In the context of variance information in the methods of this invention, the term “list” refers to one or more, preferably at least 2, 3, 4, 5, 7, or 10 variances that have been identified for a gene of potential importance in accounting for inter-individual variation in treatment response. Preferably there is a plurality of variances for the gene, preferably a plurality of variances for the particular gene. Preferably, the list is recorded in written or electronic form. For example, identified variances of identified genes are recorded for some of the genes in Tables 3, and 4; additional variances for genes in Table 1 are provided in Table 1 of Stanton et al., U.S. application Ser. No. 09/300,747 or related CIP application, and additional gene variance identification tables are provided in a form which allows comparison with other variance information. The possible additional variances in the identified genes are provided in Table 3 in Stanton et al., U.S. application Ser. No. 09/300,747.

In addition to the basic method of treatment, often the mode of administration of a given compound as a treatment for a disease or condition in a patient is significant in determining the course and/or outcome of the treatment for the patient. Thus, the invention also provides a method for selecting a method of administration of a compound to a patient suffering from a disease or condition, by determining the presence or absence of at least one variance in cells of the patient in at least one identified gene from Tables 1, 3, and 4, where such presence or absence is indicative of an appropriate method of administration of the compound. Preferably, the selection of a method of treatment (a treatment regimen) involves selecting a dosage level or frequency of administration or route of administration of the compound or combinations of those parameters. In preferred embodiments, two or more compounds are to be administered, and the selecting involves selecting a method of administration for one, two, or more than two of the compounds, jointly, concurrently, or separately. As understood by those skilled in the art, such plurality of compounds may be used in combination therapy, and thus may be formulated in a single drug, or may be separate drugs administered concurrently, serially, or separately. Other embodiments are as indicated above for selection of second treatment methods, methods of identifying variances, and methods of treatment as described for aspects above.

In another aspect, the invention provides a method for selecting a patient for administration of a method of treatment for a disease or condition, or of selecting a patient for a method of administration of a treatment, by comparing the presence or absence of at least one variance in a gene as identified above in cells of a patient, with a list of variances in the gene, where the presence or absence of the at least one variance is indicative that the treatment or method of administration will be effective in the patient. If the at least one variance is present in the patient's cells, then the patient is selected for administration of the treatment.

In preferred embodiments, the disease or the method of treatment is as described in aspects above, specifically including, for example, those described for selecting a method of treatment.

In another aspect, the invention provides a method for identifying a subset of patients with enhanced or diminished response or tolerance to a treatment method or a method of administration of a treatment where the treatment is for a disease or condition in the patient. The method involves correlating one or more variances in one or more genes as identified in aspects above in a plurality of patients with response to a treatment or a method of administration of a treatment. The correlation may be performed by determining the one or more variances in the one or more genes in the plurality of patients and correlating the presence or absence of each of the variances (alone or in various combinations) with the patient's response to treatment. The variances may be previously known to exist or may also be determined in the present method or combinations of prior information and newly determined information may be used. The enhanced or diminished response should be statistically significant, preferably such that p=0.10 or less, more preferably 0.05 or less, and most preferably 0.02 or less. A positive correlation between the presence of one or more variances and an enhanced response to treatment is indicative that the treatment is particularly effective in the group of patients having those variances. A positive correlation of the presence of the one or more variances with a diminished response to the treatment is indicative that the treatment will be less effective in the group of patients having those variances. Such information is useful, for example, for selecting or de-selecting patients for a particular treatment or method of administration of a treatment, or for demonstrating that a group of patients exists for which the treatment or method of treatment would be particularly beneficial or contra-indicated. Such demonstration can be beneficial, for example, for obtaining government regulatory approval for a new drug or a new use of a drug.

In preferred embodiments, the variances are in at least one of the identified genes listed on Tables 1, 3, and 4, or are particular variances described herein. Also, preferred embodiments include drugs, treatments, variance identification or determination, determination of effectiveness, and/or diseases as described for aspects above or otherwise described herein.

In preferred embodiments, the correlation of patient responses to therapy according to patient genotype is carried out in a clinical trial, e.g., as described herein according to any of the variations described. Detailed description of methods for associating variances with clinical outcomes using clinical trials are provided below. Further, in preferred embodiments the correlation of pharmacological effect (positive or negative) to treatment response according to genotype or haplotype in such a clinical trial is part of a regulatory submission to a government agency leading to approval of the drug. Most preferably the compound or compounds would not be approvable in the absence of the genetic information allowing identification of an optimal responder population.

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating or excluding a treatment for a patient, eliminating or excluding a method or mode of administration of a treatment to a patient, or elimination of a patient for a treatment or method of treatment.

Also, in methods involving identification and/or comparison of variances present in a gene of a patient, the methods can involve such identification or comparison for a plurality of genes. Preferably, the genes are functionally related to the same disease or condition, or to the aspect of disease pathophysiology that is being subjected to pharmacological manipulation by the treatment (e.g., a drug), or to the activation or inactivation or elimination of the drug, and more preferably the genes are involved in the same biochemical process or pathway.

In another aspect, the invention provides a method for identifying the forms of a gene in an individual, where the gene is one specified as for aspects above, by determining the presence or absence of at least one variance in the gene. In preferred embodiments, the at least one variance includes at least one variance selected from the group of variances identified in variance tables herein. Preferably, the presence or absence of the at least one variance is indicative of the effectiveness of a therapeutic treatment in a patient suffering from a disease or condition and having cells containing the at least one variance.

The presence or absence of the variances can be determined in any of a variety of ways as recognized by those skilled in the art. For example, the nucleotide sequence of at least one nucleic acid sequence which includes at least one variance site (or a complementary sequence) can be determined, such as by chain termination methods, hybridization methods or by mass spectrometric methods. Likewise, in preferred embodiments, the determining involves contacting a nucleic acid sequence or a gene product of one of one of the genes with a probe which specifically identifies the presence or absence of a form of the gene. For example, a probe, e.g., a nucleic acid probe, can be used which specifically binds, e.g., hybridizes, to a nucleic acid sequence corresponding to a portion of the gene and which includes at least one variance site under selective binding conditions. As described for other aspects, determining the presence or absence of at least two variances and their relationship on the two gene copies present in a patient can constitute determining a haplotype or haplotypes. In this and other aspects involving mass spectrometry, the method can involve detection of the mass of a fragment or fragments and can further involve inferring the genotype (e.g., the specific variance at a site) from the masses determined.

Other preferred embodiments involve variances related to types of treatment, drug responses, diseases, nucleic acid sequences, and other items related to variances and variance determination as described for aspects above.

In yet another aspect, the invention provides a pharmaceutical composition which includes a compound which has a differential effect in patients having at least one copy, or alternatively, two copies of a form of a gene as identified for aspects above and a pharmaceutically acceptable carrier, excipient, or diluent. The composition is adapted to be preferentially effective to treat a patient with cells containing the one, two, or more copies of the form of the gene.

In preferred embodiments of aspects involving pharmaceutical compositions, active compounds, or drugs, the material is subject to a regulatory limitation or restriction on approved uses or indications, e.g., by the U.S. Food and Drug Administration (FDA), recommending use in or limiting approved use of the composition to patients having at least one copy of the particular form of the gene which contains at least one variance. Alternatively, the composition is subject to a regulatory limitation or restriction or recommendation on approved uses indicating that the composition is not approved for use or should not be used in patients having at least one copy of a form of the gene including at least one variance. Also in preferred embodiments, the composition is packaged, and the packaging includes a label or insert indicating or suggesting beneficial therapeutic approved use of the composition in patients having one or two copies of a form of the gene including at least one variance. Alternatively, the label or insert recommends or limits approved use of the composition to patients having zero or one or two copies of a form of the gene including at least one variance. The latter embodiment would be likely where the presence of the at least one variance in one or two copies in cells of a patient means that the composition would be ineffective or deleterious to the patient. Also in preferred embodiments, the composition is indicated for use in treatment of a disease or condition which is one of those identified for aspects above. Also in preferred embodiments, the at least one variance includes at least one variance from those identified herein.

The term “packaged” means that the drug, compound, or composition is prepared in a manner suitable for distribution or shipping with a box, vial, pouch, bubble pack, or other protective container, which may also be used in combination. The packaging may have printing on it and/or printed material may be included in the packaging.

In preferred embodiments, the drug is selected from the drug classes or specific exemplary drugs identified in an example, in a table herein, and is subject to a regulatory limitation or suggestion or warning as described above that limits or suggests limiting approved use to patients having specific variances or variant forms of a gene identified in Examples or in the gene list provided below in order to achieve maximal benefit and avoid toxicity or other deleterious effect.

A pharmaceutical composition can be adapted to be preferentially effective in a variety of ways. In some cases, an active compound is selected which was not previously known to be differentially active, or which was not previously recognized as a potential therapeutic compound. In some cases, the concentration of an active compound which has differential activity can be adjusted such that the composition is appropriate for administration to a patient with the specified variances. For example, the presence of a specified variance may allow or require the administration of a much larger dose, which would not be practical with a previously utilized composition. Conversely, a patient may require a much lower dose, such that administration of such a dose with a prior composition would be impractical or inaccurate. Thus, the composition may be prepared in a higher or lower unit dose form, or prepared in a higher or lower concentration of the active compound or compounds. In yet other cases, the composition can include additional compounds needed to enable administration of a particular active compound in a patient with the specified variances, which was not in previous compositions, e.g., because the majority of patients did not require or benefit from the added component, or would be adversely affected by the added component(s).

The term “differential” or “differentially” generally refers to a statistically significant different level in the specified property or effect. Preferably, the difference is also functionally significant. Thus, “differential binding or hybridization” is sufficient difference in binding or hybridization to allow discrimination using an appropriate detection technique. Likewise, “differential effect” or “differentially active” in connection with a therapeutic treatment or drug refers to a difference in the level of the effect or activity which is distinguishable using relevant parameters and techniques for measuring the effect or activity being considered. Preferably the difference in effect or activity is also sufficient to be clinically significant, such that a corresponding difference in the course of treatment or treatment outcome would be expected, at least on a statistical basis.

Also usefully provided in the present invention are probes which specifically recognize a nucleic acid sequence corresponding to a variance or variances in a gene as identified in aspects above or a product expressed from the gene, and are able to distinguish a variant form of the sequence or gene or gene product from one or more other variant forms of that sequence, gene, or gene product under selective conditions. Those skilled in the art recognize and understand the identification or determination of selective conditions for particular probes or types of probes. An exemplary type of probe is a nucleic acid hybridization probe, which will selectively bind under selective binding conditions to a nucleic acid sequence or a gene product corresponding to one of the genes identified for aspects above. Another type of probe is a peptide or protein, e.g., an antibody or antibody fragment which specifically or preferentially binds to a polypeptide expressed from a particular form of a gene as characterized by the presence or absence of at least one variance. Thus, in another aspect, the invention concerns such probes. In the context of this invention, a “probe” is a molecule, commonly a nucleic acid, though also potentially a protein, carbohydrate, polymer, or small molecule, that is capable of binding to one variance or variant form of the gene to a greater extent than to a form of the gene having a different base at one or more variance sites, such that the presence of the variance or variant form of the gene can be determined. Preferably the probe distinguishes at least one variance identified in Examples, tables or lists below or in Tables 1 or 3 of Stanton et al., U.S. application Ser. No. 09/300,747.

In preferred embodiments, the probe is a nucleic acid probe, 6, 7, 8, 9, 10, 11, 12, 13, 14, or preferably at least 17 nucleotides in length, more preferably at least 20 or 22 or 25, preferably 500 or fewer nucleotides in length, more preferably 200 or 100 or fewer, still more preferably 50 or fewer, and most preferably 30 or fewer. In preferred embodiments, the probe has a length in a range from any one of the above lengths to any other of the above lengths (including endpoints). The probe specifically hybridizes under selective hybridization conditions to a nucleic acid sequence corresponding to a portion of one of the genes identified in connection with above aspects. For certain types of probes, e.g., PNA probes, the probe is often shorter, e.g., at least 6, 7, 8, 10, or 12 nucleotides in length, with the length preferably also being no more than 50, 40, 30, 20, 17, or 15 nucleotides in length. The nucleic acid sequence includes at least one variance site. Also in preferred embodiments, the probe has a detectable label, preferably a fluorescent label. A variety of other detectable labels are known to those skilled in the art. Such a nucleic acid probe can also include one or more nucleic acid analogs.

In preferred embodiments, the probe is an antibody or antibody fragment which specifically binds to a gene product expressed from a form of one of the above genes, where the form of the gene has at least one specific variance with a particular base at the variance site, and preferably a plurality of such variances.

In connection with nucleic acid probe hybridization, the term “specifically hybridizes” indicates that the probe hybridizes to a sufficiently greater degree to the target sequence than to a sequence having a mismatched base at least one variance site to allow distinguishing such hybridization. The term “specifically hybridizes” thus means that the probe hybridizes to the target sequence, and not to non-target sequences, at a level which allows ready identification of probe/target sequence hybridization under selective hybridization conditions. Thus, “selective hybridization conditions” refer to conditions that allow such differential binding. Similarly, the terms “specifically binds” and “selective binding conditions” refer to such differential binding of any type of probe, e.g., antibody probes, and to the conditions which allow such differential binding. Typically hybridization reactions to determine the status of variant sites in patient samples are carried out with two different probes, one specific for each of the (usually two) possible variant nucleotides. The complementary information derived from the two separate hybridization reactions is useful in corroborating the results.

Likewise, the invention provides an isolated, purified or enriched nucleic acid sequence of 15 to 500 nucleotides in length, preferably 15 to 100 nucleotides in length, more preferably 15 to 50 nucleotides in length, and most preferably 15 to 30 nucleotides in length, which has a sequence which corresponds to a portion of one of the genes identified for aspects above. Preferably the lower limit for the preceding ranges is 17, 20, 22, or 25 nucleotides in length. In other embodiments, the nucleic acid sequence is 30 to 300 nucleotides in length, or 45 to 200 nucleotides in length, or 45 to 100 nucleotides in length. The nucleic acid sequence includes at least one variance site. Such sequences can, for example, be amplification products of a sequence which spans or includes a variance site in a gene identified herein. Likewise, such a sequence can be a primer, or amplification oligonucleotide that is able to bind to or extend through a variance site in such a gene. Yet another example is a nucleic acid hybridization probe comprised of such a sequence. In such probes, primers, and amplification products, the nucleotide sequence can contain a sequence or site corresponding to a variance site or sites, for example, a variance site identified herein. Preferably the presence or absence of a particular variant form in the heterozygous or homozygous state is indicative of the effectiveness of a method of treatment in a patient.

Likewise, the invention provides a set of primers or amplification oligonucleotides (e.g., 2, 3, 4, 6, 8, 10 or even more) adapted for binding to or extending through at least one gene identified herein. In preferred embodiments the set includes primers or amplification oligonucleotides adapted to bind or to extend through a plurality of sequence variances in a gene(s) identified herein. The plurality of variances preferably provides a haplotype. Those skilled in the art are familiar with the use of amplification oligonucleotides (e.g., PCR primers) and the appropriate location, testing and use of such oligonucleotides. In certain embodiments, the oligonucleotides are designed and selected to provide variance-specific amplification.

In reference to nucleic acid sequences which “correspond” to a gene, the term “correspond” refers to a nucleotide sequence relationship, such that the nucleotide sequence has a nucleotide sequence which is the same as the reference gene or an indicated portion thereof, or has a nucleotide sequence which is exactly complementary in normal Watson-Crick base pairing, or is an RNA equivalent of such a sequence, e.g., an mRNA, or is a cDNA derived from an mRNA of the gene.

In another aspect, the invention provides a method for determining a genotype of an individual in relation to one or more variances in one or more of the genes identified in above aspects by using mass spectrometric determination of a nucleic acid sequence which is a portion of a gene identified for other aspects of this invention or a complementary sequence. Such mass spectrometric methods are known to those skilled in the art. In preferred embodiments, the method involves determining the presence or absence of a variance in a gene; determining the nucleotide sequence of the nucleic acid sequence; the nucleotide sequence is 100 nucleotides or less in length, preferably 50 or less, more preferably 30 or less, and still more preferably 20 nucleotides or less. In general, such a nucleotide sequence includes at least one variance site, preferably a variance site which is informative with respect to the expected response of a patient to a treatment as described for above aspects.

As indicated above, many therapeutic compounds or combinations of compounds or pharmaceutical compositions show variable efficacy and/or safety in various patients in whom the compound or compounds is administered. Thus, it is beneficial to identify variances in relevant genes, e.g., genes related to the action or toxicity of the compound or compounds. Thus, in a further aspect, the invention provides a method for determining whether a compound has a differential effect due to the presence or absence of at least one variance in a gene or a variant form of a gene, where the gene is a gene identified for aspects above.

The method involves identifying a first patient or set of patients suffering from a disease or condition whose response to a treatment differs from the response (to the same treatment) of a second patient or set of patients suffering from the same disease or condition, and then determining whether the occurrence or frequency of occurrence of at least one variance in at least one gene differs between the first patient or set of patients and the second patient or set of patients. A correlation between the presence or absence of the variance or variances and the response of the patient or patients to the treatment indicates that the variance provides information about variable patient response. In general, the method will involve identifying at least one variance in at least one gene. An alternative approach is to identify a first patient or set of patients suffering from a disease or condition and having a particular genotype, haplotype or combination of genotypes or haplotypes, and a second patient or set of patients suffering from the same disease or condition that have a genotype or haplotype or sets of genotypes or haplotypes that differ in a specific way from those of the first set of patients. Subsequently the extent and magnitude of clinical response can be compared between the first patient or set of patients and the second patient or set of patients. A correlation between the presence or absence of a variance or variances or haplotypes and the response of the patient or patients to the treatment indicates that the variance provides information about variable patient response and is useful for the present invention.

The method can utilize a variety of different informative comparisons to identify correlations. For example a plurality of pairwise comparisons of treatment response and the presence or absence of at least one variance can be performed for a plurality of patients. Likewise, the method can involve comparing the response of at least one patient homozygous for at least one variance with at least one patient homozygous for the alternative form of that variance or variances. The method can also involve comparing the response of at least one patient heterozygous for at least one variance with the response of at least one patient homozygous for the at least one variance. Preferably the heterozygous patient response is compared to both alternative homozygous forms, or the response of heterozygous patients is grouped with the response of one class of homozygous patients and said group is compared to the response of the alternative homozygous group.

Such methods can utilize either retrospective or prospective information concerning treatment response variability. Thus, in a preferred embodiment, it is previously known that patient response to the method of treatment is variable.

Also in preferred embodiments, the disease or condition is as for other aspects of this invention; for example, the treatment involves administration of a compound or pharmaceutical composition.

In preferred embodiments, the method involves a clinical trial, e.g., as described herein. Such a trial can be arranged, for example, in any of the ways described herein, e.g., in the Detailed Description.

The present invention also provides methods of treatment of a disease or condition, preferably a disease or condition related to a neurological or psychiatric disease or other neurological or psychiatric clinical symptomatology. Such methods combine identification of the presence or absence of particular variances, preferably in a gene or genes from Tables 1, 3, and 4, with the administration of a compound; identification of the presence of particular variances with selection of a method of treatment and administration of the treatment; and identification of the presence or absence of particular variances with elimination of a method of treatment based on the variance information indicating that the treatment is likely to be ineffective or contra-indicated, and thus selecting and administering an alternative treatment effective against the disease or condition. Thus, preferred embodiments of these methods incorporate preferred embodiments of such methods as described for such sub-aspects.

As used herein, a “gene” is a sequence of DNA present in a cell that directs the expression of a “biologically active” molecule or “gene product”, most commonly by transcription to produce RNA and translation to produce protein. The “gene product” is most commonly a RNA molecule or protein or a RNA or protein that is subsequently modified by reacting with, or combining with, other constituents of the cell. Such modifications may include, without limitation, modification of proteins to form glycoproteins, lipoproteins, and phosphoproteins, or other modifications known in the art. RNA may be modified without limitation by polyadenylation, splicing, capping or export from the nucleus or by covalent or noncovalent interactions with proteins. The term “gene product” refers to any product directly resulting from transcription of a gene. In particular this includes partial, precursor, and mature transcription products (i.e., pre-mRNA and mRNA), and translation products with or without further processing including, without limitation, lipidation, phosphorylation, glycosylation, or combinations of such processing.

The term “gene involved in the origin or pathogenesis of a disease or condition” refers to a gene that harbors mutations or polymorphisms that contribute to the cause of disease, or variances that affect the progression of the disease or expression of specific characteristics of the disease. The term also applies to genes involved in the synthesis, accumulation, or elimination of products that are involved in the origin or pathogenesis of a disease or condition including, without limitation, proteins, lipids, carbohydrates, hormones, or small molecules.

The term “gene involved in the action of a drug” refers to any gene whose gene product affects the efficacy or safety of the drug or affects the disease process being treated by the drug, and includes, without limitation, genes that encode gene products that are targets for drug action, gene products that are involved in the metabolism, activation or degradation of the drug, gene products that are involved in the bioavailability or elimination of the drug to the target, gene products that affect biological pathways that, in turn, affect the action of the drug such as the synthesis or degradation of competitive substrates or allosteric effectors or rate-limiting reaction, or, alternatively, gene products that affect the pathophysiology of the disease process via pathways related or unrelated to those altered by the presence of the drug compound. (Particular variances in the latter category of genes may be associated with patient groups in whom disease etiology is more or less susceptible to amelioration by the drug. For example, there are several pathophysiological mechanisms in hypertension, and depending on the dominant mechanism in a given patient, that patient may be more or less likely than the average hypertensive patient to respond to a drug that primarily targets one pathophysiological mechanism. The relative importance of different pathophysiological mechanisms in individual patients is likely to be affected by variances in genes associated with the disease pathophysiology.) The “action” of a drug refers to its effect on biological products within the body. The action of a drug also refers to its effects on the signs or symptoms of a disease or condition, or effects of the drug that are unrelated to the disease or condition leading to unanticipated effects on other processes. Such unanticipated processes often lead to adverse events or toxic effects. The terms “adverse event” or “toxic” event” are known in the art and include, without limitation, those listed in the FDA reference system for adverse events.

In accordance with the aspects above and the Detailed Description below, there is also described for this invention an approach for developing drugs that are explicitly indicated for, and/or for which approved use is restricted to individuals in the population with specific variances or combinations of variances, as determined by diagnostic tests for variances or variant forms of certain genes involved in the disease or condition or involved in the action or metabolism or transport of the drug. Such drugs may provide more effective treatment for a disease or condition in a population identified or characterized with the use of a diagnostic test for a specific variance or variant form of the gene if the gene is involved in the action of the drug or in determining a characteristic of the disease or condition. Such drugs may be developed using the diagnostic tests for specific variances or variant forms of a gene to determine the inclusion of patients in a clinical trial.

Thus, the invention also provides a method for producing a pharmaceutical composition by identifying a compound which has differential activity or effectiveness against a disease or condition in patients having at least one variance in a gene, preferably in a gene from Table 1, compounding the pharmaceutical composition by combining the compound with a pharmaceutically acceptable carrier, excipient, or diluent such that the composition is preferentially effective in patients who have at least one copy of the variance or variances. In some cases, the patient has two copies of the variance or variances. In preferred embodiments, the disease or condition, gene or genes, variances, methods of administration, or method of determining the presence or absence of variances is as described for other aspects of this invention. In preferred embodiments, the active component of the pharmaceutical composition is a compound listed in the compound tables below (tables 5-14 and 16 through 18), or a compound chemically related to one of the listed compounds.

Similarly, the invention provides a method for producing a pharmaceutical agent by identifying a compound which has differential activity against a disease or condition in patients having at least one copy of a form of a gene, preferably a gene from Table 1, having at least one variance and synthesizing the compound in an amount sufficient to provide a pharmaceutical effect in a patient suffering from the disease or condition. The compound can be identified by conventional screening methods and its activity confirmed. For example, compound libraries can be screened to identify compounds which differentially bind to products of variant forms of a particular gene product, or which differentially affect expression of variant forms of the particular gene, or which differentially affect the activity of a product expressed from such gene. Alternatively, the design of a compound can exploit knowledge of the variances provided herein to avoid significant allele specific effects, in order to reduce the likelihood of significant pharmacogenetic effects during the clinical development process. Preferred embodiments are as for the preceding aspect.

In another aspect, the invention provides a method of treating a disease or condition in a patient by selecting a patient whose cells have an allele of an identified gene, preferably a gene selected from the genes listed in Table 1. The allele contains at least one variance correlated with more effective response to a treatment of said disease or condition. The method also includes altering the level of activity in cells of the patient of a product of the allele, where the altering provides a therapeutic effect.

Preferably the allele contains a variance as shown in Tables 1, 3, and 4 or other variance table herein, or in Table 1 or 3 of Stanton et al., U.S. application Ser. No. 09/300,747. Also preferably, the altering involves administering to the patient a compound preferentially active on at least one but less than all alleles of the gene.

Preferred embodiments include those as described above for other aspects of treating a disease or condition.

As recognized by those skilled in the art, all the methods of treating described herein include administration of the treatment to a patient.

In a further aspect, the invention provides a method for determining a method of treatment effective to treat a disease or condition by altering the level of activity of a product of an allele of a gene selected from the genes listed in Table 1, and determining whether that alteration provides a differential effect to (with respect to reducing or alleviating a disease or condition, or with respect to variation in toxicity or tolerance to a treatment) in patients with at least one copy of at least one allele of the gene as compared to patients with at least one copy of one alternative allele. The presence of such a differential effect indicates that altering the level of activity of the gene provides at least part of an effective treatment for the disease or condition.

Preferably the method for determining a method of treatment is carried out in a clinical trial, e.g., as described above and/or in the Detailed Description below.

In still another aspect, the invention provides a method for performing a clinical trial or study, which includes selecting or stratifying subjects in the trial or study using a variance or variances or haplotypes from one or more genes specified in Tables 1, 3 or 4. Preferably the differential efficacy, tolerance, or safety of a treatment in a subset of patients who have a particular variance, variances, or haplotype in a gene or genes from Tables 1, 3 and 4 is determined by conducting a clinical trial and using a statistical test to assess whether a relationship exists between efficacy, tolerance, or safety and the presence or absence of any of the variances or haplotype in one or more of the genes. Results of the clinical trial or study are indicative of whether a higher or lower efficacy, tolerance, or safety of the treatment in a subset of patients is associated with any of the variance or variances or haplotype in one or more of the genes. In preferred embodiments, the clinical trial or study is a Phase I, II, III, or IV trial or study. Preferred embodiments include the stratifications and/or statistical analyses as described below in the Detailed Description.

In preferred embodiments, normal subjects or patients are prospectively stratified by genotype in different genotype-defined groups, including the use of genotype as a enrollment criterion, using a variance, variances or haplotypes from Tables 1, 3 or 4, and subsequently a biological or clinical response variable is compared between the different genotype-defined groups. In preferred embodiments, normal subjects or patients in a clinical trial or study are stratified by a biological or clinical response variable in different biologically or clinically-defined groups, and subsequently the frequency of a variance, variances or haplotypes from Tables 1, 3 or 4 is measured in the different biologically or clinically defined groups.

In preferred embodiments, e.g., of the above two analyses (and in other aspects of this invention involving patient or normal subject stratification), the normal subjects or patients in a clinical trial or study are stratified by at least one demographic characteristic selected from the groups consisting of sex, age, racial origin, ethnic origin, or geographic origin.

Generally the method will involve assigning patients or subjects to a group to receive the method of treatment or to a control group.

In yet another aspect, the invention provides experimental methods for finding additional variances in a gene provided in Tables 1, 3, and 4. A number of experimental methods can also beneficially be used to identify variances. Thus, the invention provides methods for producing cDNA (Example 7) and detecting additional variances in the genes provided in Tables 1, 3, and 4 using the single strand conformation polymorphism (SSCP) method (Example 8), the T4 Endonuclease VII method (Example 9) or DNA sequencing (Example 10) or other methods pointed out below. The application of these methods to the identified genes will provide identification of additional variances that can affect inter-individual variation in drug or other treatment response. One skilled in the art will recognize that many methods for experimental variance detection have been described (in addition to the exemplary methods of examples 8, 9, 10) and can be utilized. These additional methods include chemical cleavage of mismatches (see, e.g., Ellis T. P., et al., Chemical cleavage of mismatch: a new look at an established method. Human Mutation 11(5):345-53, 1998), denaturing gradient gel electrophoresis (see, e.g., Van Orsouw N. J., et al., Design and application of 2-D DGGE-based gene mutational scanning tests. Genet Anal. 14(5-6):205-13, 1999) and heteroduplex analysis (see, e.g., Ganguly A., et al., Conformation-sensitive gel electrophoresis for rapid detection of single-base differences in double-stranded PCR products and DNA fragments: evidence for solvent-induced bends in DNA heteroduplexes. Proc Natl Acad Sci USA. 90 (21):10325-9, 1993). Table 3 of Stanton et al., U.S. application Ser. No. 09/300,747, provides a description of the additional possible variances that could be detected by one skilled in the art by testing an identified gene in Tables 1, 3, and 4 using the variance detection methods described or other methods which are known or are developed.

The present invention provides a method for treating a patient at risk for a neurological or psychiatric disease or condition (for example to prevent or delay the onset of frank disease) or a patient already diagnosed with a neurological or psychiatric disease or a disease associated with neuropathology. The methods include identifying such a patient and determining the patient's genotype or haplotype for an identified gene or genes. The patient identification can, for example, be based on clinical evaluation using conventional clinical metrics and/or on evaluation of a genetic variance or variances in one or more genes, preferably a gene or genes from Table 1. The invention provides a method for using the patient's genotype status to determine a treatment protocol that includes a prediction of the efficacy and/or safety of a therapy.

In another related aspect, the invention provides a method for identifying a patient for participation in a clinical trial of a therapy for the treatment of a neurological or psychiatric disease or an associated neuropathological or psychiatric condition. The method involves determining the genotype or haplotype of a patient with (or at risk for) a neurological or psychiatric disease risk Preferably the genotype is for a variance in a gene from Table 1. Patients with eligible genotypes are then assigned to a treatment or placebo group, preferably by a blinded randomization procedure. In preferred embodiments, the selected patients have no copies, or at least one copy or two copies of a wild type allele of an identified gene or genes identified herein, e.g., in Table 1. Alternatively, patients selected for the clinical trial may have zero, one or two copies of an allele belonging to a set of alleles, where the set of alleles comprise a group of related alleles. One procedure for rigorously defining a set of alleles is by applying phylogenetic methods to the analysis of haplotypes. (See, for example: Templeton A. R., Crandall K. A. and C. F. Sing, A cladistic analysis of phenotypic associations with haplotypes inferred from restriction endonuclease mapping and DNA sequence data. III. Cladogram estimation. Genetics 1992 Oct. 132(2):619-33.) Regardless of the specific tools used to group alleles, the trial would then test the hypothesis that a statistically significant difference in response to a treatment can be demonstrated between two groups of patients each defined by the presence of zero, one or two alleles (or allele groups) at a gene or genes. Said response may be a desired or an undesired response. In a preferred embodiment, the treatment protocol involves a comparison of placebo vs. treatment response rates in two or more genotype-defined groups. For example, a group with no copies of an allele may be compared to a group with two copies, or a group with no copies may be compared to a group consisting of those with one or two copies. In this manner different genetic models (dominant, co-dominant, recessive) for the transmission of a treatment response trait can be tested. Alternatively, statistical methods that do not posit a specific genetic model, such as contingency tables, can be used to measure the effects of an allele on treatment response.

In another preferred embodiment, patients in a clinical trial can be grouped (at the end of the trial) according to treatment response, and statistical methods can be used to compare allele (or genotype or haplotype) frequencies in two groups. For example, responders can be compared to nonresponders, or patients suffering adverse events can be compared to those not experiencing such effects. Alternatively response data can be treated as a continuous variable and the ability of genotype to predict response can be measured. In a preferred embodiments patients who exhibit extreme phenotypes are compared with all other patients or with a group of patients who exhibit a divergent extreme phenotype. For example if there is a continuous or semi-continuous measure of treatment response (for example the Alzheimer's Disease Assessment Scale, the Mini-Mental State Examination or the Hamilton Depression Rating Scale) then the 10% of patients with the most favorable responses could be compared to the 10% with the least favorable, or the patients one standard deviation above the mean score could be compared to the remainder, or to those one standard deviation below the mean score. One useful way to select the threshold for defining a response is to examine the distribution of responses in a placebo group. If the upper end of the range of placebo responses is used as a lower threshold for an ‘outlier response’ then the outlier response group should be almost free of placebo responders. This is a useful threshold because the inclusion of placebo responders in a ‘true’ response group decreases the ability of statistical methods to detect a genetic difference between responders and nonresponders disease.

In a related aspect, the invention provides a method for developing a disease management protocol that entails diagnosing a patient with a disease or a disease susceptibility, determining the genotype of the patient at a gene or genes correlated with treatment response and then selecting an optimal treatment based on the disease and the genotype (or genotypes or haplotypes). The disease management protocol may be useful in an education program for physicians, other caregivers or pharmacists; may constitute part of a drug label; or may be useful in a marketing campaign.

By “disease management protocol” or “treatment protocol” is meant a means for devising a therapeutic plan for a patient using laboratory, clinical and genetic data, including the patient's diagnosis and genotype. The protocol clarifies therapeutic options and provides information about probable prognoses with different treatments. The treatment protocol may provide an estimate of the likelihood that a patient will respond positively or negatively to a therapeutic intervention. The treatment protocol may also provide guidance regarding optimal drug dose and administration, and likely timing of recovery or rehabilitation. A “disease management protocol” or “treatment protocol” may also be formulated for asymptomatic and healthy subjects in order to forecast future disease risks based on laboratory, clinical and genetic variables. In this setting the protocol specifies optimal preventive or prophylactic interventions, including use of compounds, changes in diet or behavior, or other measures. The treatment protocol may include the use of a computer program.

In another aspect, the invention provides a kit containing at least one probe or at least one primer (or other amplification oligonucleotide) or both (e.g., as described above) corresponding to a gene or genes listed in Tables 1, 2, and 4 or other gene related to a neurological disease or condition. The kit is preferably adapted and configured to be suitable for identification of the presence or absence of a particular variance or variances, which can include or consist of a nucleic acid sequence corresponding to a portion of a gene. A plurality of variances may comprise a haplotype of haplotypes. The kit may also contain a plurality of either or both of such probes and/or primers, e.g., 2, 3, 4, 5, 6, or more of such probes and/or primers. Preferably the plurality of probes and/or primers are adapted to provide detection of a plurality of different sequence variances in a gene or plurality of genes, e.g., in 2, 3, 4, 5, or more genes or to amplify and/or sequence a nucleic acid sequence including at least one variance site in a gene or genes. Preferably one or more of the variance or variances to be detected are correlated with variability in a treatment response or tolerance, and are preferably indicative of an effective response to a treatment. In preferred embodiments, the kit contains components (e.g., probes and/or primers) adapted or useful for detection of a plurality of variances (which may be in one or more genes) indicative of the effectiveness of at least one treatment, preferably of a plurality of different treatments for a particular disease or condition. It may also be desirable to provide a kit containing components adapted or useful to allow detection of a plurality of variances indicative of the effectiveness of a treatment or treatment against a plurality of diseases. The kit may also optionally contain other components, preferably other components adapted for identifying the presence of a particular variance or variances. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), and deoxy nucleotide triphosphates (dNTPs). Preferably a probe includes a detectable label, e.g., a fluorescent label, enzyme label, light scattering label, or other label. Preferably the kit includes a nucleic acid or polypeptide array on a solid phase substrate. The array may, for example, include a plurality of different antibodies, and/or a plurality of different nucleic acid sequences. Sites in the array can allow capture and/or detection of nucleic acid sequences or gene products corresponding to different variances in one or more different genes. Preferably the array is arranged to provide variance detection for a plurality of variances in one or more genes which correlate with the effectiveness of one or more treatments of one or more diseases, which is preferably a variance as described herein.

The kit may also optionally contain instructions for use, which can include a listing of the variances correlating with a particular treatment or treatments for a disease or diseases and/or a statement or listing of the diseases for which a particular variance or variances correlates with a treatment efficacy and/or safety.

Preferably the kit components are selected to allow detection of a variance described herein, and/or detection of a variance indicative of a treatment, e.g., administration of a drug, pointed out herein.

Additional configurations for kits of this invention will be apparent to those skilled in the art.

The invention also includes the use of such a kit to determine the genotype(s) of one or more individuals with respect to one or more variance sites in one or more genes identified herein. Such use can include providing a result or report indicating the presence and/or absence of one or more variant forms or a gene or genes which are indicative of the effectiveness of a treatment or treatments.

In another aspect, the invention provides a method for determining whether there is a genetic component to intersubject variation in a surrogate treatment response. The method involves administering the treatment to a group of related (preferably normal) subjects and a group of unrelated (preferably normal) subjects, measuring a surrogate pharmacodynamic or pharmacokinetic drug response variable in the subjects, performing a statistical test measuring the variation in response in the group of related subjects and, separately in the group of unrelated subjects, comparing the magnitude or pattern of variation in response or both between the groups to determine if the responses of the groups are different, using a predetermined statistical measure of difference. A difference in response between the groups is indicative that there is a genetic component to intersubject variation in the surrogate treatment response.

In preferred embodiments, the size of the related and unrelated groups is set in order to achieve a predetermined degree of statistical power.

In another aspect, the invention provides a method for evaluating the combined contribution of two or more variances to a surrogate drug response phenotype in subjects (preferably normal subjects) by a genotyping a set of unrelated subjects participating in a clinical trial or study, e.g., a Phase I trial, of a compound. The genotyping is for two or more variances (which can be a haplotype), thereby identifying subjects with specific genotypes, where the two or more specific genotypes define two or more genotype-defined groups. A drug is administered to subjects with two or more of said specific genotypes, and a surrogate pharmacodynamic or pharmacokinetic drug response variable is measured in the subjects. A statistical test or tests is performed to measure response in the groups separately, where the statistical tests provide a measurement of variation in response with each group. The magnitude or pattern of variation in response or both is compared between the groups to determine if the groups are different using a predetermined statistical measure of difference.

In preferred embodiments, the specific genotypes are homozygous genotypes for two variances. In preferred embodiments, the comparison is between groups of subjects differing in three or more variances, e.g., 3, 4, 5, 6, or even more variances.

In another aspect, the invention provides a method for providing contract research services to clients (preferably in the pharmaceutical and biotechnology industries), by enrolling subjects (e.g., normal and/or patient subjects) in a clinical drug trial or study unit (preferably a Phase I drug trial or study unit) for the purpose of genotyping the subjects in order to assess the contribution of genetic variation to variation in drug response, genotyping the subjects to determine the status of one or more variances in the subjects, administering a compound to the subjects and measuring a surrogate drug response variable, comparing responses between two or more genotype-defined groups of subjects to determine whether there is a genetic component to the interperson variability in response to said compound; and reporting the results of the Phase I drug trial to a contracting entity. Clearly, intermediate results, e.g., response data and/or statistical analysis of response or variation in response can also be reported.

In preferred embodiments, at least some of the subjects have disclosed that they are related to each other and the genetic analysis includes comparison of groups of related individuals. To encourage participation of sufficient numbers of related individuals, it can be advantageous to offer or provide compensation to one or more of the related individuals based on the number of subjects related to them who participate in the clinical trial, or on whether at least a minimum number of related subjects participate, e.g., at least 3, 5, 10, 20, or more.

In a related aspect, the invention provides a method for recruiting a clinical trial population for studies of the influence of genetic variation on drug response, by soliciting subjects to participate in the clinical trial, obtaining consent of each of a set of subjects for participation in the clinical trial, obtaining additional related subjects for participation in the clinical trial by compensating one or more of the related subjects for participation of their related subjects at a level based on the number of related subjects participating or based on participation of at least a minimum specified number of related subjects, e.g., at minimum levels as specified in the preceding aspect.

In the various aspects of this invention, a gene is a gene as identified in Tables 1, 3, or 4, or is a gene in a pathway as identified in Table 1, preferably a gene having a function as identified in Table 1. Some such additional genes, and exemplary variances are provided in a parent application.

In the various aspects of this invention, the drug is preferably a drug as identified in a drug table herein, or is a drug in the same chemical class.

By “pathway” or “gene pathway” is meant the group of biologically relevant genes involved in a pharmacodynamic or pharmacokinetic mechanism of drug, agent, or candidate therapeutic intervention. These mechanisms may further include any physiologic effect the drug or candidate therapeutic intervention renders. Included in this are “biochemical pathways” which is used in its usual sense to refer to a series of related biochemical processes (and the corresponding genes and gene products) involved in carrying out a reaction or series of reactions. Generally in a cell, a pathway performs a significant process in the cell.

By “pharmacological activity” used herein is meant a biochemical or physiological effect of drugs, compounds, agents, or candidate therapeutic interventions upon administration and the mechanism of action of that effect.

The pharmacological activity is then determined by interactions of drugs, compounds, agents, or candidate therapeutic interventions, or their mechanism of action, on their target proteins or macromolecular components. By “agonist” or “mimetic” or “activators” is meant a drug, agent, or compound that activate physiologic components and mimic the effects of endogenous regulatory compounds. By “antagonists”, “blockers” or “inhibitors” is meant drugs, agents, or compounds that bind to physiologic components and do not mimic endogenous regulatory compounds, or interfere with the action of endogenous regulatory compounds at physiologic components. These inhibitory compounds do not have intrinsic regulatory activity, but prevent the action of agonists. By “partial agonist” or “partial antagonist” is meant an agonist or antagonist, respectively, with limited or partial activity. By “negative agonist” or “inverse antagonists” is meant that a drug, compound, or agent that can interact with a physiologic target protein or macromolecular component and stabilizes the protein or component such that agonist-dependent conformational changes of the component do not occur and agonist mediated mechanism of physiological action is prevented. By “modulators” or “factors” is meant a drug, agent, or compound that interacts with a target protein or macromolecular component and modifies the physiological effect of an agonist.

As used herein the term “chemical class” refers to a group of compounds that share a common chemical scaffold but which differ in respect to the substituent groups linked to the scaffold. Examples of chemical classes of drugs include, for example, phenothiazines, piperidines, benzodiazepines and aminoglycosides. Members of the phenothiazine class include, for example, compounds such as chlorpromazine hydrochloride, mesoridazine besylate, thioridazine hydrochloride, acetophenazine maleate trifluoperazine hydrochloride and others, all of which share a phenothiazine backbone. Members of the piperidine class include, for example, compounds such as meperidine, diphenoxylate and loperamide, as well as phenylpiperidines such as fentanyl, sufentanil and alfentanil, all of which share the piperidine backbone. Chemical classes and their members are recognized by those skilled in the art of medicinal chemistry.

As used herein the term “surrogate marker” refers to a biological or clinical parameter that is measured in place of the biologically definitive or clinically most meaningful parameter. In comparison to definitive markers, surrogate markers are generally either more convenient, less expensive, provide earlier information or provide pharmacological or physiological information not directly obtainable with definitive markers. Examples of surrogate biological parameters: (i) testing erythrocye membrane acetylcholinesterase levels in subjects treated with an acetylcholinesterase inhibitor intended for use in Alzheimer's disease patients (where inhibition of brain acetylcholinesterase would be the definitive biological parameter); (ii) measuring levels of CD4 positive lymphocytes as a surrogate marker for response to a treatment for acquired immune deficiency syndrome (AIDS). Examples of surrogate clinical parameters: (i) performing a psychometric test on normal subjects treated for a short period of time with a candidate Alzheimer's compound in order to determine if there is a measurable effect on cognitive function. The definitive clinical test would entail measuring cognitive function in a clinical trial in Alzheimer's disease patients. (ii) Measuring blood pressure as a surrogate marker for myocardial infarction. The measurement of a surrogate marker or parameter may be an endpoint in a clinical study or clinical trial, hence “surrogate endpoint”.

As used herein the term “related” when used with respect to human subjects indicates that the subjects are known to share a common line of descent; that is, the subjects have a known ancestor in common. Examples of preferred related subjects include sibs (brothers and sisters), parents, grandparents, children, grandchildren, aunts, uncles, cousins, second cousins and third cousins. Subjects less closely related than third cousins are not sufficiently related to be useful as “related” subjects for the methods of this invention, even if they share a known ancestor, unless some related individuals that lie between the distantly related subjects are also included. Thus, for a group of related individuals, each subject shares a known ancestor within three generations or less with at least one other subject in the group, and preferably with all other subjects in the group or has at least that degree of consanguinity due to multiple known common ancestors. More preferably, subjects share a common ancestor within two generations or less, or otherwise have equivalent level of consanguinity. Conversely, as used herein the term “unrelated”, when used in respect to human subjects, refers to subjects who do not share a known ancestor within 3 generations or less, or otherwise have known relatedness at that degree.

As used herein the term “pedigree” refers to a group of related individuals, usually comprising at least two generations, such as parents and their children, but often comprising three generations (that is, including grandparents or grandchildren as well). The relation between all the subjects in the pedigree is known and can be represented in a genealogical chart.

As used herein the term “hybridization”, when used with respect to DNA fragments or polynucleotides encompasses methods including both natural polynucleotides, non-natural polynucleotides or a combination of both. Natural polynucleotides are those that are polymers of the four natural deoxynucleotides (deoxyadenosine triphosphate [dA], deoxycytosine triphosphate [dC], deoxyguanine triphosphate [dG] or deoxythymidine triphosphate [dT], usually designated simply thymidine triphosphate [T]) or polymers of the four natural ribonucleotides (adenosine triphosphate [A], cytosine triphosphate [C], guanine triphosphate [G] or uridine triphosphate [U]). Non-natural polynucleotides are made up in part or entirely of nucleotides that are not natural nucleotides; that is, they have one or more modifications. Also included among non-natural polynucleotides are molecules related to nucleic acids, such as peptide nucleic acid [PNA]). Non-natural polynucleotides may be polymers of non-natural nucleotides, polymers of natural and non-natural nucleotides (in which there is at least one non-natural nucleotide), or otherwise modified polynucleotides. Non-natural polynucleotides may be useful because their hybridization properties differ from those of natural polynucleotides. As used herein the term “complementary”, when used in respect to DNA fragments, refers to the base pairing rules established by Watson and Crick: A pairs with T or U; G pairs with C. Complementary DNA fragments have sequences that, when aligned in antiparallel orientation, conform to the Watson-Crick base pairing rules at all positions or at all positions except one. As used herein, complementary DNA fragments may be natural polynucleotides, non-natural polynucleotides, or a mixture of natural and non-natural polynucleotides.

As used herein “amplify” when used with respect to DNA refers to a family of methods for increasing the number of copies of a starting DNA fragment. Amplification of DNA is often performed to simplify subsequent determination of DNA sequence, including genotyping or haplotyping. Amplification methods include the polymerase chain reaction (PCR), the ligase chain reaction (LCR) and methods using Q beta replicase, as well as transcription-based amplification systems such as the isothermal amplification procedure known as self-sustained sequence replication (3SR, developed by T. R. Gingeras and colleagues), strand displacement amplification (SDA, developed by G. T. Walker and colleagues) and the rolling circle amplification method (developed by P. Lizardi and D. Ward).

As used herein “contract research services for a client” refers to a business arrangement wherein a client entity pays for services consisting in part or in whole of work performed using the methods described herein. The client entity may include a commercial or non-profit organization whose primary business is in the pharmaceutical, biotechnology, diagnostics, medical device or contract research organization (CRO) sector, or any combination of those sectors. Services provided to such a client may include any of the methods described herein, particularly including clinical trial services, and especially the services described in the Detailed Description relating to a Pharmacogenetic Phase I Unit. Such services are intended to allow the earliest possible assessment of the contribution of a variance or variances or haplotypes, from one or more genes, to variation in a surrogate marker in humans. The surrogate marker is generally selected to provide information on a biological or clinical response, as defined above.

As used herein, “comparing the magnitude or pattern of variation in response” between two or more groups refers to the use of a statistical procedure or procedures to measure the difference between two different distributions. For example, consider two genotype-defined groups, AA and aa, each homozygous for a different variance or haplotype in a gene believed likely to affect response to a drug. The subjects in each group are subjected to treatment with the drug and a treatment response is measured in each subject (for example a surrogate treatment response). One can then construct two distributions: the distribution of responses in the AA group and the distribution of responses in the aa group. These distributions may be compared in many ways, and the significance of any difference qualified as to its significance (often expressed as a p value), using methods known to those skilled in the art. For example, one can compare the means, medians or modes of the two distributions, or one can compare the variance or standard deviations of the two distributions. Or, if the form of the distributions is not known, one can use nonparametric statistical tests to test whether the distributions are different, and whether the difference is significant at a specified level (for example, the p<0.05 level, meaning that, by chance, the distributions would differ to the degree measured less than one in 20 similar experiments). The types of comparisons described are similar to the analysis of heritability in quantitative genetics, and would draw on standard methods from quantitative genetics to measure heritability by comparing data from related subjects.

Another type of comparison that can be usefully made is between related and unrelated groups of subjects. That is, the comparison of two or more distributions is of particular interest when one distribution is drawn from a population of related subjects and the other distribution is drawn from a group of unrelated subjects, both subjected to the same treatment. (The related subjects may consist of small groups of related subjects, each compared only to their relatives.) A comparison of the distribution of a drug response variable (e.g. a surrogate marker) between two such groups may provide information on whether the drug response variable is under genetic control. For example, a narrow distribution in the group(s) of related subjects (compared to the unrelated subjects) would tend to indicate that the measured variable is under genetic control (i.e. the related subjects, on account of their genetic homogeneity, are more similar than the unrelated individuals). The degree to which the distribution was narrower in the related individuals (compared to the unrelated individuals) would be proportionate to the degree of genetic control. The narrowness of the distribution could be quantified by, for example, computing variance or standard deviation. In other cases the shape of the distribution may not be known and nonparametric tests may be preferable. Nonparametric tests include methods for comparing medians such as the sign test, the slippage test, or the rank correlation coefficient (the nonparametric equivalent of the ordinary correlation coefficient). Pearson's Chi square test for comparing an observed set of frequencies with an expected set of frequencies can also be useful.

The present invention provides a number of advantages. For example, the methods described herein allow for use of a determination of a patient's genotype for the timely administration of the most suitable therapy for that particular patient. The methods of this invention provide a basis for successfully developing and obtaining regulatory approval for a compound even though efficacy or safety of the compound in an unstratified population is not adequate to justify approval. From the point of view of a pharmaceutical or biotechnology company, the information obtained in pharmacogenetic studies of the type described herein could be the basis of a marketing campaign for a drug. For example, a marketing campaign that emphasized the superior efficacy or safety of a compound in a genotype or haplotype restricted patient population, compared to a similar or competing compound used in an undifferentiated population of all patients with the disease. In this respect a marketing campaign could promote the use of a compound in a genetically defined subpopulation, even though the compound was not intrinsically superior to competing compounds when used in the undifferentiated population with the target disease. In fact even a compound with an inferior profile of action in the undifferentiated disease population could become superior when coupled with the appropriate pharmacogenetic test.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DETAILED DESCRIPTION OF THE PREFERRED EMBODIMENTS

First a brief description of the Tables is provided.

Table 1, the CNS Gene Table, lists genes that may be involved in pharmacological response to neurological and psychiatric therapeutics, or that may define disease subsets with different prognosis and consequent implications for treatment. The table has seven columns. Column 1, headed “Class” provides broad groupings of genes relevant to the pharmacology of CNS drugs. The categories are: small molecule neurotransmitters, peptide hormones, steroid hormones, inflammation, cardiovascular (principally vascular and circulating factors), cell growth and differentiation and cellular maintenance. Column 2, headed “Pathway”, provides a more detailed categorization of the different classes of genes by indicating the overall purpose of large groups of genes. For example, within the small molecule neurotransmitter group are the genes responsible for packaging neurotransmitters into vesicles and controlling their subsequent release from cells, as well as the genes involved in the production and control of each of the small molecule neurotransmitters (glutamate, serotonin, dopamine, etc.). These pathways contain genes implicated in the etiology or treatment response of the various diseases detailed in Table 2. Column 3, headed “Function”, further categorizes the pathways listed in column 2. To, for example, the “Pathway” category called “Packaging of Neurotransmitter into Vesicles and Release” (column 2) is divided into three categories in column 3: (i) concentration of transmitter in vesicles, (ii) neurotransmitter release and (iii) general signal transduction. These functions apply to all of the specific neurotransmitters. For each neurotransmitter listed in column 2 there is a set of four categories in column 4: (i) biosynthesis, (ii) receptors, (iii) reuptake and (iv) catabolism. Some categories in column 2 (e.g. “Clotting”) are not further categorized in column 3.

In Column 34, a commonheaded “Name”, lists the genes belonging to the class, pathway and function shown to the left (in columns 1-3). The gene names given are generally those used in the OMIM database or in GenBank, however one skilled in the art will recognize that many genes have more than one name, and that it is a straightforward task to identify synonymous names. For example, many alternate gene names are provided in the OMIM record for a gene.

In column 5, headed “OMIM”, the Online Mendelian Inheritance in Man (OMIM) record number is listed for each gene in column 4. This record number can be entered next to the words: “Enter one or more search keywords:” at the OMIM world wide web site. An OMIM record exists for most characterized human genes. The record often has useful information on the chromosome location, function, alleles, and human diseases or disorders associated with each gene.

Column 56, headed “GID”, provides the GenBank identification number (hence GID) of a genomic, cDNA, or partial sequence of the gene named in column 4. Usually the GID provides the record of a cDNA sequence. Many genes have multiple Genbank accession numbers, representing different versions of a sequence obtained by different research groups, or corrected or updated versions of a sequence. As with the gene name, one skilled in the art will recognize that alternative GenBank records related to the named record can be obtained easily. All other GenBank records listing sequences that are alternate versions of the sequences named in the table are equally suitable for the inventions described in this application. (One straightforward way to obtain additional GenBank records for a gene is on the internet. General instructions can be found at the NCBI web site. Once the GenBank record has been retrieved one can click on the “nucleotide neighbors” link and additional GenBank records from the same gene will be listed action.

Column 7, headed “locus”, provides the chromosome location of the gene listed on the same row. The chromosome location helps confirm the identity of the named gene if there is any ambiguity.

Table 2 is a matrix showing the intersection of genes and therapeutic indications—that is, which categories of genes are most likely to account for interpatient variation in response to treatments for which diseases. The Table is displayed on two pages. On each page the first two columns provide a framework for organizing the genes listed in Table 1. Column 1 is similar to the ‘Class’ column in Table 1, while column 2 is a combination of the ‘Pathway’ and ‘Function’ columns in Table 1. It is intended that the summary terms listed in columns 1 and 2 be read as referring to all the genes in the corresponding sections of Table 1. The remaining columns in Table 2 list thirteen neurological and psychiatric diseases. The information in the Table lies in the shaded boxes at the intersection of various “Pathways” (the rows) and treatment indications (the 13 columns) An intersection box is shaded when a row corresponding to a particular pathway (and by extension all the genes listed in that pathway in Table 1) intersects a column for a specific neurological or psychiatric disease such that the pathway and genes are of possible use in explaining interpatient differences in response to treatments for the neurological or psychiatric indication. Thus the Table enables one skilled in the art to identify therapeutically relevant genes in patients with one of the 13 indications for the purposes of stratification of these patients based upon genotype and subsequent correlation of genotype with drug response. The shaded intersections indicate preferred sets of genes for understanding the basis of interpatient variation in response to therapy of the indicated disease indication, and in that respect are exemplary. Any of the genes in the table may account for interpatient variation in response to treatments for any of the diseases listed. Thus, the shaded boxes indicate the gene pathways that one skilled in the art would first investigate in trying to understand interpatient variation in response to therapy for the listed neurological indications.

Table 3 lists DNA sequence variances in genes relevant to the methods described in the present invention. These variances were identified by the inventors in studies of selected genes listed in Table 1, and are provided here as useful for the methods of the present invention. The variances in Table 3 were discovered by one or more of the methods described below in the Detailed Description or Examples. Table 3 has ten columns. The column headings are spread over two rows, with five headings in the first row and three in the second row. The gene sequence variance listings in the tables have a similar organization to the column headings, with a set of nomenclature data in the first row for each gene entry, and variance data in the second and additional following rows for however many sequence variances are available for a specific gene. Column 1, the “Name” column, contains the Human Genome Organization (HUGO) identifier for the gene, or if such identifier is not provided the GenBank accession number. Column 2, the “GID” column provides the GenBank accession number of a genomic, cDNA, or partial sequence of a particular gene. Column 3, the “OMIM_ID” column contains the record number corresponding to the Online Mendelian Inheritance in Man database for the gene provided in columns 1 and 2. This record number can be entered at the OMIM world wide web site to search the OMIM record on the gene. Column 4, the VGX_Symbol column, provides an internal identifier for the gene. Column 5, the “Description” column provides a descriptive name for the gene, when available. Columns 6, 7 and 8 are on the second row of columns. Column 6, the “Variance_Start” column provides the nucleotide location of a variance with respect to the first listed nucleotide in the GenBank accession number provided in column 2. That is, the first nucleotide of the GenBank accession is counted as nucleotide 1 and the variant nucleotide is numbered accordingly. Column 7, the “variance” column provides the nucleotide location of a variance with respect to an ATG codon believed to be the authentic ATG start codon of the gene, where the A of ATG is numbered as one (1) and the immediately preceding nucleotide is numbered as minus one (−1). This reading frame is important because it allows the potential consequence of the variant nucleotide to be interpreted in the context of the gene anatomy (5′ untranslated region, protein coding sequence, 3′ untranslated region). Column 7 also provides the identity of the two variant nucleotides at the indicated position. For example, in the first entry in Table 2, AF004884, the variance is 4196T>A, indicating the presence of a T or a A at nucleotide 4196 of GenBank sequence AF004884. Column 8, the “CDS_Context” column indicates whether the variance is in a coding region but silent (S); in a coding region and results in an amino acid change (e.g., R347C, where the letters are one letter amino acid abbreviations and the number is the amino acid residue in the encoded amino acid sequence which is changed); in a sequence 5′ to the coding region (5); or in a sequence 3′ to the coding region (3). As indicated above, interpreting the location of the variance in the gene is contingent on the correct assignment of the initial ATG of the encoded protein (the translation start site). It should be recognized that assignment of the correct ATG may occasionally be incorrect in GenBank, but that one skilled in the art will know how to carry out experiments to definitively identify the correct translation initiation codon (which is not always an ATG). In the event of any potential question concerning the proper identification of a gene or part of a gene, due for example, to an error in recording an identifier or the absence of one or more of the identifiers, the priority for use to resolve the ambiguity is GenBank accession number, OMIM identification number, HUGO identifier, common name identifier. Column 9 provides the allele frequency for the variance in a given row. In this column, the allele frequency is expressed as the value times 100 and the frequency is representative of genotyping at least 10 individuals, or 20 alleles, at the site of variation. Therefore, for example, if each individual in a population was a heterozygote, the allele frequency would be 0.5 or 50 (50%). The allele frequency is listed for the second nucleotide listed in the table, for example, if the variance is listed as “120T>C 95.31” the allele frequency of a C at position 120 is 95.31 and the allele frequency of a T at position 120 is 4.69. Column 10 (values shown in parenthesis) are the predictive values for assessing whether the amino acid substitution for the listed protein is likely or not likely to have an effect on the biological activity of the protein. The predictive values were obtained using a computer-assisted method as described in U.S. patent application Ser. No. 09/614,735, attorney docket number 11926-003001, filed Jul. 12, 2000, entitled “Methods for structure-based assessment of amino acid polymorphisms”. The values for the prediction of the effect of an amino acid change on the biological function of the listed protein varies from 0 to 1, where predictive values closer to one indicates the amino acid change is likely to influence function of the protein and values closer to zero indicates the amino acid change is less likely to influence function of the protein. For some of the variances listed in the table, there is more than one predictive value attained from this computational method. In these cases, more than one modeled protein sequence was used to attain a predictive value and because the values may be representative of a specific modeled sequence, the values are listed separately. These predictive value(s) can be used for the identification and analysis of amino acid polymorphisms that affect the structure or function of proteins involved directly or indirectly in the action of pharmaceutical compounds or diagnostic agents.

If a haplotype for any of the genes listed in this table has been identified, a series of nucleotides (A, C, G, T) are listed separated by commas and to the left of each listing is the associated nucleotide location also separated by commas in brackets. For example, if the haplotype listing is T,G,C,A [12, 245, 385, 612] there is a T at position 12, a G at position 246, a C at position 385, and an A at position 612. Below this list will occur the identified variance start, variance, and CDS context for the identified single nucleotide polymorphisms as described above.

In Tables 3 and 4 at least one set of two values is associated with each polymorphism that is predicted to result in an amino acid change. Both values in each set of two values relate to the predicted impact of the amino acid change on the protein encoded by a gene having the polymorphism. The two values are shown in parenthesis next to the polymorphism with which they are associated, separated by a colon. Some polymorphisms may have more than one set of two values, each set representing a separate prediction from a distinct model as described in Chasman and Adams (Chasman and Adams, J. Mol. Biol. 2001, 307:683-706).

The first value is a decimal number between 0 (highly unlikely) and 1 (highly likely) that is an estimate of the probability that the amino acid change will alter the function of the encoded protein. This value was arrived at using the predictive methods described in Chasman and Adams. The predictive methods described in Chasman and Adams (supra) entail the use of a model amino acid in a model protein whose three-dimensional structure is known to assess the likely impact of the predicted amino acid change on a protein of interest. Thus, simplifying somewhat, if a change in the model amino acid is one which alters function of the model protein, the predicted amino acid in the protein of interest would be considered likely to alter the activity of the protein of interest. As Chasman and Adams (supra) explains, some special features of model amino acids are particularly good predictors of the effect of a change in the amino acid on the function of the protein. These special features are not incorporated into the prediction that is represented by the first value, but they are represented by the second value. This second value is either “1” or “0” and indicates whether the model amino acid in the model protein used to determine the probability that the predicted amino acid change will alter protein function has at least one of the special features that are associated with more highly reliable predictions. Thus, if the model amino acid meets one of the criteria for being associated with a special feature (buried_charge, conserved_position, interface, near_het_atom, and near_seq_prosite, all described in Chasman and Adams), the second value is “1” to indicate that the amino acid change is very likely to affect protein function. Otherwise, the second value is 0, and the probability of an effect of the amino acid change on function is simply taken as the first value. Thus, the probability that the amino acid change will alter the function of the protein encoded by a gene having the polymorphism is the larger of the first value and the second values in the set of values.

Table 3 of the present application is being submitted on CD-ROM concurrently with the present application as permitted under 37 C.F.R. §1.52(e).

Table 4 lists additional DNA sequence variances (in addition to those in Table 3) in genes relevant to the methods of the present invention (i.e. selected genes from Table 1). These variances were identified by various research groups and published in the scientific literature over the past 20 years. The inventors realized that these variances can be useful for understanding interpatient variation in response to treatment of the diseases listed in Table 2, and more generally useful for the methods of the present invention. The headings of Table 4 are as in Table 3, and therefore the descriptions of the columns in Table 3 (above) pertain to Table 4, as do the other remarks.

Table 4 of the present application is being submitted on CD-ROM concurrently with the present application as permitted under 37 C.F.R. §1.52(e).

Tables 5-14 and 16-18 provide lists of exemplary compounds in clinical development for various CNS diseases. The compounds listed in the tables are exemplary; that is, the methods of the invention will apply to other compounds as well. Each table has four columns. The first column is titled “Product Name”, the second column is titled “Chemical Name”, the third “Action” and the fourth “Indication”. Under these headings are listed rows of compounds. For each compound there is a brief summary of information about the product name, its pharmacological action and potential clinical uses. The first column, “Product Name”, provides the generic name and/or alphanumeric designation of the compound, as well as its trade name in some cases (in capital letters). The second column, “Chemical Name” provides the full chemical name of the compound. The listed compounds, or compounds chemically related to those listed (e.g. by modification of one or more chemical moieties of the listed compounds), are suitable for the methods of this invention. The third column, “Action”, summarizes in a word or phrase an important pharmacological action of the compound, or what is currently believed to be an important pharmacological action—in most cases additional pharmacological actions are known but not listed to conserve space; alternatively, subsequent studies may reveal additional or alternative pharmacological actions. (Sources listed in the detailed description will help clarify whether additional pharmacological actions have been discovered.) The fourth column, “Indication”, provides an exemplary disease or condition for which the compound is currently being, or has already been, developed. In many cases the compound is being, has already been, or will likely be developed for other indications. Again, one skilled in the art will know how to identify additional drug development programs for these compounds. For example, a compound in development for one neurodegenerative disease is likely to be evaluated in the treatment of other neurodegenerative diseases.

Table 19 lists the gene (or coding) sequence of the genes disclosed in the gene table (Table 1). Each sequence is preceeded by a line of text including (1) an internal reference number, (2) a GID (Genbank identifier) reference number, (3) the OMIM number (see above), (4) an internal unique identifier reference number, and (4) the name of the gene or coding sequence.

Table 19 of the present application is being submitted on CD-ROM concurrently with the present application as permitted under 37 C.F.R. § 1.52(e).

I. Neurological and Psychiatric Diseases

The treatment of neurological and psychiatric diseases presents a challenge to physicians and other medical practitioners because the available therapeutics are only partially effective in only a fraction of patients. Further, many currently used medicines produce serious adverse effects. Therapeutic benefits and toxic side effects have to be balanced in each patient. This requires much attention to drug selection, dosage adjustment and monitoring for potential adverse events on the part of care givers—effectively a new pharmacokinetic and pharmacodynamic study must be performed for each patient. These limitations of therapy are especially true of the most debilitating neurological and psychiatric diseases such as psychosis, depression, epilepticepilepsy, the neurodegenerative diseases including Alzheimer's disease and Parkinson's disease, migraine and cerebrovascular disease. Although these diseases have distinct clinical presentations, there is extensive overlap in pathogenetic mechanisms and symptoms.

Difficulties in treating neurological and psychiatric diseases are attributable to factors such as limited understanding of disease condition pathophysiology, lack of specificity of pathophysiologic changes (i.e. variation in pathophysiologic mechanisms in patients with similar clinical presentation) and lack of specificity of therapeutic compounds. Further, most medical therapy is directed to the amelioration of symptoms, not the arrest or reversal of underlying pathophysiologic processes. One good example of the difficulty of developing and marketing effective treatments is the history of therapeutic candidates for Alzheimer's disease. Out of dozens of candidate treatments tested in clinical trials only two products have been approved for use in the United States, and one of them (tacrine; Cognex) has been withdrawn from marketing due to safety problems. Further, the one marketed product (donezepil; Aricept) is only used by a small fraction of eligible patients because it has a reputation among caregivers and Alzheimer's disease advocacy groups as being ineffective in most patients. Thus a drug that enjoys a monopoly position in a major disease is not a great commercial success because its shortcomings are widely realized.

In summary, medical management of neurological and psychiatric diseases is empirical in nature, is only partially effective, and is associated with multiple undesirable side effects. In view of these clinical realities, the use of genetic tests to identify treatment responders, nonresponders, and/or those likely to develop undesirable side effects will have a major impact on use of existing classes of CNS drugs, as well as on the development and use of new drugs to treat diseases of the central nervous system.

II. Identification of Interpatient Variation in Response; Identification of Genes and Variances Relevant to Drug Action; Development of Diagnostic Tests; and Use of Variance Status to Determine Treatment

Development of therapeutics in man follows a course from compound discovery and analysis in a laboratory (preclinical development) to testing the candidate therapeutic intervention in human subjects (clinical development). The preclinical development of candidate therapeutic interventions for use in the treatment of human diseases, disorders, or conditions begins at the discovery stage whereby a candidate therapy is tested in vitro to achieve a desired biochemical alteration of a biochemical or physiological event. If successful, the candidate is generally tested in animals to determine toxicity, adsorption, distribution, metabolism and excretion in a living species. Occasionally, there are available animal models that mimic human diseases, disorders, and conditions in which testing the candidate therapeutic intervention can provide supportive data to warrant proceeding to test the compound in humans. It is widely recognized that preclinical data is imperfect in predicting response to a compound in man. Both safety and efficacy have to ultimately be demonstrated in humans. Therefore, given economic constraints, and considering the complexities of human clinical trials, any technical advance that increases the likelihood of successfully developing and registering a compound, or getting new indications for a compound, or marketing a compound successfully against competing compounds or treatment regimens, will find immediate use. Indeed, there has been much written about the potential of pharmacogenetics to change the practice of medicine. In this application we provide descriptions of the methods one skilled in the art would use to advance compounds through clinical trials using genetic stratification as a tool to circumvent some of the difficulties typically encountered in clinical development, such as poor efficacy or toxicity. We also provide specific genes, variation in which may account for interpatient variation in treatment response, and further we provide specific exemplary variances in those genes that may account for variation in treatment response.

The study of sequence variation in genes that mediate and modulate the action of drugs may provide advances at virtually all stages of drug development. For example, identification of amino acid variances in a drug target during preclinical development would allow development of non-allele selective agents. During early clinical development, knowledge of variation in a gene related to drug action could be used to design a clinical trial parameters in which the variances are taken account of by, for example, including secondary endpoints that incorporate an analysis of response rates in genetic subgroups. In later stages of clinical development the goal might be to first establish retrospectively whether a particular problem, such as liver toxicity, can be understood in terms of genetic subgroups, and thereby controlled using a genetic test to screen patients. Thus genetic analysis of drug response can aid successful development of therapeutic products at any stage of clinical development. Even after a compound has achieved regulatory approval its commercialization can be aided by the methods of this invention, for example by allowing identification of genetically defined responder subgroups in new indications (for which approval in the entire disease population could not be achieved) or by providing the basis for a marketing campaign that highlights the superior efficacy and/or safety of a compound coupled with a genetic test to identify preferential responders. Thus the methods of this invention will provide medical, economic and marketing advantages for products, and over the longer term increase therapeutic alternatives for patients.

There are some examples whereby there is no direct evidence that a gene or genes are involved in drug response of a candidate therapeutic intervention. In these cases, however, there is genetic data supporting a role of a variance or variances involved in the etiology, progression, or risk of the neurologic or psychiatric disease. These cases, including but excluded to anxiety, Huntington's disease, demyelinating disease, pain, Parkinson's disease, spasticity, and stroke are described below with details of current therapies and potential genetic involvement of variances in drug responses.

A. Anxiety

Description of Anxiety

Anxiety is a common, nonspecific symptom associated to a greater or lesser degree with many psychiatric diseases, including psychoses, neuroses, mood disorders and personality disorders. It is also an inevitable component of everyday life brought on by stressful events such as medical or surgical procedures. Some prominent nonspecific symptoms of anxiety include tachycardia, chest pains, or irregular heartbeat; epigastric distress; headache, dizziness, syncope, or parethesias. It is usually some combination of these physical manifestations of anxiety that impels patients to seek medical care. It has been estimated that approximately 13% of primary care visits are substantially attributable to anxiety.

There are both acute and chronic anxiety syndromes. The acute forms include panic attacks and event-related anxiety. Chronic, or generalized anxiety is a pervasive feeling of nervousness that does not subside. Because both panic-attack and generalized anxiety lead to desire for being alone or away from public places, many patients adopt agoraphobic tendencies. These patients can become housebound because of fear of having a panic attack in a public setting.

Current Therapies for Anxiety

The principal treatments for anxiety have been benzodiazepines, monoamine oxidase inhibitors, antidepressants, and β-adrenergic antagonists. In all cases, both panic attack and generalized anxiety, concurrent continued behavioral and psychological therapy is required to regain a sense of normal life function.

Limitations of Current Therapies for Anxiety

The difficulty in determining the efficacy of psychotropic drugs for the treatment of anxiety is the subjective contribution of the nonpharmacologic factors that are associated with anxiety. However, the relative safety of benzodiazepines, pharmacologic actions, and high demand make these products drugs of choice in the treatment of anxiety.

The benzodiazepines are associated with side effects due to CNS depression, drowsiness and ataxia. Other side effects are: increase in hostility or irritability, confusion, weight gain, skin rash, nausea, headache, impairment of sexual function, vertigo, and lightheadedness.

Future Drug Development for Anxiety

In Tables 3 and 4, there are listings of candidate genes and specific single nucleotide polymorphisms that may be critical for the identification and stratification of a patient population diagnosed with anxiety based upon genotype. Current pathways that have possible involvement in the therapeutic benefit of anxiety include, but are not limited to, serontonergic, GABAergic, purinergic, adrenergic, glutaminergic, dopaminergic, cholinergic, glycinergic, cholecystokinin, corticotropin releasing factor, histaminergic, opiate, taurine, oxytocin, neuropeptide Y, estrogen, hemostasis, tachykinin, vasopressinergic and second messenger intracellular cascades gene pathways that are listed in Tables 1, 3 and 4. One skilled in the art would be able to identify these pathway specific gene or genes that may be involved in the manifestation of anxiety, are likely candidate targets for novel therapeutic approaches, or are involved in mediating patient population differences in drug response to therapies for anxiety.

Below, Table 6 lists the therapies in development for anxiety categorized by the gene pathway mechanism of action as in Table 1. The listed candidate therapeutic interventions response in patients with anxiety may be affected by polymorphisms in genes as described above.

B. Huntington's Disease

Description of Huntington's Disease

Huntington's disease (HD) is an inherited disorder characterized by the gradual onset of motor incoordination and cognitive decline in mid-life. Symptoms develop insidiously either as a movement disorder manifested by brief jerk-like movements of the extremities, trunk, face, neck (choreas) or as personality changes. Fine motor incoordination and impairment of rapid eye movements are early features. Bradykinesisas and dystonia may predominate if the onset occurs early in life.

As the disorder progresses the involuntary movements become more severe and are characterized by: dysarthria, dysphagia, and impaired balance. Cognitive deficits begin by features of slowed mental processing, difficulty in organizing complex tasks, and memory deficits (family, friends, and immediate situation is unaffected). These patients have tendencies to become irritable, anxious, and clinically depressed. In rare cases there may be paranoia or delusional states. There are approximately 25,000 Americans diagnosed with HD.

Current Therapies of HD

Current therapies do not include alternatives for the treatment of the progression of the neurodegeneration. Medical management of the associated clinical symptoms includes the following categories: depression, psychosis, and choreas. In the cases of depression and psychoses, the therapies of beneficial therapeutic use a re described in examples 4 and 11, respectively.

The treatment of choreas generally includes neuroleptic agents that affect dopaminergic pathways by antagonism at the receptor level. Monoamine depleting drugs can also be used to minimize choreas.

Limitations of Current Therapies of HD

Efficacy Limitations

Conventional and atypical neuroleptic agents are not uniformly able to reduce the signs and symptoms of choreas in HD patients. Efficacy varies in the HD population in one or combination of the following ways: 1) patients are only partially responsive or 2) patients are therapy resistant. Unfortunately, limited efficacy in a HD population in light of the presence of undesirable side effects may lead to compliance issues, aberrant drug abuse behavior, and further safety issues.

Thus, a clinician when presented with a newly diagnosed HD patient, in general, follows standard neurological society or published guidelines for first line therapy. However, when faced with a partially responsive or therapy resistant patient, the clinician can choose from multiple agents, none being completely effective, has limited guidance or rationale to select one agent the other, and follows an empirical medical decision making course of action.

Toxicity Limitations

Unfortunately, conventional neuroleptic drugs are uniformly, and atypical are latently, associated with undesirable dose-dependent side effects. These include but are not exclusive to sedation, weight gain, cognitive deficits, sexual or reproductive insufficiencies, agranulocytosis, cardiovascular complications, neuroleptic malignant syndrome (parkinsonism with catatonia), jaundice, blood dyscrasias, skin reactions, epithelial keratopathy, seizures, and extrapyramidal effects. The blood dyscrasias include mild leukocytosis, leukopenia, and eosinophilia. The skin reactions include uticaria and dermititis and are usually associated with phenothiazines. Epithelial keratopathy and opacities in the cornea is associated with chlorpromazine therapy. In extreme cases these effects may impair vision. These ocular deposits tend to spontaneously disappear upon discontinuation of chlorpromazine drug therapy.

The extrapyramidal side effects of conventional neuroleptics include dystonia (facial grimacing, torticollis, oculgyric crisis), akathesia (feeling of distress or discomfort leading to restlessness or constant movement), and parkinsonian syndrome (rigidity and tremor at rest, flat facial expression). With long term usage of conventional neuroleptic drugs, tardive dyskinesias uniformly appear in HD patients.

Tardive dyskinesia is a syndrome of repetitive, painless, involuntary movements. These abnormal involuntary movements are insuppressible, stereotyped, autonomic movements that cease only during sleep, vary in intensity over time, and are dependent on the level of arousal or emotional distress. The syndrome is characterized by quick choreiform (ticlike) movements of the face, eyelids (blinks or spasms), mouth (grimaces), tongue, extremities, or trunk. These movements may have varying degrees of athetosis (twisting movements) and sustained dystonic postures. Increasing the dose of the conventional neuroleptic agent can reverse extrapyramidal effects observed in patients. However, increasing the dose ultimately leads to more severe dyskinesias. Antiparkinson agents tend to exacerbate the tardive dyskinesia symptoms and thus are not used clinically. Because dopaminergic agonists tend to worsen the symptoms and dopaminergic antagonists tend to retard the symptoms of tardive dyskinesias, the optimal alternative is to use a neuroleptic agent that has selective dopaminergic antagonist activity. This alternative therapy would manage both psychosis and dyskinesias.

Often a clinician faces the dilemma of a patient with medically managed choreas, but the dose-related tardive dyskinesias, agranulocytosis, or seizures compels the medical care personnel to opt to switch therapies to possibly those agents or drugs with fewer or less severe side effects but with substandard or limited efficacy. Under these conditions, inability to treat the psychotic or chorea symptoms with the backdrop of irreversible dyskinesias leaves the patient with few alternatives.

III. Impact of Genotyping on Drug Development for HD

There have been reports of polymorphisms in key genes that affect neuroleptic activity in schizophrenic patients. These polymorphisms may be further applicable for neuroleptic response in HD patients. For example, within the dopamine D4 receptor subtype, there are known tandem repeats in exon 3. In a recent study, schizophrenic patients on maintenance doses of chlorpromazine were stratified into two groups, one having 2 tandem base pair repeats and the other having 4 tandem base pair repeats. Thirty-four percent of group one patients and 62% of group two patients had a favorable response to chlorpromazine therapy during acute stage treatments. The presence of homogeneous four 48 base pair repeats in both alleles in exon 3 of the dopamine D4 receptor subtype thus appears to be associated with beneficial chlorpromazine response.

Recently, a study of the serotonin receptor subtype 6, polymorphism (T267T vs. C267T) in a group of patients refractory to clozapine therapy was reported. In this study, it appeared that the T267T genotype patients were more likely to respond to continued therapy that those patients with C267T genotype patients.

A recent report documented a correlation of the serotonin 5HTC2 receptor subtype biallelic polymorphism and neuroleptic efficacy. A significant number of schizophrenic patients homozygous for the allele C2 responded unsatisfactorily to antipsychotic medication as compared to control.

Three polymorphisms in the serotonergic receptors, i.e. 5HT2A (T102C); 5HT2C (cys23ser); and 5HT2A (his452tyr) have reports of positive or negative correlation with efficacy of antipsychotic therapies. This disparity in the literature will, in the future, be further examined in schizophrenic patient populations and correlation may be discovered.

V. Description of Mechanism of Action Hypotheses for Future Drug Development

The genetic basis of the disease has been identified. A gene, huntingtin, whose protein has a mechanism yet to be defined, has a series of CAG tandem repeats. The number of CAG repeats do correlate somewhat with age of onset and the severity of the disease. Cell death starts in the caudate nucleus by an unknown mechanism. The huntingtin protein is essential to life. The huntingtin protein undergoes cleavage as cells age. The mechanism of cleavage is performed in part by members of the caspase enzymatic family. As the huntingtin protein is cleaved into smaller units, the peptides become toxic, and it has been shown that the smaller fragments tend to migrate into the nuclear compartment. It has been shown that preventing huntingtin cleavage prevents cellular toxicity. Some of the cleaved huntingin fragments form aggregates which may promote or be a by-product of neuronal cell death.

The profound loss of neurons in the brains of patients with HD has lead to many development programs for the promotion of neural regeneration. These programs broadly include cytokines, growth factors, and agents that promote neural or glial cell growth. Further, consideration of preventing neuronal cell death includes apoptosis inhibition and others. Other programs include prevention of prolonged excitatory neurotransmission. These neurons switch their aerobic metabolism to anaerobic metabolism leading to glycolytic metabolism and excessive production of lactate and metabolic by-products. Excitatory neural inhibition, improvement of energy metabolism, and inhibition of cell death signals may ultimately play a critical role in preventing, retarding, or halting neurodegeneration in HD patients.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with HD based upon genotype. Current pathways that may have involvement in the therapeutic benefit of HD include glutaminergic, serontonergic, dopaminergic, cholinergic, opiates, estrogen, mitochondrial maintenance, growth, differentiation, apoptosis, and secretion gene pathways that are listed in Tables 1, 2, 3 and 4. One skilled in the art would be able to identify these pathway specific gene or genes that may be involved in the manifestation of HD, are likely candidate targets for novel therapeutic approaches, or are involved in mediating patient population differences in drug response to therapies for HD.

Below in Table 10 is a list of therapies in development for HD categorized by the gene pathway mechanism of action. The listed candidate therapeutic interventions response in patients with HD may be affected by polymorphisms in genes as described above.

C. The Demyelinating Diseases

Description of Demyelinating Disease

Primary demyelinating diseases result in loss of the myelin sheath that surrounds axons, with preservation of the axons. The main demyelinating diseases are multiple sclerosis, including its variants (Marburg and Balo variants of MS and neuoromyelitis optica), and the perivenous encephalitides, which include acute disseminated encephalomyelitis and acute necrotizing hemorrhagic leukoencephalitis.

Due to the paucity of information concerning etiology of these diseases, identification and classification is largely descriptive. The most common and best studied of these diseases is multiple sclerosis.

Description of Multiple Sclerosis

There are an estimated 300,000 Americans diagnosed with MS. Women are affected slightly more often than men (1.6:1). The estimated annual cost of care for MS is $5 billion dollars.

Clinically, MS usually starts as a relapsing illness with episodes of neurological dysfunction lasting several weeks, followed by substantial or complete improvement. This is the relapsing-remitting phase of the disease. Many patients remain in this stage of the disease for years or even decades, while others rapidly progress to the next stage, secondary progressive MS, in which, with repeated relapses, recovery becomes less and less complete. There is also a steadily progressive relapse-independent form of the disease termed primary progressive MS. This form is characterized by a steady worsening of neurological function without any recovery or improvement, and more often affects men.

Current Therapies for Multiple Sclerosis

Although the pathogenesis of MS is not understood, there is accumulating evidence that immunoregulatory mechanisms are involved. Current therapy of MS is therefore directed to modulating immune function and thereby halting or retarding myelin degeneration, or facilitating remyelination. Remyelination has been shown to occur spontaneously in response to therapeutic interventions in animals (both normals and MS models). However, in MS animal models remyelination appears to be aborted soon after it begins.

For relapsing-remitting MS the following agents are currently in use: 1) interferon beta-1b (Betaseron) reduces annual relapse rate and reduces development and progression of new lesions in relapsing-remitting MS as monitored by magnetic resonance imaging (MRI), and has been shown to reduce annual relapse rate, reduce disability progression, and delay increase of lesion volume by MRI in secondary progressive MS; 2) Interferon beta-1a (IFN-beta-1α; Avonex) treatment results in reduced disability progression, annual relapse rate, and new brain lesions, as visualized by MRI; 3) Glatiramer acetate (Copaxone; Copolymer-1; Cop-1) reduces annual relapse rate; 4) Intravenous immunoglobulin, reduces annual relapse rate, and delays disability progression; 5) High-dose methylprednisolone therapy is effective in shortening MS attacks, and may be useful in the long term treatment of secondary-progressive MS; 6) Other agents that have been used with success are mitoxantrone, azathioprine and methotrexate. The latter drug, in particular, has been shown effective in reducing disease activity, both by decreasing the number of exacerbations and by slowing clinical progression. The first four agents are of comparable efficacy in the treatment of relapsing-remitting MS. Not enough trials have been performed to reliably assess the utility of treating nonresponders to one of these treatments with a different treatment, or to assess potential markers of response.

III. Limitations of Current Therapies for Multiple Sclerosis

The available treatments have both efficacy and toxicity limitations. Further, the cost for one year of interferon treatment is approximately $11,000 and parenteral administration is inconvenient.

Partial Response to Therapy

Current therapies reduce, but do not arrest, disease progression, and only a fraction of patients benefit from treatment; approximately 30% of patients on interferons experience reductions in relapse rates. For primary progressive MS, there are currently no effective therapies available; interferon beta-1b has in fact been shown to worsen spasticity in primary progressive MS.

Undesired Side-Effects or Toxicities as a Therapeutic Limitation

All interferons are associated to varying degrees with flu-like symptoms, muscle-ache, fever, chills, and asthenia. There are also side effects that are difficult to distinguish from the course of the demyelinating illness, for example interferons may lower the seizure threshold and exacerbate depressive illnesses, two clinical problems also observed in patients without interferon therapy.

Impact of Pharmacogenomics on Drug Development for Multiple Sclerosis

Aspects of therapy for demyelinating disease that can be addressed by pharmacogenetic methods include: 1) Which patients are most likely to respond to medication? 2) Which drugs are most likely to benefit which patients? 3) What is the optimal dose and duration of treatment? 4) What is the relationship between disease type, stage and manifestations and drug response? 5) Can adverse treatment responses be predicted? As an alternative to directly correlating genetic variants with clinical responses to therapy, one could also use quantitative biochemical, immunological or anatomical measures of disease activity to assess the impact of genetic variation in candidate genes on response to medication. While it is unlikely that all therapeutic responses are under strong genetic control, it is expected that if stratification based upon genotype were performed in clinical trials a correlation between drug response and genotype will be detected for at least some treatment responses. Described below and in Tables 1 & 2 are gene pathways that affect current drug therapy as well as drugs currently in development for MS. Described in the Detailed Description are methods for the identification of candidate genes and gene pathways, patient stratification, clinical trial design and statistical analysis and genotyping for testing the impact of genetic variation on treatment response in multiple sclerosis and other demyelinating diseases.

A sample of therapies approved or in development for preventing or treating the progression of symptoms of MS currently known in the art is shown in table 12. In this table, the candidate therapeutics were sorted and listed by mechanism of action. Further, the product name, the pharmacologic mechanism of action, chemical name (if specified), and the indication is listed as well.

Mechanism of Action Hypotheses for Novel Therapies for Multiple Sclerosis: Utility of Genotyping

Several possible mechanisms by which intravenous immune globulin (IVIG) modulates the course of the disease are related to limiting the inflammatory process and repairing the damage by enhancing remyelination. The efficacy of dexamethasone (DX) and methylprednisolone (MP) at high (HD) and low (LD) dose in acute multiple sclerosis (MS) relapses was evaluated by a double-blind trial in 31 patients followed for 1 year. DX and HDMP were similarly efficacious in promoting recovery, while LDMP was ineffective in the short-term outcome and was followed by an early clinical reactivation. The different outcomes seem to be related to different immunomodulating effects, mainly on cerebrospinal fluid (CSF) IgG synthesis and on peripheral blood and CSF CD4+ lymphocyte subsets. The efficacy of interferon should be investigated in relation to other treatment options, such as immunoglobulin, copolymer I, azathioprine and methotrexate. Other promising therapeutic options (mitoxantrone, intravenous immunoglobulins, drug associations) are under evaluation.

Pathogenesis of MS

There are three current theories for the cause of MS that have been studied to effectively understand the mechanism of disease as well as establish rationale for the development of effective candidate therapeutic interventions. The three current theories are 1) viral infection, 2) genetic predisposition, 3) inflammation and autoimmunity, and 4) ion channel modulators.

Viral Infection

Indirect evidence that there is a single unique virus causing MS is the unusual geographic distribution of the disease. There is a documented north-south gradient of disease prevalence, migration studies, and reports of clustering of cases have indicated an environmental influence on disease susceptibility. Despite years of intense research including viral isolation studies from tissue samples of MS patients and controls, has not resulted in identification of an MS specific virus or viral sequence.

One virus implicated in the pathogenesis of MS is the human herpes virus type 6 (HHV-6). HHV-6 is a neurotropic virus that can establish a latent infection in man. HHV-6 protein and DNA have been isolated and identified from neuroglial cells in active MS spinal lesions. Further, HHV-6 IgM titers in MS patients and HHV-6 DNA identified in serum samples indicate a recent infection. However, to date there is no evidence that HHV-6 is the causal infectious agent of MS. Instead, a hypothesis of molecular mimicry has been proposed as a likely possibility to explain the indirect immune-mediated injury to otherwise normal tissue in the course of clearing an infectious agent. Besides HHV-6, there are other neuro-specific infectious agents that may damage the CNS through this mechanism. The molecular similarity (mimicry) between virus and myelin antigens may be permissive for immunological cross-reactivity between HHV-6 and myelin antigens. In this model, the T-cells become activated, cross the blood brain barrier and misidentify normal myelin antigens as ‘virus’ resulting in T-cell mediated cellular and tissue injury.

Genetic Susceptibility to MS

Although MS is a sporadic disease, studies have pointed to an organized familial clustering, which suggests a genetic predisposition to MS. Equally likely, these studies also suggest that there is a genetic predisposition to an environmental stress or causal event.

The most convincing evidence of a genetic predisposition to MS is derived from studies of a population-based study of twins. The risk of MS increases with the degree of shared information within a family. There is further a marked increase in concordance for MS in the comparison of monozygotic and dizygotic twins.

Inflammation and Autoimmunity in MS

While it is clear there is an inflammatory component to the lesions of MS, is it currently unclear whether the immune system plays a role in initiation of the characteristic damage of white matter.

In experimental studies of animal models of MS, there appears to be T-cell, CD4+ and CD+8, autoreactivity to several putative CNS antigens including myelin basic protein, proteolipid protein, myelin oligodendroglial glycoprotein, 2′,3′-cyclic nucleotide phophodiesterases, myelin-associated glycoproteins, and viral antigens. Further, there appears to be down regulation of cytokine production including TNF-α and IL-3.

These observations have led to the following proposed mechanism of immune-mediated injury in an MS lesion. Genetic and environmental factors (e.g. viral infection, molecular mimicry, bacterial lipopolysaccharides, superantigens, local metabolic stress, oncogene expression, or reactive metabolites) may potentiate the movement of T-cells through the blood-brain barrier to the CNS. These same genetic and environmental factors may act within the CNS to upregulate the expression of intracellular adhesion molecules on endothelial cells and the circulating T-cells which in turn enhances the rolling, binding, diapedesis, and ultimate migration of the T-cells into the CNS. The same genetic and environmental factors may activate the secretion of αβ-crystallin on the oligodendrocytes rendering these cells more susceptible to T cell recognition. The T-cells once in the CNS then secrete cytokines (TNF-β and INF-γ) activate the antigen presenting cells (astrocytes, microglia, and macrophages) enhancing (macrophage, microglia) or inhibiting (astrocytes) further immune signaling. The activated T cell then encounters the putative MS antigen or antigens in light of the MHC class II molecules on the antigen presenting cells, resulting in T-cell activation. The activated T-cells can then differentiated into Th1 or Th2 type CD4+ cells which then results in proinflammatory or anti-inflammatory cytokine signaling, respectively. It has been shown in MS patients that antibody, complement, and antibody-mediated cellular toxicity mechanisms may cause the myelin lesions.

Ion Channel Modulations in MS

Reduction of the depolarization in postsynaptic membranes by modulation of the ion channels in nerve and muscle tissue has been postulated as a mechanism to ablate aberrant neurotransmission in demeylinating neurological disease. Proposed gene targets to produce the membrane depolarization are the nicotinic acetylcholine receptor, voltage gated Na+ channels, and other ion channels.

Future Therapeutic Strategies for MS

The future strategies for the beneficial therapy of MS are borne out of the existing mechanisms of the etiology of this demyelinating disease as previously described. They are antivirals, cytokine and anticytokine strategies, immune deviation strategies to enhance Th2 cell/cytokine performance, matrix metalloproteinase inhibitors, trimolecular complex strategies, cathepsin B inhibitors, and oxygen radical scavengers.

Specifically, antivirals include valcylcovir and acyclovir. Cytokine and anticytokine strategies include TNF inhibitors, antiinflammatory cytokines, and inhibitors of proinflammatory cytokines. Immune-deviation strategies to enhance Th2 cell/cytokine predominance includes pentoxifylline, transforming growth factor-β (TGF-β), and Il-10, Il-4 alone in combination with corticosteroids. Matrix metalloproteinase inhibitors include D-penacillamine, and hydroxyamatate. Trimolecular complex strategies include anti-MHC monoclonal antibodies, MHC class II hypervariable peptide vaccines, anti-T cell monoclonal antibodies, altered peptide ligands, T cell vaccination strategies (myelin basic protein reactive T-cell, T-cell receptor peptide vaccination), co-stimulation strategies (antib7-1, CTLA-4Ig fusion proteins, CD40/CD40 ligand interactions), and adhesion molecule signaling strategies (monoclonal antibodies, or small molecules directed to these adhesion molecules).

Neural regeneration development programs will include growth factors including NGF, BDGF, CNTF, NT-3, and other cytokines, as well as other factors that are involved in the support of nerve cell viability, growth, and sustaining neural transmission.

Technological advances that reduce difficulties in determining progression of the demyelination by neuroimaging techniques will aid development of new therapies. Estimation of expected clinical and surrogate measures and patterns to identify, screen, and develop statistically derived stopping rules for efficacy and futility.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with MS based upon genotype. Current pathways that may have involvement in the therapeutic benefit of epilepsy include glutaminergic, GABAergic, opiates, corticotropin releasing hormone, potassium channel, prostaglandin, platelet activating factor, cytokines, clot formation, second messenger cascade, growth, differentiation, and apoptosis, cytoskeleton, adhesion, and myelination gene pathways that are listed in Tables 1, 2, 3 and 4. One skilled in the art would be able to identify these pathway specific gene or genes that may be involved in the manifestation of MS, are likely candidate targets for novel therapeutic approaches, or are involved in mediating patient population differences in drug response to therapies for MS.

D. Pain

Description of Pain

Chronic pain can be caused by chronic pathologic processes in somatic structures or viscera, or by prolonged dysfunction of parts the peripheral or central nervous system. In all there are approximately 70 million Americans that experience chronic pain. Chronic pain may be the result of recurrent headache, arthritis, back or spinal injuries, musculoskeletal disorders, cardiac or visceral pathologies. Chronic pain is also part of the clinical manifestation of cancer; many of these cases are medically intractable pain. Chronic pain syndromes include polyarteritis nodosa; systemic lupus erythmatosus; entrapment neuropathy; lumbar plexitis; Bell's palsy; carpal tunnel syndrome. Chronic pain can also result from peripheral neuropathies: diabetic neuropathy (neuropathic complications of diabetes mellitus include distal symmetric, sensory, autonomic, asymmetric proximal, cranial and other mononeuropathies); cervical radiculopathy; Guillain-Barre syndrome; brachial plexitis; familial amyloid neuropathy; HIV neuropathy; post spinal cord injury; and post herpetic neuralgia.

Current Therapies for Pain

Therapeutic management of chronic pain includes a three step ladder approach: non-opioid analgesics are stepwise prescribed in combination with moderate to potent opiates. The guidelines call for a determination by the patient and the physician of pain relief. Broadly speaking, the guidelines are as follows: mild pain is treated with non-opioid analgesics, moderate or persisting pain is treated with a weak opioid plus non-opioid analgesics, and severe pain that persists or increases is treated with a potent opioid plus non-opioid analgesics.

Pain management regimens include not only the use of opioids and non-opioid analgesics, but also benzodiazepines, local anesthetics, anticonvulsants, anticholinergics, serotonin norepinephrine reuptake inhibitors, neuroleptics, and barbiturates. These drugs in combination can relieve associated symptoms of chronic pain syndromes such as anxiety, acute on top of chronic pain, seizures, dry mouth, delirium, and inability to sleep, respectively.

Treatment options for chronic pain fall into the following categories: 1) general health promotion and relief from exacerbating factors; 2) nonnarcotic pharmacologic; 3) physical; 4) surgical; and 5) narcotic.

The nonnarcotic empirical therapies include tricyclic antidepressants (amitriptyline, nortriptyline, doxepin, imipramine), anticonvulsants (carbamazepine, phenyloin); GABAergic agonists (BACLOFEN®) and antipsychotics (fluphenazine). Narcotic therapies include opioid agonists (methadone and fentanyl). Devices and surgical therapies can be used in combination with drug therapy. In general these therapies have been shown to reduce pain and each are described in detail below.

Antidepressants: The tertiary amines are the most commonly used anti-depressants to manage pain associated with post-SCI. Although the exact mechanism is unknown, the interference with the re-uptake of neurotransmitters (dopamine, norepinephrine, and serotonin) may reduce pain transmission in the afferent pathways. Further, the increased quantities of these neurotransmitters in the areas of the hyperexcitable neurons, descending pain inhibitory pathways that terminate in the substantia gelatinosa of the dorsal horn, may act to reduce pain transmission. Interestingly, the dose of the tricyclics for the management of pain is approximately half that required for the management of depression. These compounds can be determined to be effective for pain management in approximately two weeks.

Anticonvulsants: Reports exist describing chronic neuropathic pain syndromes as a central neurophysiologic epileptiform activity of the uncontrolled hyperactive neurons leading to a convulsive syndrome in the spinal cord. Thus, anticonvulsant therapies are considered to stabilize the threshold against hyperexcitability of neurons and inhibiting the spread of epileptiform activity in neurons involved in nociception. Further, activation of inhibitory neurons may lead to a pain reduction. Although the data is not conclusive, it appears that anticonvulsants are more effective when given in combination with antidepressants.

Neuroleptics: The neuroleptics are thought to exert a potentiation of the antidepressants and may impart a dopaminergic antagonism. Neuroleptics are usually given in combination with an antidepressant.

GABAergic agonists: Baclofen, a GABAegeic agonist when delivered intrathecally was effective in reducing chronic pain in those patients in which the pain was of musculoskeletal origin (83% of these patients), but was ineffective in those patients with neurogenic pain (78% experienced no change).

Physical treatments: Physical treatments include transcutaneous electrical nerve stimulation (TENS) and spinal cord stimulation devices. Using TENS, some success has been reported to reduce peripheral pain. Upon placing the electrodes, peripheral sensory nerve stimulation is thought to activate pain inhibitory interneurons in the substantia gelatinosa or dorsal root entry zone of the spinal cord.

Spinal cord stimulation devices are programmable multichannel systems with electrodes that may be placed percutaneously, these systems do not require laminectomy. These stimulators have been shown to reduce chronic pain (perceived pain levels requiring intensive therapies: discomforting, distressing, horrible, and excruciating) by 50% long term. The global ratings for quality of life in these patients demonstrated similar long term improvements. The exact mechanism of how spinal cord stimulation results in a reduction of pain is unknown, but it is thought to occur through an antisympathetic effect. Further, it seems to be effective in cases in which the patient has neuropathic or an ischemic component to the pain. In patients with peripheral neuropathies (postherpetic neuralgia, intercostal neuralgia, causalgic pain, diabetic neuropathy, idiophathic neuropathy) spinal cord stimulation is able to reduce chronic pain in approximately 50% of the patients.

Surgical treatment: If conservative pharmacologic approaches have failed to relieve pain, neurosurgery can be considered. Neurosurgical treatments consist of nerve blocks, neuroablative and neuroaugmentative procedures.

Nerve blocks: Peripheral, epidural, and sympathetic nerve blocks have been attempted. However, the analgesic effect is usually short-lived and ineffective against central mechanisms of pain.

Neuroablative procedures: There are surgical procedures that are rarely performed because they have been shown to be ineffective, i.e. sympathectomies, neurolyses, dorsal rhizotomies, cordectomies, anterolateral cordectomies, mesencephalotomies, and cingulotomies. These procedures have been superseded by dorsal root entry zone (DREZ) surgery. The surgical procedure involves a laminectomy of the appropriate vertebrae, examination of the DREZ and radiofrequency lesions of the DREZ. The mechanism of this ablative surgery is thought to be due to the destruction of the secondary pain sensory neurons in the substantia gelatinosa in the dorsal horn. Success of this procedure on the reduction of pain has been reported at 60-90%.

Neuroaugmentative procedures—deep brain stimulation: Electrodes are implanted in the periventricular gray matter, specific sensory thalamic nuclei, or the internal capsule.

Limitations of Current Therapies for Pain

Limitations of Current Therapies of Pain Due to Low Efficacy

The severity of pain can be debilitating and significantly interfere with the productivity and quality of life. Existing therapies for chronic pain are often inadequate and characterized by the tendency to become ineffective with time. Potent opiates are part of an analgesic regimen, however, dose-limiting side effects and antinociceptive capacity, tolerance and potential for dependence limit their widespread use. Surgical intervention is sometimes attempted, but often such procedures are ineffective and at best provide only temporary relief.

There are many syndromes by which the above combination drug therapy is insufficient to relieve symptoms of chronic pain. There are common reasons for unrelieved pain associated with the patient or family, i.e. belief that pain in cancer is inevitable and untreatable, failure to contact a physician, patient denial, failure to take medications, noncompliance due to fear of addiction, noncompliance due to a belief that tolerance will rapidly develop and adequate pain relief then will not be available in the advanced stages, and lastly noncompliance due to the adverse side effects. Common reasons for unrelieved pain associated with the physician or nurse are: denial of the patient's pain, unawareness of pain intensity, failure to perceive patient denial, failure to treat pain aggressively, fear of patient addiction, failure to prescribe appropriate doses for analgesia, failure to monitor the patient's progress, failure to understand alternative drug combinations, and finally failure to give psychological support to the patient and family. Despite these common reasons for unrelieved chronic pain, even under positive conditions chronic pain can be intractable in a variety of diseases.

The coexistence of pain and depression in these patients is a dependent relationship, i.e. when the pain is unmanaged the depression becomes more severe, the reverse (increased depression leads to increased pain) relationship is less likely to occur. The characteristic intensity of the pain and psychological impact prompts extreme potential solutions. Some of these pain syndromes are more resistant to analgesic therapy, for example approximately half of the individuals with spinal cord injuries endure chronic pain and 30% experience severe, debilitating chronic pain. Approximately 75% of advanced stage cancer patients experience moderate to severe pain and approximately half of these individuals are refractory to standard therapy for management of pain.

Other efficacy limitations include: slow onset of symptoms (2-3 weeks) before efficacy detection for tricyclic antidepressants.

Limitations of Current Therapies of Pain Due to Toxicity or Undesired Side Effects

In the stepwise approach to therapy, physicians are able to monitor and adjust the doses to limit undesired side effects of opioids: sedation, cognitive impairment, myoclonus, addiction, and respiratory depression. Further, opiate tolerance is a well documented effect seen in routine narcotic users and abusers. These side effects may provoke a use of opioid rotation in a pain management schedule.

Although the use of opioids in acute and chronic cancer associated pain is well accepted, their use in chronic noncancer pain has been widely considered to be inappropriate due to concerns over efficacy, toxicity and addiction.

Other unwanted or undesirable side effects include tardive dyskinesias limit the use of neuroleptics in the management of chronic pain; oral baclofen is associated with drowsiness and confusion. Further, baclofen may cause hepatotoxicity. Complications of radiofrequency lesions of DREZ procedure includes cerebrospinal fluid leaking, loss of sensory/motor functions, exacerbation of bowel, bladder, or sexual dysfunction, and epidural/subcutaneous hematomas. Patients must consider the risks of this procedure, particularly the potential loss of two levels of sensation. Associated with deep brain stimulation are complications due to the release of large amounts of natural opioids leading to deafferenation and nociceptive pain.

Impact of Genotyping on Drug Development for Pain

As described above, there is evidence to suggest that there are efficacy and safety differences to drug therapy in the pain patient population. Although not all of these responses may be attributable to genotypic differences, it is expected that if stratification based upon genotype were performed, a reasonable correlation between drug response and genotype may become obvious. As described below, there are gene pathways that are involved with current drug therapy and those that may be potentially involved in the future. As described in the Detailed Description, methods for the identification of candidate genes and gene pathways, stratification, clinical trial design, and implementation of genotyping for appropriate medical management of a given disease is easily translated for pain syndromes. As described below in section V below there are likely gene pathways as are those that are outlined in the gene pathway Table 1 and matrix table 2.

For example, optimization of GABAergic, opiate, or ion channel modulation mediated therapy of pain further demonstrates the utility of selection of a potential epilepsy patient that has a predisposing genotype in which selective analgesics or agents are more effective and or are more safe. In considering an optimization protocol, one could potentially predetermine variance or variances within the GABAergic receptor, ion channel or ion channel mediated mechanisms of neurotransmission, or GABAergic receptor mediated intracellular mechanism of action that is preeminently responsible for drug response. By embarking on the previously described gene pathway approach, it is technically feasible to determine the relevant genes within such a targeted drug development program for pain.

A sample of therapies approved or in development for preventing or treating the progression of symptoms of pain currently known in the art is shown in Table 13. In this table, the candidate therapeutics were sorted and listed by mechanism of action. Further, the product name, the pharmacologic mechanism of action, chemical name (if specified), and the indication is listed as well.

Description of Mechanism of Action Hypotheses for Future Drug Development for Pain

The persistence of pain most likely involves a cascade of pathological neurochemical events that lead to abnormal sensory hyperexcitability and excitotoxicity. The persistence of hyperexcitability involves a sequence of neuroplastic events in the spinal cord. In particular, the hyperexcitability cascade involves NMDA receptor mediated intracellular calcium-dependent increase of nitric oxide (NO) and cGMP production. These signals facilitate long-term alterations in neuronal excitability and central sensitization. The altered spinal neurochemical environment results in an impairment of neural inhibitory function. In particular, inhibitory gamma-aminobutryic acid (GABA)-ergic interneurons are susceptible to excessive excitatory amino acid release. Recent studies suggest that abnormal pain sensations may be alleviated by application of GABA receptor agonists. The analgesic capacity of GABA receptor agonists has been demonstrated in numerous animal models of acute and chronic pain.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with pain based upon genotype. Current pathways that may have involvement in the therapeutic benefit of epilepsy include glutaminergic, serontonergic, dopaminergic, adrenergic, cholinergic, histaminergic, purinergic, GABAergic, glycinergic, melatonin, nitric oxide, peptide protein hormone processing, opiates, cholecystokinin, tachykinin, bradykinin, corticotropin releasing hormone, somatostatin, galanin, calcium or sodium channels, prostaglandin, cytokines, growth, differentiation, apoptosis, lipid transport/metabolism pathways that are listed in Tables 1, 2, 3 and 4. One skilled in the art would be able to identify these pathway specific gene or genes that may be involved in the manifestation of pain, are likely candidate targets for novel therapeutic approaches, or are involved in mediating patient population differences in drug response to therapies for pain.

E. Parkinson's Disease

Description of Parkinson's Disease

Parkinson's disease is one of the major neurodegenerative disorders of middle and old age. There is an estimated 500,000 patients in the U.S. with PD with an estimated annual cost of $6 billion dollars.

Parkinson's disease (PD) is a clinical syndrome that is dominated by four clinical symptoms: tremor at rest, bradykinesia, rigidity, and postural instability. There are secondary clinical signs and symptoms also associated with PD and are a result of the following manifestations: mood and intellectual disorder, oculomotor control, and autonomic and sensory dysfunction. PD can be generally categorized by the clinically predominant parkinsonian feature: 1) those patients having tremor, or 2) those patients having postural instability and or gait difficulty as the predominant clinical parkinsonian manifestation. In those patients with tremor predominant disease, the onset is earlier in life and exhibits a slower progression that those patients with gait difficulties or postural instability. In the latter case, the age of onset is later in life and is more frequently associated with bradykinesias, dementia, and the movement disorder progresses more rapidly. The stages of PD have been described and are referred to as Hoehn and Yahr stages I through V; stage I—signs and symptoms are unilateral, stage II—signs and symptoms are bilateral, stage III—signs and symptoms are bilateral and balance is impaired, stage IV—functionally disabled, and stage V—patient is confined to wheelchair or bed.

Resting tremor and bradykinesias are the hallmarks of PD. Bradykinesias are primarily responsible for the altered clinical presentation for most PD patients: retardation of activities of daily living and generalized slowing down of movements, lack of facial expression (hypomimia or masked facies), staring expression due to limited ability to blink, impaired swallowing which causes drooling, hypokinetic and hypophonic dysarthria, monotonous speech, micrographia, impaired simultaneous and repetitive movements, difficulty in standing from a chair and turning in bed, shuffling gait with short steps, decreased arm swing and other autonomic movements, start hesitation and sudden freezing of motion. Freezing of motion manifests as a sudden and often unpredictable inability to move and represents the single most disabling parkinsonian symptoms.

There are several disorders other than PD that manifests with parkinsonian symptoms. For example, acquired or symptomatic parkinsonism is the result of infectious (postencephalitic and slow virus) disease, side effects from drugs (neuroleptics (antipsychotic and antiemetic drugs), reserpine, tertabenazine, a-methyl dopa, lithium, flunarizine, cinnarizine), toxins (MPTP, carbon dioxide, manganese, mercury, cesium, methanol and ethanol), cerebrovascular insult (multi-infarct, hypotensive shock), trauma (pugilistic encephalopathy), and others (parathyroid abnormalities, hypothyroidism, hepatocerebral degeneration, cerebral tumors, normal pressure hydrocephalus, syringomesencephalia). Parkinsonism can also be the result of heredodegenerative disease, for example autosomal Lewy body disease, Huntington's disease, Wilson's disease, Hallervorden-Spatz disease, olivopontocerebellar and spinocerebellar degenerations, familial basal ganglia calcification, familial parkinsonism with peripheral neuropathy, and neuroacanthocytosis. Lastly, parkinsonism can be the result of multiple-system degenerations and include for example progressive supranuclear palsy, Shy-Drager syndrome, striatonigral degeneration, Parkinsonism-dementia-amyotrophic lateral sclerosis complex, corticobasal ganglionic degeneration, Alzheimer's disease, and hemiatrophy-parkinsonism. These non-PD parkinsonism symptoms can be clinically identified as distinct from PD due to the presence of atypical signs or symptoms of the particular dysfunction or syndrome, absence or paucity of tremor, and poor response to levodopa.

Current Therapies for PD

Pathophysiologically, idiopathic PD cases are almost uniformly identified by the absence of dopaminergic terminals and depigmentation within the substantia nigra and the presence of Lewy bodies (eosinophilic cytoplasmic inclusions in neurons consisting of aggregates of normal filaments). These abnormalities are predominantly found in the ventrolateral region of the substantia nigra which is the region that projects to the putamen. It has been estimated that at least 80% of dopaminergic neuronal loss within the substantia nigra and an equal degree of dopamine depletion within the striatum is required-before-signs and symptoms of PD is clinically observed.

There are currently four categories of drug therapies for the treatment of PD: dopaminergic replacement drugs, dopaminergic agonists, anticholinergic drugs, and monoamine oxidase inhibitors. Other therapies include surgical treatment and implantable devices for control of debilitating essential tremor.

Dopaminergic Replacement Drugs—therapy of PD is aimed at replacing the lost dopamine that has resulted in the loss of dopaminergic neurons in the substantia nigra and other brain regions. L-dopa is a prodrug that can be converted to dopamine within the existing neurons. Generally, L-dopa is beneficial in early PD, because it is effectively metabolized in presynaptic terminals and secreted in an active form. Due to the rapid decarboxylation of L-dopa in the periphery, administration of large doses is required to achieve therapeutic benefit. However, L-dopa is usually administered with carbidopa, an inhibitor of peripheral decarboxylation and thus greater concentrations of L-dopa enters the CNS. The combination of L-dopa and carbidopa reduces by 75% the amount of L-dopa required.

Dopaminergic Agonists—dopaminergic agonists can be administered in the early stages of the disease, examples include parlodel and permax.

Anticholinergic Drugs—anticholinergic agents are prescribed for the management of tremor or inordinate movements associated with PD, examples include artane, and cogentin. The majority of the anticholinergic therapies for the adjunct treatment of PD are long-acting medications thus relief of symptoms may continue through the night when patients have difficulty turning in their bed, and to rise in the morning.

Monoamine Oxidase Inhibitors—inhibition of the metabolism of dopamine by monoamine oxidase can be achieved to increase the synaptic levels of dopamine. An example is selegiline.

Others—catechol-o-methyl transferase inhibitors may be prescribed for the adjunctive treatment of PD, example is Tasmar. An antiviral, symmetrel, has been used for the relief of tremors, rigidity, and bradykinesia. Some β-adrenergic antagonists have been shown to reduce tremors, example is inderal.

Prior to the advent of levodopa therapy, the most effective means of treating disabling tremors associated with PD were thalamotomy and pallidotomy. These ablative surgical procedures are associated with improved tremor and in certain cases, bradykinesias. Recent advances in neurosurgery, e.g. devices to specifically record from the globus pallidus for enhanced localization, have been employed and there is renewed clinical interest in considering these therapies for the treatment of PD. This therapy has the advantage of single procedure therapeutic intervention of disabling tremors.

Another therapeutic alternative for the treatment of essential tremor, a device for deep brain stimulation, is approved for unilateral implantation in the ventral intermediate nucleus of the thalamus. A programmable, implantable pulse generator is implanted just below the clavicle. The implanted device has been shown to be effective in 20% of the patients, bilateral implantation and stimulation is under investigation.

Limitations of Current Therapies for PD

Although there are therapeutic alternatives for the early intervention of PD, there are few alternatives for the later stages and for the side effects that develop after long term therapy. These limitations are discussed below.

Limitations of Current Therapies Due to Low Efficacy

All anti-Parkinson drugs have two qualities that limit the efficiency of treatment regimens. First, the drugs are relatively short acting. A single administration does not relieve symptoms for the duration of waking hours, and multiple administrations are required. The second is that these drugs are all centrally acting drugs and starting dosage is low and slowly increased. Abrupt withdrawal or reductions of any of these medications can lead to deleterious side effects.

L-dopa therapy of PD has therapeutic benefit in the early stages of the disease. However, as the movement disorder progresses, the dopaminergic terminals are lost and the prodrug is no longer converted to the active form. The therapeutic benefit is then limited to the level and extent of the intact postsynaptic neurons.

Long-term therapy with levodopa is associated with dose dependent side effects including inefficacy, “on-off” phenomena, and dyskinesias. Response fluctuations occur in approximately 80% of the patients. These fluctuations consist of wearing-off phenomena, a gradual loss of effectiveness of levodopa related to the timing of administration of the drug, and the on-off phenomena, which is an abrupt loss of the effectiveness of levodopa that is not related to the timing of administration.

Dyskinesias, consisting of chorea and dystonia, occur in approximately 40% of patients treated with levodopa. These dyskinesias are most frequently observed when plasma levels of L-dopa are high.

For patients with preexisting history of psychiatric illness, anticholinergic therapies are less likely to be administered and further if prescribed are less likely to be effective.

Thalamotomy and pallidotomy are two surgical procedures that can only be performed once per side. Thus, refractory cases or cases whereby surgery was not sufficient to alter the essential tremor, additional surgery is unavailable.

Deep brain stimulation is only 20% effective, requires extensive follow-up, and is associated with a surgical morbidity of 5%.

Animal model studies of growth factors, GDNF, affected sprouting of peripheral neurons and those in the spinal cord. Unregulated neural sprouting can be deleterious to neurological function.

Limitation of Current Therapies Due to Toxicity or Undesired Side Effects

Limitations due to toxicity or undesired side effects for the above discussed treatments of PD are as described below for each of the treatment strategies.

Dopaminergic replacement drugs—as described above, L-dopa is a prodrug that can be of therapeutic benefit to patients with PD. However there are side effects and toxicities associated with L-dopa therapy, they are choreiform and dystonic dyskinesias and other involuntary movements, adverse mental changes such as paranoid ideation, psychotic episodes, depression, and cognitive impairments (dementia). Dyskinesias associated with levodopa, can be debilitating and as uncomfortable as the rigidity and akinesia of PD.

Reductions or withdrawals of L-dopa therapy have been associated with neuroleptic malignant syndrome (NMS). NMS is an uncommon but life-threatening syndrome characterized by fever or hyperthermia, muscle rigidity, involuntary movements, altered consciousness, autonomic dysfunction, tachycardia, tachyapnea, sweating, and hyper- or hypotension.

Dopaminergic agonists—as described above, dopaminergic agonists are useful for the activation of post synaptic dopaminergic receptors. The side effects and toxicities associated with dopaminergic agonists are: abnormal involuntary movements, hallucinations, “on-off” phenomena, dizziness, fainting, visual disturbances, ataxia, insomnia, depression, hypotension, constipation, vertigo, and shortness of breath. It has been observed clinical laboratory transient elevations of blood sera urea and nitrogen, SGOT, SGPT, GGPT, CPK, alkaline phosphatase, and uric acid.

Anticholinergic drugs—the predominant affect afforded by the anticholinergic drugs is to treat the extrapyramidal effects that develop with long-term dopaminergic therapies. This therapy is thus via the anticholinergic and antihistaminergic effects. However, there are adverse reactions that are associated with anticholinergic therapies, they are tachycardia, paralytic ileus, constipation, dry mouth, toxic psychosis (confusion, disorientation, memory impairment, visual hallucinations, possible exacerbation of pre-existing psychiatric symptoms or syndromes, blurred vision, dysuria, and urinary retention.

Monoamine oxidase inhibitors—selective inhibition of monoamine oxidase type B (MAO-B) enzyme activity is a useful adjunctive therapy to increase concentrations of dopamine in regions of the brain. Since MAO-B is predominantly found in the brain, fewer systemic side effects occur. Despite this selectivity, there are side effects that are undesirable, they are exacerbation of L-dopa or other dopamine agonist mediated side effects. For example, dyskinesias are enhanced as well as the others listed above.

MAO-B inhibition can be deleterious if administered with a tricyclic antidepressant. Further, a combination of MAO-B inhibitor and merperidine (an opioid narcotic) has lead to stupor, muscle rigidity, severe agitation, and hyperthermia. Thus, concomitant administration of these two types of drugs is avoided.

Others—inhibition of COMT as described above is a useful therapeutic alternative to many PD patients. However, there are associated side effects and toxicities associated with this drug family. In some patients there is a clinical liver enzyme elevation that requires monthly monitoring and liver function tests are routinely administered every 6 weeks for the first three months of therapy. Liver impairment can result in the reduction of drug detoxification mechanisms, and clinically as jaundice.

Because COMT and monoamine oxidase are the two predominant metabolizing enzymes for catecholamines, concurrent therapy of a COMT and a non-selective monoamine oxidase inhibitor may result in aberrant neuroexcitoxicity. However, selective monoamine oxidase inhibitors of MAO-B may be administered together.

Other side effects include dykinesias, nausea, sleep disorders, dystonia, excessive dreaming, anorexia, muscle cramps, and orthostatic hypotension.

Surgical treatment and implantable devices—both pallidotomy and thalamotomy are routinely considered for the treatment of refractory essential tremor. The extent and level of surgical success is dependent on accurate localization of the globus pallidus or the thalamus. Surgery that includes either of these two methods is a one attempt procedure, too much surrounding brain tissue may be lost in subsequent procedures. A side effect may be loss of cerebral function in surrounding areas that may or not result in clinical relevant or observable disease.

Impact of Genotyping on Drug Development for PD

For Parkinson's disease, there is evidence to suggest that there are efficacy and safety differences to drug therapy in the PD patient population. Although not all of these responses may be attributable to genotypic differences, it is expected that if stratification based upon genotype were performed, a reasonable correlation between drug response and genotype may become obvious. As described below, there are gene pathways that are involved with current drug therapy and those that may be potentially involved in the future. As described in the Detailed Description, methods for the identification of candidate genes and gene pathways, stratification, clinical trial design, and implementation of genotyping for appropriate medical management of a given disease is easily translated for PD. As described below in section V. below there are likely gene pathways as are those that are outlined in the gene pathway table 1 and the matrix table 2.

Description of Mechanism of Action Hypotheses for Future Drug Development

Motor symptoms of PD result primarily from the degeneration of dopaminergic innervation within the putamen and the caudate nucleus. Further dopaminergic degeneration within the mesocortical and mesolimbic systems may be responsible for the cognitive deficits and neurobehavioral symptoms. Autonomic dysfunction often observed in PD patients may be the result of loss of dopaminergic function in the hypopthalamus. Although dopaminergic pathways have been studied extensively in post mortem PD patients loss of neurotransmitter pathways that may be responsible for additional clinical symptomology. For example, loss of noradrenergic innervation in the locus ceruleus may contribute to the sudden and unpredictable freezing of motion and degeneration of cholinergic neurons in cortical areas may lead to observed dementia in PD patients.

There have been recent proposals for the mechanism of selective neuronal cell death and functional loss. The proposed mechanisms involved in the progressive degeneration of dopaminergic neurons are oxidative stress, mitochondrial dysfunction, excitotoxic damage, cell death. Below each is described, with proposed gene targets.

Oxidative Stress

In oxidative stress, generation of reactive oxygen species, part of the normal cellular metabolism, is aberrant and levels exceed the regulated cellular metabolism or scavenging mechanisms. The free radicals are generated by the conversion of superoxide ions to hydrogen peroxide via the enzyme superoxide dismutase and the reaction of hydrogen peroxide with reduced glutathione to produce water under the control of glutathione peroxidase. Since it has been documented a 60% reduction in the available reduced glutathione as well as a increased generation of iron associated with neuromelanin, there is a potential shift in the balance of the capacity to scavenge hydrogen peroxide radicals.

Oxidative stress may also be part of circuitous pathway leading to cell death that is as follows: generated free radicals lead to mitochondrial damage, which leads to neuron excitotoxicity, which leads to increased concentrations of intracellular calcium which increases the generation of free radicals. All four pathways (free radicals, mitochondrial damage, neural excitotoxicity, and increased intracellular calcium) can independently lead to neuron cell death. Neuroprotective agents, antioxidative agents, and those agents having effects of halting, retarding, or preventing progression of neurodegeneration may affect one or more of these pathways leading to therapeutically relevant agents.

Mitochondrial Damage

In mitochondrial damage, the evidence is born out of the experiments of the specific neurotoxin, MPTP. MPTP is a protoxin, its active form MPP+ has been shown to result form its inhibition of mitochondrial respiration at the level of complex I, the complex that controls the transfer of one electron from NADH to co-enzyme Q and the transfer of two protons to the mitochondrial inner space, both are then used to synthesize ATP from ADP. In addition, MPP+ is thought to increase leakage of electrons at complex I, thereby increasing the generation of superoxide. Since the association of MPTP and the evolution of PD in intravenous drug users, it has been shown that there is a decrease in complex I activity in the substantia nigra in PD patients and is relatively unique to PD than other neurodegenerative disorders.

Excitotoxic Damage

In excitotoxic damage, the theory posits there is an excess glutaminergic signal from the neocortex and the subthalamic nucleus to the substantia nigra. The excess signal, by acting at NMDA receptors, changes the permeability of the neural cells to calcium which leads to aberrant post synaptic membrane potentials, enhanced propensity for depolarization and latent repolarization, and activation of nitric oxide synthase (NOS). Activation of NOS leads to the generation of free oxygen radicals through the peroxynitrite reaction. Since the discovery that output neurons of the subthalamic nucleus provide a glutaminergic excitatory input to the substantia nigra, increased calcium influx into the cells and increased formation of nitric oxide via the activation of NOS, may be particularly harmful in PD due to the defect in mitochondiral complex I (see above). Excitotoxic damage to the substantia nigra, thus potentially stems from the integrity of the substantia nigra and or overactivity of the subthalamic nucleus. Thus, strategies aimed at dual actions of enhancing dopaminergic status (dopamine agonism) in the substantia nigra and reducing subthalamic overactivity (glutaminergic antagonism).

Cell Death

In neural cell death, neurons in the substantia nigra undergo death signals via necrosis and apoptosis. In studies involving double labeling with the TUNEL assay (apoptosis) to determine DNA fragmentation and cyanine dye labeling to determine cell structural detail, it was shown that DNA fragmentation and chromatin condensation occurs in the same nuclei of neurons in substantia nigra in patients with PD. Therefore, it appears that the number of apoptotic nuclei in the substantia nigra in PD is greater than that seen in normal aging, consistent with the 10-fold higher rate of cell loss observed in patients with PD. Thus, antiapoptotic agents or therapies may halt, retard, or prevent the progression of neurodegeneration.

Neuroprotection afforded by growth factors in general or specific to neurons have been considered. Growth factors including but not limited to BDNF, GDNF, bFGF have been studied in preclinical animal models of PD. Furthermore, GDNF has been tested in clinical trials.

Alternative neurotrophic agents are a group of ligand called the immunophilins. These ligands have been shown to have neurite growth promoting and neuroprotective effects. Although these effects were first described from results of experiments of the immunosuppressive agents, cyclosporine and FK-506, nonimmunosuppressive analogues have been generated to have neuroprotective capacity while having none of the immunosuppresive qualities. These low molecular weight ligands may hold promise for the medical management of PD.

Based upon these varying hypotheses as stated above, there are many products in development for PD. Table 14 below lists current therapies that are in development for PD.

F. Spasticity

Description of Spasticity

Spasticity is a complication that occurs in patients with diagnosed neurodegenerative diseases or cerebral insults such as multiple sclerosis, cerebral palsy, tetanus, traumatic brain injury, post traumatic spinal cord injury, amyotrophic lateral sclerosis, dystonic syndromes (axial dystonia), and stroke. Together there are approximately 1.8 million individuals with spasticity in the U.S. Spasticity is a term that generally refers to one of a variety of forms of muscle hypertonicity, hyperactive muscle stretch reflexes, exaggerated tendon reflexes, and clonus and flexor spasms. Spasticity is commonly described as an isokinetic movement disorder distinguished by velocity-dependent increase in muscle tone characterized by hyperactive stretch reflexes. Patients with spasticity have impaired voluntary control of skeletal muscles, difficulty relaxing muscles once movement has stopped, difficulty initiating rapid movements, and an inability to regulate controlled movement.

Clinically, there are three types of spasticity 1) mild, characterized by hyperactive reflexes and unsustained myoclonus; 2) moderate, characterized by involuntary, uncontrolled contractions, sustained myoclonus neither of which affects activities of daily living; and 3) marked or severe, characterized by unpredictable, uncontrolled paroxysms of spasm and involuntary clonus; these can throw the patient from a wheelchair and often the patient cannot lie in bed quietly; these patients have difficulties using a wheelchair, and transfers (for example: from bed to chair) are problematic.

Broadly speaking there are two groups of spasticity patients: cerebral origin spasticity (etiologies resulting from congenital or acquired injuries such as trauma (traumatic brain injury), anoxia (cerebral palsy), or stroke); spinal origin spasticity (etiologies include spinal cord injury and multiple sclerosis). Uncontrolled spasticity exacerbates physical disabilities, increases the cost of care, and profoundly impacts the quality of life for the patient and family.

Current Therapies for Spasticity

Mild to moderate spasticity is medically managed with the available treatments. Little to no data are available with respect to waning of efficacy or progression of the spasticity to more severe forms. With prolonged marked spasticity, contractures (static muscle shortening due to chronic spasm) may develop so that neither lying nor sitting occurs without undue pressure on bony prominences which lead to chronic pressure sores.

As the severity of the spasticity is a continuum, so are the therapies. Spasticity may not require treatment until it becomes painful, bothersome to the patient, or interferes with the activities of daily living. Existing treatments for spasticity may be categorized as systemic or locally acting.

Systemic Oral Medications

These are dantrolene (interferes with the excitation-contraction coupling mechanism by interfering with Ca⁺⁺ (dantirum), baclofen (GABAB agonist, lioresal), diazepam (GABA agonist, valium), tizanidine hydrochloride (β₂-agonist, zanaflex). Back-up medication is the α-agonist, clonidine.

Locally Acting Treatments

Locally acting treatments include intrathecal baclofen, surgical or chemical rhizotomy, and nerve motor point blocks.

Intrathecal Baclofen

Oral Baclofen is associated with undesirable side effects, however, Baclofen can be delivered to the subarachnoid space attached to a subcutaneous pump. Intrathecal baclofen is a convenient therapy and this form of drug delivery poses fewer central side effects. Further, intrathecal baclofen has shown to reduce spasticity, improve functional capabilities, and increases functional range of passive movement.

Surgical Intervention

This category includes rhizotomy, which has been most successful in the treatment of spasticity in children with cerebral palsy. In elderly patients that may have stroke induced spasticity, rhizotomy is uncommon and virtually not considered. Another surgical procedure, tendon lengthening, can be considered in those patients in which the lower extremities are affected. This procedure can be considered in those stroke patients who have developed spasticity.

Chemical Rhizotomy

Chemodenervation is performed via injections of phenol (or ethanol) or botulinum toxin. In phenol injections, there is neurolysis of the motor nerve. This nerve block technique is useful for motor neuron associated spasticity, and is generally avoided in cases where sensory and motor neurons are hyperactive. The improvement of spasticity after phenol injections may last for a few weeks to years. Botulinum toxin (BTX) injection into motor neurons has proven useful in the treatment of spasticity. This potent neurotoxin isolated from Clostrium botulinum, acts by binding to receptors at the neuromuscular junctions. The binding to the type A toxin is highly specific. The deactivation of intracellular presynaptic vesicles to release acetylcholine in the synaptic cleft can re-establish normal muscle tone and contractility. Intramuscular delivery of BTX has the advantages of lack of sensory effects, lack of caustic chemicals such as phenol, ability to target specific muscle groups through the use of electromyography, and an ability to weaken muscles in a graded fashion.

Limitations of Current Therapies for Spasticity: Efficacy and Toxicity

Systemic Local Medications

With the exception of dantrolene (which acts on directly on muscle), all of the other oral medications act on the central nervous system and there are unwanted effects from the medications, i.e. drowsiness and confusion. Dantrolene and baclofen may cause hepatotoxicity, and dantrolene may cause weakness in other muscle groups. Further, the systemic treatments are highly nonselective. As listed above, there are some indications that these oral medications are less likely reduce the spasticity; outcomes of oral medications in the treatment of cerebral origin spasticity are poor as compared to good outcomes in patients with spinal origin spasticity. Often combination regimens are used to attempt to curb the myoclonus.

Locally Acting Treatments

Intrathecal Baclofen—The limitations of this method of delivery are numerous: pump failure, infection, catheter migration, and the need to refill the reservoir. The half-life for ITB is 4-5 hours, and the pump must be refilled at least every 90 days.

Chemodenervation this technique is dependent on the proficiency of the surgeon and the accuracy of motor stimulation electromyography (EMG). Phenol injection close to a sensory nerve can result in causalgia due to injury of the myelin sheath of the sensory nerve.

BTX— There are studies that demonstrate a resistance to the toxin, these studies have shown that an antibody titer to the toxin prevents full potency.

Impact of Pharmacogenomics on Drug Development for Spasticity

As described above, there is evidence to suggest that there are efficacy and safety differences to drug therapy in the spasticity patient population. Although not all of these responses may be attributable to genotypic differences, it is expected that if stratification based upon genotype were performed, a reasonable correlation between drug response and genotype may become obvious. As described below, there are gene pathways that are involved with current drug therapy and those that may be potentially involved in the future. As described in the Detailed Description, methods for the identification of candidate genes and gene pathways, stratification, clinical trial design, and implementation of genotyping for appropriate medical management of a given disease is easily translated for spasticity. As described below in section V. below there are likely gene pathways as are those that are outlined in the gene pathway table, Table 1, and the gene pathway/indication matrix table, Table 2.

Optimization of GABAergic or ion channel modulation mediated therapy of spasticity further demonstrates the utility of selection of a potential spasticity patient that has a predisposing genotype in which selective antispasticity or agents are more effective and or are more safe. In considering an optimization protocol, one could potentially predetermine variance or variances within the GABAergic receptor, ion channel or ion channel mediated mechanisms of neurotransmission, or GABAergic receptor mediated intracellular mechanism of action that is preeminently responsible for drug response. By embarking on the previously described gene pathway approach, it is technically feasible to determine the relevant genes within such a targeted drug development program for spasticity.

A sample of therapies approved or in development for preventing or treating the progression of symptoms of spasticity currently known in the art is shown in table 17. In this table, the candidate therapeutics were sorted and listed by mechanism of action. Further, the product name, the pharmacologic mechanism of action, chemical name (if specified), and the indication is listed as well.

Description of Mechanism of Action Hypotheses for Future Drug Development for Spasticity

Although the exact mechanism of neurodegeneration-induced spasticity is unknown, the pathophysiology centers on the inadequate release of the inhibitory neurotransmitter, GABA within the spinal cord. Cerebral damage or localized damage within the spinal cord can influence the descending neurons that normally release GABA. However, the afferent input to the spinal cord from the muscle spindles is unaffected causing a relative increase of excitatory neurotransmitters, particularly glutamate. The consequence is excessive stimulation of the alpha motor neurons resulting in spasticity. Spasticity arising from cerebral damage may only affect certain modulatory inhibitory signals resulting in a variability of spasticity within each and among patients. Since all muscle groups may not be affected equally, management may be complicated.

Spastic paresis or spastic dystonia appear to arise from an imbalance of inhibition and excitation occurring at the level of the motor neuron. The most basic component is the abnormal intraspinal response to sensory input. Since modulation of the local spinal cord activity (peripheral segmental reflex arcs and the anterior horn cells) occurs via the descending pathways, loss of the GABA interneurons can affect the balance of excitation/inhibition and leads to hyperexcitable cells that result in an increase in activity of by the extrafusal muscle fibers.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with spasticity based upon genotype. Current pathways that may have involvement in the therapeutic benefit of epilepsy include glutaminergic, adrenergic, cholinergic, GABAergic, calcium channel, mitochondrial maintenance, adhesion, and myelination gene pathways that are listed in Tables 1, 3 and 4. One skilled in the art would be able to identify these pathway specific gene or genes that may be involved in the manifestation of spasticity, are likely candidate targets for novel therapeutic approaches, or are involved in mediating patient population differences in drug response to therapies for spasticity.

G. Ischemic Cerebrovascular Disease

Description of Stroke

Ischemic cerebrovascular disease is a result of an imbalance of the oxygen supply and the oxygen demand of brain tissue. Stroke is a series of clinical manifestations of reduction of blood supply to the cerebrovascular bed. The signs and symptoms may be complex and depend on the location and extent of the infarct. Ischemic cerebrovascular disease is divided into thrombotic and hemorrhagic stroke. There are approximately 700,000 individuals who suffer a first time stroke, approximately 160,000 will die, and the rest will join the 1 million Americans that have residual permanent disability. Stroke is the leading cause of long-term disability in the U.S. and is estimated to impose an annual cost of $25 billion dollars.

Thrombotic Strokes

Strokes are the result of reduced blood flow supplied by one or more or of the major cerebral arteries. Blockage or reduction of blood volume to these main arteries manifests as identifiable neurological symptoms. For example, occlusion of the middle cerebral artery results in contralateral hemiparesis, expressive aphasia, anosognosia and spatial disorientation, contralateral inferior quadrantanopsia, contralateral hemiparesis, sensory loss, contralateral homonymous hemianopsia, or superior quadrantanopsia. Blockage or reduction of the inner carotid artery, anterior cerebral artery, vertebral or basilar arteries, or the posterior artery can result in similarly clinically distinct neurological symptoms.

Transient ischemic attacks (TIA) are similar to a thrombotic stroke in that neurological deficit lasts for a brief period and is generally treated with potent platelet aggregation inhibitors.

Thrombotic strokes are the result of focal blockage of one or more of the cerebral arteries or branches resulting in neurological signs and symptoms lasting greater than one hour. Artherosclerotic plaques in extracranial or intracranial arteries cause approximately two thirds of thrombotic strokes. Embolization, stenosis, or occlusion of one or more of the cerebral arteries or branches may cause thrombotic strokes. Emboli can be of cardiac origin (e.g. mural thrombi, valvular heart disease, arrythmias (atrial fibrillation), cardiac myxoma, and paradoxical emboli (venous origin). Focal ischemia may also be the result of inflammation and necrosis of extracranial or intracranial blood vessels, i.e. vasculitides (e.g. primary cerebral arteritis, giant cell vasculitis, infectious vasculitis) or the result of hematologic abnormalities (hemoglobinopathy, hyperviscosity syndrome, hypercoagulable states, protein C or S deficiency, the presence of antiphospholipid antibodies). Strokes may be drug related, for example illicit drugs (cocaine, “crack”, amphetamines, lysergic acid, phencyclidine, methylphenidate, sympathomimetics, heroin, and pentazocine), ethanol, and oral contraceptives. Lastly there are other diseases that may predispose an individual to a stroke, for example fibromuscular dysplasia, arterial dissection, homocystinuria, migraine, subarachnoid hemorrhage, vasospasm, emboli of other origin (fat, bone, and air), and moyamoya.

Hemorrhagic Strokes

Approximately 20% of all strokes are the result of intracranial hemorrhage. Approximately half of these cases are into the subarachnoid space and the other half directly in the cerebral tissue. The acute rise in intracerebral pressure generally results in loss of consciousness and many die of cerebral herniation. Similar to thrombotic strokes, hemorrhagic strokes can be considered diffuse or focal, depending on the extent of the vessel disruption. Causes of spontaneous intracranial hemorrhage include arterial aneurysms (berry aneurysms, fusiform aneurysm, mycotic aneurysm, and aneurysm with vasculitis), cerebrovascular malformations, hypertensive-artherosclerotic hemorrhage, hemorrhage into a brain tumor, systemic bleeding diatheses, hemorrhage with vasculopathies, hemmorhage with intracranial venous infarction. Subarachnoid hemorrhage is caused by rupture of surface arteries (aneurysms, vascular formations, head trauma) with blood limited to the cerebrospinal fluid space between the pial and the arachnoid membranes.

Current Therapies for Stroke

If a hemorrhagic stroke is clear on the CCT, gradual reduction of systemic BP is achieved by standard vascular dilatation medications. Angiography can be useful to identify the source of the hemorrhage. Surgical management of the hemorrhage may be required.

If an ischemic stroke is identified and focal neurological impairments subside over time, a transient ischemic attack (TIA) is suspected. TIA has a high rate of recurrent stroke within a short time frame. Platelet aggregation inhibition is standard therapy; aspirin or ticlopidine. Ticlopidine is associated with neutropenia and agranulocytosis which may be life threatening. Because of these severe side effects, Ticlopidine is reserved for patients who are intolerant to aspirin therapy.

If angiographic review a clearly defined clot is detected, TIA may be surgically treated with endarterectomy.

For the treatment of thrombotic or embolic strokes, each case is independently assessed for surgical management or anticoagulant therapy. The success of thrombotic therapy, e.g. tissue plasminogen activator (tPA), streptokinase, urokinase, relies on timely reperfusion. The therapeutic window for tPA has been shown to be within three hours of onset of symptoms. Hypothermia has been shown to decrease mortality and improve outcomes. Hyperthermia has been shown to worsen both mortality rates and outcomes.

Significant neurologic improvement has been shown to occur within the first three months after stroke symptoms. A clear focus on intensive rehabilitation during this critical time frame has been shown to enhance the eventual outcome for survivors of stroke.

Limitations of Current Therapies for Stroke

The single most limiting factor of stroke therapy is the rapid identification of stroke symptoms and urgency of intervention within a short time.

Limitations of Stroke Therapy Due to Low Efficacy and Deleterious Side Effects

Guidelines for the use of tPA in acute ischemic stroke call for the administration of the thrombolytic agents within the first three hours from the onset of symptoms. After three hours four probable deleterious effects have been proven in animal studies and are as follows: 1) cerebral and extracerebral hemorrhage, 2) reperfusion injury, 3) fragmentation of clots, and 4) reocclusion of reperfused vessels.

In both animal models and in humans, reperfusion therapy must be administered within three hours of symptom onset. After three hours deleterious reperfusion injury may occur. Mortality at three months was 17% in the tPA group and 21% in the placebo group (p=0.30). Tissue plasminogen activator (tPA), streptokinase, heparin, and urokinase have specific restrictions: tPA has a 6% rate of cerebral hemorrhage; streptokinase is generally not used for thrombotic strokes because of serious side effects and limited quantifiable efficacy, urokinase is generally delivered near the site of the clot or obstruction. Factors influencing the best medical treatment of ischemic stroke must weigh the benefits and limitations of each of these therapies.

Impact of Genotyping on Drug Development for Stroke

As described above, there is evidence to suggest that there are efficacy and safety differences to drug therapy in the stroke patient population. Although not all of these responses may be attributable to genotypic differences, it is expected that if stratification based upon genotype were performed, a reasonable correlation between drug response and genotype may become obvious. As described below, there are gene pathways that are involved with current drug therapy and those that may be potentially involved in the future. As described in the Detailed Description, methods for the identification of candidate genes and gene pathways, stratification, clinical trial design, and implementation of genotyping for appropriate medical management of a given disease is easily translated for stroke patients. As described below in section V. below there are likely gene pathways as are those that are outlined in the gene pathway Table 1 and matrix Table 2.

A sample of therapies approved or in development for preventing or treating the progression of symptoms of stroke currently known in the art is shown in Table 18. In this table, the candidate therapeutics were sorted and listed by mechanism of action. Further, the product name, the pharmacologic mechanism of action, chemical name (if specified), and the indication is listed as well.

Mechanism of Action Hypotheses for Novel Therapies for Stroke: Utility of Genotyping

There are two categories of genotyping that provided insight on the selection of candidate genes for polymorphic genotypic studies of drug response. One set of likely candidates come from disease etiology or linkage studies. These data may provide input on the genetic etiology or aberrant mechanisms of strokes. Another set are those genes involved in the biochemical or molecular mechanisms of drugs, agents, or candidate therapeutic interventions.

Genes Involved in the Etiology of Stroke

Studies have demonstrated that there is a genetic component to thrombotic stroke. These genetic factors may predispose by an individual to thrombotic stroke by inheriting one or more of the following 1) low threshold for aberrant formation of artherosclerotic plaques in intracranial blood vessels; 2) traits that underlie certain specific etiology of stroke; and 3) a disease, disorder, or pathophysiologic process of the CNS in which there are associated molecular or structural disturbances that predispose individuals to strokes. These genetic influences mediating stroke may be candidates for genotyping assays and directly linked to pharmacogenomic programs.

Genes Involved in the Mechanism of Drug Action

There are also the biochemical, or molecular mechanisms of drug or candidate therapeutic action that may affect drug action. As described above there is an urgent need for the discovery and development of therapeutic alternatives for the medical management of strokes in which therapy commences beyond the therapeutic windows of thrombolytics.

Recent research and development programs have included the following pathways: 1) glutamate neurotransmitter pathway has been implicated in aberrant excitatory neurotransmission; 2) inflammation is a mechanism that may lead to profound neural cell loss, 3) carnosine pathway, 4) cell adhesion pathways, 5) oxidative stress pathways, 6) growth factor mediated differentiation and rescue of ischemic tissue, and protein maturation and degradation.

Ischemic Penumbra, Site of Infarct-Tissue at Risk

Ischemic penumbra is the tissue immediately adjacent to the infarct zone that is viable and morphologically intact but functionally impaired due to the restricted blood flow. Once the blood flow decreases to a certain threshold, this penumbra tissue can be classified as “misery-perfused” because oxygen consumption is preserved and increased oxygen extraction occurs. Ischemic penumbra is, thus, a dynamic process of impaired perfusion and unstable energy metabolism. Since necrosis naturally follows the continued oxygen deprivation, it has been reported that final cerebral infarct size is infarct zone plus the unrecoverable penumbra.

Functional imaging of the cerebral infarct can detect the penumbra tissue, and in some reports the penumbra tissue can be identified up to 48 hours. There is controversy whether the penumbra tissue can be rescued and what is the appropriate time from symptom onset to rescue by reperfusion. Rescue and time to rescue by reperfusion is dependent on the extent of occlusion and severity of metabolic disturbances. Based upon the hypothesis that early, immediate reperfusion can restore blood flow, the therapeutic window for successful intervention to restore the metabolic alterations has been postulated and proven to be within the first three hours from symptom onset. Other therapies include restoration of the cytokine, neurotransmitter, and Ca⁺² concentrations within the infarct zone (see therapy for stroke below).

Since the therapeutic window for victims of stroke is narrow and the debilitating effects of an ischemic stroke can be both costly and severely impact health-related quality of life, there is demand for candidate therapeutic interventions that can halt, retard, prevent neural destruction. Furthermore, there is a demand to develop further candidate therapeutic interventions that can assist in the rehabilitation and ultimately improve the health-related quality of life indices.

Advantages of Pharmacogenomic Clinical Development of Novel Candidate Therapeutic Interventions for Neurologic and Psychiatric Disease

The evidence that a variance in a gene involved in a pathway that affects drug response, indicates and supports the theory that there is a likelihood that other genes have similar qualities to various degrees. As drug research and development proceeds to identify more lead candidate therapeutic interventions for neurologic and psychiatric disease, there is possible utility in stratifying patients based upon their genotype for these yet to be correlated variances. Further, as described in the Detailed Description, methods for the identification of candidate genes and gene pathways, stratification, clinical trial design, and implementation of genotyping for appropriate medical management of a given disease is easily translated for patients with neurologic and psychiatric disease. As described below there are likely gene pathways as are those that are outlined in the gene pathway Table 1 and the matrix Table 2.

The advantages of a clinical research and drug development program that include the use of polymorphic genotyping for the stratification of patients for the appropriate selection of candidate therapeutic intervention includes 1) identification of patients that may respond earlier to therapy, 2) identification of the primary gene and relevant polymorphic variance that directly affects efficacy, safety, or both, 3) identification of pathophysiologic relevant variance or variances and potential therapies affecting those allelic genotypes or haplotypes, and 4) identification of allelic variances or haplotypes in genes that indirectly affects efficacy, safety or both.

Based upon these advantages, designing and performing a clinical trial, either prospective or retrospective, which includes a genotype stratification arm will incorporate analysis of clinical outcomes and potential genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of anxiety.

By identifying subsets of patients diagnosed with anxiety that respond earlier to agents, optimal candidate therapeutic interventions may reduce the lag time prior to relief of psychiatric symptoms. Appropriate genotyping and correlation to dosing regimen would be beneficial to the patient, caregivers, medical personnel, and the patient's loved ones.

As an example of identification of the primary gene and relevant polymorphic variance that directly affects efficacy, safety, or both one could select a gene pathway as described in the Detailed Description, and determine the effect of genetic polymorphism and therapy efficacy, safety, or both within that given pathway. By embarking on the previously described gene pathway approach, it is technically feasible to determine the relevant genes within such a targeted drug development program for neurologic or psychiatric disease.

Identification of pathophysiologic relevant variance or variances and potential therapies affecting those allelic genotypes or haplotypes will speed the drug development. There is a need for therapies that are targeted to the disease and symptom management with limited or no undesirable side effects. Identification of a specific variance or variances within genes involved in the pathophysiologic manifestation of anxiety and specific genetic polymorphisms of these critical genes can assist the development of novel anxiolytic agents and the identification of those patients that may best benefit from therapy of these candidate therapeutic alternatives.

By identifying allelic variances or haplotypes in genes that indirectly affects efficacy, safety or both one could target specific secondary drug or agent therapeutic actions that affect the overall therapeutic action of conventional, atypical, or novel action.

In Tables 3 and 4, there is a listing of candidate genes and specific single nucleotide polymorphisms that may be critical for the identification and stratification of an anxiety patient population based upon genotype. In Table 1 one skilled in the art would be able to identify these pathway specific genes or other genes listed in Table 1 that may be involved in the manifestation of neurologic or psychiatric disease or are likely candidate targets for therapeutic approaches described in this invention.

A sample of therapies approved or in development for preventing or treating the progression of symptoms of neurologic and psychiatric disease currently known in the art is shown in Tables 5-14, 16, 17, 18. In these tables, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

For each of the described neurologic or psychiatric disease indications one skilled in the art can identify novel candidate therapeutic interventions that may be used to treat the disease or symptoms and/or proceed with a regimen of palliative care. For compounds that have yet to achieve approval, or are still in development one skilled in the art can determine those candidate therapeutic interventions that may be of therapeutic benefit.

Exemplary Compounds in Development for Neurological or Psychiatric Disease

There are many sources for obtaining information on drugs approved for human therapeutic use an for those compounds under clinical or preclinical investigation, as well as for compounds which have been identified as having a particular pharmacological activity. For products, which have been approved, the PDR contains a listing of the package inserts for all of the products available for human therapeutic intervention. The Merck Index can be used as an additional text to supplement information gathered on the candidate therapeutic interventions. For products that are under clinical or preclinical development, there are databases cataloging information on those candidate therapeutic interventions. Generally that information includes aspects of the drug development process, such as phase of development, identified therapeutic indications, name of manufacturer, mechanistic and pharmacological activities of the product. These databases are available for a fee, and include: PharmaProjects and R&D Focus. One skilled in the art can readily utilize these sources to determine appropriate candidate therapeutic intervention for the identified disease, disorder or condition.

Since there are a large number of candidate therapeutic interventions that are either approved for human therapeutic use or under clinical or preclinical investigation, one skilled in the art could search through publicly available or fee-for-access databases for interventions that may be of therapeutic benefit for a particular disease, disorder, or condition, and determine whether variances in particular genes correlate with interpatient variation in response to one or more of those therapeutic interventions. An example of the results of such searching is provided in Tables 5-14, 16, 17, 18. In these tables, the disease, disorder or condition is listed. In order to generate a table or other compendium of information as listed in the table, one skilled in the art can search, for example, in databases for products having the indication “schizophrenia”. Alternatively, one can search for alternative indications or co-morbidities, e.g., pyschoses, neuroleptic, neurological to arrive at a more complete list of the available products. In the table, the candidate therapeutics were sorted and listed by pharmacologic mechanism of action (action). Further, the product name, chemical name (if specified), as well as the indication considered for clinical development. If the candidate therapeutic interventions are approved for therapeutic use, then one skilled in the art can obtain dosing, adverse events, pharmacologic parameters (both pharmacokinetic and pharmacodynamic), and clinical data or information by referring to the PDR. If the candidate therapeutic intervention are in clinical or preclinical stages of drug development, then one skilled in the art would gather data on dosing, adverse events, pharmacologic parameters (both pharmacokinetic and pharmacodynamic), and clinical data or information for the drug or product sponsor. In both cases, selection of a candidate therapeutic intervention for retrospective or prospective pharmacogenetic clinical studies would use an analysis of the likelihood that there is a phenomenological or statistical support for the review of the data to ascertain whether the candidate therapeutic intervention (approved or in development) efficacy or safety profiles can be grouped based upon the individual's genotype or phenotype. In this way, a gene or genes selected, e.g., from a pathway involving the cellular or more broadly the pharmacological mechanism of actions, can be identified and genotyping can be performed in order to determine the allelic variance, variances, for haplotype. Further, one could group the individuals by such genetic variances and further by the therapeutic outcome determinants.

Pharmacogenomics studies for these drugs, as well as other agents, drugs, compounds or candidate therapeutic interventions, can be performed by identifying genes that are involved in the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with neurologic or psychiatric disease based upon genotype. Current pathways that may have involvement in the therapeutic benefit of neurologic or psychiatric disease are listed as gene pathways and are listed in Tables 1, 2, 3 and 4. One skilled in the art would be able to identify these pathway specific gene or genes that may be involved in the manifestation of the described neurological or psychiatric disease, are likely candidate targets for novel therapeutic approaches, or are involved in mediating patient population differences in drug response to therapies for neurological or psychiatric disease described in the present invention.

As indicated in the Summary above, certain aspects of the present invention typically involve the following process, which need not occur separately or in the order stated. Not all of these described processes must be present in a particular method, or need be performed by a single entity or organization or person. Additionally, if certain of the information is available from other sources, that information can be utilized in the present invention. The processes are as follows: a) variability between patients in the response to a particular treatment is observed; b) at least a portion of the variable response is correlated with the presence or absence of at least one variance in at least one gene; c) an analytical or diagnostic test is provided to determine the presence or absence of the at least one variance in individual patients; d) the presence or absence of the variance or variances is used to select a patient for a treatment or to select a treatment for a patient, or the variance information is used in other methods described herein.

A. Identification of Interpatient Variability in Response to a Treatment

Interpatient variability is the rule, not the exception, in clinical therapeutics. One of the best sources of information on interpatient variability is the nurses and physicians supervising the clinical trial who accumulate a body of first hand observations of physiological responses to the drug in different normal subjects or patients. Evidence of interpatient variation in response can also be measured statistically, and may be best assessed by descriptive statistical measures that examine variation in response (beneficial or adverse) across a large number of subjects, including in different patient subgroups (men vs. women; whites vs. blacks; Northern Europeans vs. Southern Europeans, etc.).

In accord with the other portions of this description, the present invention concerns DNA sequence variances that can affect one or more of:

i. The susceptibility of individuals to a disease;

ii. The course or natural history of a disease;

iii. The response of a patient with a disease to a medical intervention, such as, for example, a drug, a biologic substance, physical energy such as radiation therapy, or a specific dietary regimen. (The terms ‘drug’, ‘compound’ or ‘treatment’ as used herein may refer to any of the foregoing medical interventions.) The ability to predict either beneficial or detrimental responses is medically useful.

Thus variation in any of these three parameters may constitute the basis for initiating a pharmacogenetic study directed to the identification of the genetic sources of interpatient variation. The effect of a DNA sequence variance or variances on disease susceptibility or natural history (i and ii, above) are of particular interest as the variances can be used to define patient subsets which behave differently in response to medical interventions such as those described in (iii). The methods of this invention are also useful in a clinical development program where there is not yet evidence of interpatient variation (perhaps because the compound is just entering clinical trials) but such variation in response can be reliably anticipated. It is more economical to design pharmacogenetic studies from the beginning of a clinical development program than to start at a later stage when the costs of any delay are likely to be high given the resources typically committed to such a program.

In other words, a variance can be useful for customizing medical therapy at least for either of two reasons. First, the variance may be associated with a specific disease subset that behaves differently with respect to one or more therapeutic interventions (i and ii above); second, the variance may affect response to a specific therapeutic intervention (iii above). Consider for exemplary purposes pharmacological therapeutic interventions. In the first case, there may be no effect of a particular gene sequence variance on the observable pharmacological action of a drug, yet the disease subsets defined by the variance or variances differ in their response to the drug because, for example, the drug acts on a pathway that is more relevant to disease pathophysiology in one variance-defined patient subset than in another variance-defined patient subset. The second type of useful gene sequence variance affects the pharmacological action of a drug or other treatment. Effects on pharmacological responses fall generally into two categories; pharmacokinetic and pharmacodynamic effects. These effects have been defined as follows in Goodman and Gilman's Pharmacologic Basis of Therapeutics (ninth edition, McGraw Hill, New York, 1986): “Pharmacokinetics” deals with the absorption, distribution, biotransformations and excretion of drugs. The study of the biochemical and physiological effects of drugs and their mechanisms of action is termed “pharmacodynamics.”

Useful gene sequence variances for this invention can be described as variances which partition patients into two or more groups that respond differently to a therapy or that correlate with differences in disease susceptibility or progression, regardless of the reason for the difference, and regardless of whether the reason for the difference is known. The latter is true because it is possible, with genetic methods, to establish reliable associations even in the absence of a pathophysiological hypothesis linking a gene to a phenotype, such as a pharmacological response, disease susceptibility or disease prognosis.

B. Identification of Specific Genes and Correlation of Variances in Those Genes with Response to Treatment of Diseases or Conditions

It is useful to identify particular genes which do or are likely to mediate the efficacy or safety of a treatment method for a disease or condition, particularly in view of the large number of genes which have been identified and which continue to be identified in humans. As is further discussed in section C below, this correlation can proceed by different paths. One exemplary method utilizes prior information on the pharmacology or pharmacokinetics or pharmacodynamics of a treatment method, e.g., the action of a drug, which indicates that a particular gene is, or is likely to be, involved in the action of the treatment method, and further suggests that variances in the gene may contribute to variable response to the treatment method. For example if a compound is known to be glucuronidated then a glucuronyltransferase is likely involved. If the compound is a phenol, the likely glucuronyltransferase is UGT1 (either the UGT1*1 or UGT1*6 transcripts, both of which catalyze the conjugation of planar phenols with glucuronic acid). Similar inferences can be made for many other biotransformation reactions.

Alternatively, if such information is not known, variances in a gene can be correlated empirically with treatment response. In this method, variances in a gene which exist in a population can be identified. The presence of the different variances or haplotypes in individuals of a study group, which is preferably representative of a population or populations of known geographic, ethnic and/or racial background, is determined. This variance information is then correlated with treatment response of the various individuals as an indication that genetic variability in the gene is at least partially responsible for differential treatment response. It may be useful to independently analyze variances in the different geographic, ethnic and/or racial groups as the presence of different genetic variances in these groups (i.e. different genetic background) may influence the effect of a specific variance. That is, there may be a gene×gene interaction involving one unstudied gene, however the indicated demographic variables may act as a surrogate for the unstudied allele. Statistical measures known to those skilled in the art are preferably used to measure the fraction of interpatient variation attributable to any one variance, or to measure the response rates in different subgroups defined genetically or defined by some combination of genetic, demographic and clinical criteria.

Useful methods for identifying genes relevant to the pharmacological action of a drug or other treatment are known to those skilled in the art, and include review of the scientific literature combined with inteferential or deductive reasoning that one skilled in the art of molecular pharmacology and molecular biology would be capable of; large scale analysis of gene expression in cells treated with the drug compared to control cells; large scale analysis of the protein expression pattern in treated vs. untreated cells, or the use of techniques for identification of interacting proteins or ligand-protein interactions, such as yeast two-hybrid systems.

C. Development of a Diagnostic Test to Determine Variance Status

In accordance with the description in the Summary above, the present invention generally concerns the identification of variances in genes which are indicative of the effectiveness of a treatment in a patient. The identification of specific variances, in effect, can be used as a diagnostic or prognostic test. Correlation of treatment efficacy and/or toxicity with particular genes and gene families or pathways is provided in Stanton et al., U.S. Provisional Application 60/093,484, filed Jul. 20, 1998, entitled GENE SEQUENCE VARIANCES WITH UTILITY IN DETERMINING THE TREATMENT OF DISEASE (concerns the safety and efficacy of compounds active on folate or pyrimidine metabolism or action) and Stanton, U.S. Provisional Application No. 60/121,047, filed Feb. 22, 1999, entitled GENE SEQUENCE VARIANCES WITH UTILITY IN DETERMINING THE TREATMENT OF DISEASE (concerning Alzheimer's disease and other dementias and cognitive disorders), which are hereby incorporated by reference in their entireties including drawings.

Genes identified in the examples below and in the Tables and Figures can be used in the methods of the present invention. A variety of genes which the inventors realize may account for interpatient variation in response to treatments for neurological and psychiatric diseases, conditions, disorders, and/or the development of same are listed in Tables 1, 3, and 4. Gene sequence variances in said genes are particularly useful for aspects of the present invention.

Methods for diagnostic tests are well known in the art. Generally in this invention, the diagnostic test involves determining whether an individual has a variance or variant form of a gene that is involved in the disease or condition or the action of the drug or other treatment or effects of such treatment. Such a variance or variant form of the gene is preferably one of several different variances or forms of the gene that have been identified within the population and are known to be present at a certain frequency. In an exemplary method, the diagnostic test involves determining the sequence of at least one variance in at least one gene after amplifying a segment of said gene using a DNA amplification method such as the polymerase chain reaction (PCR). In this method DNA for analysis is obtained by amplifying a segment of DNA or RNA (generally after converting the RNA to cDNA) spanning one or more variances in the gene sequence. Preferably, the amplified segment is <500 bases in length, in an alternative embodiment the amplified segment is <100 bases in length, most preferably <45 bases in length.

In some cases it will be desirable to determine a haplotype instead of a genotype. In such a case the diagnostic test is performed by amplifying a segment of DNA or RNA (cDNA) spanning more than one variance in the gene sequence and preferably maintaining the phase of the variances on each allele. The term “phase” refers to the relationship of variances on a single chromosomal copy of the gene, such as the copy transmitted from the mother (maternal copy or maternal allele) or the father (paternal copy or paternal allele). The haplotyping test may take part in two phases, where first genotyping tests at two or more variant sites reveal which sites are heterozygous in each patient or normal subject. Subsequently the phase of the two or more variant sites can be determined. In performing a haplotyping test preferably the amplified segment is >500 bases in length, more preferably it is >1,000 bases in length, and most preferably it is >2,500 bases in length. One way of preserving phase is to amplify one strand in the PCR reaction. This can be done using one or a pair of oligonucleotide primers that terminate (i.e. have a 3′ end that stops) opposite the variant site, such that one primer is perfectly complementary to one variant form and the other primer is perfectly complementary to the other variant form. Other than the difference in the 3′ most nucleotide the two primers are identical (forming an allelic primer pair). Only one of the allelic primers is used in any PCR reaction, depending on which strand is being amplified. The primer for the opposite strand may also be an allelic primer, or it may prime from a non-polymorphic region of the template. This method exploits the requirement of most polymerases for perfect complementarity at the 3′ terminus of the primer in a primer-template complex. See, for example: Lo Y M, Patel P, Newton C R, Markham A F, Fleming K A and J S Wainscoat. (1991) Direct haplotype determination by double ARMS: specificity, sensitivity and genetic applications. Nucleic Acids Res July 11; 19(13):3561-7.

It is apparent that such diagnostic tests are performed after initial identification of variances within the gene, which allows selection of appropriate allele specific primers.

Diagnostic genetic tests useful for practicing this invention belong to two types: genotyping tests and haplotyping tests. A genotyping test simply provides the status of a variance or variances in a subject or patient. For example suppose nucleotide 150 of hypothetical gene X on an autosomal chromosome is an adenine (A) or a guanine (G) base. The possible genotypes in any individual are AA, AG or GG at nucleotide 150 of gene X.

In a haplotyping test there is at least one additional variance in gene X, say at nucleotide 810, which varies in the population as cytosine (C) or thymine (T). Thus a particular copy of gene X may have any of the following combinations of nucleotides at positions 150 and 810: 150A-810C, 150A-810T, 150G-810C or 150G-810T. Each of the four possibilities is a unique haplotype. If the two nucleotides interact in either RNA or protein, then knowing the haplotype can be important. The point of a haplotyping test is to determine the haplotypes present in a DNA or cDNA sample (e.g. from a patient). In the example provided there are only four possible haplotypes, but, depending on the number of variances in the gene and their distribution in human populations there may be three, four, five, six or more haplotypes at a given gene. The most useful haplotypes for this invention are those which occur commonly in the population being treated for a disease or condition. Preferably such haplotypes occur in at least 5% of the population, more preferably in at least 10%, still more preferably in at least 20% of the population and most preferably in at least 30% or more of the population. Conversely, when the goal of a pharmacogenetic program is to identify a relatively rare population that has an adverse reaction to a treatment, the most useful haplotypes may be rare haplotypes, which may occur in less than 5%, less than 2%, or even in less than 1% of the population. One skilled in the art will recognize that the frequency of the adverse reaction provides a useful guide to the likely frequency of salient causative haplotypes.

Based on the identification of variances or variant forms of a gene, a diagnostic test utilizing methods known in the art can be used to determine whether a particular form of the gene, containing specific variances or haplotypes, or combinations of variances and haplotypes, is present in at least one copy, one copy, or more than one copy in an individual. Such tests are commonly performed using DNA or RNA collected from blood, cells, tissue scrapings or other cellular materials, and can be performed by a variety of methods including, but not limited to, PCR based methods, hybridization with allele-specific probes, enzymatic mutation detection, chemical cleavage of mismatches, mass spectrometry or DNA sequencing, including minisequencing. Methods for haplotyping are described above. In particular embodiments, hybridization with allele specific probes can be conducted in two formats: (1) allele specific oligonucleotides bound to a solid phase (glass, silicon, nylon membranes) and the labeled sample in solution, as in many DNA chip applications, or (2) bound sample (often cloned DNA or PCR amplified DNA) and labeled oligonucleotides in solution (either allele specific or short—e.g. 7mers or 8mers—so as to allow sequencing by hybridization). Preferred methods for diagnosing testing of variances are described in four patent applications Stantori et al, entitled A METHOD FOR ANALYZING POLYNUCLEOTIDES, Ser. Nos. 09/394,467; 09/394,457; 09/394,774; and 09/394,387; all filed Sep. 10, 1999. The application of such diagnostic tests is possible after identification of variances that occur in the population. Diagnostic tests may involve a panel of variances from one or more genes, often on a solid support, which enables the simultaneous determination of more than one variance in one or more genes.

D. Use of Variance Status to Determine Treatment

The present disclosure describes exemplary gene sequence variances in genes identified in a gene table herein (e.g., Tables 3 and 4), and variant forms of these gene that may be determined using diagnostic tests. As indicated in the Summary, such a variance-based diagnostic test can be used to determine whether or not to administer a specific drug or other treatment to a patient for treatment of a disease or condition. Preferably such diagnostic tests are incorporated in texts such as are described in Clinical Diagnosis and Management by Laboratory Methods (19th Ed) by John B. Henry (Editor) W B Saunders Company, 1996; Clinical Laboratory Medicine: Clinical Application of Laboratory Data, (6th edition) by R. Ravel, Mosby-Year Book, 1995, or other medical textbooks including, without limitation, textbooks of medicine, laboratory medicine, therapeutics, pharmacy, pharmacology, nutrition, allopathic, homeopathic, and osteopathic medicine; preferably such a test is developed as a ‘home brew’ method by a certified diagnostic laboratory; most preferably such a diagnostic test is approved by regulatory authorities, e.g., by the U.S. Food and Drug Administration, and is incorporated in the label or insert for a therapeutic compound, as well as in the Physicians Desk Reference.

In such cases, the procedure for using the drug is restricted or limited on the basis of a diagnostic test for determining the presence of a variance or variant form of a gene. Alternatively the use of a genetic test may be advised as best medical practice, but not absolutely required, or it may be required in a subset of patients, e.g. those using one or more other drugs, or those with impaired liver or kidney function. The procedure that is dictated or recommended based on genotype may include the route of administration of the drug, the dosage form, dosage, schedule of administration or use with other drugs; any or all of these may require selecting or determination consistent with the results of the diagnostic test or a plurality of such tests. Preferably the use of such diagnostic tests to determine the procedure for administration of a drug is incorporated in a text such as those listed above, or medical textbooks, for example, textbooks of medicine, laboratory medicine, therapeutics, pharmacy, pharmacology, nutrition, allopathic, homeopathic, and osteopathic medicine. As previously stated, preferably such a diagnostic test or tests are required by regulatory authorities and are incorporated in the label or insert as well as the Physicians Desk Reference.

Variances and variant forms of genes useful in conjunction with treatment methods may be associated with the origin or the pathogenesis of a disease or condition. In many useful cases, the variant form of the gene is associated with a specific characteristic of the disease or condition that is the target of a treatment, most preferably response to specific drugs or other treatments. Examples of diseases or conditions ameliorable by the methods of this invention are identified in the Examples and tables below; in general treatment of disease with current methods, particularly drug treatment, always involves some unknown element (involving efficacy or toxicity or both) that can be reduced by appropriate diagnostic methods.

Alternatively, the gene is involved in drug action, and the variant forms of the gene are associated with variability in the action of the drug. For example, in some cases, one variant form of the gene is associated with the action of the drug such that the drug will be effective in an individual who inherits one or two copies of that form of the gene. Alternatively, a variant form of the gene is associated with the action of the drug such that the drug will be toxic or otherwise contra-indicated in an individual who inherits one or two copies of that form of the gene.

In accord with this invention, diagnostic tests for variances and variant forms of genes as described above can be used in clinical trials to demonstrate the safety and efficacy of a drug in a specific population. As a result, in the case of drugs which show variability in patient response correlated with the presence or absence of a variance or variances, it is preferable that such drug is approved for sale or use by regulatory agencies with the recommendation or requirement that a diagnostic test be performed for a specific variance or variant form of a gene which identifies specific populations in which the drug will be safe and/or effective. For example, the drug may be approved for sale or use by regulatory agencies with the specification that a diagnostic test be performed for a specific variance or variant form of a gene which identifies specific populations in which the drug will be toxic. Thus, approved use of the drug, or the procedure for use of the drug, can be limited by a diagnostic test for such variances or variant forms of a gene; or such a diagnostic test may be considered good medical practice, but not absolutely required for use of the drug.

As indicated, diagnostic tests for variances as described in this invention may be used in clinical trials to establish the safety and efficacy of a drug. Methods for such clinical trials are described below and/or are known in the art and are described in standard textbooks. For example, diagnostic tests for a specific variance or variant form of a gene may be incorporated in the clinical trial protocol as inclusion or exclusion criteria for enrollment in the trial, to allocate certain patients to treatment or control groups within the clinical trial or to assign patients to different treatment cohorts. Alternatively, diagnostic tests for specific variances may be performed on all patients within a clinical trial, and statistical analysis performed comparing and contrasting the efficacy or safety of a drug between individuals with different variances or variant forms of the gene or genes. Preferred embodiments involving clinical trials include the genetic stratification strategies, phases, statistical analyses, sizes, and other parameters as described herein.

Similarly, diagnostic tests for variances can be performed on groups of patients known to have efficacious responses to the drug to identify differences in the frequency of variances between responders and non-responders. Likewise, in other cases, diagnostic tests for variance are performed on groups of patients known to have toxic responses to the drug to identify differences in the frequency of the variance between those having adverse events and those not having adverse events. Such outlier analyses may be particularly useful if a limited number of patient samples are available for analysis. It is apparent that such clinical trials can be or are performed after identifying specific variances or variant forms of the gene in the population. In defining outliers it is useful to examine the distribution of responses in the placebo group; outliers should preferably have responses that exceed in magnitude the extreme responses in the placebo group.

The identification and confirmation of genetic variances is described in certain patents and patent applications. The description therein is useful in the identification of variances in the present invention. For example, a strategy for the development of anticancer agents having a high therapeutic index is described in Housman, International Application PCT/US/94 08473 and Housman, INHIBITORS OF ALTERNATIVE ALLELES OF GENES ENCODING PROTEINS VITAL FOR CELL VIABILITY OR CELL GROWTH AS A BASIS FOR CANCER THERAPEUTIC AGENTS, U.S. Pat. No. 5,702,890, issued Dec. 30, 1997, which are hereby incorporated by reference in their entireties. Also, a number of gene targets and associated variances are identified in Housman et al., U.S. patent application Ser. No. 09/045,053, entitled TARGET ALLELES FOR ALLELE-SPECIFIC DRUGS, filed Mar. 19, 1998, which is hereby incorporated by reference in its entirety, including drawings.

The described approach and techniques are applicable to a variety of other diseases, conditions, and/or treatments and to genes associated with the etiology and pathogenesis of such other diseases and conditions and the efficacy and safety of such other treatments.

Useful variances for this invention can be described generally as variances which partition patients into two or more groups that respond differently to a therapy (a therapeutic intervention), regardless of the reason for the difference, and regardless of whether the reason for the difference is known.

III. From Variance List to Clinical Trial: Identifying Genes and Gene Variances that Account for Variable Responses to Treatment

There are a variety of useful methods for identifying a subset of genes from a large set of candidate genes that should be prioritized for further investigation with respect to their influence on inter-individual variation in disease predisposition or response to a particular drug. These methods include for example, (1) searching the biomedical literature to identify genes relevant to a disease or the action of a drug, (2) screening the genes identified in step 1 for variances. A large set of exemplary variances are provided in Tables 3 and 4. Other methods include (3) using computational tools to predict the functional effects of variances in specific genes, (4) using in vitro or in vivo experiments to identify genes which may participate in the response to a drug or treatment, and to determine the variances which affect gene, RNA or protein function, and may therefore be important genetic variables affecting disease manifestations or drug response, and (5) retrospective or prospective clinical trials. Computational tools are described in U.S. patent application, Stanton et al., Ser. No. 09/300,747, filed Apr. 26, 1999, entitled GENE SEQUENCE VARIANCES WITH UTILITY IN DETERMINING THE TREATMENT OF DISEASE, and in Stanton et al., Ser. No. 09/419,705, filed Oct. 14, 1999, entitled VARIANCE SCANNING METHOD FOR IDENTIFYING GENE SEQUENCE VARIANCES, which are hereby incorporated by reference in their entireties, including drawings. Other methods are considered below in some detail.
(1) To begin, one preferably identifies, for a given treatment, a set of candidate genes that are likely to affect disease phenotype or drug response. This can be accomplished most efficiently by first assembling the relevant medical, pharmacological and biological data from available sources (e.g., public databases and publications). One skilled in the art can review the literature (textbooks, monographs, journal articles) and online sources (databases) to identify genes most relevant to the action of a specific drug or other treatment, particularly with respect to its utility for treating a specific disease, as this beneficially allows the set of genes to be analyzed ultimately in clinical trials to be reduced from an initial large set. Specific strategies for conducting such searches are described below. In some instances the literature may provide adequate information to select genes to be studied in a clinical trial, but in other cases additional experimental investigations of the sort described below will be preferable to maximize the likelihood that the salient genes and variances are moved forward into clinical studies. Specific genes relevant to understanding interpatient variation in response to treatments for major neurological and psychiatric diseases are listed in Table 1. In Table 2 preferred sets of genes for analysis of variable therapeutic response in specific diseases are highlighted. These genes are exemplary; they do not constitute a complete set of genes that may account for variation in clinical response. Experimental data are also useful in establishing a list of candidate genes, as described below.
(2) Having assembled a list of candidate genes generally the second step is to screen for variances in each candidate gene. Experimental and computational methods for variance detection are described in this invention, and tables of exemplary variances are provided (Tables 3, and 4) as well as methods for identifying additional variances and a written description of such possible additional variances in the cDNAs of genes that may affect drug action (see Stanton et al., application Ser. No. 09/300,747, filed Apr. 26, 1999, entitled GENE SEQUENCE VARIANCES WITH UTILITY IN DETERMINING THE TREATMENT OF DISEASE, incorporated in its entirety.
(3) Having identified variances in candidate genes the next step is to assess their likely contribution to clinical variation in patient response to therapy, preferably by using informatics-based approaches such as DNA and protein sequence analysis and protein modeling. The literature and informatics-based approaches provide the basis for prioritization of candidate genes, however it may in some cases be desirable to further narrow the list of candidate genes, or to measure experimentally the phenotype associated with specific variances or sets of variances (e.g. haplotypes).
(4) Thus, as a third step in candidate gene analysis, one skilled in the art may elect to perform in vitro or in vivo experiments to assess the functional importance of gene variances, using either biochemical or genetic tests. (Certain kinds of experiments—for example gene expression profiling and proteome analysis—may not only allow refinement of a candidate gene list but may also lead to identification of additional candidate genes.) Combination of two or all of the three above methods will provide sufficient information to narrow and prioritize the set of candidate genes and variances to a number that can be studied in a clinical trial with adequate statistical power.
(5) The fourth step is to design retrospective or prospective human clinical trials to test whether the identified allelic variance, variances, or haplotypes or combination thereof influence the efficacy or toxicity profiles for a given drug or other therapeutic intervention. It should be recognized that this fourth step is the crucial step in producing the type of data that would justify introducing a diagnostic test for at least one variance into clinical use. Thus while each of the above four steps are useful in particular instances of the invention, this final step is indispensable. Further guidance and examples of how to perform these five steps are provided below.
(6) A fifth (optional) step entails methods for using a genotyping test in the promotion and marketing of a treatment method. It is widely appreciated that there is a tendency in the pharmaceutical industry to develop many compounds for well established therapeutic targets. Examples include beta adrenergic blockers, hydroxymethylglutaryl (HMG) CoA reductase inhibitors (statins), dopamine D2 receptor antagonists and serotonin transporter inhibitors. Frequently the pharmacology of these compounds is quite similar in terms of efficacy and side effects. Therefore the marketing of one compound vs. other members of the class is a challenging problem for drug companies, and is reflected in the lesser success that late products typically achieve compared to the first and second approved products. It occurred to the inventors that genetic stratification can provide the basis for identifying a patient population with a superior response rate or improved safety to one member of a class of drugs, and that this information can be the basis for commercialization of that compound. Such a commercialization campaign can be directed at caregivers, particularly physicians, or at patients and their families, or both.
   1. Identification of Candidate Genes Relevant to the Action of a Drug

Practice of this invention will often begin with identification of a specific pharmaceutical product, for example a drug, that would benefit from improved efficacy or reduced toxicity or both, and the recognition that pharmacogenetic investigations as described herein provide a basis for achieving such improved characteristics. The question then becomes which genes and variances, such as those provided in this application in Tables 1, 3, and 4, would be most relevant to interpatient variation in response to the drug. As discussed above, the set of relevant genes includes both genes involved in the disease process and genes involved in the interaction of the patient and the treatment—for example genes involved in pharmacokinetic and pharmacodynamic action of a drug. The biological and biomedical literature and online databases provide useful guidance in selecting such genes. Specific guidance in the use of these resources is provided below.

Review the Literature and Online Sources

One way to find genes that affect response to a drug in a particular disease setting is to review the published literature and available online databases regarding the pathophysiology of the disease and the pharmacology of the drug. Literature or online sources can provide specific genes involved in the disease process or drug response, or describe biochemical pathways involving multiple genes, each of which may affect the disease process or drug response.

Alternatively, biochemical or pathological changes characteristic of the disease may be described; such information can be used by one skilled in the art to infer a set of genes that can account for the biochemical or pathologic changes. For example, to understand variation in response to a drug that modulates serotonin levels in a central nervous system (CNS) disorder associated with altered levels of serotonin one would preferably study, at a minimum, variances in genes responsible for serotonin biosynthesis, release from the cell, receptor binding, presynaptic reuptake, and degradation or metabolism. Genes responsible for each of these functions should be examined for variation that may account for interpatient differences in drug response or disease manifestations. As recognized by those skilled in the art, a comprehensive list of such genes can be obtained from textbooks, monographs and the literature.

There are several types of scientific information, described in some detail below, that are valuable for identifying a set of candidate genes to be investigated with respect to a specific disease and therapeutic intervention. First there is the medical literature, which provides basic information on disease pathophysiology and therapeutic interventions. A subset of this literature is devoted to specific description of pathologic conditions. Second there is the pharmacology literature, which will provide additional information on the mechanism of action of a drug (pharmacodynamics) as well as its principal routes of metabolic transformation (pharmacokinetics) and the responsible proteins. Third there is the biomedical literature (principally genetics, physiology, biochemistry and molecular biology), which provides more detailed information on metabolic pathways, protein structure and function and gene structure. Fourth, there are a variety of online databases that provide additional information on metabolic pathways, gene families, protein function and other subjects relevant to selecting a set of genes that are likely to affect the response to a treatment.

Medical Literature

A good starting place for information on molecular pathophysiology of a specific disease is a general medical textbook such as Harrison's Principles of Internal Medicine, 14th edition, (2 Vol Set) by A. S. Fauci, E. Braunwald, K. J. Isselbacher, et al. (editors), McGraw Hill, 1997, or Cecil Textbook of Medicine (20th Ed) by R. L. Cecil, F. Plum and J. C. Bennett (Editors) W B Saunders Co., 1996. For pediatric diseases texts such as Nelson Textbook of Pediatrics (15th edition) by R. E. Behrman, R. M. Kliegman, A. M. Arvin and W. E. Nelson (Editors), W B Saunders Co., 1995 or Oski's Principles and Practice of Pediatrics (3^{rd} Edition) by J. A. Mamillan & F. A. Oski Lippincott-Raven, 1999 are useful introductions. For obstetrical and gynecological disorders texts such as Williams Obstetrics (20th Ed) by F. G. Cunningham, N. F. Gant, P. C. McDonald et al. (Editors), Appleton & Lange, 1997 provide general information on disease pathophysiology. For psychiatric disorders texts such as the Comprehensive Textbook of Psychiatry, VI (2 Vols) by H. I. Kaplan and B. J. Sadock (Editors), Lippincott, Williams & Wilkins, 1995, or The American Psychiatric Press Textbook of Psychiatry (3^{rd} edition) by R. E. Hales, S. C. Yudofsky and J. A. Talbott (Editors) Amer Psychiatric Press, 1999 provide an overview of disease nosology, pathophysiological mechanisms and treatment regimens.

In addition to these general texts, there are a variety of more specialized medical texts that provide greater detail about specific disorders which can be utilized in developing a list of candidate genes and variances relevant to interpatient variation in response to a treatment. For example, within the field of medicine there are standard textbooks for each of the subspecialties. Some specific examples include:
Heart Disease: A Textbook of Cardiovascular Medicine (2 Volume set) by E. Braunwald (Editor), W B Saunders Co., 1996.
Hurst's the Heart, Arteries and Veins (9th Ed) (2 Vol Set) by R. W. Alexander, R. C. Schlant, V. Fuster, W. Alexander and E. H. Sonnenblick (Editors) McGraw Hill, 1998.
Principles of Neurology (6th edition) by R. D. Adams, M. Victor (editors), and A. H. Ropper (Contributor), McGraw Hill, 1996.
Sleisenger & Fordtran's Gastrointestinal and Liver Disease: Pathophysiology, Diagnosis, Management (6th edition) by M. Feldman, B. F. Scharschmidt and M. Sleisenger (Editors), W B Saunders Co., 1997.
Textbook of Rheumatology (5th edition) by W. N. Kelley, S. Ruddy, E. D. Harris Jr. and C. B. Sledge (Editors) (2 volume set) W B Saunders Co., 1997.
Williams Textbook of Endocrinology (9th edition) by J. D. Wilson, D. W. Foster, H. M. Kronenberg and Larsen (Editors), W B Saunders Co., 1998.
Wintrobe's Clinical Hematology (10th Ed) by G. R. Lee, J. Foerster (Editor) and J. Lukens (Editors) (2 Volumes) Lippincott, Williams & Wilkins, 1998.
Cancer: Principles &Practice of Oncology (5th edition) by V. T. Devita, S. A. Rosenberg and S. Hellman (editors), Lippincott-Raven Publishers, 1997.
Principles of Pulmonary Medicine (3rd edition) by S. E. Weinberger & J Fletcher (Editors), W B Saunders Co., 1998.
Diagnosis and Management of Renal Disease and Hypertension (2nd edition) by A. K. Mandal & J. C. Jennette (Editors), Carolina Academic Press, 1994. Massry & Glassock's Textbook of Nephrology (3rd edition) by S. G. Massry & R. J. Glassock (editors) Williams & Wilkins, 1995.
The Management of Pain by J. J. Bonica, Lea and Febiger, 1992
Ophthalmology by M. Yanoff & J. S. Duker, Mosby Year Book, 1998
Clinical Ophthalmology: A Systemic Approach by J. J. Kanski, Butterworth-Heineman, 1994. Essential Otolaryngology by J. K. Lee Appleton and Lange 1998.

In addition to these subspecialty texts there are many textbooks and monographs that concern more restricted disease areas, or specific diseases. Such books provide more extensive coverage of pathophysiologic mechanisms and therapeutic options. The number of such books is too great to provide examples for all but a few diseases, however one skilled in the art will be able to readily identify relevant texts. One simple way to search for relevant titles is to use the search engine of an online bookseller using the disease or drug (or the group of diseases or drugs to which they belong) as search terms. For example a search for asthma would turn up titles such as Asthma: Basic Mechanisms and Clinical Management (3rd edition) by P. J. Bames, I. W. Rodger and N. C. Thomson (Editors), Academic Press, 1998 and Airways and Vascular Remodeling in Asthma and Cardiovascular Disease: Implications for Therapeutic Intervention, by C. Page & J. Black (Editors), Academic Press, 1994.

Pathology Literature

In addition to medical texts there are texts that specifically address disease etiology and pathologic changes associated with disease. A good general pathology text is Robbins Pathologic Basis of Disease (6th edition) by R. S. Cotran, V. Kumar, T. Collins and S. L. Robbins, W B Saunders Co., 1998. Specialized pathology texts exist for each organ system and for specific diseases, similar to medical texts. These texts are useful sources of information for one skilled in the art for developing lists of genes that may account for some of the known pathologic changes in disease tissue. Exemplary texts are as follows:
Bone Marrow Pathology 2^{nd} edition, by B. J. Bain, I. Lampert. & D. Clark, Blackwell Science, 1996
Atlas of Renal Pathology by F. G. Silva, W.B. Saunders, 1999.
Fundamentals of Toxicologic Pathology by W. M. Haschek and C. G. Rousseaux, Academic Press, 1997.
Gastrointestinal Pathology by P. Chandrasoma, Appleton and Lange, 1998.
Ophthalmic Pathology with Clinical Correlations by J. Sassani, Lippincott-Raven, 1997.
Pathology of Bone and Joint Disorders by F. McCarthy, F. J. Frassica and A. Ross, W. B. Saunders, 1998.
Pulmonary Pathology by M. A. Grippi, Lippicott-Raven, 1995.
Neuropathology by D. Ellison, L. Chimelli, B. Harding, S. Love & J. Lowe, Mosby Year Book, 1997.
Greenfield's Neuropathology 6^{th} edition by J. G. Greenfield, P. L. Lantos & D. I. Graham, Edward Arnold, 1997.
   Pharmacology, Pharmacogenetics and Pharmacy Literature

There are also both general and specialized texts and monographs on pharmacology that provide data on pharmacokinetics and pharmacodynamics of drugs. The discussion of pharmacodynamics (mechanism of action of the drug) in such texts is often supported by a review of the biochemical pathway or pathways that are affected by the drug. Also, proteins related to the target protein are often listed; it is important to account for variation in such proteins as the related proteins may be involved in drug pharmacology. For example, there are 14 known serotonin receptors. Various pharmacological serotonin agonists or antagonists have different affinities for these different receptors. Variation in a specific receptor may affect the pharmacology not only of drugs targeted to that receptor, but also drugs that are principally agonists or antagonists of different receptors. Such compounds may produce different effects on two allelic forms of a non-targeted receptor; for example on variant form may bind the compound with higher affinity than the other, or a compound that is principally an antagonist for one allele may be a partial agonist for another allele. Thus genes encoding proteins structurally related to the target protein should be screened for variance in order to successfully realize the methods of the present invention. A good general pharmacology text is Goodman & Gilman's the Pharmacological Basis of Therapeutics (9th Ed) by J. G. Hardman, L. E. Limbird, P. B. Molinoff, R. W. Ruddon and A. G. Gilman (Editors) McGraw Hill, 1996. There are also texts that focus on the pharmacology of drugs for specific disease areas, or specific classes of drugs (e.g. natural products) or adverse drug interactions, among other subjects. Specific examples include:
The American Psychiatric Press Textbook of Psychopharmacology (2nd edition) by A. F. Schatzberg & C. B. Nemeroff (Editors), American Psychiatric Press, 1998. Essential Psychopharmacology: Neuroscientific Basis and Practical Applications by N. Muntner and S. M. Stahl, Cambridge Univ Press, 1996.
   There are also texts on pharmacogenetics which are particularly useful for identifying genes which may contribute to variable pharmacokinetic response. In addition there are texts on some of the major xenobiotic metabolizing proteins, such as the cytochrome P450 genes.
Pharmacogenetics of Drug Metabolism (International Encyclopedia of Pharmacology and Therapeutics) by Werner Kalow (Editor) Pergamon Press, 1992.
Genetic Factors in Drug Therapy: Clinical and Molecular Pharmacogenetics by D. A Price Evans, Cambridge Univ Press, 1993.
Pharmacogenetics (Oxford Monographs on Medical Genetics, 32) by W. W. Weber, Oxford Univ Press, 1997.
Cytochrome P450: Structure, Mechanism, and Biochemistry by P. R. Ortiz de Montellano (Editor), Plenum Publishing Corp, 1995.
Appleton & Lange's Review of Pharmacy, 6^{th} edition, (Appleton & Lange's Review Series) by G. D. Hall & B. S. Reiss, Appleton & Lange, 1997.
   Genetics, Biochemistry and Molecular Biology Literature

In addition to the medical, pathology, and pharmacology texts listed above there are several information sources that one skilled in the art will turn to for information on the genetic, physiologic, biochemical, and molecular biological aspects of the disease, disorder or condition or the effect of the therapeutic intervention on specific physiologic processes. The biomedical literature may include information on nonhuman organisms that is relevant to understanding the likely disease or pharmacological pathways in man.

Also provided below are illustrative texts which will aid in the identification of a pathway or pathways, and a gene or genes that may be relevant to interindividual variation in response to a therapy. Textbooks of biochemistry, genetics and physiology are often useful sources for such pathway information. In order to ascertain the appropriate methods to analyze the effects of an alleleic variance, variances, or haplotypes in vitro, one skilled in the art will review existing information on molecular biology, cell biology, genetics, biochemistry; and physiology. Such texts are useful sources for general and specific information on the genetic and biochemical processes involved in disease and in drug action, as well as experimental procedures that may be useful in performing in vitro research on an allelic variance, variances, or haplotye.

Texts on gene structure and function and RNA biochemistry will be useful in evaluating the consequences of variances that do not change the coding sequence (silent variances). Such variances may alter the interaction of RNA with proteins or other regulatory molecules affecting RNA processing, polyadenylation, or export.

Molecular and Cellular Biology

Molecular Cell Biology by H. Lodish, D. Baltimore, A. Berk, L. Zipurksy & J. Darnell, W H Freeman & Co., 1995.
Essentials of Molecular Biology, D. Freifelder and MalacinskiJones and Bartlett, 1993.
Genes and Genomes: A Changing Perspective, M. Singer and P. Berg, 1991. University Science Books
Gene Structure and Expression, J. D. Hawkins, 1996. Cambridge University Press
Molecular Biology of the Cell, 2nd edition, B. Alberts et al., Garland Publishing, 1994.
   Molecular Genetics
The Metabolic and Molecular Bases of Inherited Disease by C. R. Scriver, A. L. Beaudet, W. S. Sly (Editors), 7th edition, McGraw Hill, 1995
Genetics and Molecular Biology, R. Schleif, 1994. 2nd edition, Johns Hopkins University Press
Genetics, P. J. Russell, 1996. 4th edition, Harper Collins
An Introduction to Genetic Analysis, Griffiths et al. 1993. 5th edition, W.H. Freeman and Company
Understanding Genetics: A molecular approach, Rothwell, 1993. Wiley-Liss
   General Biochemistry
Biochemistry, L. Stryer, 1995. W.H. Freeman and Company
Biochemistry, D. Voet and J. G. Voet, 1995. John Wiley and Sons
Principles of Biochemistry, A. L. Lehninger, D. L. Nelson, and M. M. Cox, 1993. Worth Publishers
Biochemistry, G. Zubay, 1998. Wm. C. Brown Communications
Biochemistry, C. K. Mathews and K. E. van Holde, 1990. Benjamin/Cummings
   Transcription
Eukaryotic Transcription Factors, D. S. Latchman, 1995. Academic Press
Eukaryotic Gene Transcription, S. Goodbourn (ed.), 1996. Oxford University Press.
Transcription Factors and DNA Replication, D. S. Pederson and N. H. Heintz, 1994. CRC Press/R.G. Landes Company Transcriptional Regulation, S. L. McKnight and K. Yamamoto (eds.), 1992. 2 volumes, Cold Spring Harbor Laboratory Press
   RNA
Control of Messenger RNA Stability, J. Belasco and G. Brawerman (eds.), 1993. Academic Press
RNA-Protein Interactions, Nagai and Mattaj (eds.), 1994. Oxford University Press mRNA Metabolism and Post-transcriptional Gene Regulation, Harford and Morris (eds.), 1997. Wiley-Liss
   Translation
Translational Control, J. W. B. Hershey, M. B. Mathews, and N. Sonenberg (eds.), 1995. Cold Spring Harbor Laboratory Press
   General Physiology
Textbook of Medical Physiology 9^{th} Edition by A. C. Guyton and J. E. Hall W.B. Saunders, 1997
Review of Medical Physiology, 18^{th} Edition by W. F. Ganong, Appleton and Lange, 1997
   Online Databases

Those skilled in the art are familiar with how to search the biomedical literature, such as, e.g., libraries, online PubMed, abstract listings, and online mutation databases. One particularly useful resource is maintained at the web site of the National Center for Biotechnology Information (ncbi). From the ncbi site one can access Online Mendelian Inheritance in Man (OMIM). OMIM is a medically oriented database of genetic information with entries for thousands of genes. The OMIM record number is provided for many of the genes in Tables 1, 3, and 4 (see column 3), and constitutes an excellent entry point for identification of references that point to the broader literature. Another useful site at NCBI is the Entrez browser. One can search genomes, polynucleotides, proteins, 3D structures, taxonomy or the biomedical literature (PubMed) via the Entrez site. More generally links to a number of useful sites with biomedical or genetic data are maintained at sites such as Med Web at the Emory University Health Sciences Center Library; Riken, a Japanese web site links to DNA sequence, structural, molecular biology, bioinformatics, and other databases; at the Oak Ridge National Laboratory web site; or at the Yahoo website of Diseases and Conditions. Each of the indicated web sites has additional useful links to other sites.

Another type of database with utility in selecting the genes on a biochemical pathway that may affect the response to a drug are databases that provide information on biochemical pathways. Examples of such databases include the Kyoto Encyclopedia of Genes and Genomes (KEGG). This site has pictures of many biochemical pathways, as well as links to other metabolic databases such as the well known Boehringer Mannheim biochemical pathways charts. The metabolic charts at the latter site are comprehensive, and excellent starting points for working out the salient enzymes on any given pathway.

Each of the web sites mentioned above has links to other useful web sites, which in turn can lead to additional sites with useful information.

Research Libraries

Those skilled in the art will often require information found only at large libraries. The National Library of Medicine is the largest medical library in the world and its catalogs can be searched online. Other libraries, such as university or medical school libraries are also useful to conduct searches. Biomedical books such as those referred to above can often be obtained from online bookstores as described above.

Biomedical Literature

To obtain up to date information on drugs and their mechanism of action and biotransformation; disease pathophysiology; biochemical pathways relevant to drug action and disease pathophysiology; and genes that encode proteins relevant to drug action and disease one skilled in the art will consult the biomedical literature. A widely used, publicly accessible web site for searching published journal articles is PubMed. At this site, one can search for the most recent articles (within the last 1-2 months) or older literature (back to 1966). Many Journals also have their own sites on the world wide web and can be searched online. For example see the IDEAL web site. This site is an online library, featuring full text journals from Academic Press and selected journals from W.B. Saunders and Churchill Livingstone. The site provides access (for a fee) to nearly 2000 scientific, technical, and medical journals.

Experimental Methods for Identification of Genes Involved in the Action of a Drug

There are a number of experimental methods for identifying genes and gene products that mediate or modulate the effects of a drug or other treatment. They encompass analyses of RNA and protein expression as well as methods for detecting protein—protein interactions and protein—ligand interactions. Two preferred experimental methods for identification of genes that may be involved in the action of a drug are (1) methods for measuring the expression levels of many mRNA transcripts in cells or organisms treated with the drug (2) methods for measuring the expression levels of many proteins in cells or organisms treated with the drug.

RNA transcripts or proteins that are substantially increased or decreased in drug treated cells or tissues relative to control cells or tissues are candidates for mediating the action of the drug. Preferably the level of an mRNA is at least 30% higher or lower in drug treated cells, more preferably at least 50% higher or lower, and most preferably two fold higher or lower than levels in non-drug treated control cells. The analysis of RNA levels can be performed on total RNA or on polyadenylated RNA selected by oligodT affinity. Further, RNA from different cell compartments can be analyzed independently—for example nuclear vs. cytoplasmic RNA. In addition to RNA levels, RNA kinetics can be examined, or the pool of RNAs currently being translated can be analyzed by isolation of RNA from polysomes. Other useful experimental methods include protein interaction methods such as the yeast two hybrid system and variants thereof which facilitate the detection of protein—protein interactions. Preferably one of the interacting proteins is the drug target or another protein strongly implicated in the action of the compound being assessed.

The pool of RNAs expressed in a cell is sometimes referred to as the transcriptome. Methods for measuring the transcriptome, or some part of it, are known in the art. A recent collection of articles summarizing some current methods appeared as a supplement to the journal Nature Genetics. (The Chipping Forecast. Nature Genetics supplement, volume 21, January 1999.) A preferred method for measuring expression levels of mRNAs is to spot PCR products corresponding to a large number of specific genes on a nylon membrane such as Hybond N Plus (Amersham-Pharmacia). Total cellular mRNA is then isolated, labeled by random oligonucleotide priming in the presence of a detectable label (e.g. alpha 33P labeled radionucleotides or dye labeled nucleotides), and hybridized with the filter containing the PCR products. The resulting signals can be analyzed by commercially available software, such as can be obtained from Clontech/Molecular Dynamics or Research Genetics, Inc.

Experiments have been described in model systems that demonstrate the utility of measuring changes in the transcriptome before and after changing the growth conditions of cells, for example by changing the nutrient environment. The changes in gene expression help reveal the network of genes that mediate physiological responses to the altered growth condition. Similarly, the addition of a drug to the cellular or in vivo environment, followed by monitoring the changes in gene expression can aid in identification of gene networks that mediate pharmacological responses.

The pool of proteins expressed in a cell is sometimes referred to as the proteome. Studies of the proteome may include not only protein abundance but also protein subcellular localization and protein-protein interaction. Methods for measuring the proteome, or some part of it, are known in the art. One widely used method is to extract total cellular protein and separate it in two dimensions, for example first by size and then by isoelectric point. The resulting protein spots can be stained and quantitated, and individual spots can be excised and analyzed by mass spectrometry to provide definitive identification. The results can be compared from two or more cell lines or tissues, at least one of which has been treated with a drug. The differential up or down modulation of specific proteins in response to drug treatment may indicate their role in mediating the pharmacologic actions of the drug. Another way to identify the network of proteins that mediate the actions of a drug is to exploit methods for identifying interacting proteins. By starting with a protein known to be involved in the action of a drug—for example the drug target—one can use systems such as the yeast two hybrid system and variants thereof (known to those skilled in the art; see Ausubel et al., Current Protocols in Molecular Biology, op. cit.) to identify additional proteins in the network of proteins that mediate drug action. The genes encoding such proteins would be useful for screening for DNA sequence variances, which in turn may be useful for analysis of interpatient variation in response to treatments. For example, the protein 5-lipoxygenase (5LO) is an enzyme which is at the beginning of the leukotriene biosynthetic pathway and is a target for anti-inflammatory drugs used to treat asthma and other diseases. In order to detect proteins that interact with 5-lipoxygenase the two-hybrid system was recently used to isolate three different proteins, none previously known to interact with 5LO. (Provost et al., Interaction of 5-lipoxygenase with cellular proteins. Proc. Natl. Acad. Sci. U.S.A. 96: 1881-1885, 1999.) A recent collection of articles summarizing some current methods in proteomics appeared in the August 1998 issue of the journal Electrophoresis (volume 19, number 11). Other useful articles include: Blackstock W P, et al. Proteomics: quantitative and physical mapping of cellular proteins. Trends Biotechnol. 17 (3): p. 121-7, 1999, and Patton W. F., Proteome analysis II. Protein subcellular redistribution: linking physiology to genomics via the proteome and separation technologies involved. J Chromatogr B Biomed Sci App. 722(1-2):203-23. 1999.

Since many of these methods can also be used to assess whether specific polymorphisms are likely to have biological effects, they are also relevant in section 3, below, concerning methods for assessing the likely contribution of variances in candidate genes to clinical variation in patient responses to therapy.

2. Screen for Variances in Genes that may be Related to Therapeutic Response

Having identified a set of genes that may affect response to a drug the next step is to screen the genes for variances that may account for interindividual variation in response to the drug. There are a variety of levels at which a gene can be screened for variances, and a variety of methods for variance screening. The two main levels of variance screening are genomic DNA screening and cDNA screening. Genomic variance detection may include screening the entire genomic segment spanning the gene from 2 kb to 10 kb upstream of the transcription start site to the polyadenylation site, or 2 to 10 kb beyond the polyadenylation site. Alternatively genomic variance detection may (for intron containing genes) include the exons and some region around them containing the splicing signals, for example, but not all of the intronic sequences. In addition to screening introns and exons for variances it is generally desirable to screen regulatory DNA sequences for variances. Promoter, enhancer, silencer and other regulatory elements have been described in human genes. The promoter is generally proximal to the transcription start site, although there may be several promoters and several transcription start sites. Enhancer, silencer and other regulatory elements may be intragenic or may lie outside the introns and exons, possibly at a considerable distance, such as 100 kb away. Variances in such sequences may affect basal gene expression or regulation of gene expression. In either case such variation may affect the response of an individual patient to a therapeutic intervention, for example a drug, as described in the examples. Thus in practicing the present invention it is useful to screen regulatory sequences as well as transcribed sequences, in order to identify variances that may affect gene transcription. Frequently the genomic sequence of a gene can be found in the sources above, particularly by searching GenBank or Medline (PubMed). The name of the gene can be entered at a site such as Entrez. Using the genomic sequence and information from the biomedical literature one skilled in the art can perform a variance detection procedure such as those described in examples 15, 16 and 17.

Variance detection is often first performed on the cDNA of a gene for several reasons. First, available data on functional sequence variances suggests that variances in the transcribed portion of a gene may be most likely to have functional consequences as they can affect the interaction of the transcript with a wide variety of cellular factors during the complex processes of RNA transcription, processing and translation, with consequent effects on RNA splicing, stability, translational efficiency or other processes. Second, as a practical matter the cDNA sequence of a gene is often available before the genomic structure is known, although the reverse will be true in the future as the sequence of the human genome is determined. Third, the cDNA is often compact compared to the genomic locus, and can be screened for variances with much less effort. If the genomic structure is not known then only the cDNA sequence can be scanned for variances. Methods for preparing cDNA are described in Example 7. Methods for variance detection on cDNA are described below and in the examples.

In general it is preferable to catalog genetic variation at the genomic DNA level because there are an increasing number of well documented instances of functionally important variances that lie outside of transcribed sequence. Also, to properly use optimal genetic methods to assess the contribution of a candidate gene to variation in a phenotype of interest it is desirable to understand the character of sequence variation in the candidate gene: what is the nature of linkage disequilibrium between different variances in the gene; are there sites of recombination within the gene; what is the extent of homoplasy in the gene (i.e. occurrence of two variant sites that are identical by state but not identical by descent because the same variance arose at least twice in human evolutionary history on two different haplotypes); what are the different haplotypes and how can they be grouped to increase the power of genetic analysis?

Methods for variance screening have been described, including DNA sequencing. See for example: U.S. Pat. No. 5,698,400: Detection of mutation by resolvase cleavage; U.S. Pat. No. 5,217,863: Detection of mutations in nucleic acids; and U.S. Pat. No. 5,750,335: Screening for genetic variation, as well as the examples and references cited therein for examples of useful variance detection procedures. Detailed variance detection procedures are also described in examples 15, 16 and 17. One skilled in the art will recognize that depending on the specific aims of a variance detection project (number of genes being screened, number of individuals being screened, total length of DNA being screened) one of the above cited methods may be preferable to the others, or yet another procedure may be optimal. A preferred method of variance detection is chain terminating DNA sequencing using dye labeled primers, cycle sequencing and software for assessing the quality of the DNA sequence as well as specialized software for calling heterozygotes. The use of such procedures has been described by Nickerson and colleagues. See for example: Rieder M. J., et al. Automating the identification of DNA variations using quality-based fluorescence re-sequencing: analysis of the human mitochondrial genome. Nucleic Acids Res. 26 (4):967-73, 1998, and: Nickerson D. A., et al. PolyPhred: automating the detection and genotyping of single nucleotide substitutions using fluorescence-based resequencing. Nucleic Acids Res. 25 (14):2745-51, 1997. Although the variances provided in Tables 3, and 4 consist principally of cDNA variances, it is an aspect of this invention that detection of genomic variances is also a useful method for identification of variances that may account for interpatient variation in response to a therapy.

Another important aspect of variance detection is the use of DNA from a panel of human subjects that represents a known population. For example, if the subjects are being screened for variances relevant to a specific drug development program it is desirable to include both subjects with the target disease and healthy subjects in the panel, because certain variances may occur at different frequencies in the healthy and disease populations and can only be reliably detected by screening both populations. Also, for example, if the drug development program is taking place in Japan, it is important to include Japanese individuals in the screening population. In general, it is always desirable to include subjects of known geographic, racial or ethnic identity in a variance screening experiment so the results can be interpreted appropriately for different patient populations, if necessary. Also, in order to select optimal sets of variances for genetic analysis of a gene locus it is desirable to know which variances have occurred recently—perhaps on multiple different chromosomes—and which are ancient. Inclusion of one or more apes or monkeys in the variance screening panel is one way of gaining insight into the evolutionary history of variances. Chimpanzees are preferred subjects for inclusion in a variance screening panel.

3. Assess the Likely Contribution of Variances in Candidate Genes to Clinical Variation in Patient Responses to Therapy

Once a set of genes likely to affect disease pathophysiology or drug action has been identified, and those genes have been screened for variances, said variances (e.g., provided in Tables 3, and 4) can be assessed for their contribution to variation in the pharmacological or toxicological phenotypes of interest. Such studies are useful for reducing a large number of candidate variances to a smaller number of variances to be tested in clinical trials. There are several methods which can be used in the present invention for assessing the medical and pharmaceutical implications of a DNA sequence variance. They range from computational methods to in vitro and/or in vivo experimental methods, to prospective human clinical trials, and also include a variety of other laboratory and clinical measures that can provide evidence of the medical consequences of a variance. In general, human clinical trials constitute the highest standard of proof that a variance or set of variances is useful for selecting a method of treatment, however, computational and in vitro data, or retrospective analysis of human clinical data may provide strong evidence that a particular variance will affect response to a given therapy, often at lower cost and in less time than a prospective clinical trial. Moreover, at an early stage in the analysis when there are many possible hypotheses to explain interpatient variation in treatment response, the use of informatics-based approaches to evaluate the likely functional effects of specific variances is an efficient way to proceed.

Informatics-based approaches to the prediction of the likely functional effects of variances include DNA and protein sequence analysis (phylogenetic approaches and motif searching) and protein modeling (based on coordinates in the protein database, or pdb. See, for example: Kawabata et al. The Protein Mutant Database. Nucleic Acids Research 27: 355-357, 1999 also available online. Such analyses can be performed quickly and inexpensively, and the results may allow selection of certain genes for more extensive in vitro or in vivo studies or for more variance detection or both.

The three dimensional structure of many medically and pharmaceutically important proteins, or homologs of such proteins in other species, or examples of domains present in such proteins, is known as a result of x-ray crystallography studies and, increasingly, nuclear magnetic resonance studies. Further, there are increasingly powerful tools for modeling the structure of proteins with unsolved structure, particularly if there is a related (homologous) protein with known structure. (For reviews see: Rost et al., Protein fold recognition by prediction-based threading, J. Mol. Biol. 270:471-480, 1997; Firestine et al., Threading your way to protein function, Chem. Biol. 3:779-783, 1996) There are also powerful methods for identifying conserved domains and vital amino acid residues of proteins of unknown structure by analysis of phylogenetic relationships. (Deleage et al., Protein structure prediction: Implications for the biologist, Biochimie 79:681-686, 1997; Taylor et al., Multiple protein structure alignment, Protein Sci. 3:1858-1870, 1994) These methods can permit the prediction of functionally important variances, either on the basis of structure or evolutionary conservation. For example, a crystal structure can reveal which amino acids comprise a small molecule binding site. The identification of a polymorphic amino acid variance in the topological neighborhood of such a site, and, in particular, the demonstration that at least one variant form of the protein has a variant amino acid which impinges on (or which may otherwise affect the chemical environment around) the small molecule binding pocket differently from another variant form, provides strong evidence that the variance may affect the function of the protein. From this it follows that the interaction of the protein with a treatment method, such an administered compound, will likely be variable between different patients. One skilled in the art will recognize that the application of computational tools to the identification of functionally consequential variances involves applying the knowledge and tools of medicinal chemistry and physiology to the analysis.

Phylogenetic approaches to understanding sequence variation are also useful. Thus if a sequence variance occurs at a nucleotide or encoded amino acid residue where there is usually little or no variation in homologs of the protein of interest from non-human species, particularly evolutionarily remote species, then the variance is more likely to affect function of the RNA or protein. Computational methods for phylogenetic analysis are known in the art, (see below for citations of some methods).

Computational methods are also useful for analyzing DNA polymorphisms in transcriptional regulatory sequences, including promoters and enhancers. One useful approach is to compare variances in potential or proven transcriptional regulatory sequences to a catalog of all known transcriptional regulatory sequences, including consensus binding domains for all transcription factor binding domains. See, for example, the databases cited in: Burks, C. Molecular Biology Database List. Nucleic Acids Research 27: 1-9, 1999, and links to useful databases on the internet. In particular see the Transcription Factor Database (Heinemeyer, T., et al. (1999) Expanding the TRANSFAC database towards an expert system of regulatory molecular mechanisms. Nucleic Acids Res. 27: 318-322, or on the internet at: http://1193.175.244.40/TRANSFAC/index.html). Any sequence variances in transcriptional regulatory sequences can be assessed for their effects on mRNA levels using standard methods, either by making plasmid constructs with the different allelic forms of the sequence, transfecting them into cells and measuring the output of a reporter transcript, or by assays of cells with different endogenous alleles of variances. One example of a polymorphism in a transcriptional regulatory element that has a pharmacogenetic effect is described by Drazen et al. (1999) Pharmacogenetic association between ALOX5 promoter genotype and the response to anti-asthma treatment. Nature Genetics 22: 168-170. Drazen and co-workers found that a polymorphism in an Sp1-transcription factor binding domain, which varied among subjects from 3-6 tandem copies, accounted for varied expression levels of the 5-lipoxygenase gene when assayed in vitro in reporter construct assays. This effect would have been flagged by an informatics analysis that surveyed the 5-lipoxygenase candidate promoter region for transcriptional regulatory sequences (resulting in discovery of polymorphism in the Sp1 motif).

4. Perform In Vitro or In Vivo Experiments to Assess the Functional Importance of Gene Variances

There are two broad types of studies useful for assessing the likely importance of variances: analysis of RNA or protein abundance (as described above in the context of methods for identifying candidate genes for explaining interpatient variation in treatment response) or analysis of functional differences in different variant forms of a gene, mRNA or protein. Studies of functional differences may involve direct measurements of biochemical activity of different variant forms of an mRNA or protein, or may involve assaying the influence of a variance or variances on various cell properties, including both tissue culture and in vivo studies.

The selection of an appropriate experimental program for testing the medical consequences of a variance may differ depending on the nature of the variance, the gene, and the disease. For example if there is already evidence that a protein is involved in the pharmacologic action of a drug, then the in vitro or in vivo demonstration that an amino acid variance in the protein affects its biochemical activity is strong evidence that the variance will have an effect on the pharmacology of the drug in patients, and therefore that patients with different variant forms of the gene may have different responses to the same dose of drug. If the variance is silent with respect to protein coding information, or if it lies in a noncoding portion of the gene (e.g., a promoter, an intron, or a 5′- or 3′-untranslated region) then the appropriate biochemical assay may be to assess mRNA abundance, half life, or translational efficiency. If, on the other hand, there is no substantial evidence that the protein encoded by a particular gene is relevant to drug pharmacology, but instead is a candidate gene on account of its involvement in disease pathophysiology, then the optimal test may be a clinical study addressing whether two patient groups distinguished on the basis of the variance respond differently to a therapeutic intervention. This approach reflects the current reality that biologists do not sufficiently understand gene regulation, gene expression and gene function to consistently make accurate inferences about the consequences of DNA sequence variances for pharmacological responses.

In summary, if there is a plausible hypothesis regarding the effect of a protein on the action of a drug, then in vitro and in vivo approaches, including those described below, will be useful to predict whether a given variance is therapeutically consequential. If, on the other hand, there is no evidence of such an effect, then the preferred test is an empirical clinical measure of the impact to the variance on efficacy or toxicity in vivo (which requires no evidence or assumptions regarding the mechanism by which the variance may exert an effect on a therapeutic response). However, given the expense and statistical constraints of clinical trials, it is preferable to limit clinical testing to variances for which there is at least some experimental or computational evidence of a functional effect.

Experimental Methods: Genomic DNA Analysis

Variances in DNA may affect the basal transcription or regulated transcription of a gene locus. Such variances may be located in any part of the gene but are most likely to be located in the promoter region, the first intron, or in 5′ or 3′ flanking DNA, where enhancer or silencer elements may be located. Methods for analyzing transcription are well known to those skilled in the art and exemplary methods are briefly described above and in some of the texts cited elsewhere in this application. Transcriptional run off assay is one useful method. Detailed protocols can be found in texts such as: Current Protocols in Molecular Biology edited by: F. M. Ausubel, et al. John Wiley & Sons, Inc, 1999, or: Molecular Cloning: A Laboratory Manual by J. Sambrook, E. F. Fritsch and T Maniatis. 1989. 3 vols, 2nd edition, Cold Spring Harbor Laboratory Press.

Experimental Methods: RNA Analysis

RNA variances may affect a wide range of processes including RNA splicing, polyadenylation, capping, export from the nucleus, interaction with translation initiation, elongation or termination factors, or the ribosome, or interaction with cellular factors including regulatory proteins, or factors that may affect mRNA half life. However, the effect of most RNA sequence variances on RNA function, if any, should ultimately be measurable as an effect on RNA or protein levels—either basal levels or regulated levels or levels in some abnormal cell state, such as cells from patients with a disease. Therefore, one preferred method for assessing the effect of RNA variances on RNA function is to measure the levels of RNA produced by different alleles in one or more conditions of cell or tissue growth. Said measuring can be done by conventional methods such as Northern blots or RNAase protection assays (kits available from Ambion, Inc.), or by methods such as the Taqman assay (developed by the Applied Biosystems Division of the Perkin Elmer Corporation), or by using arrays of oligonucleotides or arrays of cDNAs attached to solid surfaces. Systems for arraying cDNAs are available commercially from companies such as Nanogen and General Scanning. Complete systems for gene expression analysis are available from companies such as Molecular Dynamics. For recent reviews of systems for high throughput RNA expression analysis see the supplement to volume 21 of Nature Genetics entitled “The Chipping Forecast”, especially articles beginning on pages 9, 15, 20 and 25.

Additional methods for analyzing the effect of variances on RNA include secondary structure probing, and direct measurement of half life or turnover. Secondary structure can be determined by techniques such as enzymatic probing (using enzymes such as T1, T2 and S1 nuclease), chemical probing or RNAase H probing using oligonucleotides. Most RNA structural assays are performed in vitro, however some techniques can be performed on cell extracts or even in living cells, using fluorescence resonance energy transfer to monitor the state of RNA probe molecules.

Experimental Methods: Protein Analysis

There are a variety of experimental methods for investigating the effect of an amino acid variance on response of a patient to a treatment. The preferred method will depend on the availability of cells expressing a particular protein, and the feasibility of a cell-based assay vs. assays on cell extracts, on proteins produced in a foreign host, or on proteins prepared by in vitro translation.

For example, the methods and systems listed below can be utilized to demonstrate differential expression, stability and/or activity of different variant forms of a protein, or in phenotype/genotype correlations in a model system.

For the determination of protein levels or protein activity a variety of techniques are available. The in vitro protein activity can be determined by transcription or translation in bacteria, yeast, baculovirus, COS cells (transient), Chinese Hamster Ovary (CHO) cells, or studied directly in human cells, or other cell systems can be used. Further, one can perform pulse chase experiments to determine if there are changes in protein stability (half-life).

One skilled in the art can construct cell based assays of protein function, and then perform the assays in cells with different genotypes or haplotypes. For example, identification of cells with different genotypes, e.g. cell lines established from families and subsequent determination of relevant protein phenotypes (e.g. expression levels, post translational modifications, activity assays) may be performed using standard methods.

Assays of protein levels or function can also be performed on cell lines (or extracts from cell lines) derived from pedigrees in order to determine whether there is a genetic component to variation in protein levels or function. The experimental analysis is as above for RNAs, except the assays are different. Experiments can be performed on naive cells or on cells subjected to various treatments, including pharmacological treatments.

In another approach to the study of amino acid variances one can express genes corresponding to different alleles in experimental organisms and examine effects on disease phenotype (if relevant in the animal model), or on response to the presence of a compound. Such experiments may be performed in animals that have disrupted copies of the homologous gene (e.g. gene knockout animals engineered to be deficient in a target gene), or variant forms of the human gene may be introduced into germ cells by transgenic methods, or a combination of approaches may be used. To create animal strains with targeted gene disruptions a DNA construct is created (using DNA sequence information from the host animal) that will undergo homologous recombination when inserted into the nucleus of an embryonic stem cell. The targeted gene is effectively inactivated due to the insertion of non-natural sequence—for example a translation stop codon or a marker gene sequence that interrupts the reading frame. Well known PCR based methods are then used to screen for those cells in which the desired homologous recombination event has occurred. Gene knockouts can be accomplished in worms, drosophila, mice or other organisms. Once the knockout cells are created (in whatever species) the candidate therapeutic intervention can be administered to the animal and pharmacological or biological responses measured, including gene expression levels. If variant forms of the gene are useful in explaining interpatient variation in response to the compound in man, then complete absence of the gene in an experimental organism should have a major effect on drug response. As a next step various human forms of the gene can be introduced into the knockout organism (a technique sometimes referred to as a knock-in). Again, pharmacological studies can be performed to assess the impact of different human variances on drug response. Methods relevant to the experimental approaches described above can be found in the following exemplary texts:

General Molecular Biology Methods

Molecular Biology: A project approach, S. J. Karcher, Fall 1995. Academic Press
DNA Cloning: A Practical Approach, D. M. Glover and B. D. Hayes (eds). 1995. IRL/Oxford University Press. Vol. 1—Core Techniques; Vol 2—Expression Systems; Vol. 3—Complex Genomes; Vol. 4—Mammalian Systems.
Short Protocols in Molecular Biology, Ausubel et al. October 1995. 3rd edition, John Wiley and Sons
Current Protocols in Molecular Biology Edited by: F. M. Ausubel, R. Brent, R. E. Kingston, D. D. Moore, J. G. Seidman, K. Struhl, (Series Edition: V. B. Chanda), 1988
Molecular Cloning: A laboratory manual, J. Sambrook, E. F. Fritsch. 1989. 3 vols, 2nd edition, Cold Spring Harbor Laboratory Press
   Polymerase Chain Reaction (PCR)
PCR Primer: A laboratory manual, C. W. Diffenbach and G. S. Dveksler (eds.). 1995. Cold Spring Harbor Laboratory Press.
The Polymerase Chain Reaction, K. B. Mullis et al. (eds.), 1994. Birkhauser
PCR Strategies, M. A. Innis, D. H. Gelf, and J. J. Sninsky (eds.), 1995. Academic Press
   General Procedures for Discipline Specific Studies
Current Protocols in Neuroscience Edited by: J. Crawley, C. Gerfen, R. McKay, M. Rogawski, D. Sibley, P. Skolnick, (Series Editor: G. Taylor), 1997.
Current Protocols in Pharmacology Edited by: S. J. Enna/M. Williams, J. W. Ferkany, T. Kenakin, R. E. Porsolt, J.P. Sullivan, (Series Editor: G. Taylor), 1998.
Current Protocols in Protein Science Edited by: J. E. Coligan, B. M. Dunn, H. L. Ploegh, D. W. Speicher, P. T. Wingfield, (Series Editor: Virginia Benson Chanda), 1995.
Current Protocols in Cell Biology Edited by: J. S. Bonifacino, M. Dasso, J. Lippincott-Schwartz, J. B. Harford, K. M. Yamada, (Series Editor: K. Morgan) 1999.
Current Protocols in Cytometry Managing Editor: J. P. Robinson, Z. Darzynkiewicz (ed)/P. Dean (ed), A. Orfao (ed), P. Rabinovitch (ed), C. Stewart (ed), H. Tanke (ed), L. Wheeless (ed), (Series Editor: J. Paul Robinson), 1997.
Current Protocols in Human Genetics Edited by: N. C. Dracopoli, J. L. Haines, B. R. Korf, et al., (Series Editor: A. Boyle), 1994.
Current Protocols in Immunology Edited by: J. E. Coligan, A. M. Kruisbeek, D. H. Margulies, E. M. Shevach, W. Strober, (Series Editor: R. Coico), 1991.
   IV. Clinical Trials

A clinical trial is the definitive test of the utility of a variance or variances for the selection of optimal therapy. A clinical trial in which an interaction of gene variances and clinical outcomes (desired or undesired) is explored will be referred to herein as a “pharmacogenetic clinical trial”. Pharmacogenetic clinical trials require no knowledge of the biological function of the gene containing the variance or variances to be assessed, nor any knowledge of how the therapeutic intervention to be assessed works at a biochemical level. The pharmacogenetics effects of a variance can be addressed at a purely statistical level: either a particular variance or set of variances is consistently associated with a significant difference in a salient drug response parameter (e.g. response rate, effective dose, side effect rate, etc.) or not. On the other hand, if there is information about either the biochemical basis of a therapeutic intervention or the biochemical effects of a variance, then a pharmacogenetic clinical trial can be designed to test a specific hypothesis. In preferred embodiments of the methods of this application the mechanism of action of the compound to be genetically analyzed is at least partially understood.

Methods for performing clinical trials are well known in the art. (see e.g. Guide to Clinical Trials by Bert Spilker, Raven Press, 1991; The Randomized Clinical Trial and Therapeutic Decisions by Niels Tygstrup (Editor), Marcel Dekker; Recent Advances in Clinical Trial Design and Analysis (Cancer Treatment and Research, Ctar 75) by Peter F. Thall (Editor) Kluwer Academic Pub, 1995. Clinical Trials: A Methodologic Perspective by Steven Piantadosi, Wiley Series in Probability and Statistics, 1997). However, performing a clinical trial to test the genetic contribution to interpatient variation in drug response entails additional design considerations, including (i) defining the genetic hypothesis or hypotheses, (ii) devising an analytical strategy for testing the hypothesis, including determination of how many patients will need to be enrolled to have adequate statistical power to measure an effect of a specified magnitude (power analysis), (iii) definition of any primary or secondary genetic endpoints, and (iv) definition of methods of statistical genetic analysis, as well as other aspects. In the outline below some of the major types of genetic hypothesis testing, power analysis and statistical testing and their application in different stages of the drug development process are reviewed. One skilled in the art will recognize that certain of the methods will be best suited to specific clinical situations, and that additional methods are known and can be used in particular instances.

A. Performing a Clinical Trial: Overview

As used herein, a “clinical trial” is the testing of a therapeutic intervention in a volunteer human population for the purpose of determining whether it is safe and/or efficacious in the treatment of a disease, disorder, or condition. The present invention describes methods for achieving superior efficacy and/or safety in a genetically defined subgroup defined by the presence or absence of at least one gene sequence variance, compared to the effect that could be obtained in a conventional trial (without genetic stratification).

A “clinical study” is that part of a clinical trial that involves determination of the effect of a candidate therapeutic intervention on human subjects. It includes clinical evaluation of physiologic responses including pharmacokinetic (bioavailability as affected by drug absorption, distribution, metabolism and excretion) and pharmacodynamic (physiologic response and efficacy) parameters. A pharmacogenetic clinical study (or clinical trial) is a clinical study that involves testing of one or more specific hypotheses regarding the interaction of a genetic variance or variances (or set of variances, i.e. haplotype or haplotypes) on response to a therapeutic intervention. Pharmacogenetic hypotheses are formulated before the study, and may be articulated in the study protocol in the form of primary or secondary endpoints. For example an endpoint may be that in a particular genetic subgroup the rate of objectively defined responses exceeds the response rate in a control group (either the entire control group or the subgroup of controls with the same genetic signature as the treatment subgroup they are being compared to) or exceeds that in the whole treatment group (i.e. without genetic stratification) by some predefined relative or absolute amount.

For a clinical study to commence enrollment and proceed to treat subjects at an institution that receives any federal support (most medical institutions in the US), an application that describes in detail the scientific premise for the therapeutic intervention and the procedures involved in the study, including the endpoints and analytical methods to be used in evaluating the data, must be reviewed and accepted by a review panel, often termed an Institutional Review Board (IRB). Similarly any clinical study that will ultimately be evaluated by the FDA as part of a new drug or product application (or other application as described below), must be reviewed and approved by an IRB. The IRB is responsible for determining that the trial protocol is safe, conforms to established ethical principles and guidelines, has risks proportional to any expected benefits, assures equitable selection of patients, provides sufficient information to patients (via a consent form) to insure that they can make an informed decision about participation, and insures the privacy of participants and the confidentiality of any data collected. (See the report of the National Commission for Protection of Human Subjects of Biomedical and Behavioral Research (1978). The Belmont Report: Ethical Principles and Guidelines for the Protection of Human Subjects of Research. Washington, D.C.: DHEW Publication Number (OS) 78-0012. For a recent review see: Coughlin, S. S. (ed.) (1995) Ethics in Epidemiology and Clinical Research. Epidemiology Resources, Newton, Mass.) The European counterpart of the US FDA is the European Medicines Evaluation Agency (EMEA). Similar agencies exist in other countries and are responsible for insuring, via the regulatory process, that clinical trials conform to similar standards as are required in the US. The documents reviewed by an IRB include a clinical protocol containing the information described above, and a consent form.

It is also customary, but not required, to prepare an investigator's brochure which describes the scientific hypothesis for the proposed therapeutic intervention, the preclinical data, and the clinical protocol. The brochure is made available to any physician participating in the proposed or ongoing trial.

The supporting preclinical data is a report of all the in vitro, in vivo animal or previous human trial or other data that supports the safety and/or efficacy of a given therapeutic intervention. In a pharmacogenetic clinical trial the preclinical data may also include a description of the effect of a specific genetic variance or variances on biochemical or physiologic experimental variables in vitro or in vivo, or on treatment outcomes, as determined by in vivo studies in animals or humans (for example in an earlier trial), or by retrospective genetic analysis of clinical trial or other medical data (see below) used to formulate or strengthen a pharmacogenetic hypothesis. For example, case reports of unusual pharmacological responses in individuals with rare alleles (e.g. mutant alleles), or the observation of clustering of pharmacological responses in family members may provide the rationale for a pharmacogenetic clinical trial.

The clinical protocol provides the relevant scientific and therapeutic introductory information, describes the inclusion and exclusion criteria for human subject enrollment, including genetic criteria if relevant (e.g. if genotype is to be among the enrollment criteria), describes in detail the exact procedure or procedures for treatment using the candidate therapeutic intervention, describes laboratory analyses to be performed during the study period, and further describes the risks (both known and possible) involving the use of the experimental candidate therapeutic intervention. In a clinical protocol for a pharmacogenetic clinical trial, the clinical protocol will further describe the genetic variance and/or variances hypothesized to account for differential responses in the normal human subjects or patients and supporting preclinical data, if any, a description of the methods for genotyping, genetic data collection and data handling as well as a description of the genetic statistical analysis to be performed to measure the interaction of the variance or variances with treatment response. Further, the clinical protocol for a pharmacogenetic clinical trial will include a description of the genetic study design. For example patients may be stratified by genotype and the response rates in the different groups compared, or patients may be segregated by response and the genotype frequencies in the different responder or nonresponder groups measured. One or more gene sequence variances or a combination of variances and/or haplotypes may be studied.

The informed consent document is a description of the therapeutic intervention and the clinical protocol in simple language (e.g. third grade level) for the patient to read, understand, and, if willing, agree to participate in the study by signing the document. In a pharmacogenetic clinical study the informed consent document will describe, in simple language, the use of a genetic test or a limited set of genetic tests to determine the subject or patient's genotype at a particular gene variance or variances, and to further ascertain whether, in the study population, particular variances are associated with particular clinical or physiological responses. The consent form should also describe procedures for assuring privacy and confidentiality of genetic information.

The US FDA reviews proposed clinical trials through the process of an Investigational New Drug Application (IND). The IND is composed of the investigator's brochure, the supporting in vitro and in vivo animal or previous human data, the clinical protocol, and the informed consent forms. In each of the sections of the IND, a specific description of a single allelic variance or a number of variances to be tested in the clinical study will be included. For example, in the investigator's brochure a description of the gene or genes hypothesized to account, at least in part, for differential responses will be included as well as a description of a genetic variance or variances in one or more candidate genes. Further, the preclinical data may include a description of in vivo, in vitro or in silico studies of the biochemical or physiologic effects of a variance or variances (e.g., haplotype) in a candidate gene or genes, as well as the predicted effects of the variance or variances on efficacy or toxicology of the candidate therapeutic intervention. The results of retrospective genetic analysis of response data in patients treated with the candidate therapy may be the basis for formulating the genetic hypotheses to be tested in the prospective trial. The US FDA reviews applications with particular attention to safety and toxicological data to ascertain whether candidate compounds should be tested in humans.

The established phases of clinical development are Phase I, II, III, and IV. The fundamental objectives for each phase become increasingly complex as the stages of clinical development progress. In Phase I, safety in humans is the primary focus. In these studies, dose-ranging designs establish whether the candidate therapeutic intervention is safe in the suspected therapeutic concentration range. However, it is common practice to obtain information about surrogate markers of efficacy even in phase I clinical trials. In a pharmacogenetic clinical trial there may be an analysis of the effect of a variance or variances on Phase I safety or surrogate efficacy parameters. At the same time, evaluation of pharmacokinetic parameters (e.g., adsorption, distribution, metabolism, and excretion) may be a secondary objective; again, in a pharmacogenetic clinical study there may be an analysis of the effect of sequence variation in genes that affect absorption, distribution, metabolism and excretion of the candidate compound on pharmacokinetic parameters such as peak blood levels, half life or tissue distribution of the compound. As clinical development stages progress, trial objectives focus on the appropriate dose and method of administration required to elicit a clinically relevant therapeutic response. In a pharmacogenetic clinical trial, there may be a comparison of the effectiveness of several doses of a compound in patients with different genotypes, in order to identify interactions between genotype and optimal dose. For this purpose the doses selected for late stage clinical testing may be greater, equal or less than those chosen based upon preclinical safety and efficacy determinations. Data on the function of different alleles of genes affecting pharmacokinetic parameters could provide the basis for selecting an optimal dose or range or doses of a compound or biological. Genes involved in drug metabolism may be particularly useful to study in relation to understanding interpatient variation in optimal dose. Genes involved in drug metabolism include the cytochrome P450s, especially 2D6, 3A4, 2C9, 2E1, 2A6 and 1A1; the glucuronyltransferases; the acetyltransferases; the methyltransferases; the sulfotransferases; the glutathione system; the flavine monooxygenases and other enzymes known in the art.

An additional objective in the latter stages of clinical development is demonstration of the effect of the therapeutic intervention on a broad population. In phase III trials, the number of individuals enrolled is dictated by a power analysis. The number of patients required for a given pharmacogenetic clinical trial will be determined by prior knowledge of variance or haplotype frequency in the study population, likely response rate in the treated population, expected magnitude of pharmacogenetic effect (for example, the ratio of response rates between a genetic subgroup and the unfractionated population, or between two different genetic subgroups); nature of the genetic effect, if known (e.g. dominant effect, codominant effect, recessive effect); and number of genetic hypotheses to be evaluated (including number of genes and/or variances to be studied, number of gene or variance interactions to be studied). Other considerations will likely arise in the design of specific trials.

Clinical trials should be designed to blind both human subjects and study coordinators from biasing that may otherwise occur during the testing of a candidate therapeutic invention. Often the candidate therapeutic intervention is compared to best medical treatment, or a placebo (a compound, agent, device, or procedure that appears identical to the candidate therapeutic intervention but is therapeutically inert). The combination of a placebo group and blind controls for potentially confounding factors such as prejudice on the part of study participants or investigators, insures that real, rather than perceived or expected, effects of the candidate therapeutic intervention are measured in the trials. Ideally blinding extends not only to trial subjects and investigators but also to data review committees, ancillary personnel, statisticians, and clinical trial monitors.

In pharmacogenetic clinical studies, a placebo arm or best medical control group may be required in order to ascertain the effect of the allelic variance or variances on the efficacy or toxicology of the candidate therapeutic intervention as well as placebo or best medical therapy. It will be important to assure that the composition of the control and test populations are matched, to the degree possible, with respect to genetic background and allele frequencies. This is particularly true if the variances being investigated may have an effect on disease manifestations (in addition to a hypothesized effect on response to treatment). It is likely that standard clinical trial procedures such as insuring that treatment and control groups are balanced for race, sex and age composition and other non-genetic factors relevant to disease will be sufficient to assure that genetic background is controlled, however a preferred practice is to explicitly test for genetic stratification between test and control groups. Methods for minimizing the possibility of spurious results attributable to genetic stratification between two comparison groups include the use of surrogate markers of geographic, racial and/or ethnic background, such as have been described by Rannala and coworkers. (See, for example: Rannala B, and J L Mountain. 1997 Detecting immigration by using multilocus genotypes. Proc Natl Acad Sci USA August 19; 94(17):9197-201.) One procedure would be to assure that surrogate markers of genetic background (such as those described by Rannala and Mountain) occur at comparable frequency in two comparison groups.

Open label trials are unblinded; in single blind trials patients are kept unaware of treatment assignments; in double blind trials both patients and investigators are unaware of the treatment groups; a combination of these procedures may be instituted during the trial period. Pharmacogenetic clinical trial design may include one or a combination of open label, single blind, or double blind clinical trial designs. Reduction of biases attributable to the knowledge of either the type of treatment or the genotype of the normal subjects or patients is an important aspect of study design. So, for example, even in a study that is single blind with respect to treatment, it should be possible to keep both patients and caregivers blinded to genotype during the study.

In designing a clinical trial it is important to include termination endpoints such as adverse clinical events, inadequate study participation either in the form of lack of adherence to the clinical protocol or loss to follow up, (e.g. such that adequate power is no longer assured), lack of adherence on the part of trial investigators to the trial protocol, or lack of efficacy or positive response within the test group. In a pharmacogenetic clinical trial these considerations obtain not only in the entire treatment group, but also in the genetically defined subgroups. That is, if a dangerous toxic effect manifests itself predominantly or exclusively in a genetically defined subpopulation of the total treatment population it may be deemed inappropriate to continue treating that genetically defined subgroup. Such decisions are typically made by a data safety monitoring committee, a group of experts not including the investigators, and generally not blinded to the analysis, who review the data from an ongoing trial on a regular basis.

It is important to note that medicine is a conservative field, and clinicians are unlikely to change their behavior on the basis of a single clinical trial. Thus it is likely that, in most instances, two or more clinical trials will be required to convince physicians that they should change their prescribing habits in view of genetic information. Large scale trials represent one approach to providing increased data supporting the utility of a genetic stratification. In such trials the stringent clinical and laboratory data collection characteristic of traditional trials is often relaxed in exchange for a larger patient population. Important goals in large scale pharmacogenetic trials will include establishing whether a pharmacogenetic effect is detectable in all segments of a population. For example, in the North American population one might seek to demonstrate a pharmacogenetic effect in people of African, Asian, European and Hispanic (i.e. Mexican and Puerto Rican) origin, as well as in native American people. (It generally will riot be practical to segment patients by geographical origin in a standard clinical trial, due to loss of power.) Another goal of a large scale clinical trial may be to measure more precisely, and with greater confidence, the magnitude of a pharmacogenetic effect first identified in a smaller trial. Yet another undertaking in a large scale clinical trial may be to examine the interaction of an established pharmacogenetic variable (e.g. a variance in gene A, shown to affect treatment response in a smaller trial) with other genetic variances (either in gene A or in other candidate genes). A large scale trial provides the statistical power necessary to test such interactions.

In designing all of the above stages of clinical testing investigators must be attentive to the statistical problems raised by testing multiple different hypotheses, including multiple genetic hypotheses, in subsets of patients. Bonferroni's correction or other suitable statistical methods for taking account of multiple hypothesis testing will need to be judiciously applied. However, in the early stages of clinical testing, when the main goal is to reduce the large number of potential hypotheses that could be tested to a few that will be tested, based on limited data, it may be impractical to rigidly apply the multiple testing correction.

B. Phase I Clinical Trials

1. Introduction

Phase I clinical trials are generally designed primarily to establish a safe dose and schedule of administration for a new compound. At the same time, Phase I is the first opportunity to study the clinical pharmacology of a new compound in man. Relevant studies may include aspects of pharmacokinetic behavior, side effects and toxicity. In addition to these well established purposes, Phase I trials are increasingly being used to gather information relevant to early assessment of efficacy. Such information can be useful in making an early yes/no decision about the further development of a compound, or a family of related compounds all being tested simultaneously in Phase I trials. Since Phase I trials are typically conducted in normal volunteers (compounds for cancer and some other terminal disease are an exception) surrogate markers of drug effect are measured, rather than disease response. The development of sophisticated surrogate markers of pharmacodynamic effects has allowed more information on efficacy to be gathered n Phase I, and this trend will almost certainly continue as basic understanding of disease pathophysiology increases, and as more products are developed for disease prophylaxis.

Phase I studies are typically performed on a small number (<60) of healthy volunteers. Consequently, Phase I studies as currently designed are not amenable to genetic analysis: the number of subjects is simply too small to detect, with adequate statistical certainty, any genetic effects on drug response that are short of all or none in magnitude. In fact, no genetic analyses of Phase I have been published or described in public meetings.

As described in detail elsewhere in this application, it is highly desirable to gather the information necessary to make informed decisions about clinical development as early as possible in the development process, particularly once human testing has begun and costs therefore mount quickly. Timely information may allow a drug to be killed early, or may result in an accelerated program of clinical trials. In addition to information about efficacy and safety, it is useful to have information about the existence and magnitude of genetic effects on efficacy and toxicity at the earliest possible stage. If properly managed, genetically determined heterogeneity in drug response may not be an obstacle to development. On the contrary, it may provide the basis for identification of a patient population in whom both high efficacy and safety can be achieved. Clear delineation of such a population may facilitate smaller, more targeted trials and more rapid clinical development. Consequently, the early identification of genetic determinants of drug response will, in the future, increasingly become a priority of clinical development.

Phase I trials are not necessarily confined to the initial stages of human clinical development. It is not unusual for Phase I trials to be initiated at a later stage of clinical development in order to, for example, clarify basic questions about clinical pharmacology that have arisen as a result of Phase II data. It may be that the most efficient way to advance the genetic understanding of pharmacological responses to a compound in Phase II is to perform a Phase I trial using specific genetic design, as described below.

2. Phase I Trials Designed for Genetic Analysis

In this invention we describe two exemplary novel methods for organization of Phase I trials that will facilitate identification and measurement of the genetic component of variation in treatment response using modest numbers of subjects. We describe how these methods can be practiced by selectively enrolling subjects who share genetic characteristics, either as a result of a familial relationship or as a result of genetic homogeneity at candidate loci believed to affect response to the candidate treatment. We show how the analysis of such individuals substantially increases the power of genetic analysis compared to analysis of unrelated individuals. We also describe methods for operating a Phase I unit capable of carrying out the novel genetic analyses.

The two types of Pharmacogenetic Phase I Units described in this application will be referred to as the Pharmacogenetic Phase I Relatives Unit and the Pharmacogenetic Phase I Outliers Unit, or the Relatives Unit and the Outliers Unit for short. The term Pharmacogenetic Phase I Unit will be used to refer to both types of Phase I Unit. The Relatives Unit requires a population comprised of groups of related individuals. The related individuals may be parents and offspring, groups of sibs, or of cousins, or any mixture of these or other groups of related individuals. The Outliers Unit requires the initial enrollment of a large number of unrelated volunteers (at least several hundreds of subjects, preferably at least one thousand, more preferably at least five thousand, and most preferably ten thousand or more individuals) willing to provide DNA for genotyping on an as-needed basis (many of these volunteers will never participate in a trial). Subsequently, small numbers of individuals are drawn from this large population for specific clinical trials, based on their genetic homogeneity at candidate loci believed likely to account for intersubject variation in response to the candidate compound.

The concept underlying these two types of Pharmacogenetic Phase I Units is similar: the idea is to recruit multiple small groups of subjects who are genetically more homogeneous than would be possible with standard nongenetic recruitment criteria. If there is a genetic component to treatment response then there should be more intragroup homogeneity and more intergroup heterogeneity in drug response measures (e.g. surrogate measures of drug response) than would be expected by chance, and there should be statistically significant differences in drug response measures between the different groups. The magnitude of such differences can provide an estimate of the magnitude of the genetic component of intersubject variation in drug response.

3. Pharmacogenetic Phase I Relatives Unit

In the Pharmacogenetic Phase I Relatives Unit, one is comparing groups of related individuals to each other and to other groups of related individuals. The underlying assumption is that one can assess the magnitude of the genetic component of variation in drug response (if any) by comparing drug response traits in related individuals with those of unrelated individuals. Two types of effect would suggest the presence of a genetic component to variation in drug response measures. First, the distribution of drug responses in related individuals may be different from that observed in the entire group, or in a group comprised of unrelated individuals. For example, a statistically significant narrowing of the distribution (e.g. smaller standard deviation in groups of related individuals compared to unrelated individuals) would indicate that individuals who share alleles are more similar to each other than individuals who do not share (as many) alleles, implying that the drug response trait is partially affected by a heritable factor or factors. Second, the mean value of the drug response measure (whether blood pressure or a cognitive test) many vary between groups of related individuals, indicating that different alleles at loci relevant to drug response are present in the different families. (Note that the relevant trait is not blood pressure or cognition, but the response of blood pressure or cognition to a pharmacological intervention.)

Individuals can be related in any of several ways, most preferably as parent and child or as siblings. Parent-child pairs, in particular, enable one to use simple statistical techniques (e.g. regression) in order to assess the degree to which response to surrogate markers is influenced by genetic differences among individuals. However, parent-child pairs may be less suitable for some surrogate markers, especially those related to candidate drugs used to treat age-related disorders. In such a context, one can readily use clusters of siblings and/or cousins, uncle/nephew pairs or other groups of related individuals to assess the degree of genetic determination of response to a surrogate marker.

An attractive aspect of the Pharmacogenetic Phase I Relatives Unit (unlike the Outliers Unit) is that it does not require any laboratory tests to implement. One infers the degree of gene sharing between individuals from their relationship to each other. A parent is 50% genetically identical to each of his or her children; sibs are 50% genetically identical to each other on average; uncles/aunts are 25% identical to nieces/nephews on average, and so forth. Thus the degree to which two related individuals are expected to be similar as a result of genetic factors is known. Therefore no tests to determine genetic status are required (i.e. no genotyping); in fact, no knowledge of the relevant candidate loci is required at all (albeit knowledge of the relevant genes is required to develop a useful genetic diagnostic test at a later stage). Thus, the Relatives Unit provides a clear picture of the importance of heredity factors in determining drug response, regardless of our understanding of the mechanism of action of the drug, or any other aspect of drug pharmacology.

The rationale is as follows: if a surrogate drug response trait (i.e., a surrogate marker of pharmacodynamic effect that can be measured in normal subjects) is under genetic control, then related individuals, such as sibs (who share 50% of their alleles at autosomal loci on average), should have more similar responses than unrelated individuals, who share a much smaller fraction of alleles. In other words, individuals who share more alleles at the loci that affect drug response should be more similar to each other than individuals who, on average, share fewer alleles. By using statistical methods known in the art the distribution of traits of related individuals can be compared to the degree of variation in a set of unrelated individuals. The potential for insight from this kind of analysis is reflected in the fact that twin studies (in which traits of identical twins are compared to those of fraternal twins) indicate that differences among individuals in pharmacokinetic variables (e.g. compound half life, peak concentration) can be strongly genetically determined. (For a summary of such pharmacokinetic studies, see Propping, P. [1978] Pharmacogenetics. Rev. Physiol. Biochem. Pharmacol. 83: 123-173.) Such studies are important because they clearly reveal genetic determination of pharmacogenetic traits (although they may overestimate its degree; see Falconer, D. S. and Mackay, T. [1996] Introduction to Quantitative Genetics, Addison Wesley Longman Ltd.).

The type of study proposed here, whether it involves comparison of parents and offspring, groups of sibs, or other groups of relatives, will also reveal the extent of genetic determination, and without requiring twins. This is two-fold advantage; pairs of twins are more difficult to obtain than parent-child or sib-sib pairs, and one avoids the uncertainty about the genetic inferences gained from twin analysis.

Drug responses among related and unrelated individuals may be continuously or discretely distributed. In the former case, it is likely that many loci have some effect on the trait, while in the latter case, variation could be attributable to Mendelian segregation of alleles in a family (or families) with, for example, AA homozygotes giving one phenotype and Aa heterozygotes and aa homozygotes giving a second phenotype, all in the context of a relatively homogeneous genetic background.

There is a wealth of analytical techniques known in the art that can be used to assess the mode of inheritance for a particular trait and to determine the degree to which differences among individuals are genetically determined. These techniques include cluster analysis and discriminant analysis used to define traits with variable expression and the fitting of a variety of genetic models to the data, including generalized single-locus models, mixed models in which a trait is determined by a major locus and by many minor loci, and a so-called polygenic model in which many loci contribute variation to the trait, the result being a continuously-distributed phenotype. (For further details, see Eaves, L. J. [1977] Inferring the causes of human variation, Journal of the Royal Statistical Society A 140: 324-355 and Cloninger, C. R. [1988] Complex Human Traits Pp. 312-317 in: Proceedings of the Second International Conference on Quantitative Genetics, eds., B. S. Weir, E. J. Eisen, M. M. Goodman, and G. Namkoong, Sinauer Associates, Inc.). Specific statistical techniques involved in the fitting and analysis of these genetic models are also well known in the art; they include parametric and nonparametric correlation, regression, and one-way and two-way analysis of variance (For further details, see Mather, K. and Jinks, J. L. [1977] Introduction to Biometrical Genetics, Cornell University Press and Falconer, D. S. and Mackay, T. [1996] Introduction to Quantitative Genetics, Addison Wesley Longman Ltd.)

Many, perhaps most, traits of pharmacogenetic interest will be continuously-distributed. In this context, the central statistical comparison is one between the differences among average traits of different families (say, group of sibs), or among all the members of several such families, as compared to the differences among traits within families (among sibs). If such differences in so-called mean squares are large enough (as compared to the differences expected under the null hypothesis of no family differences), one can infer that there is a genetic component to differences among families.

Standard theory known in the art indicates that there is an inverse relationship between study size and the ability to detect a given genetic effect. So, for example, assume that the 50% of the variation among individuals is due to genetic differences. A Phase 1 trial composed of sixty individuals consisting of thirty parent-child pairs may or may not allow one to detect such a genetic effect, given the standard criterion for statistical significance (P<0.05), depending on assumptions one makes about the number of loci that have major effects. However, a trial composed of 120 individuals consisting of sixty parent-child pairs would likely be sufficient to provide statistically significant evidence for a 50% heritable drug response effect. Once one parent-child pair is recruited, it is generally advantageous statistically to add additional parent-child combinations as opposed to adding additional children for a given parent.

If 75% or more of the variation in drug response among individuals is due to genetic differences, a Phase I trial composed of sixty individuals consisting of thirty parent-child pairs would allow one to detect such a genetic effect, given the standard criterion for statistical significance (P<0.05).

Similar calculations can be made if one analyzes siblings in a Phase I trial, instead of using parent-child pairs. These calculations indicate that the more powerful approach for a Relatives Unit is generally to focus on parent-child pairs as opposed to the use of groups of siblings, especially if minimizing the number of subjects is an objective of the study. However, the use of groups of siblings may be necessary or preferable, especially if the trait in question is manifested only at a specific age. In such a case, one can readily use standard theory to compare alternative designs for the study. The overall point is that the statistical framework associated with the Relatives Unit will allow one to choose the approach that is best-suited for a given trait.

In general, techniques for measuring whether pharmacodynamic traits are under genetic control using surrogate markers of drug efficacy will be useful in obtaining an early assessment of the extent of genetically determined variation in drug response for a given therapeutic compound. Such information provides an informed basis for either stopping development at the earliest possible stage, or preferably, continuing development, but with a plan to identify and control for genetic variation so as to allow rapid progression through the regulatory approval process.

For example, it is well known that clinical trials to assess the efficacy of candidate drugs for Alzheimer's disease are long and expensive, and most such drugs are only effective in a fraction of patients. Using surrogate measures of response in normals drawn from a population of related individuals might help to assess the contribution of genetic variation to variation in treatment response. For an acetylcholinesterase inhibitor, relevant surrogate pharmacodynamic measures might include testing erythrocyte membrane acetylcholinesterase levels in drug tested normal subjects, or testing performance on a psychometric test of short term memory, or other measures that are affected by treatment (and ideally that correlate with clinical efficacy).

Similarly, antidepressant drugs can produce a variety of effects on mood in normal subjects. Careful measurement and statistical analysis of such responses in related and unrelated normal subjects could provide an early indication of whether there is a genetic component to drug response (and hence clinical efficacy). The observation of significant variation among families would provide evidence of a pharmacogenetic effect and justify the substantial expenditure necessary for a full pharmacogenetic drug development program. Conversely, the absence of any significant familial influence on drug response in a Pharmacogenetics Relatives Unit could provide an early termination point for pharmacogenetic studies.

Again, the proposed studies do not require any knowledge of candidate loci, nor is DNA collection or genotyping required. One needs only a reliable surrogate pharmacodynamic assay and groups of related normal individuals. Standard statistical methods should permit the magnitude of the pharmacogenetic effect to be estimated. It should be a criteria for deciding whether to proceed with more intensive, gene-focused pharmacogenetic analysis during later stages of development.

4. Pharmacogenetic Phase I Outliers Unit

The prerequisites for a Pharmacogenetic Phase I Outliers Unit, as well as the type of information that can be obtained, differ in several respects from a Pharmacogenetic Phase I Relatives Unit. First, the Outliers Unit requires some knowledge of the molecular pharmacology of the candidate compound—enough knowledge to select at least one candidate gene. Second, the Outliers Unit provides information on the effect, if any, of known genetic variation in the candidate gene or genes on variation in the drug response measures. This is advantageous in that it sets the stage for pharmacogenetic analysis in later stages of clinical development. Third, the Outliers Unit does not require recruitment of relatives. Instead, one initially recruits a large population of individuals from which small subsets are drawn as necessary for specific trials based on their genotypes.

All of the individuals in the large population are initially asked to provide DNA samples (from blood or other readily available tissue such as buccal mucosa) which can subsequently by genotyped at candidate loci of potential relevance to a particular candidate drug of interest. Over time a database of genotypes can be assembled, potentially reducing the need for genotyping later. From this large collection of subjects one then selects a group of individuals with genotypes expected to homogeneous for the drug response trait of interest (assuming that the candidate gene(s) play a significant role in drug response). The individuals with identical (and preferably homozygous) genotypes at the candidate gene(s) might comprise a collection of the common genotypes or haplotypes, or they may include some rare genotypes/haplotypes as well. The main point is that one can recruit groups consisting of any mixture of genotypes or haplotypes in order to assess the role that variation in the candidate gene(s) may play in trait determination. In this method, then, one recruits a population for clinical genetic investigation utilizing methods in statistical genetics to optimize the size and genetic composition of the population.

The mechanics of an Outlier Unit are as follows. Several thousand subjects are enrolled in the Outlier Unit with the understanding that they provide a blood sample from which DNA is extracted and stored. Each time a new outlier study is performed their sample may be genotyped. (It will not be necessary to genotype all subjects for all trials—just enough to identify subjects with the desired genotypes or haplotypes. Subjects may be paid a fee for each genotyping analysis done on their sample, regardless of whether the sample is used.) Only rarely will a particular subject have a genotype that meets the criteria for a specific outlier study (see below). When a match occurs, that subject will be invited to participate in that study. The genotyping done to identify subjects for a study will be determined by the candidate genes deemed relevant to pharmacology of the candidate drug, and by the polymorphisms or haplotypes in those candidate genes. Ideally DNA samples from several thousand subjects will be arrayed in 96 or 384 well plates so that the genotyping or haplotyping of large numbers of subjects can be performed using automated methods. Any highly accurate and inexpensive genotyping procedure will suffice, such as the methods described elsewhere in this application. Clearly it is desirable to have a stable population for genotyping, given the investment required to recruit subjects, isolate and array DNA, and accumulate a database of genotype data. Since most subjects will only rarely be invited to participate in clinical trials, the ongoing participation of subjects in the Outliers Unit must be assured by other means—for example, by a modest annual payment for remaining in the Outliers Unit, plus a fee for each occasion on which their sample is genotyped.

The power of the Outliers Unit lies in the ability to rapidly enroll individuals with virtually any desired genotype in a Phase I clinical trial. Suppose, for example, that one wants to determine the drug response phenotype of individuals homozygous for rare alleles at candidate loci. Consider a compound for which there are two loci believed likely to influence response to treatment. The first locus has alleles A and a, while the second has alleles B and b. If these loci do in fact contribute significantly to treatment response then homozygotes would be expected to exhibit the most extreme responses (assuming a dominant or codominant model). One could also measure epistatic (gene X gene) interactions on the presumption that drug response measures might be extreme in individuals homozygous for specific alleles of the two candidate genes. So, for example, one would perform a Phase I study consisting of measuring a surrogate drug response in individuals with genotypes AA/BB, aa/BB, AA/bb and aa/bb and then statistically comparing the distribution of a trait in each of these groups with the distribution of the same trait in the other groups and/or in the unfractionated (total) population. The statistical techniques for such comparisons are known in the art and include parametric and nonparametric analyses to detect differences in population averages, such as the t-test and the Mann-Whitney U test. If individuals of a given rare genotype do have significantly different surrogate drug responses when compared to each other, or when compared to the rest of the population, one can infer that the locus likely affects the trait.

The size requirements of the source population of individuals will depend on the range of allele frequencies to be analyzed. For example, if the allele frequencies for A and a are, say, 0.15 and 0.85, and for B and b are 0.2 and 0.8 then the frequency of AA homozygotes is expected to be 2.25% and BB homozygotes 4%. In the absence of any linkage between the loci, the frequency of AA/BB double homozygotes is expected to be 0.0225×0.04=0.0009 or about one subject in 1000. At least five subjects of each genotype should be recruited for the Outlier Unit, and preferably at least ten subjects. Thus, for studies of two loci in which the minor allele frequency for both loci is in the 0.15-0.20 range, the recruitment of individuals that are potential outliers for the trait under investigation (i.e., homozygotes at the candidate loci) will require at least 1,000 individuals and preferably 5,000 or more.

One of the most useful aspects of the Outlier Unit is that individuals with rare genotypes can be pharmacologically assessed in a small study. This addresses a serious limitation of conventional clinical trials with respect to the investigation of polygenic traits or the effect of rare alleles. Even conventional Phase III studies, which typically have the largest number of patients, are usually of insufficient size to address simple one-locus hypotheses about efficacy or toxicity with adequate statistical power (e.g. 80% or 90% power). The problem is that for each new allele that must be considered (e.g. five common haplotypes at a candidate locus) the comparison groups are reduced and statistical power is diminished. It is therefore an especially challenging problem to test the effect of multiple alleles at a single locus, let alone interaction of alleles at several loci in determining drug response. The Outlier-Unit provides a way to efficiently test for the effects of multiple alleles at a candidate locus (e.g. haplotypes), or to test for interactions between two or more candidate loci by allowing ready identification of groups of individuals who, on account of being homozygous at one or several loci of interest, should be outliers for the drug response traits of interest.

The information that can be gained from an Outliers Unit is of great value in designing subsequent efficacy trials, as it provides a basis for constraining the number of hypotheses to be tested. In lieu of such information, one is compelled to statistically test a variety of genetic models for a number of candidate loci. The correction for multiple testing necessitated by such uncertainty about the genetic model is frequently large enough to put statistically significant results beyond reach. On the other hand, if the phenotypic effect of each allele at a locus (or the effect of at least some alleles) is known from the Outliers Unit study, one is then able to design a Phase II or Phase III study that tests a relatively small number of genetic hypotheses, thereby considerably improving the statistical power of the genetic analysis in efficacy trials.

Consider a locus with two alleles, one with frequency 0.95 and the other 0.05, as revealed by genotyping the individuals in the large source population for the Outliers Unit. The two alleles combine to make three genotypes which are observed to differ in their response to a candidate compound of interest. There are several statistical comparisons that one can undertake in order to determine whether different alleles at this locus are associated with differences in response. One is to compare the average response of, say, individuals who are homozygous for the rare allele with the average response of individuals chosen at random from the source population. In this instance, the Outlier Unit is composed of a group of individuals with the rare genotype and an equal-sized group composed of random genotypes (including the rare genotype). (In general, equal group sizes are statistically more efficient; they are not necessary, however, which is fortunate since some alleles of interest might be so rare that finding, say, even ten individuals who are homozygous would be difficult.) A second kind of statistical comparison would be to compare equal-sized groups of the three genotypes (AA, Aa, aa), in order to determine whether the presence or absence of a particular allele has a significant effect on the drug response trait. In this instance, the Outlier Unit is preferably composed of equal-sized groups of the three genotypes.

Assume that being a homozygote for the rare allele of the locus described in the preceding paragraph causes a 15% average difference in a pharmacokinetic parameter (e.g., the area under curve of drug concentration in blood) as compared to random individuals. Assume further that the Outliers Unit has a total of sixty individuals, including thirty individuals of the rare genotype and thirty individuals chosen at random. Finally, assume that the variance of individual responses is identical within the two groups and that it is equal to 0.1. Standard statistical theory indicates that thirty individuals per group is not adequate to statistically prove that there is a significant difference in average uptake rate between the groups (P<0.05). Instead, with an increase to 108 individuals in each group, one would be able to provide statistical evidence for this effect. However, if we assume that homozygosity for an allele at the candidate locus causes a 30% difference in area under curve then the number of individuals required to provide statistical evidence for a difference between the two groups (for P<0.05 and holding all other assumptions constant) is only twenty-seven. The number of individuals required to detect a 60% difference in area under curve (all other assumptions constant) is only seven. This calculation assumes that the loci in question affect only the average trait in each of the two groups and that the shapes of the trait distribution are identical in the two groups. While conclusions based upon such an assumption are biologically meaningful and statistically robust, in some circumstances there may be differences in the shape of the trait distributions associated with different genotypes. In particular, one or more classes of homozygous genotypes may have a narrower trait distribution (smaller variance) than another, or than the population as a whole. Such a difference can be accounted for in the analysis; in fact, it would be expected to reduce the number of subjects needed for the Outliers Unit trial (since the smaller variance of one distribution reduces the overlap between it and the other trait distribution[s] to which it is being compared). In fact, the assumption of identical variances in the homozygote and total groups is not necessarily the biologically most likely case: it is reasonable to expect that the variance of the trait is the genetically more homogeneous group may be less (if the locus in question in fact contributes to variation in the drug response trait). This effect would result in a smaller population being adequate to show a genetically determined component to the difference in treatment effect between the two groups.

Serious adverse effects occurring at low frequency are often detected in the later stages of drug development. In some cases such effects have a significant genetic component. To address this issue preemptively, an Outlier Unit can perform trials in which subjects are selected to represent only the rare alleles at one or more loci that are candidates for influencing the response to treatment. For example, variances occurring at 5% allele frequency are expected to occur in homozygous form in 0.25% of the population (0.05×0.05), and therefore may rarely, if ever, be encountered in early clinical development. Yet such subjects could readily be identified by genotyping the hundreds to thousands of patients enrolled in a Phase I Outliers Unit.

Alternatively, by insuring that all common genotypes are represented in an Outlier Unit study the contribution of a major candidate locus can be tested with a powerful statistical design. Consider a locus with five haplotypes, A, B, C, D and E, with frequencies 0.3, 0.25, 0.2, 0.15, and 0.05 (plus several additional alleles with frequency lower than 0.05). A comparison of groups of homozygous for each of the haplotypes—that is AA, BB, CC, DD and EE homozygotes—each group of equal size, provides a powerful design to measure the contribution of variation at the candidate locus to variation in drug response. In this case, determination of sample sizes rests upon assumptions about the differences in average trait values for each haplotype. All other things being equal, detecting a difference is easiest when a subset of the haplotypes appears to be appreciably distinct from the rest. Such a situation allows one to make a reasonably principled decision to lump haplotypes so that one compares, say, one haplotype with all of the others. In such a circumstance, sample size calculations for testing a difference in average responses would be roughly similar to those described above. More generally, one can assess the overall heterogeneity of the traits associated with each haplotype (say, with a parametric or nonparametric analysis of variance) and one can also make individual comparisons between haplotypes (by using a multiple comparison procedure if the initial analysis of variance reveals significant heterogeneity). The identification of genetically determined phenotypic variation at such a locus then can reduce the likelihood of discrepant results due to genetic stratification in later trials.

In another embodiment of the invention, it would be useful to prospectively determine the status of polymorphisms at genes that are involved in the pharmacokinetic or pharmacodynamic action of many drugs. This would save genotyping the large Outliers Unit population each time a new project is initiated. Demand for genotyped groups of patients can be anticipated from pharmaceutical and biotechnology companies and contract research organizations (CROs). Genotyping might initially focus on common pharmacological targets such as estrogen receptors or other nuclear receptors, or on adrenergic receptors, serotonin receptors, dopamine receptors and other G protein coupled receptors. The pre-genotyped Outlier Unit population could be part of a package of services (along with genotyping assay development capability, high-throughput genotyping capacity and software and expertise in statistical genetics) designed to accelerate pharmacogenetic Phase I studies. Eventually, as the databank of genotypes is expanded, individuals with virtually any genotype or combination of genotypes can be called in for precisely designed physiological or toxicological studies designed to test for pharmacogenetic effects.

As noted earlier, the Pharmacogenetic Phase I Relatives Unit and the Pharmacogenetic Phase I Outlier Unit can provide useful information at almost any stage of clinical development. It is not unusual, for example, for a product in Phase II or even Phase III testing to be remanded to Phase I in order to clarify some aspect of toxicology or physiology. In this context, either or both of the Pharmacogenetic Phase I Units would be extremely useful to a drug development company, as studies in groups of related individuals (Relatives Unit) or in defined genetic subgroups drawn from a large genotyped population (Outliers Unit) would be an economical and efficient way to clarify the nature and extent of pharmacogenetic effects, if any, thereby paving the way for future rational development of the compound.

5. Surrogate Endpoints

As explained above, some of the most attractive applications of Pharmacogenetic Phase I Units depend on the availability of surrogate markers for pharmacodynamic drug action. The most useful surrogate markers are those which can be used in normal subjects in Phase I; which can be measured easily, inexpensively and accurately, and for which there is compelling data linking the surrogate marker with some clinically important aspect of disease biology, such as disease manifestations in various organ systems, disease progression, disease morbidity or mortality, or disparate other clinical indices known in the art. The utility of surrogate markers increases in proportion to the difficulty and cost of clinical development. Thus for a disease like Alzheimer's, where long trials involving many patients are standard, the use of surrogate measures of, for example, cognitive ability, are highly desirable.

The standard endpoints of Phase I trials are also useful measures for analysis in a Pharmacogenetic Phase I Unit. For example, studies of compound adsorption, distribution, metabolism, excretion and bioavailability may be analyzed for their genetic component. Similarly, toxic responses and dose-related side effects may be analyzed by the pharmacogenetic methods of this invention.

6. Establishing and Operating a Phase I Pharmacogenetic Relatives Unit

First, it should be noted that the information that can be gained from a Pharmacogenetic Phase I Unit provides for substantial cost savings in later stages of clinical development. Therefore its is to be expected that even if the cost of operating a Pharmacogenetic Phase I Unit exceeds the cost of operating a conventional Phase I Unit, the overall costs of clinical development are likely to be lower, thereby justifying the costs of the Pharmacogenetic Phase I Unit. Nonetheless, it is clearly desirable to operate a Pharmacogenetic Phase I Unit as efficiently as possible. In order to make a Phase I unit an effective business operation it is useful to (i) use statistical genetic methods to design studies that require the minimal number of subjects to achieve adequate statistical power (e.g. power of 80% to detect an effect at the P<0.05 level), in order to keep subject costs at a minimum, (ii) take measures to reduce the turnover of participating subjects, in view of the long term investment made in patient recruitment and (in the case of the Outliers Unit) genotyping. This may be accomplished by offering subjects financial or other incentives to encourage sustained participation in the Pharmacogenetic Phase I Unit. The types of incentives that would be useful differ between the two types of Phase I Units (see below), (iii) Secure rights to reuse genotype data and, ideally, phenotypic data collected during each Pharmacogenetic Phase I Unit trial, in order to create a database that over time will save costs by eliminating the need to repetitively genotype the same loci, and may eventually produce information of broad utility in clinical pharmacology research: namely a database on the heritability of phenotypic responses to various broad classes of compounds (benzodiazepines, statins, taxanes, etc.) and the major classes of genes involved. Such a database could become a product.

In order to efficiently set up a Phase I Pharmacogenetic Relatives Unit family participation can be encouraged by appropriate incentive compensation. For example, subjects with no participating family members might be paid $200 for participation in a study; two sibs participating in the same study might each be paid $300; if they could encourage another sib (or cousin) to participate the three related individuals might each be paid $350 for each study; parent-sib pairs might be paid $400 for each study, and so forth. This type of compensation would encourage subjects to recruit their relatives to participate in Phase I studies. To the extent that certain types of blood relationship are more useful for efficient genetical analysis, those types of related individuals could be compensated most highly. This type of compensation would increase the cost of studies, however the increased speed of setting up the Relatives Unit, and the increased retention of subjects, would compensate over time. The optimal location to establish a Pharmacogenetic Relatives Unit is in a city with a stable population, many large families, and a open attitudes toward modern technology. The size of a Relatives Unit need be little more than 150 subjects, though 250 would allow greater flexibility in drawing related subjects from different racial or ethnic groups (see below), and allow for more trials to be performed simultaneously. 400-500 subjects would be most preferable. Greater than 500 subjects would provide little benefit while increasing costs substantially.

Ideally subjects in the pharmacogenetic Phase I unit are of known ethnic/racial/geographic background and willing to participate in Phase I studies, for pay, over a period of years. For specific studies in a Relatives Unit subjects from one or more racial, ethnic or geographically defined group may be analyzed in order to (i) mirror the population in which Phase II or Phase III trials are to be conducted; (ii) determine if there are measurable differences in pharmacogenetic effects in different racial, ethnic or geographically defined groups; (iii) study the most homogeneous group possible in order to increase the chances of detecting a particular type of genetic effect.

Ideally consent for genotyping should be obtained at the same time that subjects are enrolled. Appropriate consent forms will be drafted and approved by an independent review board. It would be most efficient if blanket consent for genotyping any polymorphic site or sites deemed relevant to the pharmacology of any candidate drug could be obtained. However, if this somewhat broad type of consent is deemed inappropriate by the review board then consent could be somewhat narrowed by adding the qualification that any loci that are genotyped be relevant to a customer project. A third, more onerous arrangement would be obtain consent to genotype polymorphic sites in loci relevant to specific families of compounds, or to obtain consent for genotyping a specific list of genes. Another, still less desirable solution would be to obtain consent for genotyping on a project-by-project basis (for example by mailing out reply cards to all subjects for each study), after the specific polymorphic sites to be genotyped have been selected.

Another essential element of operating a Relatives Unit is having adequate quality control measures. One crucial aspect of quality control is an independent testing method to confirm the relatedness of the recruited subjects. This can be accomplished by genotyping multiple (10-50) highly polymorphic loci, such as short tandem repeat sequences, in individuals believed to be rated. By comparing the degree of genetic identity observed with that expected from the purported relation (e.g. 50% in the case of sibs) it is possible to ensure with considerable certainty that all related individuals are in fact related as they believe themselves to be. (Inconsistency between genotyping and reported relationship would be dealt with simply by not enrolling the unrelated individuals in any trials.)

As indicated above, methods for retention of subjects in a Phase I Outliers Unit preferably consist of making modest payments for continuing participation (i.e. continued permission to genotype under the limits of the consent); additional payments for genotyping analysis, whether or not it results in a request to participate in a clinical study; and, of course, generous compensation for participation in each Outliers Unit clinical study.

Phase I of clinical development is generally focused on safety, although drug companies are increasingly obtaining information on pharmacokinetics and surrogate pharmacodynamic markers in early trials. Phase I studies are typically performed with a small number (<60) of normal, healthy volunteers usually at single institutions. The primary endpoints in these studies usually relate to pharmacokinetic parameters (i.e. adsorption, distribution, metabolism and bioavailability), and dose-related side effects. In a Phase I pharmacogenetic clinical trial, stratification based upon allelic variance or variances of a candidate gene or genes related to pharmacokinetic parameters may allow early assessment of potential genetic interactions with treatment.

Phase I studies of some diseases (e.g. cancer or other medically intractable diseases for which no effective medical alternative exists) may include patients who satisfy specified inclusion criteria. These safety/limited-efficacy studies can be conducted at multiple institutions to ensure rapid enrollment of patients. In a pharmacogenetic Phase I study that includes patients, or a mixture of patients and normals, the status of a variance or variances suspected to affect the efficacy of the candidate therapeutic intervention may be used as part of the inclusion criteria. Alternatively, analysis of variances or haplotypes in patients with different treatment responses may be among the endpoints. It is not unusual for such a Phase I study design to include a double-blind, balanced, random-order, crossover sequence (separated by washout periods), with multiple doses on separate occasions and both pharmacokinetic and pharmacodynamic endpoints.

2. Phase I Trials with Subjects Drawn from Large Populations and/or from Related Volunteer Subjects: the Pharmacogenetic Phase I Unit Concept

In general it is useful to be able to assess the contribution of genetic variation to treatment response at the earliest possible stage of clinical development. Such an assessment, if accurate, will allow efficient prioritization of candidate compounds for subsequent detailed pharmacogenetic studies; only those treatments where there is early evidence of a significant interaction of genetic variation with treatment response would be advanced to pharmacogenetic studies in later stages of development. In this invention we describe methods for achieving early insight—in Phase I—into the contribution of genetic variation to variation in surrogate treatment response variables. It occurred to the inventors that this can be accomplished by bringing the power of genetic linkage analysis and outlier analysis to Phase I testing via the recruitment of a very large Phase I population including a large number of individuals who have consented in advance to genetic studies (occasionally referred to hereinafter as a Pharmacogenetic Phase I Unit). In one embodiment of a Pharmacogenetic Phase I Unit many of the subjects are related to each other by blood. (Currently Phase I trials are performed in unrelated individuals, and there is no consideration of genetic recruitment criteria, or of genetic analysis of surrogate markers.) There are several novel ways in which a large population, or a population comprised at least in part of related individuals, could be useful in early clinical trials. Some of the most attractive applications depend on the availability of surrogate markers for pharmacodynamic drug action which can be used early in clinical development, preferably in normal subjects in Phase I. Such surrogate markers are increasingly used in Phase I, as drug development companies seek to make early yes/no decisions about compounds.

Recruitment of a population optimized for clinical genetic investigation may entail utilization of methods in statistical genetics to select the size and composition of the population. For example powerful methods for detecting and mapping quantitative trait loci in sibpairs have been developed. These methods can provide some estimate of the statistical power derived from a given number of groups of closely related individuals. Ideally subjects in the pharmacogenetic Phase I unit are of known ethnic/racial/geographic background and willing to participate in Phase I studies, for pay, over a period of years. The population is preferably selected to achieve a specified degree of statistical power for genetic association studies, or is selected in order to be able to reliably identify a certain number of individuals with rare genotypes, as discussed below. Family participation could be encouraged by appropriate incentive compensation. For example, individual subjects might be paid $200 for participation in a study; two sibs participating in the same study might each be paid $300; if they could encourage another sib (or cousin) to participate the three related individuals might each be paid $350, and so forth. This type of compensation would encourage subjects to recruit their relatives to participate in Phase I studies. (It would also increase the cost of studies, however the type of data that can be obtained can not be duplicated with conventional approaches.) The optimal location to establish such a Phase I unit is a city with a stable population, many large families, and a positive attitude about gene technology. The Pharmacogenetic Phase I Unit population can then be used to test for the existence of genetic variation in response to any drug as a first step in deciding whether to proceed with extensive pharmacogenetic studies in later stages of clinical development. Specific uses of a large Phase I unit in which some or all subjects are related include:

a. It should be possible, for virtually any compound, to assess the magnitude of the genetic contribution to variation in drug response (if any) by comparing variation in drug response traits among related vs. non-related individuals. The rationale is as follows: if a surrogate drug response trait (i.e. a surrogate marker of pharmacodynamic effect that can be measured in normal subjects) is under strong genetic control then related individuals, who share 25% (cousins) or 50% (sibs) of their alleles, should have less divergent responses (less intragroup variance) than unrelated individuals, who share a much smaller fraction of alleles. That is, individuals who share alleles at the genes that affect drug response should be more similar to each other (i.e. have a narrower distribution of responses, whether measured by variance, standard deviation or other means) than individuals who, on average, share very few alleles. By using statistical methods known in the art the degree of variation in a set of data from related individuals (each individual would only be compared with his/her relatives, but such comparisons would be performed within each group of relatives and a summary statistic developed) could be compared to the degree of variation in a set of unrelated individuals (the same subjects could be used, but the second comparison would be across related groups). Account would be taken of the degree of similarity expected between related individuals, based on the fraction of the genome they shared by descent. Thus the extent of variation in the surrogate response marker between identical twins should be less than between sibs, which should be less than between first cousins, which should be less than that between second cousins, and so forth, if there is a genetic component to the variation. It is well known from twin studies (in which, for example, variation between identical twins is compared to variation between fraternal twins) that pharmacokinetic variables (e.g. compound half life, peak concentration) are frequently over 90% heritable; the type of study proposed here (comparison of variation within groups of sibs and cousins to variation between unrelated subjects) would also show this genetic effect, without requiring the recruitment of monozygotic twins. For a summary of pharmacokinetic studies in twins see: Propping, Paul (1978) Pharmacogenetics. Rev. Physiol. Biochem. Pharmacol. 83: 123-173.

It may be that the pattern of drug responses that distinguishes related individuals from non-related individuals is more complex than, for example, variance or standard deviation. For example, there may be two discrete phenotypes characteristic of intrafamilial variation (a bimodal distribution) that are not a feature of variation between unrelated individuals (where, for example, variation might be more nearly continuous). Such a pattern could be attributable to Mendelian inheritance operating on a restricted set of alleles in a family (or families) with, for example, AA homozygotes giving one phenotype and AB heterozygotes and BB homozygotes giving a second phenotype, all in the context of a relatively homogeneous genetic background. In contrast, variation among non-related subjects would be less discrete due to a greater degree of variation in genetic background and the presence of additional alleles C, D and E at the candidate locus. Statistical measures of the significance of such differences in distribution, including nonparametric methods such as chi square and contingency tables, are known in the art.

The methods described herein for measuring whether pharmacodynamic traits are under genetic control, using surrogate markers of drug efficacy in phase I studies which include groups of related individuals, will be useful in obtaining an early assessment of the extent of genetically determined variation in drug response for a given therapeutic compound. Such information provides an informed basis for either stopping development at the earliest possible stage or, preferably, continuing with development but with a plan for identifying and controlling for genetic variation so as to allow rapid progression through the regulatory approval process.

For example, it is well known that Alzheimer's trials are long and expensive, and most drugs are only effective in a fraction of patients. Using surrogate measures of response in normals drawn from a population of related individuals would help to assess the contribution of genetic variation to variation in treatment response. For an acetylcholinesterase inhibitor, relevant surrogate pharmacodynamic measures could include testing erythrocye membrane acetylcholinesterase levels in drug treated normal subjects, or performing psychometric tests that are affected by treatment (and ideally that correlate with clinical efficacy) and measuring the effect of treatment. As another example, antidepressant drugs can produce a variety of effects on mood in normal subjects—or no effect at all. Careful monitoring and measurement of such responses in related vs. unrelated normal subjects, and statistical comparison of the degree of variation in each group, could provide an early readout on whether there is a genetic component to drug response (and hence clinical efficacy). The observation of similar effects in family members, and comparatively dissimilar effects in unrelated subjects would provide compelling evidence of a pharmacogenetic effect and justify the substantial expenditure necessary for a full pharmacogenetic drug development program. Conversely, the absence of any significant family influence on drug response would provide an early termination point for pharmacogenetic studies. Note that the proposed studies do not require any knowledge of candidate genes, nor is DNA collection or genotyping required—simply a reliable surrogate pharmacodynamic assay and small groups of related normal individuals. Refined statistical methods should permit the magnitude of the pharmacogenetic effect to be measured, which could be a further criteria for deciding whether to proceed with pharmacogenetic analysis. The greater the differential in magnitude or pattern of variance between the related and the unrelated subjects, the greater the extent of genetic control of the trait.

Not all drug response traits are under the predominant control of one locus. Many such traits are under the control of multiple genes, and may be referred to as quantitative trait loci. It is then desirable to identify the major loci contributing to variation in the drug response trait. This can be done for example, to map quantitative trait loci in a population of drug treated related normals. Either a candidate gene approach or a genome wide scanning approach can be used. (For review of some relevant methods see: Hsu L, Aragaki C, Quiaoit F. (1999) A genome-wide scan for a simulated data set using two newly developed methods. Genet Epidemiol 17 Suppl 1:S621-6; Zhao L P, Aragaki C, Hsu L, Quiaoit F. (1998) Mapping of complex traits by single-nucleotide polymorphisms. Am J Hum Genet 63(1):225-40; Stoesz M R, Cohen J C, Mooser V, et al. (1997) Extension of the Haseman-Elston method to multiple alleles and multiple loci: theory and practice for candidate genes. Ann Hum Genet 61 (Pt 3):263-74.)) However, this method would require at least 100 patients (preferably 200, and still more preferably >300) to have adequate statistical power, and each patient would have to be genotyped at a few polymorphic loci (candidate gene approach) or hundreds of polymorphic loci (genome scanning approach).

b. With a large Phase I population of normal subjects that need not be related (a second type of Pharmacogenetic Phase I Unit) it is possible to efficiently identify and recruit for any Phase I trial a set of individuals comprising virtually any combination of genotypes present in a population (for example, all common genotypes, or a group of genotypes expected to represent outliers for a drug response trait of interest). This method preferably entails obtaining blood or other tissue (e.g. buccal smear) in advance from a large number of the subjects in the Phase I unit. Ideally consent for genotyping would be obtained at the same time. It would be most efficient if blanket consent for genotyping any polymorphic site or sites could be obtained. Second best would be consent for testing any site relevant to any customer project (not specific at the time of initial consent). Third best would be consent to genotype polymorphic sites relevant to specific disease areas. Another, less desirable, solution would be to obtain consent for genotyping on a project by project basis (for example by mailing out reply cards), after the specific polymorphic sites to be genotyped are known.

One useful way to screen for pharmacogenetic effects in Phase I is to recruit homozygotes for a variance or variances of interest in one or more candidate genes. For example, consider a compound for which there are two genes that are strong candidates for influencing response to treatment. Gene X has alleles A and A′, while gene Y has alleles B and B′. If these genes do in fact contribute significantly to response then one would expect that, regardless of the mode of inheritance (recessive, codominant, dominant, polygenic) homozygotes would exhibit the most extreme responses. One would also expect epistatic interactions, if any, to be most extreme in double homozygotes. Thus one would ideally perform a surrogate drug response test in Phase I volunteers doubly homozygous at both X and Y. That is, test AA/BB, A′A′/BB, AA/B′B′ and A′A′/B′B′ subjects. If the allele frequencies for A and A′ are 0.15 and 0.85, and for B and B′ 0.2 and 0.8 then the frequency of AA homozygotes is expected to be 2.25% and BB homozygotes 4%. In the absence of any linkage between the genes, the frequency of AA/BB double homozygotes is expected to be 0.0225×0.04=0.0009 or 0.09%, or about 1 subject in 1000. Ideally at least 5 subjects of each genotype are recruited for the Phase I study, and preferably at least 10 subject. Thus, even for variances of moderately low allele frequency (15%, 20%), the identification of potential outliers (i.e. homozygotes) for the candidate genes of interest will require a large population. Preferably the Phase I unit has enrolled at least 1,000 normal individuals, more preferably 2,000, still more preferably 5,000 and most preferably 10,000 or more. In another application of the large, genotyped Phase I population it may be useful to identify individuals with rare variances in candidates genes (either homozygous or heterozygous), in order to determine whether those variances are predisposing to extreme pharmacological responses to the compound. For example, variances occurring at 5% allele frequency are expected to occur in homozygous form in 0.25% of the population (0.05×0.05), and therefore may rarely, if ever, be encountered in early clinical development. Yet it may be serious adverse effects occurring in just such a small group that create problems in later stages of drug development. In yet another application of the large genotyped Phase I population, subjects may be selected to represent the known common variances in one or more genes that are candidates for influencing the response to treatment. By insuring that all common genotypes are represented in a Phase I trial the likelihood of misleading results due to genetic stratification (resulting in discrepancy with results of later, larger trials can be reduced.

It would be useful to prospectively genotype the large Phase I population for variances that are commonly the source of interpatient variation in drug response, since demand for genotyped groups of such patients can be anticipated from pharmaceutical companies and contract research organizations (CROs). For example, genotyping might initially focus on common pharmacological targets such as estrogen receptors, adrenergic receptors, or serotonin receptors. The pre-genotyped Phase I population could be part of a package of services (along with genotyping assay development capability, high throughput genotyping capacity and software and expertise in statistical genetics) designed to accelerate pharmacogenetic Phase I studies. Eventually, as the databank of genotypes built up, individuals with virtually any genotype or combination of genotypes could be called in for precisely designed physiological or toxicological studies designed to test for pharmacogenetic effects.

One of the most useful aspects of the Pharmacogenetic Phase I Unit is that subjects with rare genotypes can be pharmacologically assessed in a small study. This addresses a serious limitation of conventional clinical trials with respect to the investigation of polygenic traits or the effect of rare alleles. Unfortunately even Phase III studies, as currently performed, are often barely powered to address simple one variance hypotheses about efficacy or toxicity. The problem, of course, is that each time a new genetic variable is introduced the comparison groups are cut in halves or thirds (or even smaller groups if there are multiple haplotypes at each gene). It is therefore a challenging problem to test the interaction of several genes in determining drug response. Yet the character of drug response data in populations—there is often a continuous distribution of responses among different individuals—suggests that drug responses may often be mediated by several genes. (On the other hand, there are an increasing number of well documented single gene, or even single variance, pharmacogenetic effects in the literature, showing that it is possible to detect the effect of a single variance.) One approach to identifying pharmacogenetic effects is to focus on finding the single gene variances that have the largest effects. This approach can be undertaken within the scale of current clinical trials. However, in order to develop a test which predicts a large fraction of the quantitative variation in a drug response trait it may be desirable to test the effect of multiple genes, including the interaction of variances at different genes, which may be non-additive (referred to as epistasis). The Pharmacogenetic Phase I Unit provides a way to efficiently test for gene interactions or multigene effects by, for example, allowing easy identification of individuals who, on account of being homozygous at several loci of interest, should be outliers for the drug response phenotypes of interest if there is a gene×gene interaction. Testing drug response in a small number of such individuals will provide a quick read on gene interaction. Obtaining genetic data on the pharmacodynamic action of a compound in Phase I should also provide a crude measure of allele affects—which variances or haplotypes increase pharmacological responses and which decrease them. This information is of great value in designing subsequent trials, as it constrains the number of hypotheses to be tested, thereby enabling powerful statistical designs. This is because when the effect of variances on drug response measures is unknown one is forced to statistically test all the possible effects of each allele (e.g. two tailed tests). As the number of genetically defined groups increases (e.g. as a result of multiple variances or haplotypes) there is a loss of statistical power due to multiple testing correction. On the other hand, if the relative phenotypic effect of each allele at a locus is known (or can be hypothesized) from Phase I data then each individual in a subsequent clinical trial contributes useful information—there is a specific prediction of response based on that individuals combination of genotypes or haplotypes, and testing the fit of the actual data to those predictions provides for powerful statistical designs. (It is also possible to measure allele effects biochemically, of course, to establish which alleles have positive and which negative effects, but at considerable cost.)

It is important to note that Phase I trials can provide useful information at almost any stage of clinical development. It is not unusual, for example, for a product in Phase II or even Phase III testing to be remanded to Phase I in order to clarify some aspect of toxicology or physiology. In this context a Pharmacogenetic Phase I Unit would be extremely useful to a drug development company. Phase I studies in defined genetic subgroups drawn from a large genotyped population, or in groups of related individuals, would be the most economical and efficient way to clarify the existence of pharmacogenetic effects, if any, paving the way for future rational development of the product.

C. Phase II Clinical Trials

Phase II studies generally include a limited number of patients (<100) who satisfy a set of predefined inclusion criteria and do not satisfy any predefined exclusion criteria of the trial protocol. Phase II studies can be conducted at single or multiple institutions. Inclusion/exclusion criteria may include historical, clinical and laboratory parameters for a disease, disorder, or condition; age; gender; reproductive status (i.e. pre- or postmenopausal); coexisting medical conditions; psychological, emotional or cognitive state, or other objective measures known to those skilled in the art. In a pharmacogenetic Phase II trial the inclusion/exclusion criteria may include one or more genotypes or haplotypes. Alternatively, genetic analysis may be performed at the end of the trial. The primary goals in Phase II testing may include (i) identification of the optimal medical indication for the compound, (ii) definition of an optimal dose or range or doses, balancing safety and efficacy considerations (dose-finding studies), (iii) extended safety studies (complementing Phase I safety studies), (iv) evaluation of efficacy in patients with the targeted disease or condition, either in comparison to placebo or to current best therapy. To some extent these goals may be achieved by performing multiple trials with different goals. Likewise, Phase II trials may be designed specifically to evaluate pharmacogenetic aspects of the drug candidate. Primary efficacy endpoints typically focus on clinical benefit, while surrogate endpoints may measure treatment response variables such as clinical or laboratory parameters that track the progress or extent of disease, often at lesser time, cost or difficulty than the definitive endpoints. A good surrogate marker must be convincingly associated with the definitive outcome. Examples of surrogate endpoints include tumor size as a surrogate for survival in cancer trials, and cholesterol levels as a surrogate for heart disease (e.g. myocardial infarction) in trials of lipid lowering cardiovascular drugs. Secondary endpoints supplement the primary endpoint and may be selected to help guide further clinical studies.

In a pharmacogenetic Phase II clinical trial, retrospective or prospective design will include the stratification of patients based upon a variance or variances in a gene or genes suspected of affecting treatment response. The gene or genes may be involved in mediating pharmacodynamic or pharmacokinetic response to the candidate therapeutic intervention. The parameters evaluated in the genetically stratified trial population may include primary, secondary or surrogate endpoints. Pharmacokinetic parameters—for example, dosage, absorption, toxicity, metabolism, or excretion—may also be evaluated in genetically stratified groups. Other parameters that may be assessed in parallel with genetic stratification include gender, race, ethnic or geographic origin (population history) or other demographic factors.

While it is optimal to initiate pharmacogenetic studies in phase I, as described above, it may be the case that pharmacogenetic studies are not considered until phase II, when problems relating either to efficacy or toxicity are first encountered. It is highly desirable to initiate pharmacogenetic studies no later than Phase II of a clinical development plan because (1) phase III studies tend to be large and expensive—not an optimal setting in which to explore untested pharmacogenetic hypotheses; (2) phase III studies are typically designed to test one fairly narrow hypothesis regarding efficacy of one or a few dose levels in a specific disease or condition. Phase II studies are often numerous, and are intended to provide a broad picture of the pharmacology of the candidate compound. This is a good setting for initial pharmacogenetic studies. Several pharmacogenetic hypotheses may be tested in phase II, with the goal of eliminating all but one or two.

D. Phase III Clinical Trials

Phase III studies are generally designed to measure efficacy of a new treatment in comparison to placebo or to an established treatment method. Phase II studies are often performed at multiple sites. The design of this type of trial includes power analysis to ensure the sufficient data will be gathered to demonstrate the anticipated effect, making assumptions about response rate based on earlier trials. As a result Phase III trials frequently include large numbers of patients (up to 5,000). Primary endpoints in Phase III studies may include reduction or arrest of disease progression, improvement of symptoms, increased longevity or increased disease-free longevity, or other clinical measures known in the art. In a pharmacogenetic Phase III clinical study, the endpoints may include determination of efficacy or toxicity in genetically defined subgroups. Preferably the genetic analysis of outcomes will be confined to an assessment of the impact of a small number of variances or haplotypes at a small number of genes, said variances having already been statistically associated with outcomes in earlier trials. Most preferably variances at only one or two genes will be assessed.

After successful completion of one or more Phase III studies, the data and information from all trials conducted to test a new treatment method are compiled into a New Drug Application (NDA) and submitted for review by the US FDA, which has authority to grant marketing approval in the US and its territories. The NDA includes the raw (unanalyzed) clinical data, i.e. the patient by patient measurements of primary and secondary endpoints, a statistical analysis of all of the included data, a document describing in detail any observed side effects, tabulation of all patients who dropped-out of trials and detailed reasons for their termination, and any other available data pertaining to ongoing in vitro or in vivo studies since the submission of the investigational new drug (IND) application. If pharmacoeconomic objectives are a part of the clinical trial design then data supporting cost or economic analyses are included in the NDA. In a pharmacogenetic clinical study, the pharmacoeconomic analyses may include genetically stratified assessment of the candidate therapeutic intervention in a cost benefit analysis, cost of illness study, cost minimization study, or cost utility analysis. The analysis may also be simultaneously stratified by standard criteria such as race/ethnicity/geographic origin, sex, age or other criteria. Data from a genetically stratified analysis may be used to support an application for approval for marketing of the candidate therapeutic intervention.

E. Phase IV Clinical Trials

Phase IV studies occur after a therapeutic intervention has been approved for marketing, and are typically conducted for surveillance of safety, particularly occurrence of rare side effects. The other principal reason for Phase IV studies is to produce information and relationships useful for marketing a drug. In this regard pharmacogenetic analysis may be very useful in Phase IV trials. Consider, for example, a drug that is the fourth or fifth member of a drug class (say statins, or thiazidinediones or fluoropyrimidines) to obtain marketing approval, and which does not differ significantly in clinical effects—efficacy or safety—from other members of the drug class. The first, second and third drugs in the class will likely have a dominant market position (based on their earlier introduction into the marketplace) that is difficult to overcome, particularly in the absence of differentiating clinical effects. However, it is possible that the new drug produces a superior clinical effect—for example, higher response rate, greater magnitude of response or fewer side effects—in a genetically defined subgroup. The genetic subgroup with superior response may constitute a larger fraction of the total patient population than the new drug would likely achieve otherwise. In this instance, there is a clear rationale for performing a Phase IV pharmacogenetic trial to identify a variance or variances that mark a patient population with superior clinical response. Subsequently a marketing campaign can be designed to alert patients, physicians, pharmacy managers, managed care organizations and other parties that, with the use of a rapid and inexpensive genetic test to identify eligible patients, the new drug is superior to other members of the class (including the market leading first, second and third drugs introduced). The high responder subgroup defined by a variance or variances may also exhibit a superior response to other drugs in the class (a class pharmacogenetic effect), or the superior efficacy in the genetic subgroup may be specific to the drug tested (a compound-specific pharmacogenetic effect).

In a Phase IV pharmacogenetic clinical trial, both retrospective and prospective analysis can be performed. In both cases, the key element is genetic stratification based on a variance or variances or haplotype. Phase IV trials will often have adequate sample size to test more than one pharmacogenetic hypothesis in a statistically sound way.

F. Unconventional Clinical Development

Although the above listed phases of clinical development are well-established, there are cases where strict Phase I, II, III development does not occur, for example, in the clinical development of candidate therapeutic interventions for debilitating or life threatening diseases, or for diseases where there is presently no available treatment. Some of the mechanisms established by the FDA for such studies include Treatment INDs, Fast-Track or Accelerated reviews, and Orphan Drug Status. In a clinical development program for a candidate therapeutic of this type there is a useful role for pharmacogenetic analysis, in that the candidate therapeutic may not produce a sufficient benefit in all patients to justify FDA approval, however analysis of outcome in genetic subgroups may lead to identification of a variance or variances that predict a response rate sufficient for FDA approval.

As used herein, “supplemental applications” are those in which a candidate therapeutic intervention is tested in a human clinical trial in order to gain an expanded label indication, expanding recommended use to new medical indications. In these applications, previous clinical studies of the therapeutic intervention, i.e. preclinical safety and Phase I human safety studies can be used to support the testing of the therapeutic intervention in a new indication. Pharmacogenetic analysis is also useful in the context of clinical trials to support supplemental applications. Since these are, by definition, focused on diseases not selected for initial development the overall efficacy may not be as great as for the leading indication(s). The identification of genetic subgroups with high response rates may enable the rapid approval of supplemental applications for expanded label indications. In such instances part of the label indication may be a description of the variance or variances that define the group with superior response.

As used herein, “outcomes” or “therapeutic outcomes” describe the results and value of healthcare intervention. Outcomes can be multi-dimensional, and may include improvement of symptoms; regression of a disease, disorder, or condition; prevention of a disease or symptom; cost savings or other measures.

Pharmacoeconomics is the analysis of a therapeutic intervention in a population of patients diagnosed with a disease, disorder, or condition that includes at least one of the following studies: cost of illness study (COI); cost benefit analysis (CBA), cost minimization analysis (CMA), or cost utility analysis (CUA), or an analysis comparing the relative costs of a therapeutic intervention with one or a group of other therapeutic interventions. In each of these studies, the cost of the treatment of a disease, disorder, or condition is compared among treatment groups. Costs have both direct (therapeutic interventions, hospitalization) and indirect (loss of productivity) components. Pharmacoeconomic factors may provide the motivation for pharmacogenetic analysis, particularly for expensive therapies that benefit only a fraction of patients. For example, interferon alpha is the only treatment that can cure hepatitis C virus infection, however viral infection is completely and permanently eliminated in less than a quarter of patients. Nearly half of patients receive virtually no benefit from alfa interferon, but may suffer significant side effects. Treatment costs are ˜$10,000 per course. A pharmacogenetic test that could predict responders would save much of the cost of treating patients not able to benefit from interferon alpha therapy, and could provide the rationale for treating a population in a cost efficient manner, where treatment would otherwise be unaffordable.

As used herein, “health-related quality of life” is a measure of the impact of a disease, disorder, or condition on a patient's activities of daily living. An analysis of the health-related quality of life is often included in pharmacoeconomic studies.

As used herein, the term “stratification” refers to the partitioning of patients into groups on the basis of clinical or laboratory characteristics of the patient. “Genetic stratification” refers to the partitioning of patients or normal subjects into groups based on the presence or absence of a variance or variances in one or more genes. The stratification may be performed at the end of the trial, as part of the data analysis, or may come at the beginning of a trial, resulting in creation of distinct groups for statistical or other purposes.

G. Power Analysis in Pharmacogenetic Clinical Trials

The basic goal of power calculations in clinical trial design is to insure that trials have adequate patients and controls to fairly assess, with statistical significance, whether the candidate therapeutic intervention produces a clinically significant benefit.

Power calculations in clinical trials are related to the degree of variability of the drug response phenotypes measured and the treatment difference expected between comparison groups (e.g. between a treatment group and a control group). The smaller the variance within each group being compared, and the greater the difference in response between the two groups, the fewer patients are required to produce convincing evidence of an effect of treatment. These two factors (variance and treatment difference) determine the degree of precision required to answer a specific clinical question.

The degree of precision may be expressed in terms of the maximal acceptable standard error of a measurement, the magnitude of variation in which the 95% confidence interval must be confined or the minimal magnitude of difference in a clinical or laboratory value that must be detectable (at a statistically significant level, and with a specified power for detection) in a comparison to be performed at the end of the trial (hypothesis test). The minimal magnitude is generally set at the level that represents the minimal difference that would be considered of clinical importance.

In pharmacogenetic clinical trials there are two countervailing effects with respect to power. First, the comparison groups are reduced in size (compared to a conventional trial) due to genetic partitioning of both the treatment and control groups into two or more subgroups. However, it is reasonable to expect that variability for a trait is smaller within groups that are genetically homogeneous with respect to gene variances affecting the trait. If this is the case then power is increased as a function of the reduction in variability within (genetically defined) groups.

In general it is preferable to power a pharmacogenetic clinical trial to see an effect in the largest genetically defined subgroups. For example, for a variance with allele frequencies of 0.7 and 0.3 the common homozygote group will comprise 49% of all patients (0.7×0.7×100). It is most desirable to power the trial to observe an effect (either positive or a negative) in this group. If it is desirable to measure an effect of therapy in a small genetic group (for example, the 9% of patients homozygous for the rare allele) then genotyping should be considered as an enrollment criterion to insure a sufficient number of patients are enrolled to perform an adequately powered study.

Statistical methods for powering clinical trials are known in the art. See, for example: Shuster, J. J. (1990) Handbook of Sample Size Guidelines for Clinical Trials. CRC Press, Boca Raton, Fla.; Machin, D. and M. J. Campbell (1987) Statistical Tables for the Design of Clinical Trials. Blackwell, Oxford, UK; Donner, A. (1984) Approaches to Sample Size Estimation in the Design of Clinical Trials—A Review. Statistics in Medicine 3: 199-214.

H. Statistical Analysis of Clinical Trial Data

There are a variety of statistical methods for measuring the difference between two or more groups in a clinical trial. One skilled in the art will recognize that different methods are suited to different data sets. In general, there is a family of methods customarily used in clinical trials, and another family of methods customarily used in genetic epidemiological studies. Methods in quantitative and population genetics designed to measure the association between genotypes and phenotypes, and to map and measure the effect of quantitative trait loci are also relevant to the task of measuring the impact of a variance on response to a treatment. Methods from any of these disciplines may be suitable for performing statistical analysis of pharmacogenetic clinical trial data, as is known to those skilled in the art.

Conventional clinical trial statistics include hypothesis testing and descriptive methods, as elaborated below. Guidance in the selection of appropriate statistical tests for a particular data set is provided in texts such as: Biostatistics: A Foundation for Analysis in the Health Sciences, 7th edition (Wiley Series in Probability and Mathematical Statistics, Applied Probability and statistics) by Wayne W. Daniel, John Wiley & Sons, 1998; Bayesian Methods and Ethics in a Clinical Trial Design (Wiley Series in Probability and Mathematical Statistics. Applied Probability Section) by J. B. Kadane (Editor), John Wiley & Sons, 1996. Examples of specific hypothesis testing and descriptive statistical procedures that may be useful in analyzing clinical trial data are listed below.

A. Hypothesis Testing Statistical Procedures

(1) One-sample procedures (binomial confidence interval, Wilcoxon signed rank test, permutation test with general scores, generation of exact permutational distributions)

(2) Two-sample procedures (t-test, Wilcoxon-Mann-Whitney test, Normal score test, Median test, Van der Waerden test, Savage test, Logrank test for censored survival data, Wilcoxon-Gehan test for censored survival data, Cochran-Armitage trend test, permutation test with general scores, generation of exact permutational distributions)

(3) R×C contingency-tables (Fisher's exact test, Pearson's chi-squared test, Likelihood ratio test, Kruskal-Wallis test, Jonckheere-Terpstra test, Linear-by linear association test, McNemar's test, marginal homogeneity test for matched pairs)

(4) Stratified 2×2 contingency tables (test of homogeneity for odds ratio, test of unity for the common odds ratio, confidence interval for the common odds ratio)

(5) Stratified 2×C contingency tables (all two-sample procedures listed above with stratification, confidence intervals for the odds ratios and trend, generation of exact permutational distributions)

(6) General linear models (simple regression, multiple regression, analysis of variance -ANOVA-, analysis of covariance, response-surface models, weighted regression, polynomial regression, partial correlation, multiple analysis of variance -MANOVA-, repeated measures analysis of variance).

(7) Analysis of variance and covariance with a nested (hierarchical) structure.

(8) Designs and randomized plans for nested and crossed experiments (completely randomized design for two treatment, split-splot design, hierarchical design, incomplete block design, latin square design)

(9) Nonlinear regression models

(10) Logistic regression for unstratified or stratified data, for binary or ordinal response data, using the logit link function, the normit function or the complementary log-log function.

(11) Probit, logit, ordinal logistic and gompit regression models.

(12) Fitting parametric models to failure time data that may be right-, left-, or interval-censored. Tested distributions can include extreme value, normal and logistic distributions, and, by using a log transformation, exponential, Weibull, lognormal, loglogistic and gamma distributions.

(13) Compute non-parametric estimates of survival distribution with right-censored data and compute rank tests for association of the response variable with other variables.

B. Descriptive statistical methods

Factor analysis with rotations

Canonical correlation

Principal component analysis for quantitative variables.

Principal component analysis for qualitative data.

Hierarchical and dynamic clustering methods to create tree structure, dendrogram or phenogram.

Simple and multiple correspondence analysis using a contingency table as input or raw categorical data.

Specific instructions and computer programs for performing the above calculations can be obtained from companies such as: SAS/STAT Software, SAS Institute Inc., Cary, N.C., USA; BMDP Statistical Software, BMDP Statistical Software Inc., Los Angeles, Calif., USA; SYSTAT software, SPSS Inc., Chicago, Ill., USA; StatXact & LogXact, CYTEL Software Corporation, Cambridge, Mass., USA.

C. Statistical Genetic Methods Useful for Analysis of Pharmacogenetic Data

A computational method useful in determining the effect of a variance or variances on a protein or proteins is described in U.S. patent application Ser. No. 09/614,735, attorney docket number 11926-003001, filed Jul. 12, 2000, entitled “Methods for structure-based assessment of amino acid polymorphisms”. This method describes variance modeling and prediction methods for the assessment of whether an amino acid variation at a selected amino acid residue is likely or not likely to affect the biological function of the modeled protein. Evaluation of the amino acid changes as a result of genetic polymorphism can be a useful tool for the potential functional consequences of the identified amino acid variation. Further, such predictive computational methods can be useful in analyses including consideration of allele frequency and relative importance of a gene in a pathway of a pharmaceutical compound response in a individual or population with the identified variance. The predictive values listed in Tables 3 and 4 and allele frequency data may be used to select certain variances and may have utility in determining a response to a pharmaceutical compound or for a diagnostic agent. For example, those variances with an allele frequency of at least 5%, more preferably 25%, or preferably 50% or higher, or those with a computationally derived predictive value of at least 0.5, more preferably 0.75 or those with a predictive value of 0.8 or higher may be chosen as being of particular interest. Each pharmaceutical compound and diagnostic agent may have a different set of parameters, for example, a compound eliciting a toxic or undesirable side effect may only affect a small fraction of the population taking the compound, therefore the values considered for this type of compound or agent may fall into the range of an allele frequency of less than 10% with a computationally derived predictive value of at least 0.5, more preferably 0.75 or those with a predictive value of 0.8 or higher. Thus, one skilled in the art may use the information contained in the tables to best select the gene(s) and variances involved in a subject's drug response for further analysis as described herein

A wide spectrum of mathematical and statistical tools may be useful in the analysis of data produced in pharmacogenetic clinical trials, including methods employed in molecular, population, and quantitative genetics, as well as genetic epidemiology. Methods developed for plant and animal breeding may be useful as well, particularly methods relating to the genetic analysis of quantitative traits.

Analytical methods useful in the analysis of genetic variation among individuals, populations and species of various organisms are described in the following texts: Molecular Evolution, by W-H. Li, Sinauer Associates, Inc., 1997; Principles of Population Genetics, by D. L. Hartl and A. G. Clark, 1996; Genetics and Analysis of Quantitative Traits, By M. Lynch and B. Walsh, Sinauer Associates, Inc., Principles of Quantitative Genetics, by D. S. Falconer and T. F. C. Mackay, Longman, 1996; Genetic Variation and Human Disease, by K. M. Weiss, Cambridge University Press, 1993; Fundamentals of Genetic Epidemiology, by M. J. Khoury, T. H. Beaty, and B. H. Cohen, Oxford University Press, 1993; Handbook of Genetic Linkage, by J. Terwilliger J. Ott, Johns Hopkins University Press, 1994.

The types of statistical analysis performed in different branches of genetics are outlined below as a guide to the relevant literature and publicly available software, some of which is cited.

Molecular Evolutionary Genetics

Patterns of nucleotide variation among individuals, families/populations and across species and genera,
Alignment of sequences and description of variation/polymorphisms among the aligned sequences, amounts of similarities and dissimilarities,
Measurement of molecular variation among various regions of a gene, testing of neutrality models,
Rates of nucleotide changes among coding and the non-coding regions within and among populations,
Construction of phylogenetic trees using methods such as neighborhood joining and maximum parsimony; estimation of ages of variances using coalescent models,
   Population Genetics
Patterns of distribution of genes among genotypes and populations. Hardy-Weinberg equilibrium, departures form the equilibrium
Genotype and haplotype frequencies, levels of heterozygosities, polymorphism information contents of genes, estimation of haplotypes from genotypes; the E-M algorithm, and parsimony methods
Estimation of linkage disequilibrium and recombination
Hierarchical structure of populations, the F-statistics, estimation of inbreeding, selection and drift
Genetic admixture/migration and mutation frequencies
Spatial distribution of genotypes using spatial autocorrelation methods
Kin-structured maintenance of variation and migration
   Quantitative Genetics
Phenotype as the product of the interaction between genotype and environment
Additive, dominance and epistatic variance on the phenotype
Effects of homozygosity, heterozygosity and developmental homeostasis
Estimation of heritability: broad sense and narrow sense
Determination of number of genes governing a character
Determination of quantitative trait loci (QTLs) using family information or population information, and using linkage and/or association studies
Determination of quantitative trait nucleotide (QTN) using a combination linkage disequilibrium methods and cladistic approaches
Determination of individual causal nucleotide in the diploid or haploid state on the phenotype using the method of measured genotype approaches, and combined effects or synergistic interaction of the causal mutations on the phenotype
Determination of relative importance of each of the mutations on a given phenotype using multivariate methods, such as discriminant function, principal component and step-wise regression methods
Determination of direct and indirect effect of polymorphisms on a complex phenotype using path analysis (partial regression) methods
Determination of the effects of specific environment on a given genotype—genotype x environment interactions using joint regression and additive and multiplicative parameter methods.
   Genetic Epidemiology
Determination of sample size based on the disease and the marker frequency in the “case” and in the “control” populations
Stratification of study population based on gender, ethnic, socio-economic variation
Establishing a “causal relationship” between genotype and disease, using, using various association and linkage approaches—viz., case-control designs, family studies (if available), transmission disequilibrium tests etc.,
Linkage analysis between markers and a candidate locus using two-point and multipoint approaches.
   Computer programs used for genetic analysis are: Dna SP version 3.0, by Juilo Rozas, University of Barcelona, Spain; Arlequin 1.1 by S. Schnieder, J-M Kueffer, D. Roessli and L. Excoffier. University of Geneva, Switzerland. PAUP*4, by D. L. Swofford, Sinauer Associates, Inc., 1999. SYSTAT software, SPSS Inc., Chicago, Ill., 1998; Linkage User's Guide, by J. Ott, Rockefeller University.

Guidance in the selection of appropriate genetic statistical tests for analysis of data can be obtained from texts such as: Fundamentals of Genetic Epidemiology (Monographs in Epidemiology and Biostatistics, Vol 22) by M. J. Khoury, B. H. Cohen & T. H. Beaty, Oxford Univ Press, 1993; Methods in Genetic Epidemiology by Newton E. Morton, S. Karger Publishing, 1983; Methods in Observational Epidemiology, 2nd edition (Monographs in Epidemiology and Biostatistics, V.26) by J. L. Kelsey (Editor), A. S. Whittemore & A. S. Evans, 1996; Clinical Trials: Design, Conduct, and Analysis (Monographs in Epidemiology and Biostatistics, Vol 8) by C. L. Meinert & S. Tonascia, 1986)

I. Retrospective Clinical Trials.

In general the goal of retrospective clinical trials is to test and refine hypotheses regarding genetic factors that are associated with drug responses. The best supported hypotheses can subsequently be tested in prospective clinical trials, and data from the prospective trials will likely comprise the main basis for an application to register the drug and predictive genetic test with the appropriate regulatory body. In some cases, however, it may become acceptable to use data from retrospective trials to support regulatory filings. Exemplary strategies and criteria for stratifying patients in a retrospective clinical trial are provided below.

Clinical Trials to Study the Effect of One Gene Locus on Drug Response

A. Stratify patients by genotype at one candidate variance in the candidate gene locus.
   1. Genetic stratification of patients can be accomplished in several ways, including the following (where ‘A’ is the more frequent form of the variance being assessed and ‘a’ is the less frequent form):
   (a) AA vs. aa
   (b) AA vs. Aa vs. aa
   (c) AA vs. (Aa+aa)
   (d) (AA+Aa) vs. aa.
   2. The effect of genotype on drug response phenotype may be affected by a variety of nongenetic factors. Therefore it may be beneficial to measure the effect of genetic stratification in a subgroup of the overall clinical trial population. Subgroups can be defined in a number of ways including, for example, biological, clinical, pathological or environmental criteria. For example, the predictive value of genetic stratification can be assessed in a subgroup or subgroups defined by:
   a. Biological criteria:
   i. gender (males vs. females)
   ii. age (for example above 60 years of age). Two, three or more age groups may be useful for defining subgroups for the genetic analysis.
   iii. hormonal status and reproductive history, including pre- vs. post-menopausal status of women, or multiparous vs. nulliparous women
   iv. ethnic, racial or geographic origin, or surrogate markers of ethnic, racial or geographic origin. (For a description of genetic markers that serve as surrogates of racial/ethnic origin see, for example: Rannala, B. and J. L. Mountain, Detecting immigration by using multilocus genotypes. Proc Natl Acad Sci USA, 94(17): 9197-9201, 1997. Other surrogate markers could be used, including biochemical markers.)
   b. Clinical criteria:
   i. Disease status. There are clinical grading scales for many diseases. For example, the status of Alzheimer's Disease patients is often measured by cognitive assessment scales such as the mini-mental status exam (MMSE) or the Alzheimer's Disease Assessment Scale (ADAS), which includes a cognitive component (ADAS-COG). There are also clinical assessment scales for many other diseases, including cancer.
   ii. Disease manifestations (clinical presentation).
   iii. Radiological staging criteria.
   c. Pathological criteria:
   i. Histopathologic features of disease tissue, or pathological diagnosis. (For example there are many varieties of lung cancer: squamous cell carcinoma, adenocarcinoma, small cell carcinoma, bronchoalveolar carcinoma, etc., each of which may—which, in combination with genetic variation, may correlate with
   ii. Pathological stage. A variety of diseases, particularly cancer, have pathological staging schemes
   iii. Loss of heterozygosity (LOH)
   iv. Pathology studies such as measuring levels of a marker protein
   v. Laboratory studies such as hormone levels, protein levels, small molecule levels
   3. Measure frequency of responders in each genetic subgroup. Subgroups may be defined in several ways.
   i. more than two age groups
   ii. reproductive status such as pre or post-menopausal
   4. Stratify by haplotype at one candidate locus where the haplotype is made up of two variances, three variances or greater than three variances.
      Data from already completed clinical trials can be retrospectively reanalyzed. Since the questions are new, the data can be treated as if it were a prospective trial, with identified variances or haplotypes as stratification criteria or endpoints in clinically stratified data (e.g. what is the frequency of a particular variance in a response group compared to nonresponders). Care should be taken to in studying a population in which there may be a link between drug-related genes and disease-related genes.

Retrospective pharmacogenetic trials can be conducted at each of the phases of clinical development, if sufficient data is available to correlate the physiologic effect of the candidate therapeutic intervention and the allelic variance or variances within the treatment population. In the case of a retrospective trial, the data collected from the trial can be re-analyzed by imposing the additional stratification on groups of patients by specific allelic variances that may exist in the treatment groups. Retrospective trials can be useful to ascertain whether a hypothesis that a specific variance has a significant effect on the efficacy or toxicity profile for a candidate therapeutic intervention.

A prospective clinical trial has the advantage that the trial can be designed to ensure the trial objectives can be met with statistical certainty. In these cases, power analysis, which includes the parameters of allelic variance frequency, number of treatment groups, and ability to detect positive outcomes can ensure that the trial objectives are met.

In designing a pharmacogenetic trial, retrospective analysis of Phase II or Phase III clinical data can indicate trial variables for which further analysis is beneficial. For example, surrogate endpoints, pharmacokinetic parameters, dosage, efficacy endpoints, ethnic and gender differences, and toxicological parameters may result in data that would require further analysis and reexamination through the design of an additional trial. In these cases, analysis involving statistics, genetics, clinical outcomes, and economic parameters may be considered prior to proceeding to the stage of designing any additional trials. Factors involved in the consideration of statistical significance may include Bonferroni analysis, permutation testing, with multiple testing correction resulting in a difference among the treatment groups that has occurred as a result of a chance of no greater than 20%, i.e. p<0.20. Factors included in determining clinical outcomes to be relevant for additional testing may include, for example, consideration of the target indication, the trial endpoints, progression of the disease, disorder, or condition during the trial study period, biochemical or pathophysiologic relevance of the candidate therapeutic intervention, and other variables that were not included or anticipated in the initial study design or clinical protocol. Factors to be included in the economic significance in determining additional testing parameters include sample size, accrual rate, number of clinical sites or institutions required, additional or other available medical or therapeutic interventions approved for human use, and additional or other available medical or therapeutic interventions concurrently or anticipated to enter human clinical testing. Further, there may be patients within the treatment categories that present data that fall outside of the average or mean values, or there may be an indication of multiple allelic loci that are involved in the responses to the candidate therapeutic intervention. In these cases, one could propose a prospective clinical trial having an objective to determine the significance of the variable or parameter and its effect on the outcome of the parent Phase II trial. In the case of a pharmacogenetic difference, i.e. a single or multiple allelic difference, a population could be selected based upon the distribution of genotypes. The candidate therapeutic intervention could then be tested in this group of volunteers to test for efficacy or toxicity. The repeat prospective study could be a Phase I limited study in which the subjects would be healthy human volunteers, or a Phase II limited efficacy study in which patients which satisfy the inclusion criteria could be enrolled. In either case, the second, confirmatory trial could then be used to systematically ensure an adequate number of patients with appropriate phenotype is enrolled in a Phase III trial.

A placebo controlled pharmacogenetics clinical trial design will be one in which target allelic variance or variances will be identified and a diagnostic test will be performed to stratify the patients based upon presence, absence, or combination thereof of these variances. In the Phase II or Phase III stage of clinical development, determination of a specific sample size of a prospective trial will be described to include factors such as expected differences between a placebo and treatment on the primary or secondary endpoints and a consideration of the allelic frequencies.

The design of a pharmacogenetics clinical trial will include a description of the allelic variance impact on the observed efficacy between the treatment groups. Using this type of design, the type of genetic and phenotypic relationship display of the efficacy response to a candidate therapeutic intervention will be analyzed. For example, a genotypically dominant allelic variance or variances will be those in which both heterozygotes and homozygotes will demonstrate a specific phenotypic efficacy response different from the homozygous recessive genotypic group. A pharmacogenetic approach is useful for clinicians and public health professionals to include or eliminate small groups of responders or non-responders from treatment in order to avoid unjustified side-effects. Further, adjustment of dosages when clear clinical difference between heterozygous and homozygous individuals may be beneficial for therapy with the candidate therapeutic intervention.

In another example, a recessive allelic variance or variances will be those in which only the homozygote recessive for that or those variances will demonstrate a specific phenotypic efficacy response different from the heterozygotes or homozygous dominants. An extension of these examples may include allelic variance or variances organized by haplotypes from additional gene or genes.

V. Variance Identification and Use

A. Initial Identification of Variances in Genes

Selection of Population Size and Composition

Prior to testing to identify the presence of sequence variances in a particular gene or genes, it is useful to understand how many individuals should be screened to provide confidence that most or nearly all pharmacogenetically relevant variances will be found. The answer depends on the frequencies of the phenotypes of interest and what assumptions we make about heterogeneity and magnitude of genetic effects. Prior to testing to identify the presence of sequence variances in a particular gene or genes, it is useful to understand how many individuals should be screened to provide confidence that most or nearly all pharmacogenetically relevant variances will be found. The answer depends on the frequencies of the phenotypes of interest and what assumptions we make about heterogeneity and magnitude of genetic effects. At the beginning we only know phenotype frequencies (e.g. responders vs. nonresponders, frequency of various side effects, etc.).

The most conservative assumption (resulting in the lowest estimate of allele frequency, and consequently the largest suggested screening population) is (i) that the phenotype (e.g. toxicity or efficacy) is multifactorial (i.e. can be caused by two or more variances or combinations of variances), (ii) that the variance of interest has a high degree of penetrance (i.e. is consistently associated with the phenotype), and (iii) that the mode of transmission is Mendelian dominant. Consider a pharmacogenetic study designed to identify predictors of efficacy for a compound that produces a 15% response rate in a nonstratified population. If half the response is substantially attributable to a given variance, and the variance is consistently associated with a positive response (in 80% of cases) and the variance need only be present in one copy to produce a positive result then ˜10% of the subjects are likely heterozygotes for the variance that produces the response. The Hardy-Weinberg equation can be used to infer an allele frequency in the range of 5% from these assumptions (given allele frequencies of 5%/95% then: 2×0.05×0.95=0.095, or 9.5% heterozygotes are expected, and 0.05×0.05=0.0025, or 0.25% homozygotes are expected. They sum to 9.5%+0.25%=9.75% likely responders, 80% of whom, or 7.6%, are likely real responders due to presence of the positive response allele. Thus about half of the 15% responders are accounted for.). From the Table it can be seen that, in order to have a 99% chance of detecting an allele present at a frequency of 5% nearly 50 subjects should be screened for variances, assuming that the variances occur in the screening population at the same frequency as they occur in the patient population. Similar analyses can be performed for other assumptions regarding likely magnitude of effect, penetrance and mode of genetic transmission.

At the beginning we only know phenotype frequencies (e.g. responders vs. nonresponders, frequency of various side effects, etc.). As an example, the occurrence of serious 5-FU/FA toxicity—e.g. toxicity requiring hospitalization is often >10%. The occurrence of life threatening toxicity is in the 1-3% range (Buroker et al. 1994). The occurrence of complete remissions is on the order of 2-8%. The lowest frequency phenotypes are thus on the order of ˜2%. If we assume that (i) homogeneous genetic effects are responsible for half the phenotypes of interest and (ii) for the most part the extreme phenotypes represent recessive genotypes, then we need to detect alleles that will be present at ˜10% frequency (0.1×0.1=0.01, or 1% frequency of homozygotes) if the population is at Hardy-Weinberg equilibrium. To have a ˜99% chance of identifying such alleles would require searching a population of 22 individuals (see Table below). If the major phenotypes are associated with heterozygous genotypes then we need to detect alleles present at ˜0.5% frequency (2×0.005×0.995=0.00995, or ˜1% frequency of heterozygotes). A 99% chance of detecting such alleles would require ˜40 individuals (Table below). Given the heterogeneity of the North American population we cannot assume that all genotypes are present in Hardy-Weinberg proportions, therefore a substantial oversampling may be done to increase the chances of detecting relevant variances: For our initial screening, usually, 62 individuals of known race/ethnicity are screened for variance. Variance detection studies can be extended to outliers for the phenotypes of interest to cover the possibility that important variances were missed in the normal population screening.

| Allele | | | | | | | | |
|---|---|---|---|---|---|---|---|---|
| frequencies | n = 5 | n = 10 | n = 15 | n = 20 | n = 25 | n = 30 | n = 35 | n = 50 |
| p = .99, | 9.56 | 18.21 | 26.03 | 33.10 | 39.50 | 45.28 | 50.52 | 63.40 |
| p = .97, | 26.26 | 45.62 | 59.90 | 70.43 | 78.19 | 83.92 | 88.14 | 95.24 |
| p = .95, | 40.13 | 64.15 | 78.53 | 87.15 | 92.30 | 95.39 | 97.24 | 99.65 |
| p = .93, | 51.60 | 76.58 | 88.66 | 94.51 | 97.34 | 98.71 | 99.38 | 99.93 |
| p = .9, q = | 65.13 | 87.84 | 95.76 | 98.52 | 99.48 | 99.82 | 99.94 | >99.9 |
| p = .8, q = | 89.26 | 98.84 | 99.88 | 99.99 | >99.9 | >99.9 | >99.9 | >99.9 |
| p = .7, q = | 97.17 | 99.92 | 99.99 | >99.9 | >99.9 | >99.9 | >99.9 | >99.9 |

Likelihood of Detecting Polymorphism in a Population as a Function of Allele Frequency & Number of Individuals Genotyped

The table above shows the probability (expressed as percent) of detecting both alleles (i.e. detecting heterozygotes) at a biallelic locus as a function of (i) the allele frequencies and (ii) the number of individuals genotyped. The chances of detecting heterozygotes increases as the frequencies of the two alleles approach 0.5 (down a column), and as the number of individuals genotyped increases (to the right along a row). The numbers in the table are given by the formula: 1−(p)²ⁿ−(q)²ⁿ. Allele frequencies are designated p and q and the number of individuals tested is designated n. (Since humans are diploid, the number of alleles tested is twice the number of individuals, or 2n.)

While it is preferable that numbers of individuals, or independent sequence samples, are screened to identify variances in a gene, it is also very beneficial to identify variances using smaller numbers of individuals or sequence samples. For example, even a comparison between the sequences of two samples or individuals can reveal sequence variances between them. Preferably, 5, 10, or more samples or individuals are screened.

Source of Nucleic Acid Samples

Nucleic acid samples, for example for use in variance identification, can be obtained from a variety of sources as known to those skilled in the art, or can be obtained from genomic or cDNA sources by known methods. For example, the Coriell Cell Repository (Camden, N.J.) maintains over 6,000 human cell cultures, mostly fibroblast and lymphoblast cell lines comprising the NIGMS Human Genetic Mutant Cell Repository. A catalog (http://locus.umdnj.edu/nigms) provides racial or ethnic identifiers for many of the cell lines. It is preferable to perform polymorphism discovery on a population that mimics the population to be evaluated in a clinical trial, both in terms of racial/ethnic/geographic background and in terms of disease status. Otherwise, it is generally preferable to include a broad population sample including, for example, (for trials in the United States): Caucasians of Northern, Central and Southern European origin, Africans or African-Americans, Hispanics or Mexicans, Chinese, Japanese, American Indian, East Indian, Arabs and Koreans.

Source of Human DNA, RNA and cDNA Samples

PCR based screening for DNA polymorphism can be carried out using either genomic DNA or cDNA produced from mRNA. For many genes, only cDNA sequences have been published, therefore the analysis of those genes is, at least initially, at the cDNA level since the determination of intron-exon boundaries and the isolation of flanking sequences is a laborious process. However, screening genomic DNA has the advantage that variances can be identified in promoter, intron and flanking regions. Such variances may be biologically relevant. Therefore preferably, when variance analysis of patients with outlier responses is performed, analysis of selected loci at the genomic level is also performed. Such analysis would be contingent on the availability of a genomic sequence or intron-exon boundary sequences, and would also depend on the anticipated biological importance of the gene in connection with the particular response.

When cDNA is to be analyzed it is very beneficial to establish a tissue source in which the genes of interest are expressed at sufficient levels that cDNA can be readily produced by RT-PCR. Preliminary PCR optimization efforts for 19 of the 29 genes in Table 2 reveal that all 19 can be amplified from lymphoblastoid cell mRNA. The 7 untested genes belong on the same pathways and are expected to also be PCR amplifiable.

PCR Optimization

Primers for amplifying a particular sequence can be designed by methods known to those skilled in the art, including by the use of computer programs such as the PRIMER software available from Whitehead Institute/MIT Genome Center. In some cases it is preferable to optimize the amplification process according to parameters and methods known to those skilled in the art; optimization of PCR reactions based on a limited array of temperature, buffer and primer concentration conditions is utilized. New primers are obtained if optimization fails with a particular primer set.

Variance Detection Using T4 Endonuclease VII Mismatch Cleavage

Method

Any of a variety of different methods for detecting variances in a particular gene can be utilized, such as those described in the patents and applications cited in section A above. An exemplary method is a T4 EndoVII method. The enzyme T4 endonuclease VII (T4E7) is derived from the bacteriophage T4. T4E7 specifically cleaves heteroduplex DNA containing single base mismatches, deletions or insertions. The site of cleavage is 1 to 6 nucleotides 3′ of the mismatch. This activity has been exploited to develop a general method for detecting DNA sequence variances (Youil et al. 1995; Mashal and Sklar, 1995). A quality controlled T4E7 variance detection procedure based on the T4E7 patent of R. G. H. Cotton and co-workers. (Del Tito et al., in press) is preferably utilized. T4E7 has the advantages of being rapid, inexpensive, sensitive and selective. Further, since the enzyme pinpoints the site of sequence variation, sequencing effort can be confined to a 25-30 nucleotide segment.

The major steps in identifying sequence variations in candidate genes using T4E7 are: (1) PCR amplify 400-600 bp segments from a panel of DNA samples; (2) mix a fluorescently-labeled probe DNA with the sample DNA; (3) heat and cool the samples to allow the formation of heteroduplexes; (4) add T4E7 enzyme to the samples and incubate for 30 minutes at 37° C., during which cleavage occurs at sequence variance mismatches; (5) run the samples on an ABI 377 sequencing apparatus to identify cleavage bands, which indicate the presence and location of variances in the sequence; (6) a subset of PCR fragments showing cleavage are sequenced to identify the exact location and identity of each variance.

The T4E7 Variance Imaging procedure has been used to screen particular genes. The efficiency of the T4E7 enzyme to recognize and cleave at all mismatches has been tested and reported in the literature. One group reported detection of 81 of 81 known mutations (Youil et al. 1995) while another group reported detection of 16 of 17 known mutations (Mashal and Sklar, 1995). Thus, the T4E7 method provides highly efficient variance detection.

DNA Sequencing

A subset of the samples containing each unique T4E7 cleavage site is selected for sequencing. DNA sequencing can, for example, be performed on ABI 377 automated DNA sequencers using BigDye chemistry and cycle sequencing. Analysis of the sequencing runs will be limited to the 30-40 bases pinpointed by the T4E7 procedure as containing the variance. This provides the rapid identification of the altered base or bases.

In some cases, the presence of variances can be inferred from published articles which describe Restriction Fragment Length Polymorphisms (RFLP). The sequence variances or polymorphisms creating those RFLPs can be readily determined using convention techniques, for example in the following manner. If the RFLP was initially discovered by the hybridization of a cDNA, then the molecular sequence of the RFLP can be determined by restricting the cDNA probe into fragments and separately hybridizing to a Southern blot consisting of the restriction digestion with the enzyme which reveals the polymorphic site, identifying the sub-fragment which hybridizes to the polymorphic restriction fragment, obtaining a genomic clone of the gene (e.g., from commercial services such as Genome Systems (Saint Louis, Mo.) or Research Genetics (Alabama) which will provide appropriate genomic clones on receipt of appropriate primer pairs). Using the genomic clone, restrict the genomic clone with the restriction enzyme which revealed the polymorphism and isolate the fragment which contains the polymorphism, e.g., identifying by hybridization to the cDNA which detected the polymorphism. The fragment is then sequenced across the polymorphic site. A copy of the other allele can be obtained by PCT from addition samples.

Variance Detection Using Sequence Scanning

In addition to the physical methods, e.g., those described above and others known to those skilled in the art (see, e.g., Housman, U.S. Pat. No. 5,702,890; Housman et al., U.S. patent application Ser. No. 09/045,053), variances can be detected using computational methods, involving computer comparison of sequences from two or more different biological sources, which can be obtained in various ways, for example from public sequence databases. The term “variance scanning” refers to a process of identifying sequence variances using computer-based comparison and analysis of multiple representations of at least a portion of one or more genes. Computational variance detection involves a process to distinguish true variances from sequencing errors or other artifacts, and thus does not require perfectly accurate sequences. Such scanning can be performed in a variety of ways, preferably, for example, as described in Stanton et al., filed Oct. 14, 1999, Ser. No. 09/419,705.

While the utilization of complete cDNA sequences is highly preferred, it is also possible to utilize genomic sequences. Such analysis may be desired where the detection of variances in or near splice sites is sought. Such sequences may represent full or partial genomic DNA sequences for a gene or genes. Also, as previously indicated, partial cDNA sequences can also be utilized although this is less preferred. As described below, the variance scanning analysis can simply utilize sequence overlap regions, even from partial sequences. Also, while the present description is provided by reference to DNA, e.g., cDNA, some sequences may be provided as RNA sequences, e.g., mRNA sequences. Such RNA sequences may be converted to the corresponding DNA sequences, or the analysis may use the RNA sequences directly.

B. Determination of Presence or Absence of Known Variances

The identification of the presence of previously identified variances in cells of an individual, usually a particular patient, can be performed by a number of different techniques as indicated in the Summary above. Such methods include methods utilizing a probe which specifically recognizes the presence of a particular nucleic acid or amino acid sequence in a sample. Common types of probes include nucleic acid hybridization probes and antibodies, for example, monoclonal antibodies, which can differentially bind to nucleic acid sequences differing in one or more variance sites or to polypeptides which differ in one or more amino acid residues as a result of the nucleic acid sequence variance or variances. Generation and use of such probes is well-known in the art and so is not described in detail herein.

Preferably, however, the presence or absence of a variance is determined using nucleotide sequencing of a short sequence spanning a previously identified variance site. This will utilize validated genotyping assays for the polymorphisms previously identified. Since both normal and tumor cell genotypes can be measured, and since tumor material will frequently only be available as paraffin embedded sections (from which RNA cannot be isolated), it will be necessary to utilize genotyping assays that will work on genomic DNA. Thus PCR reactions will be designed, optimized, and validated to accommodate the intron-exon structure of each of the genes. If the gene structure has been published (as it has for some of the listed genes), PCR primers can be designed directly. However, if the gene structure is unknown, the PCR primers may need to be moved around in order to both span the variance and avoid exon-intron boundaries. In some cases one-sided PCR methods such as bubble PCR (Ausubel et al. 1997) may be useful to obtain flanking intronic DNA for sequence analysis.

Using such amplification procedures, the standard method used to genotype normal and tumor tissues will be DNA sequencing. PCR fragments encompassing the variances will be cycle sequenced on ABI 377 automated sequencers using Big Dye chemistry

C. Correlation of the Presence or Absence of Specific Variances with Differential Treatment Response

Prior to establishment of a diagnostic test for use in the selection of a treatment method or elimination of a treatment method, the presence or absence of one or more specific variances in a gene or in multiple genes is correlated with a differential treatment response. (As discussed above, usually the existence of a variable response and the correlation of such a response to a particular gene is performed first.) Such a differential response can be determined using prospective and/or retrospective data. Thus, in some cases, published reports will indicate that the course of treatment will vary depending on the presence or absence of particular variances. That information can be utilized to create a diagnostic test and/or incorporated in a treatment method as an efficacy or safety determination step.

Usually, however, the effect of one or more variances is separately determined. The determination can be performed by analyzing the presence or absence of particular variances in patients who have previously been treated with a particular treatment method, and correlating the variance presence or absence with the observed course, outcome, and/or development of adverse events in those patients. This approach is useful in cases in which observation of treatment effects was clearly recorded and cell samples are available or can be obtained. Alternatively, the analysis can be performed prospectively, where the presence or absence of the variance or variances in an individual is determined and the course, outcome, and/or development of adverse events in those patients is subsequently or concurrently observed and then correlated with the variance determination.

Analysis of Haplotypes Increases Power of Genetic Analysis

In some cases, variation in activity due to a single gene or a single genetic variance in a single gene may not be sufficient to account for a clinically significant fraction of the observed variation in patient response to a treatment, e.g., a drug, there may be other factors that account for some of the variation in patient response. Drug response phenotypes may vary continuously, and such (quantitative) traits may be influenced by a number of genes (Falconer and Mackay, Quantitative Genetics, 1997). Although it is impossible to determine a priori the number of genes influencing a quantitative trait, potentially only one or a few loci have large effects, where a large effect is 5-20% of total variation in the phenotype (Mackay, 1995).

Having identified genetic variation in enzymes that may affect action of a specific drug, it is useful to efficiently address its relation to phenotypic variation. The sequential testing for correlation between phenotypes of interest and single nucleotide polymorphisms may be adequate to detect associations if there are major effects associated with single nucleotide changes; certainly it is useful to this type of analysis. However there is no way to know in advance whether there are major phenotypic effects associated with single nucleotide changes and, even if there are, there is no way to be sure that the salient variance has been identified by screening cDNAs. A more powerful way to address the question of genotype-phenotype correlation is to assort genotypes into haplotypes. (A haplotype is the cis arrangement of polymorphic nucleotides on a particular chromosome.) Haplotype analysis has several advantages compared to the serial analysis of individual polymorphisms at a locus with multiple polymorphic sites.

(1) Of all the possible haplotypes at a locus (2ⁿ haplotypes are theoretically possible at a locus with n binary polymorphic sites) only a small fraction will generally occur at a significant frequency in human populations. Thus, association studies of haplotypes and phenotypes will involve testing fewer hypotheses. As a result there is a smaller probability of Type I errors, that is, false inferences that a particular variant is associated with a given phenotype.

(2) The biological effect of each variance at a locus may be different both in magnitude and direction. For example, a polymorphism in the 5′ UTR may affect translational efficiency, a coding sequence polymorphism may affect protein activity, a polymorphism in the 3′ UTR may affect mRNA folding and half life, and so on. Further, there may be interactions between variances: two neighboring polymorphic amino acids in the same domain—say cys/arg at residue 29 and met/val at residue 166—may, when combined in one sequence, for example, 29cys-166val, have a deleterious effect, whereas 29cys-166met, 29arg-166met and 29arg-166val proteins may be nearly equal in activity. Haplotype analysis is the best method for assessing the interaction of variances at a locus.

(3) Templeton and colleagues have developed powerful methods for assorting haplotypes and analyzing haplotype/phenotype associations (Templeton et al., 1987). Alleles which share common ancestry are arranged into a tree structure (cladogram) according to their (inferred) time of origin in a population (that is, according to the principle of parsimony). Haplotypes that are evolutionarily ancient will be at the center of the branching structure and new ones (reflecting recent mutations) will be represented at the periphery, with the links representing intermediate steps in evolution. The cladogram defines which haplotype-phenotype association tests should be performed to most efficiently exploit the available degrees of freedom, focusing attention on those comparisons most likely to define functionally different haplotypes (Haviland et al., 1995). This type of analysis has been used to define interactions between heart disease and the apolipoprotein gene cluster (Haviland et al 1995) and Alzheimer's Disease and the Apo-E locus (Templeton 1995) among other studies, using populations as small as 50 to 100 individuals. The methods of Templeton have also been applied to measure the genetic determinants of variation in the angiotensin-I converting enzyme gene. (Keavney, B., McKenzie, C. A., Connoll, J. M. C., et al. Measured haplotype analysis of the angiotensin-I converting enzyme gene. Human Molecular Genetics 7: 1745-1751.)

Methods for Determining Haplotypes

The goal of haplotyping is to identify the common haplotypes at selected loci that have multiple sites of variance. Haplotypes are usually determined at the cDNA level. Several general approaches to identification of haplotyes can be employed. Haplotypes may also be estimated using computational methods or determined definitively using experimental approaches. Computational approaches generally include an expectation maximization (E-M) algorithm (see, for example: Excoffier and Slatkin, Mol. Biol. Evol. 1995) or a combination of Parsimony (see below) and E-M methods.

Haplotypes can be determined experimentally without requirement of a haplotyping method by genotyping samples from a set of pedigrees and observing the segregation of haplotypes. For example families collected by the Centre d'Etude du Polymorphisme Humaine (CEPH) can be used. Cell lines from these families are available from the Coriell Repository. This approach will be useful for cataloging common haplotypes and for validating methods on samples with known haplotypes. The set of haplotypes determined by pedigree analysis can be useful in computational methods, including those utilizing the E-M algorithm.

Haplotypes can also be determined directly from cDNA using the T4E7 procedure. T4E7 cleaves mismatched heteroduplex DNA at the site of the mismatch. If a heteroduplex contains only one mismatch, cleavage will result in the generation of two fragments. However, if a single heteroduplex (allele) contains two mismatches, cleavage will occur at two different sites resulting in the generation of three fragments. The appearance of a fragment whose size corresponds to the distance between the two cleavage sites is diagnostic of the two mismatches being present on the same strand (allele). Thus, T4E7 can be used to determine haplotypes in diploid cells.

An alternative method, allele specific PCR, may be used for haplotyping. The utility of allele specific PCR for haplotyping has already been established (Michalatos-Beloin et al., 1996; Chang et al. 1997). Opposing PCR primers are designed to cover two sites of variance (either adjacent sites or sites spanning one or more internal variances). Two versions of each primer are synthesized, identical to each other except for the 3′ terminal nucleotide. The 3′ terminal nucleotide is designed so that it will hybridize to one but not the other variant base. PCR amplification is then attempted with all four possible primer combinations in separate wells. Because Taq polymerase is very inefficient at extending 3′ mismatches, the only samples which will be amplified will be the ones in which the two primers are perfectly matched for sequences on the same strand (allele). The presence or absence of PCR product allows haplotyping of diploid cell lines. At most two of four possible reactions should yield products. This procedure has been successfully applied, for example, to haplotype the DPD amino acid polymorphisms.

Parsimony methods are also useful for classifying DNA sequences, haplotypes or phenotypic characters. Parsimony principle maintains that the best explanation for the observed differences among sequences, phenotypes (individuals, species) etc., is provided by the smallest number of evolutionary changes. Alternatively, simpler hypotheses are preferable to explain a set of data or patterns, than more complicated ones, and ad hoc hypotheses should be avoided whenever possible (Molecular Systematics, Hillis et al., 1996). Parsimony methods thus operate by minimizing the number of evolutionary steps or mutations (changes from one sequence/character) required to account for a given set of data.

For example, supposing we want to obtain relationships among a set of sequences and construct a structure (tree/topology), we first count the minimum number of mutations that are required for explaining the observed evolutionary changes among a set of sequences. A structure (topology) is constructed based on this number. When once this number is obtained, another structure is tried. This process is continued for all reasonable number of structures. Finally, the structure that required the smallest number of mutational steps is chosen as the likely structure/evolutionary tree for the sequences studied.

For haplotypes identified herein, haplotypes were identified by examining genotypes from each cell line. This list of genotypes was optimized to remove variance sites/individuals with incomplete information, and the genotype from each remaining cell line was examined in turn. The number of heterozygotes in the genotype were counted, and those genotypes containing more than one heterozygote were discarded, and the rest were gathered in a list for storage and display.

D. Selection of Treatment Method Using Variance Information

1. General

Once the presence or absence of a variance or variances in a gene or genes is shown to correlate with the efficacy or safety of a treatment method, that information can be used to select an appropriate treatment method for a particular patient. In the case of a treatment which is more likely to be effective when administered to a patient who has at least one copy of a gene with a particular variance or variances (in some cases the correlation with effective treatment is for patients who are homozygous for a variance or set of variances in a gene) than in patients with a different variance or set of variances, a method of treatment is selected (and/or a method of administration) which correlates positively with the particular variance presence or absence which provides the indication of effectiveness. As indicated in the Summary, such selection can involve a variety of different choices, and the correlation can involve a variety of different types of treatments, or choices of methods of treatment. In some cases, the selection may include choices between treatments or methods of administration where more than one method is likely to be effective, or where there is a range of expected effectiveness or different expected levels of contra-indication or deleterious effects. In such cases the selection is preferably performed to select a treatment which will be as effective or more effective than other methods, while having a comparatively low level of deleterious effects. Similarly, where the selection is between method with differing levels of deleterious effects, preferably a method is selected which has low such effects but which is expected to be effective in the patient.

Alternatively, in cases where the presence or absence of the particular variance or variances is indicative that a treatment or method of administration is more likely to be ineffective or contra-indicated in a patient with that variance or variances, then such treatment or method of administration is generally eliminated for use in that patient.

2. Diagnostic Methods

Once a correlation between the presence and absence of at least one variance in a gene or genes and an indication of the effectiveness of a treatment, the determination of the presence or absence of that at least one variance provides diagnostic methods, which can be used as indicated in the Summary above to select methods of treatment, methods of administration of a treatment, methods of selecting a patient or patients for a treatment and others aspects in which the determination of the presence or absence of those variances provides useful information for selecting or designing or preparing methods or materials for medical use in the aspects of this invention. As previously stated, such variance determination or diagnostic methods can be performed in various ways as understood by those skilled in the art.

In certain variance determination methods, it is necessary or advantageous to amplify one or more nucleotide sequences in one or more of the genes identified herein. Such amplification can be performed by conventional methods, e.g., using polymerase chain reaction (PCR) amplification. Such amplification methods are well-known to those skilled in the art and will not be specifically described herein. For most applications relevant to the present invention, a sequence to be amplified includes at least one variance site, which is preferably a site or sites which provide variance information indicative of the effectiveness of a method of treatment or method of administration of a treatment, or effectiveness of a second method of treatment which reduces a deleterious effect of a first treatment method, or which enhances the effectiveness of a first method of treatment. Thus, for PCR, such amplification generally utilizes primer oligonucleotides which bind to or extent through at least one such variance site under amplification conditions.

For convenient use of the amplified sequence, e.g., for sequencing, it is beneficial that the amplified sequence be of limited length, but still long enough to allow convenient and specific amplification. Thus, preferably the amplified sequence has a length as described in the Summary.

Also, in certain variance determination, it is useful to sequence one or more portions of a gene or genes, in particular, portions of the genes identified in this disclosure. As understood by persons familiar with nucleic acid sequencing, there are a variety of effective methods. In particular, sequencing can utilize dye termination methods and mass spectrometric methods. The sequencing generally involves a nucleic acid sequence which includes a variance site as indicated above in connection with amplification. Such sequencing can directly provide determination of the presence or absence of a particular variance or set of variances, e.g., a haplotype, by inspection of the sequence (visually or by computer). Such sequencing is generally conducted on PCR amplified sequences in order to provide sufficient signal for practical or reliable sequence determination.

Likewise, in certain variance determinations, it is useful to utilize a probe or probes. As previously described, such probes can be of a variety of different types.

VI. Pharmaceutical Compositions, Including Pharmaceutical Compositions Adapted to be Preferentially Effective in Patients Having Particular Genetic Characteristics

A. General

The methods of the present invention, in many cases will utilize conventional pharmaceutical compositions, but will allow more advantageous and beneficial use of those compositions due to the ability to identify patients who are likely to benefit from a particular treatment or to identify patients for whom a particular treatment is less likely to be effective or for whom a particular treatment is likely to produce undesirable or intolerable effects. However, in some cases, it is advantageous to utilize compositions which are adapted to be preferentially effective in patients who possess particular genetic characteristics, i.e., in whom a particular variance or variances in one or more genes is present or absent (depending on whether the presence or the absence of the variance or variances in a patient is correlated with an increased expectation of beneficial response). Thus, for example, the presence of a particular variance or variances may indicate that a patient can beneficially receive a significantly higher dosage of a drug than a patient having a different ______?

B. Regulatory Indications and Restrictions

The sale and use of drugs and the use of other treatment methods usually are subject to certain restrictions by a government regulatory agency charged with ensuring the safety and efficacy of drugs and treatment methods for medical use, and approval is based on particular indications. In the present invention it is found that variability in patient response or patient tolerance of a drug or other treatment often correlates with the presence or absence of particular variances in particular genes. Thus, it is expected that such a regulatory agency may indicate that the approved indications for use of a drug with a variance-related variable response or toleration include use only in patients in whom the drug will be effective, and/or for whom the administration of the drug will not have intolerable deleterious effects, such as excessive toxicity or unacceptable side-effects. Conversely, the drug may be given for an indication that it may be used in the treatment of a particular disease or condition where the patient has at least one copy of a particular variance, variances, or variant form of a gene. Even if the approved indications are not narrowed to such groups, the regulatory agency may suggest use limited to particular groups or excluding particular groups or may state advantages of use or exclusion of such groups or may state a warning on the use of the drug in certain groups. Consistent with such suggestions and indications, such an agency may suggest or recommend the use of a diagnostic test to identify the presence or absence of the relevant variances in the prospective patient. Such diagnostic methods are described in this description. Generally, such regulatory suggestion or indication is provided in a product insert or label, and is generally reproduced in references such as the Physician's Desk Reference (PDR). Thus, this invention also includes drugs or pharmaceutical compositions which carry such a suggestion or statement of indication or warning or suggestion for a diagnostic test, and which may also be packaged with an insert or label stating the suggestion or indication or warning or suggestion for a diagnostic test.

In accord with the possible variable treatment responses, an indication or suggestion can specify that a patient be heterozygous, or alternatively, homozygous for a particular variance or variances or variant form of a gene. Alternatively, an indication or suggestion may specify that a patient have no more than one copy, or zero copies, of a particular variance, variances, or variant form of a gene.

A regulatory indication or suggestion may concern the variances or variant forms of a gene in normal cells of a patient and/or in cells involved in the disease or condition. For example, in the case of a cancer treatment, the response of the cancer cells can depend on the form of a gene remaining in cancer cells following loss of heterozygosity affecting that gene. Thus, even though normal cells of the patient may contain a form of the gene which correlates with effective treatment response, the absence of that form in cancer cells will mean that the treatment would be less likely to be effective in that patient than in another patient who retained in cancer cells the form of the gene which correlated with effective treatment response. Those skilled in the art will understand whether the variances or gene forms in normal or disease cells are most indicative of the expected treatment response, and will generally utilize a diagnostic test with respect to the appropriate cells. Such a cell type indication or suggestion may also be contained in a regulatory statement, e.g., on a label or in a product insert.

C. Preparation and Administration of Drugs and Pharmaceutical Compositions Including Pharmaceutical Compositions Adapted to be Preferentially Effective in Patients Having Particular Genetic Characteristics

A particular compound useful in this invention can be administered to a patient either by itself, or in pharmaceutical compositions where it is mixed with suitable carriers or excipient(s). In treating a patient exhibiting a disorder of interest, a therapeutically effective amount of a agent or agents such as these is administered. A therapeutically effective dose refers to that amount of the compound that results in amelioration of one or more symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et. al., in The Pharmacological Basis of Therapeutics, 1975, Ch. 1 p.1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of disorder of interest will vary with the severity of the condition to be treated and the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The invention described herein features methods for determining the appropriate identification of a patient diagnosed with a neurological disease or neurological dysfunction based on an analysis of the patient's allele status for a gene listed in Tables 1, 3, and 4. Specifically, the presence of at least one allele indicates that a patient will respond to a candidate therapeutic intervention aimed at treating a neurological clinical symptoms. In a preferred approach, the patient's allele status is rapidly diagnosed using a sensitive PCR assay and a treatment protocol is rendered. The invention also provides a method for forecasting patient outcome and the suitability of the patient for entering a clinical drug trial for the testing of a candidate therapeutic intervention for a neurological disease, condition, or dysfunction.

The findings described herein indicate the predictive value of the target allele in identifying patients at risk for neurologic disease or neurologic dysfunction. In addition, because the underlying mechanism influenced by the allele status is not disease-specific, the allele status is suitable for making patient predictions for diseases not affected by the pathway as well.

The following examples, which describe exemplary techniques and experimental results, are provided for the purpose of illustrating the invention, and should not be construed as limiting.

### EXAMPLE 1

Amyotrophic Lateral Sclerosis

I. Description of Amyotrophic Lateral Sclerosis

Amyotrophic Lateral Sclerosis (ALS) is a degenerative neurological disease that primarily involves the motor neuron system. The disease is characterized by muscular atrophy, progressive weakness, fasciculations, spasticity, disarthria, dysphagia, and respiratory compromise. Sensory, cognitive, oculomotor, and autonomic functions are spared. There are approximately 30,000 individuals with ALS in the U.S. with an estimated annual cost of $300 million dollars. The majority of cases are sporadic and of unknown etiology, however approximately 5%-10% of ALS cases are inherited as an autosomal dominant trait (familial ALS). Superoxide dismutase 1 (SOD1) gene mutations are responsible for about 20% of familial ALS cases.

II. Current Therapies for ALS

There are no compounds that halt or prevent the progressive neurodegeneration of ALS. Riluzole (RILUTEK®), a benzothiazole derivative, is approved for treatment of ALS based on data that it slows disease progression and modestly increases survival time and ventilator-free time. Riluzole's mechanism of action is not completely understood, however pharmacological properties include: 1) an inhibitory effect on glutamate release, 2) inactivation of voltage dependent sodium channels, 3) downmodulation of signalling via excitatory amino acid receptors, particularly glutamate receptors. Unfortunately riluzole, which was introduced in 1996, produces a benefit in only a fraction of patients, and the effect is modest. For example, despite the increase in longevity there is no consistent increase in muscular strenght or pulmonary function. Thus patients do not experience significant relief from symptoms. Patients and care givers quickly understood these limitations, and consequently the use of the drug has been limited. A 1997 study, conducted during the first 8 months after commercialization of riluzole, found that only 37% of patients (17 of 46) eligible for riluzole were interested in trying the drug. The most common reason given for not wanting to try riluzole was insufficient benefit.

III. Limitations of Current Therapies for ALS

As noted above, despite therapy with riluzole, in most ALS patients the disease progresses to debilitating and ultimately life-threatening symptoms. However, since there are no therapeutic alternatives, riluzole is frequently administered despite the modest efficacy. This practice increases the cost of ALS care significantly. In addition to unimpressive efficacy, riluzole therapy has been associated with elevation of serum ALT levels. Thus patients on riluzole should be monitored bimonthly for elevated liver enzymes, at significant cost. Other side effects, which occur infrequently, include neutropenia, asthenia, nausea, dizziness, decreased lung function, diarrhea, abdominal pain, pneumonia, vomiting, vertigo, paresthesia, anorexia, and somolence. Attending to these iatrogenic effects further increases the costs associated with rizulin therapy.

IV. Potential Impact of Genotyping on Drug Development for ALS

There is already a well established genetic cause of some familial ALS cases: mutation of the SOD-1 gene. It is likely that genetic factors play a role in the pathogenesis of sporadic ALS and non-SOD1 linked familial ALS. Strong candidate genes include, for example, other scavengers of superoxide, the entire glutamate signal transduction pathway, calcium channels and genes involved in the production and degradation of neurofilaments. Stratification of clinical trial patients by allelic variation in these or other candidate genes may reveal differences in response rate, duration or quality of response, or adverse events that would be useful in the development of a compound. Provided in this invention are additional genetic pathways implicated in the disease process or response to candidate therapies. Variation in these genes may account for the observed variability in treatment response. Exemplary variations in the candidate genes are provided in tables 3 and 4. The Detailed Description above describes how one skilled in the art would identify a candidate gene or genes, identify sequence variances, stratify patients, design clinical trials, and obtain regulatory approval of a pharmacogenetic test for optimal responders to an ALS treatment. Gene pathways including most preferably, but not limited to, those genes that are listed in the gene pathway Table 1, and pathway matrix Table 2 and discussed in Section V. below are candidates for the genetic analysis and product development strategies described above.

Advantages of Pharmacogenetic Clinical Development of Agents for ALS

In view of the limitations of present therapy, the advantages of an ALS clinical development program that includes genetic stratification of patients in the analysis of response to candidate therapeutic interventions are numerous. First, it may be possible to identify a subpopulation that responds to a treatment at a higher rate than the whole ALS population. This would address the demonstrated disinclination of ALS patients to expose themselves to therapies of limited effectiveness. It might also allow regulatory approval of therapies that do not produce a sufficient response in the unstratified population to justify approval. Second, it may be possible to identify patients who respond to a treatment only at higher doses than most patients require, or respond preferentially to an altered dosing route or schedule. Such customization of therapy to individual genetic and biochemical differences may allow a higher overall response rate to be achieved, without requiring totally empirical dose adjustment in each patient. Third, it may be possible to identify patients in whom side effects are likely to occur. Such patients could be offered alternative treatments. It is also worth noting that the type of benefit afforded by drugs such as RILUTEK®—a slowing of deterioration—will likely be most useful if the drug is started very early, before large numbers of neurons are gone. However the long term prophylactic use of medicines in well, or nearly well, individuals entails a different cost-benefit analysis than in already sick individuals. Identification of patients that respond well to early neuroprotective therapy may be aided by the analysis of genetic determinants of treatment response. Additional uses of genetic stratification in clinical development have been described above.

As an example of a candidate gene with DNA sequence variances potentially relevant to drug efficacy, safety, or both consider the glutamate aspartate receptor NMDA 2C, a member of the glutamate pathway. Described in this application are novel NMDA 2C DNA sequence variances that the inventors have recognized may affect response to drugs. (Diseases in which the glutamate pathway is likely to play a role are summarized in Table 2.) Six DNA sequence variances have been identified in the NMDA 2C gene, five of which alter the encoded amino acid sequence. Several of the amino acid variances are nonconservative, including phenylalanine-valine, glycine-arginine and arginine-serine (see Table 3 for details). Seven DNA sequence variances are described in the NMDA 2A receptor (Table 3). The effect of one or more of these genetic polymorphisms on efficacy or safety of an ALS treatment could be tested in a clinical trial. For example, the goal could be optimization of patient selection for glutamate channel antagonist therapy of ALS by determining whether an ALS patient has a NMDA 2A or 2C receptor genotype against which a glutamate antagonist is more effective or safer.

Similarly, for genes belonging to the other pathways relevant to treatment of ALS (see tables 1 and 2) and polymorphisms in those genes (tables 3 and 4) a strong argument can be made that said polymorphisms (or sets of polymorphisms, or haplotypes) may affect efficacy or safety of drugs active against ALS, including, but not limited to, drugs listed below in Table 5 and related compounds. The candidate genes include, but are not limited to, modulators of glutaminergic, serotonergic, GABAergic, melatonergic and opiate pathways, as well as calcium channels, cytokines, factors that mediate growth, differentiation and apoptosis, the coagulation cascade, second messenger systems, detoxification genes, particularly relating to superoxide, protein degradation and cytoskeleton genes.

V. Therapeutic Strategies for ALS

The etiology of most ALS cases is unknown but may involve autoimmune responses, for example to calcium channels, injury due to excessive excitotoxic stimulation (especially via aspartate, glutamate and GABA receptors), impaired clearance of free radicals, imbalance of neurofilament turnover or possibly viral mediated destruction of motor neurons (e.g. herpes virus). A number of drug development programs are aimed at these postulated pathophysiologic mechanisms. For example, there are candidate therapeutic agents that down modulate immune reactivity, block or dampen excitatory neurotransmitter signalling, alleviate free radical injury, and interfere with a hypothesized viral infection of motor neurons. Beyond the specific mechanisms of action enumerated above, there are many compounds in development that are intended to halt, retard, or prevent neural cell degeneration, or promote neural cell regeneration. Many such compounds are in clinical development programs for multiple neurological diseases. For example, gabapentin is a compound with complex and incompletely understood pharmacology, but its shows anticonvulsant, antinociceptive, anxiolytic and neuroprotective activity in animal models. In ALS animal models gabapentin prevents neuronal death. One of its actions may be inhibition of glutamate synthesis by branched-chain amino acid aminotransferase (BCAA-t). Other compounds in development for ALS target proteins involved in growth control and differentiation, protein processing, intracellular second messenger cascades and cytoskeletal proteins (see Table 5 below for specific compounds and Table 1 for the candidate genes that may affect response to those compounds).

Below in Table 5 the therapies in development for ALS catagorized by mechanism of action. The listed candidate therapeutic intervention response in patients with ALS may be affected by polymorphisms in genes as described above in the Detailed Description.

### EXAMPLE 2

Dementia

I. Description of Dementia

Dementia is a general term for mental deterioration. clinical state characterized by a significant loss of function in multiple cognitive domains, not due to an impaired level of confusion. Diagnosis of dementia requires 1) assessment of an individual's current level of cognitive function with the ability to compare to past intellectual function, and 2) documenting a decline in intellectual function by serial examinations over time. A comprehensive, reliable, and universally accepted clinical classification of the clinical and neuropathological characteristics of senile dementia has been described. However, definitive diagnosis is obtainable only with pathological findings upon autopsy. Based upon these diagnoses, there are an estimated 4 million Americans with Alzheimer's disease (AD) and 10 million Americans with dementia of all types.

Besides AD, there are categories of dementia that include vascular dementia, lewy body disease, frontal lobe dementia, mixed dementia, and post-traumatic dementia. A number of different diseases or conditions are characterized by or involve loss of cholinergic function and/or defects in neuronal remodeling repair and may result in clinical symptoms of dementia. Among these are diseases such as Alzheimer's disease (AD), Huntington's disease, Parkinson's disease, and amyotrophic lateral sclerosis (ALS). Dementia can further be a complication of the following: depression, drug intoxication, metabolic disorders, normal pressure hydrocephalus, subdural hematomas, and cerebrovascular insufficiency.

II. Current Therapies for Dementia

Current therapies for the treatment of dementia include enhancement of cortical cholinergic function. In general, approaches to replacement of cholinergic function can be characterized as either: 1) therapies that compensate for existing damage; and 2) therapies that halt, retard, or prevent cerebral damage. Ideally, a therapy targeting both mechanisms could potentially reverse existing damage. There are two broad mechanisms to enhance cerebral cholinergic function; 1) to block metabolism of acetylcholine via an acetylcholinesterase inhibitor, or 2) agonists at muscarinic or nicotinic receptors.

Acetylcholinesterase inhibitors have recently been approved for the use in patients with mild to moderate Alzheimer's disease. These agents (donepezil, Tacrine) selectively inhibit the acetylcholinesterase enzyme and increases levels of cortical acetylcholine. In randomized controlled clinical trials, donepezil was shown to improve both cognitive performance and global functioning. The improvements are modest and may not be apparent until up to three months after commencement of treatment.

III. Limitations of Current Therapies for Dementia

Despite the introduction of pharmacologic agents for the treatment of dementia, the mainstay of therapeutic management continues to be education, and support for caregivers, and treatment of complications. This is in part because the available acetylcholinesterase inhibitor (donepezil) has limited efficacy and has undesirable side effects. Thus, the clinician is faced with the dilemma of limited therapeutic alternatives and weighing the benefits against the side effects.

Limitations of Acetylcholinesterase Therapy Due to Low Efficacy

Acetylcholinesterase inhibitors have limited efficacy; only a fraction (modest improvement in 40-50%) of patients respond to therapy. The extent and progression of loss of cortical cholinergic neurons limit the therapeutic benefit of acetylcholinesterase inhibitors. Long-term benefit of inhibition of acetylcholinesterase activity is unproven. Further, there is no clinical evidence supporting the use of acetylcholinesterase inhibitors in the prevention of AD or in the treatment of more severe stages.

An additional efficacy concern of the acetylcholinesterase inhibitor is the latent period before demonstrable clinical benefit. In the same period there may be concurrent neurodegeneration. Thus, the clinician has limited therapeutic alternatives, the patient may have limited response to therapy, and the disease progresses. In many cases, medical management of dementia is reduced to treatment of complications or supportive care.

Limitation of Acetylcholinesterase Therapy Due to Toxicity or Undesired Side Effects

Toxicities associated with the use of acetylcholinesterase inhibitors are 1) vagotonic effect on the myocardium resulting in bradycardia and complications of other myocardial syndromes, 2) gastrointestinal complications such as nausea, vomiting, diarrhea, 3) lowering of seizure threshold (since seizures can be a complication of AD, this side effect may be confused with the progression of the disease).

Other acetylcholinesterase inhibitors have been shown to have a severe hepatotoxic effect, those products have been removed from the market or clinical development programs.

IV. Impact of Genotyping on Drug Development for Dementia

As previously indicated, the pathways and genes emphasize the relationship with Alzheimer's disease. In connection with the development of Alzheimers, it had been found that the presence of the ApoE4 allele was associated with an earlier development of the disease than other alleles, and further was associated with a decreased response to present acetylcholinesterase inhibitors, such as tacrine The ε4 allele of Apolipoprotein E (ApoE) is a well-established risk factor for late onset Alzheimer's disease. The work of Poirier (1995) and Farlow (1998) suggests there are significant interactions between sex, ApoE genotype, and therapeutic response (ADAS-Cog scores) to the acetylcholinesterase inhibitor tacrine, with the ε4 allele generally associated with poor response and the effect being more notable in women than in men. ApoE is only part of the brain lipid transport pathway, however, and the interaction of allelic variation at other components of this pathway with drug response can also contribute to variation in therapeutic responses.

Sequence variance in the butyrylcholinesterase (BCHE) gene has been found to correlate with the development of Alzheimer's disease, as well as with treatment efficacy of both cholinomimetic and non-cholinomimetic drug therapies. In this case, the presence of at least one BCHE-k allele is predictive of the development of Alzheimer's disease and is negatively correlated with treatment efficacy of tacrine (a colinestrase inhibitor) and an experimental vasopressinergic drug (a non-cholinomimetic drug). The BCHE-k allele has a point mutation at nucleotide 1828 (a G to A subsitution) which results in an ala539thr change. This polymorphism can be readily detected by PCR amplifying a region surrounding the variance site and sequencing the amplification product to determine the nucleotide at the particular site.

A group of patients was treated with an experimental vasopressinergic drug (n=91) and compared to patients administered a placebo (n=108) without segregation or stratification by BCHE or other allelic status. As evaluated using the Mini Mental State Examination (MMSE) over a twelve-week treatment period, no statistically significant improvement was shown for the treatment group. However, when the treatment group was stratified according to the presence or absence of a BCHE-k allele, those patients without such an allele showed a statistically significant improvement while those having at least one of the BCHE-k alleles did not. Thus, the analysis provides an example of a gene where a patient sub-population was identified where a treatment showed a positive response even though no such positive response was found for the overall patient population. Indeed, those patients not having a k-allele are approximately three times more likely to respond to the vasopressinergic drug than are patients having at least one k-allele.

The response of Alzheimer's disease patients treated with the cholinomimetic drug, tactrine, was also determined. Similar to the above, the MMSE test was utilized as an indicator of a positive response. The positive response rate was approximately two-fold higher in those patients not having a k-allele than in those patients having at least one k-allele.

In addition, it was found that the presence of either or both of a BCHE-k allele and an apoE-4 allele was positively correlated with the development of Alzheimer's disease. For example, in patients over 75 years of age, the odds ratio of a patient having a BCHE-k allele was 2.3, the odds ratio for having a apoE-4 allele was 2.0, and the odds ratio for the joint occurrence of both alleles was 17.5. Thus, the BCHE-k allele is an example where the presence of a variant allele is negatively correlated with the efficacy of treatment with drugs from multiple drug categories, and which is further positively correlated with the development of a particular disease. Thus, the variance status of such a gene is useful both as a prognostic tool for disease risk, as well as for identifying likely drug responders versus non-responders for drug development and/or treatment selection.

The evidence that a variance in a gene involved in a pathway that affects drug response in patients with dementia, indicates and supports the theory that there is a likelihood that other genes have similar qualities to various degrees. As drug research and development proceeds to identify more lead candidate therapeutic interventions for dementia, there is possible utility in stratifying patients based upon their genotype for these yet to be correlated variances. Further, as described in the Detailed Description, methods for the identification of candidate genes and gene pathways, stratification, clinical trial design, and implementation of genotyping for appropriate medical management of a given disease is easily translated for dementia. As described below in section V. below there are likely gene pathways as are those that are outlined in the gene pathway Table 1 and matrix Table 2.

Advantages of Pharmacogenomic Clinical Development of Therapies for Dementia

The advantages of a clinical research and drug development program that includes the use of polymorphic genotyping for the stratification of patients for the appropriate selection of candidate therapeutic intervention includes 1) identification of patients that may respond earlier to therapy, 2) identification of the primary gene and relevant polymorphic variance that directly affects efficacy, safety, or both, 3) identification of pathophysiologic relevant variance or variances and potential therapies affecting those allelic genotypes or haplotypes, and 4) identification of allelic variances or haplotypes in genes that indirectly affects efficacy, safety or both.

Based upon these advantages, designing and performing a clinical trial, either prospective or retrospective, which includes a genotype stratification arm will incorporate analysis of clinical outcomes and genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of dementia.

By identifying subsets of patients with mild to moderate dementia that respond earlier to drugs or agents or experience enhanced efficacy, optimal candidate therapeutic interventions may reduce the period of time prior to relief of cognitive impairments. Appropriate genotyping and correlation to dosing regimen would be beneficial to the patient, caregivers, medical personnel, and the patient's loved ones.

Optimization of cholinomimetic mediated therapy of dementia further demonstrates the utility of selection of a potential dementia patient that has a predisposing genotype in which selective cholinomimetic are more effective and or are more safe. In considering an optimization protocol, one could potentially predetermine variance or variances within the muscarinic cholinergic receptor, nicotinic cholinergic receptor, modulatory mechanisms of cholinergic neurotransmission, or cholinergic receptor mediated intracellular mechanism of action that is preeminently responsible for drug response. By embarking on the previously described gene pathway approach, it is technically feasible to determine the relevant genes within such a targeted drug development program for dementia.

V. Description of Mechanism of Action Hypotheses for Future Drug Development for the Therapy of Dementia

Drug development programs for the identification of novel drug or candidate therapeutic interventions are aimed at the underlying pathophysiologic mechanisms of the disease leading to clinical signs and symptoms of dementia. Current hypotheses include, but are not limited to, therapeutic development in one of the following areas: 1) replacement of cholinergic function, 2) acetylcholine pathway: biosynthesis, secretion, degradation, reuptake, and receptor binding, 3) CNS lipid transport/membrane repair pathway and gene identification, 4) inflammatory mediators, e.g. prostaglandin, prostacylin, and thromboxane pathway, and 5) constituents of AD lesions and AD genes. These are described in detail below.

A. Therapeutic Approaches for Replacement of Cholinergic Function

Because dementia is apparently related to a loss of cholinergic function in the neocortex and forebrain arising from death or atrophy of basal forebrain cholinergic neurons, replacement of cholinergic function has been shown to have therapeutic benefit. In general, novel approaches for the replacement of cholinergic function can be characterized as either: 1) therapies that compensate for existing damage; and 2) therapies that halt, retard, or prevent cerebral damage.

B. Therapeutic Approaches that Compensate for Existing Damage

The therapeutic approaches that may compensate for existing damage include modulating cholinergic deficit, modulating other neurotransmitter deficits, modulating immune or inflammatory mechanisms of neural damage, and modulation of metabolism of specific neurotransmitters. Although these novel therapies are aimed at existing or damage yet to occur, the underlying course of the disease will remain.

Potential therapies for the compensation for cholinergic deficit are 1) increase presynaptic production of acetylcholine, 2) enhance release of acetylcholine, 3) stimulate choline reuptake, 4) selective muscarinic agonists, 5) anticholinesterase inhibitors, 6) mixed action anticholinesterases and muscarinic receptor ligands, and 6) nicotinic receptor agonists.

Potential therapies for the compensation for modulating other neurotransmitters are 1) selective NMDA agonists, and 2) other disorders of neurotransmitter function.

Potential therapies for the compensation for modulating immune or inflammatory mechanisms of neural damage are 1) antiinflammatory agents that suppress inflammation and 2) inhibition of amyloid precursor protein (APP) degradation.

Potential therapies that compensate for monoamine metabolite deficits are agents that affect monoamine oxidase type B enzyme activity, therapy for behavioral symptoms of neurotransmitter function in dementia, and compensate for immune or inflammatory mechanisms involved in neural cell destruction.

C. Therapeutic Approaches that Halt, Retard, or Prevent Cerebral Damage

In general therapeutic approaches that halt, retard, or prevent cerebral neural damage are currently either growth factors or modulation of the deposition of aberrent pathological depositions of metabolic by-products. These approaches include promotion of the growth and regeneration of cholinergic neurons and generally include growth factors that act on neurons, neural precursors, or glial cells. Growth factors include but are not limited to nerve growth factor (NGF), brain-derived growth factor (BDGF), neurotrophins, and leukemia inhibitory factor (LIF).

Prevention of amyloid plaque deposition includes modulation of APP gene expression, prevention of the development of amyloidogenic peptide, inhibition of amyloid aggregation/secretion, and APP antagonists. Prevention of the formation of neurofibrillary tangles includes modulation of the phosphorylation of tau proteins.

D. CNS Lipid Transport/Membrane Repair Pathway and Gene Identification

Brain Apolipoproteins: The six apolipoproteins known to be expressed in the brain are listed below. They are present on the surface of three major types of lipoproteins, one class enriched in A-I, but also containing most of the D, E, and J protein in the brain; one class composed principally of E with minor amounts of A-I, A-IV, D, and J; and a third minor class containing the majority of A-IV. Variation in the structure or expression of these apolipoproteins can modulate lipid transport and brain remodeling.

Lipoprotein Receptors: Six brain receptors for lipoproteins have been identified in man. These include the low density lipoprotein receptor (LDL-R), the LDL receptor-related protein (LRP), the very low density lipoprotein receptor (VLDL-R), and the class A macrophage scavenger receptor, all of which are also expressed outside the brain. Two new protein with LDL receptor-like domains have recently been identified in human brain: Apolipoprotein E receptor type 2, and the SorLA-1 receptor. Alterations in the structure or expression of those receptors can affect binding of ApoE alleles (ApoE2, for example, has reduced affinity for the LDL receptor), and more generally will modulate the biology of lipid transport.

Lipoprotein docking and lipid mobilization: Heparin sulfate proteoglyans (HSPG) are responsible for initial binding of ApoE-bearing lipoproteins to cells. Removal of HSPGs with heparinase blocks binding, even in the presence of receptor (LDL-R or LRP). Therefore variations in biosynthetic enzymes of the HSPG pathway will influence lipoprotein uptake. Lipid hydrolysis by cholesterol ester transfer protein (CETP) effects the transport of lipids from lipoproteins into cells.

Cholesterol Metabolism: Acyl CoA:cholesterol acyltransferase and HMG CoA reductase are responsible for the metabolism of cholesterol, therefore variations in the metabolic pathway of cholesterol will influence availability of cholesterol.

Hormonal control of lipoproteins and lipoprotein receptors: The expression of lipoproteins and their receptors is under hormonal control. Clinical studies of tacrine for Alzheimer's disease have also shown reduced incidence of AD in women taking estrogen supplements post menopausally. Therefore, variation in hormone levels, hormone receptors, or hormone receptor signaling pathways will modulate response to acetylcholinesterase inhibitors, e.g., by affecting lipid transport and cholinergic remodeling or by other means. Hormone receptors that bind their physiologic ligand within the cytoplasm then become activated and cross the nuclear membrane include but are not limited to growth hormone, prolactin, estrogen, retinoic acid receptor, thyrotropin releasing hormone. Associated transcriptional co-activators include but are not limited to SRC-1, SRC-2 (TIF-2), SRC-3 (p/CIP:AIB1), P/CAF, CBP, E6-AP, TRIP230, SMRT, SRA, and N-CoR.

E. Prostaglandin, Prostacylin, and Thromboxane Pathway

Inflammatory mediators, and in particular the products of arachidonic acid metabolism, play a role in the development of AD neuropathology.

There are several lines of evidence supporting the role of inflammatory or immunological processes in the pathogenesis of Alzheimer's disease. First, neurodegeneration in AD is accompanied by manifestations of immune reaction including activation of the complement cascade, accumulation and activation of microglia and presence of inflammatory cytokines and acute phase reactants in tissue of AD brains. Second, epidemiological studies suggest that use of non-steroidal anti-inflammatory drugs (NSAIDs) delays the clinical expression of Alzheimer's disease. The development of selective COX inhibitors has led to renewed interest in the therapeutic potential of NSAIDs in AD.

Arachidonic acid formation pathway genes include phospholipase A2, phospholipase C β3, and diacylglycerol lipase. PGG2 formation pathway genes include cyclooxygenase I, cyclooxygenase II. PGH2 formation pathway genes include PGG2 reductase. PGH2 metabolizing enzymes include PGH2 reductase, PGD2 reductase, PGH-PGE isomerase, and thromboxane A2 synthase. Receptors include PGF1a receptor, PGD2 receptor, PGE2 receptor, PG12 receptor, and thromboxane A receptor. Exemplary variances for genes above are shown in Tables 3 and 4.

F. Constituents of Alzheimer's Disease Lesions and AD Genes

The relative contribution of different pathogenetic mechanisms to the development of AD in specific patients can affect the degree of cholinergic impairment and hence the response to acetylcholinesterase inhibitors.

There is clear evidence that different pathogenetic mechanisms affect the onset and rate of progression of AD. The possible effects of such are several lines of evidence supporting the role of inflammatory or immunological processes in the pathogenesis of Alzheimer's disease. First, neurodegeneration in AD is accompanied by manifestations of immune reaction including activation of the complement cascade, accumulation and activation of microglia and presence of inflammatory cytokines and acute phase reactants in tissue of AD brains. Second, epidemiological studies suggest that use of non-steroidal anti-inflammatory drugs (NSAIDs) delays the clinical expression of Alzheimer's disease. The development of selective COX inhibitors has led to renewed interest in the therapeutic potential of NSAIDS in AD. Pathway genes include Tau protein, amyloid precursor protein, presenilin 1, and presenilin 2.

In Tables 3 and 4, there are listings of candidate genes and specific single nucleotide polymorphisms that may be critical for the identification and stratification of a patient population diagnosed with dementia based upon genotype. Current pathways that may have involvement in the therapeutic benefit of dementia include, but are not limited to, glutaminergic, serontonergic, dopaminergic, adrenergic, cholinergic, histaminergic, purinergic, GABAergic, glycinergic, nitric oxide, peptide protein processing, opiates, cholecystokinin, corticotropin releasing factor, thyroid stimulating hormone, somatostatin, adrenocorticotropic hormone, vasoactive intestinal peptide, calsium or potassium channels, prostaglandin, cytokines, estrogen, clot formation, hemostasis, oxygenstress, mitochondrial maintenance, protein maturation and degradation, second messenger cascade, growth, differentiation and apoptosis, cytoskeleton, secretion, amyloid processing, and lipid transport or metabolism gene pathways that are listed in Tables 1, 3 and 4. One skilled in the art would be able to identify these pathway specific gene or genes that may be involved in the manifestation of dementia, are likely candidate targets for novel therapeutic approaches, or are involved in mediating patient population differences in drug response to therapies for dementia.

A sample of therapies approved or in development for preventing or treating the progression of symptoms of dementia currently known in the art is shown in table 7. In this table, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, that are therefore also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

Based upon these varying approaches there are many products in development for dementia. In Table 7 below lists current therapies that are in development for U.S. marketing approval. Identification of genes in specific pathways and the link to specific agents or drugs may be useful to conduct clinical trials and achieve higher degrees of safety and efficacy. The listed candidate therapeutic interventions response in patients with dementia may be affected by polymorphisms in genes as described above.

### EXAMPLE 3

Depression

I. Description of Depression

The incidence of depression in the U.S. is 82-201/100,000 males and 247-598/100,000 females indicating a 2 to 1 distribution based upon gender. The prevalence is 3/100 males and ranges from 4 to 9 per 100 females. The estimated annual cost associated with depression is $16 billion dollars.

Major depression is a psychiatric disorder distinguishable from normal grief, sadness, and disappointment as well as the dysphoria and demoralization often associated with medical illness. Depressive disorders are characterized by abnormally long term depressed mood and may be accompanied by delusions and hallucinations. Individuals suffering from depression have feelings of despair and intense sadness, exhibit mental slowing and loss of concentration, are preoccupied with pessimistic worry and inner self, and are agitated and tend toward self-deprecation. In some depressive disorders, mania is present usually in episodic intervals and in these cases depressed mood is replaced with feelings of grandiosity and may be accompanied by incoherent speech. Clinically, unipolar or bipolar depression are terms used to describe the two broad categories of depressive disorders characterized by the absence or presence of episodic mania, respectively.

II. Current Medical Management Strategies for Depression

Unipolar Depression

Depression is a wide-spread disease that requires improved therapeutic alternatives to the conventional agents that have been available since the 1960s. Current therapeutic candidates of unipolar or bipolar depression are as follows: tricyclic antidepressants, tetracyclic antidepressants, lithium, monoamine oxidase (MAO) inhibitors, electroconvulsive therapy (ECT) and atypical agents such as PROZAC®, WELLBUTRIN®, and trazodone.

Bipolar Depression

Despite the difficulties of medical management of bipolar depression, advances have changed therapeutic outcomes. Therapies such as lithium, valproate, and carbamazepine, clozapine, and ECT have made a positive impact on the patient outcomes. Further, the importance of psychosocial issues for understanding patients illnesses and factors affecting treatment compliance are more fully realized.

For bipolar depression, mood stabilizers are the first line therapy and include: lithium, valproate, and carbamazepine. Adjunct therapies are used for the treatment of agitation, insomnia, or aggressive behaviors and include benzidiazepines and antipyschotics. ECT is useful as an alternative therapy in patients who are pregnant or are trying to conceive, unresponsive to standard therapy, unable to tolerate first line therapies, or are refractory to first line or adjunct therapies. ECT has been shown to be effective as stated above, as well as 54% effective in refractory patients.

There are additional therapies that have been used for the treatment of bipolar depression. For example, off-label use of clozapine, Ca++ channel antagonists, gabapentin, and lamotrigine in diagnosed bipolar patients have been demonstrated to be effective at stabilizing mood. Gabapentin, has a higher safety profile during pregnancy, but has side effects of ataxia, fatigue and somnolence. Lamotrigine, by effectively lowering glutamine release is effective at stabilizing mood, but is associated with dizziness, headache, double vision, somnolence, headache, and rash. Other medications include valproate for euphoric mania, valproate for dysphoric mania or mixed mania, and clozapine with lithium or valproate for patients with rapid-cycling episodes.

III. Limitation of Current Therapies for Depression

Frequently, depression is undiagnosed and if detected, treatment often is inadequate. Therapy of depression is associated with undesirable side effects and/or simply fails to adequately manage the symptoms of the condition. Thus, there is a need for ongoing improved development of antidepressant therapeutic alternatives to the currently available products.

Limitations of Current Therapies for Unipolar Depression

Although these agents or therapies are efficacious (e.g. 80% improvement following ECT; lithium effectively prevents relapses in 60% of patients) there are significant limitations to their use and are 1) the onset of action of antidepressant drugs is latent, 2) responsivity and efficacy is not uniform, 3) long-term treatment can lead to symptoms of drug resistance, 4) there is perceived inhibition of creativity and decreased energy, and 5) there are patients with refractory depression with no therapeutic alternatives.

Limitations of Current Therapies for Bipolar Depression

Bipolar depression patients have additional therapeutic concerns as compared to unipolar depression patients. For bipolar patients there is the added difficulty of treating depression episodes. The efficacy of antidepressants is not well founded or documented in bipolar depression. Further, antidepressants have been documented to induce manic or hypomanic symptoms. Therefore, mood stabilizers are the first line therapy with adjunct therapies during manic or depression episodes.

An additional therapeutic issue associated with bipolar patients is that many comorbid psychiatric disorders occur within the same patient not only hindering a diagnosis, but also therapy. For example, substance abuse disorders, panic disorders, obsessive-compulsive disorders, and impulsive control disorders are often present and potentially mask symptoms of bipolar depression.

IV. Impact of Pharmacogenomics on Drug Development for Depression

There are two genes that have been described having polymorphisms that affect antidepression drug response, the serotonin transporter gene and the angiotension converting enzyme that affects the metabolism of substance P. These two examples are described below.

The Serotonin Transporter Gene

The serotonin transporter gene (5-HTT) polymorphism provides an example of a recessive SNP polymorphism in the non-coding region with an impact on inefficacy of a 5-HTT selective drug.

The serotonin transporter (5-HTT) plays a critical role in the termination of the serotonin (5-HT) neurotransmission and represent the prime target for selective serotonin reuptake inhibitors (SSRIs). A functional polymorphism in the transcriptional control region upstream of the 5-HTT coding sequence has been reported. It consists of a 44-base pair insertion (long variant) or deletion (short variant). It has been demonstrated that the long (1) and short (s) variants of this 5-HTT gene-linked polymorphic region had different transcriptional efficiencies. In vitro studies showed that the difference in 5-HTT mRNA synthesis result in different 5-HTT expression and 5-HT cellular uptake (Lesch et al. Science 1996 274:1527-153). Recently, it has been shown that an SSRI (fluvoxamine) efficacy in delusional depression seems to be related to allelic variation within the promoter of the 5-HTT gene (Smeraldi et al. Mol. Psychiatry 1998; 3:508-511). Both homozygotes for the long variant (l/l) of the 5-HTT promoter and heterozygotes (l/s) showed a better response to fluvoxamine than homozygotes for the short variant (s/s). Interestingly, the addition of pindolol (a mixed adrenoreceptor and 5-HT1A antagonist) has been proposed as an augmentation therapy for non-responders or partial responders to SSRIs, and it appears that in the group treated with fluvoxamine plus pindolol all the genotypes acted like l/l treated with fluvoxamine alone. This supports the hypothesis that the effect of pindolol is related to its ability to block 5-HT1A autoreceptors, thus preventing a negative feed-back of 5-HT at somatodendritic level. Furthermore, the activation of 5-HT1A autoreceptors could modulate the clinical effect of the SSRIs-induced 5-HTT blockade.

The 5-HTT polymorphism represents an example of a gene allelic variance that affects the transcriptional control, and ultimately, the amount of available transporter protein. In these cases, the gene product concentration or protein availability affects the function of the native mechanism and ultimately the ability of the drug to intervene with physiological function. One skilled in the art, upon utilizing the techniques described in the detailed description, would be able to identify known variances within a candidate gene, provide a diagnostic test to identify individuals with that variance or variances, group the individuals based upon the identified genotype, and design and implement a clinical study to test the effect a candidate drug has on the the groups. In this example, the allelic differences may affect transcriptional or translational control of the 5-HTT gene. A skilled practitioner will be able to utilize the techniques known in the art to determine the effects of a variance or variances within a gene promoter region to be able to study the impact those allelic differences have on the safety or efficacy of SSRIs or any other candidate drugs affecting the 5-HT pathway. Further, this example underscores the ability of a skilled practitioner to be able to utilize methods known in the art to design a pharmacogenomics clinical trial when the allelic difference is within the gene promoter region.

The Angiotensin Converting Enzyme Gene and Substance P

The localization of substance P in brain regions that coordinate stress responses and receive convergent monoaminergic innervation suggested that substance P antagonists might have psychotherapeutic properties. Similar to clinically used antidepressant and anxiolytic drugs, substance P antagonists suppress isolation-induced vocalizations in guinea pigs. In a placebo-controlled trial in patients with moderate to severe major depression, robust antidepressant effects of the substance P antagonist MK-869 were consistently observed. In preclinical studies, substance P antagonists did not interact with monoamine systems in the manner seen with established antidepressant drugs. These findings suggest that substance P may play an important role in psychiatric disorders.

Substance P is highly metabolized by ACE (angiotensin converting enzyme) which is a good actual example of pharmacogenetics: It has a high allele frequency in normal individuals (D: 34%, I: 66%) and there are clinical studies clearly demonstrating its impact on ACE inhibitors.

Moreover, it has been shown that DD homozygous patients (11%) have a higher brain level of substance P than II homozygous patients (43%), with an intermediate level for heterozygous patients (46%).

Using results of the initial phase II trial, we expect that a substance P antagonist will have more impact on patients with high brain level of substance P (actually, the DD patients who are more at risk for affective disorders). As measure of response rate, starting with the standard measure of response defined as ≧50% change from baseline to week 6 in total HAM-D21 score, 54% of the patients improved with MK-869 and 28% patients improved with placebo in the phase II trial.

In a recent clinical trial of MK-869 versus placebo, a similar response rate was observed for both groups (54% and 48% respectively). If the ACE variance is considered as a dominant SNP with regard to substance P metabolism, calculation of an unequivocal positive response rate in the DD subgroup (i.e., 100%) would require an equally similar response rate in the II subgroup, while assuming the DI subgroup response rate remains similar to placebo (i.e., 48%). In this case, MK-869 would be positive (100%) only in a fraction of the patients, e.g., one out of every five.

Approximately 25% of the responders should be DD homozygous; if not, the hypothesis is not valid. Then, if 25% are DD, the number of patients included in the failed trial should be enough to see a statistically significant difference between the DD subgroup and other patients, since we would need at least 56 patients to test for such a high relative risk (100%/48%=2).

This approach exemplifies the utility of high allele frequency polymorphisms. Further, when the treatment is not efficacious for all individuals (i.e. response rates vary between treatment groups is less than 15%) the allele frequency of a potentially interacting recessive SNP polymorphism should be relatively high (e.g. from 30% for a 15% difference in response rate to 60%). This corresponds to 16% or less of total patients (see example 18 and table below).

The evidence that a variance in a gene involved in a pathway affects antidepressive drug response, indicates and supports the idea that other genes have similar qualities to various degrees. As drug research and development proceeds to identify more lead candidate therapeutic interventions for depression, there is utility in stratifying patients based upon their genotype for these yet to be correlated variances. Further, as described in the Detailed Description, methods for the identification of candidate genes and gene pathways, stratification, clinical trial design, and implementation of genotyping for appropriate medical management of a given disease is easily utilized for depression. As described below in section V. below there are likely gene pathways such as those outlined in the gene pathway Table 1 and matrix Table 2.

Advantages of Pharmacogenomic Clinical Development of Therapies for Depression

The advantages of a clinical research and drug development program that includes the use of polymorphic genotyping for the stratification of patients for the appropriate selection of candidate therapeutic intervention includes 1) identification of patients that may respond earlier to antidepressant therapy, 2) identification of the primary gene and relevant polymorphic variance that directly affects efficacy, safety, or both, 3) identification of pathophysiologic relevant variance or variances and potential therapies affecting those allelic genotypes or haplotypes, and 4) identification of allelic variances or haplotypes in genes that indirectly affects efficacy, safety or both.

Based upon these advantages, designing and performing a clinical trial, either prospective or retrospective, which includes a genotype stratification arm will incorporate analysis of clinical outcomes and genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of unipolar or bipolar depression.

By identifying subsets of patients with depression that respond earlier to antidepressant drugs or agents, optimal candidate therapeutic interventions may reduce the period of time prior to relief of seizure frequency and severity. Appropriate genotyping and correlation to dosing regimen would be beneficial to the patient, caregivers, medical personnel, and the patient's loved ones.

Optimization of adrenergic control or ion channel modulation mediated therapy of epilepsy further demonstrates the utility of selection of a potential epilepsy patient that has a predisposing genotype in which selective adrenergic or agents are more effective and or are safer. In considering an optimization protocol, one can potentially predetermine variance or variances within the adrenergic receptor, ion channel or ion channel mediated mechanisms of neurotransmission, or adrenergic receptor mediated intracellular mechanism of action that is preeminently responsible for drug response. By embarking on the previously described gene pathway approach, it is technically feasible to determine the relevant genes within such a targeted drug development program for depression.

A sample of therapies in development for preventing or treating the progression of symptoms of depression currently known in the art is shown in table 8. In this table, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

V. Mechanism of Action Hypotheses for Novel Therapies for Depression: Utility of Genotyping

Unipolar Depression

Unfortunately, to date the biological mechanism of major unipolar depression is unclear. However, studies of endocrine systems, neurotransmission, and neuroelectrophysiology have provided the basis for the generation of pathophysiologic hypotheses. These hypotheses have been supported by clinical data stemming from the success of conventional treatment of depression.

One such hypothesis is that there is pituitary-hypothalamic dysfunction in depressed patients. It has been observed that depressed patients commonly have elevated levels of cortical steroids in their urine and blood. Further, 50% of the patients with clinical depression will not secrete cortisol when subjected to the dexamethasone suppression test. Additionally, thyrotropin releasing hormone (TRH) stimulation of thyrotropin stimulating hormone (TSH) release is aberrant in depressed patients without an alteration of serum T3 or T4 concentrations and growth hormone, prolactin, gonadal hormones, corticotropin releasing factor (CRF), and melatonin have diminished physiologic responses.

Another hypothesis of the biological dysfunction of depression is that there is a neurotransmitter dysfunction due to a catecholamine-indolamine imbalance. This theory postulates that there is a required level of catecholamines and receptor sensitivity required for normal mood. In depression, there may be aberrant receptor insensitivity, depletion of amines, or a depletion of their synthesis or storage that leads to depression. Supporting this theory is that monoamine oxidase inhibitors increase the availability of catecholamines and indolamines and have been used clinically for the management of depression.

The cholinergic neurotransmitter system has been implicated in the manifestation of depression. In has been postulated that there is an imbalance of adrenergic and cholinergic control of neural transmission in patients with depression.

Electrophysiologic studies have shown that patients with depression have altered rapid eye movement (REM) sleep patterns, i.e. shortened REM latency, than non-depressed patients. Other studies have documented a correlation of the circadian rhythm and precipitation of depressive episodes during autumn and winter months and diminished ambient light during those times during the year.

In each of the theories posited and described above, satisfactory conclusions are limited. Conventional therapy of depression with tricyclic antidepressants has demonstrated that this treatment affects more than one neurotransmitter system due to either modification or alteration of the regulation of neurotransmitter receptors signaling pathways rather than acting solely at neurotransmitter receptor binding.

Novel therapies of unipolar depression include venlafaxine and mirtazapine. Both of these compounds show promise in clinical trials for the treatment of depression. Venlafaxine is a mixed serotonergic and noradrenergic reuptake inhibitor. Mirtazapine has noradrenergic and serotonergic antidepressant mechanism of action. These two products have what looks to be superior action over tricyclic antidepressants or selective serotonergic inhibitors (SSRIs).

Bipolar Depression

Theories for the mechanism have been described. In one model, electrophysiological kindling and behavior sensitization underlie bipolar disorders and further increasing frequencies of episodes over time. In another model, there appears to be a desynchronization of circadian rhythm in bipolar patients.

As for depression, the catecholamine hypothesis presumes that mania is due to an excess of catecholamines and depression is due to their depletion. Noradrenergic and dopaminergic dysfunction have both been linked to depression. In both cases of dysfunction, there appears to be causal links, i.e. aberrant noradrenergic neurotransmission and L-dopa induced hypomania among bipolar patients, respectively. Amphetamines can produce hypomania in bipolar patients and dopaminergic antagonists are effective for severe mania.

The serotonergic hypothesis generalizes that low serotonergic transmission is responsible for mania and depression because low serotonergic inputs may result in defective neuromodulation. Other hypotheses include neurotransmitters, enzymes, neuropeptides, and theories involving endocrine and immunological systems. As in many other complex disorders of psychological function, these models fall short of adequately describing the disturbance. Future studies and drug development may provide insights to refined biological mechanism of bipolar depression.

In Tables 3 and 4, there are listings of candidate genes and specific single nucleotide polymorphisms that may be critical for the identification and stratification of a patient population diagnosed with depression based upon genotype. Current pathways that may have involvement in the therapeutic benefit of depression include glutaminergic, serontonergic, dopaminergic, adrenergic, cholinergic, purinergic, GABAergic, melatonin, peptide protein processing, opiates, oxytocin, neuropeptide Y, calcitonin/calcitonin gene related peptide, tachykinin, corticotropin releasing factor, vasopressin, calcium or potassium channels, prostaglandin, testosterone, oxygen stress, second messenger cascade, folate metabolism pathways that are listed in Tables 1, 2, 3 and 4. One skilled in the art would be able to identify these pathway specific gene or genes that may be involved in the manifestation of depression, are likely candidate targets for novel therapeutic approaches, or are involved in mediating patient population differences in drug response to therapies for depression.

In Table 8 below is a list of the available candidate therapeutic alternatives available or in development for depression. There are listed by therapeutic approach are defined and listed in Table 1. The listed candidate therapeutic interventions response in patients with depression may be affected by polymorphism in genes as described above.

### EXAMPLE 4

Epilepsy

I. Description of Epilepsy

Epilepsy is a neurological disorder affecting an estimated 1.8 million Americans with estimated direct and indirect costs of illness to be approximately $3 billion dollars. Epilepsy is characterized by the behavioral consequences of recurrent, spontaneous, transient paroxysms of abnormal brain activity. An epileptic attack or seizure may result in impaired consciousness, involuntary movements, autonomic disturbances, psychic or sensory disturbances. The fundamental etiology of epilepsy is thought to occur within the cerebral cortex or limbic cortex (hippocampus). Chronic epilepsy is the syndrome in which recurrent neuronal paroxysms that underlie ictal events are transient expression of more permanently physiological disordered cortex. In ascertaining the location and the diagnosis of epilepsy, one can determine patterns of uncoordinated cortex by examination of ictal and interictal EEG recordings. Interictal recordings of epilepsy patients have an appearance of brief discharges that can be recorded from the scalp. There is a noticeable spike-wave complex that is evident and is characterized by sharp negative transients followed by a slower wave. The EEG spike-wave complex reflects a summation of highly synchronized abnormal neuronal membrane potentials that upon inspection appear as large paroxsymal depolarization shifts followed by prolonged after depolarizations.

Epilepsy can be divided into the following categories based upon etiology: 1) primary epilepsy which is an intrinsic, nonprogressive, hereditary group of cerebral disturbances, 2) secondary epilepsy which is symptomatic of some know pathologic processes affecting the brain, and 3) reactive seizures which are characterized by natural reaction to physiologic stress or transient ischemic injury.

Epilepsy can be categorized into the following categories: partial seizures, generalized seizures, and seizures of unknown origin. Partial seizures are initiated (uni- or bilaterally) in discrete focal areas in the cortex and remain focal lesions. Generalized seizures begin either uni- or bilaterally and spread throughout the cortical tissue. In either case the mechanism of epileptogenic activity is to date unknown. However, there is evidence suggesting the etiology of epilepsy.

Partial seizures can be further subcategorized into: 1) simple partial seizure disorders, consciousness not impaired (with motor signs or symptoms, with somatosensory or special sensory symptoms (e.g. simple hallucinations, such as tingling, light flashes, buzzing), with autonomic signs and symptoms (e.g. epigastric sensation, pallor, sweating, flushing, piloerection and pupilary dilation), with psychic symptoms (e.g. disturbances of higher cerebral function (déjà vu, fear, distortion of time perception)); or 2) complex partial seizure disorders (simple partial onset following impairment of consciousness, impairment of consciousness at onset); or 3) partial seizures evolving to generalized tonic clonic seizures (simple partial seizures evolving to generalized seizures, complex partial seizures evolving to generalized seizures, and simple partial seizures evolving to complex partial seizures and further evolving to generalized seizures). A key feature of partial epilepsy is auras. These somatosensory or special sensory symptoms manifest as sensations described above and precede the seizure. There are cases whereby pharmacotherapy reduces the frequency and severity of partial seizures but may have little to no effect on aura sensation in partial epilepsy patients.

Generalized seizures are divided into 1) nonconvulsive seizures (absence seizures, atypical seizures, myoclonic seizures, or atonic seizures), or 2) convulsive seizures (tonic-clonic seizures, tonic seizures, or clonic seizures). Other seizure disorders that do not fit into the above categories are some cases of neonatal and infantile seizures.

There are other factors that one must consider when diagnosing seizure disorders. A generalized seizure may be the result sleep deprivation, alcohol or sedative drug withdrawal, use of convulsant drugs, fever, or acute head trauma. Furthermore, reversible toxic, infectious, or metabolic processes may induce recurrent generalized convulsions. Infantile febrile convulsions are an example of infancy and early childhood seizures that may or may not be indicative of a future epilepsy diagnosis.

Acquired epilepsy may be the result of congenital lesions, head trauma, infectious processes, brain tumors, cerebrovascular disease, systemic toxic and metabolic disturbances, hippocampal sclerosis, and miscellaneous disorders (collagen vascular disease, blood dyscrasias, cerebral gray matter degenerating diseases (allergic encephalopathy), presenile or senile dementias).

Epilepsy may be confused with clinical signs and symptoms of syncope, migraine, or pseudoseizures (nonepileptic psychogenic seizures). Usually, video/EEG monitoring of the patient during ictal and interictal periods allows trained personnel to distinguish epilepsy from these other clinical presentations.

II. Current Medical Management of Epilepsy

For the majority of patients, epileptic seizures can be controlled with antiepileptic drug therapy (in many cases, monotherapy) and may be withdrawn if the patient is seizure free for an extended period, usually 2 years. Some patients do not become free of seizures, despite therapy compliance. Persistent epilepsy, aside from deleterious effects on health, has psychosocial, behavioral, and cognitive consequences, which often impose financial burdens to patients, their loved ones, and society.

Based upon accurate diagnosis of the seizure type and seizure-associated physiology, appropriate therapy to reduce seizure frequency, severity, and epilepsy-associated behaviors can be identified. Diagnosis of epilepsy involves both identification of the epileptic syndrome and the type of seizure. Syndromes are identified based upon age of onset, EEG recording analysis, location of the epileptic region or site of epileptogenesis, type of seizure. The drugs available for medical management of epilepsy are divided by their use in the clinic; common forms of epilepsy are treated differently that partial or secondarily generalized tonic-clonic seizures disorders.

The current pharmacotherapy has three main mechanisms of action: 1) reduction of sustained repetitive firing of a neuron by promoting the inactivation state of voltage-activated Na+ channels; 2) enhanced GABAergic mediated presynaptic or postsynaptic inhibition of neural transmission; or 3) limiting the activation of specific voltage-activated Ca++ channels (T current). Following these general mechanism of action, current anticonvulsant drugs act by 1) prolonging the inactivation of the Na+ channels thereby reducing the ability of neurons to fire at high frequencies, 2) affecting GABAergic neurotransmission by reducing the metabolism of GABA, acting at the GABA receptor, enhancing the Cl⁻ influx in response to GABA postsynaptically, or promoting presynaptic GABA release, or 3) reducing the flow Ca++ T-type calcium channels reducing the pacemaker current that underlies the thalmic rhythm in spikes and waves in generalized absence seizures.

There are generally accepted first- and second-line drugs for each of the types of epilepsies and associated syndromes. For partial seizures they are carbamazepine and phenyloin (first-line) and gabapentin, lamotrigine, phenobarbital, primidone, tagabine, topiramate and valproic acid (second-line). For generalized seizures they are: absence seizures ethosuximide and valproic acid (first-line); lamotrigine (second-line); myoclonic seizures, valproic acid (first-line), acetazolamide, clonazepam, lamotrigine, or primidone (second-line)); tonic-clonic seizures valproic acid, carbamazepine, phenyloin (first-line), lamotrigine, phenobarbital, primidone (second-line); absence epilepsy with onset in childhood ethosuximide (first-line), valproic acid, lamotrigine (second-line); absence seizures with onset in adolescence valproic acid (first-line), ethosuximide, lamotrigine (second-line)); juvenile myoclonic epilepsy valproic acid (first-line), acetazolamide, clonazepam, primidone, lamotrigine (second-line); infantile spasms (West's syndrome corticotropin (first-line), clonazepam, valproic acid)); Lennox-Gastaut syndrome valproic acid, lamotrigine (first-line), carbamazepine (second-line).

Because there is greater risk for refractory epilepsy in partial epilepsy patients, there has been greater demand for the development of novel treatment alternatives. Since 1993 and as stated above, the introduction of lamotrigine, topiramate, tiagabine, and gabapentin have changed the medical management of partial epilepsy. Although carbamazepine and phenyloin remain the mainstay therapies, these additions to the antiepileptic arsenal have provided therapeutic alternatives to this subset population of epilepsy patients.

In addition to AEDs, refractory epilepsy may benefit from surgical therapy to remove the site of epileptogenesis or implantation of a device to stimulate the vagus nerve. Surgical removal of cortical tissue can be successful therapy in up to two thirds of certain selected epilepsy and can reduce the seizure frequency and severity in others. However, surgical therapy of refractory epilepsy is underused, and is often a delayed procedure. It has been estimated that there are approximately 50,000 epilepsy patients that could benefit from resective surgery, however, there are only an estimated 1,500 surgeries performed each year. Potential reasons for the profound difference in the potential number of surgical candidates and the number of procedures include: limited number of surgical teams performing the resective surgery; failure of primary physicians to identify potential candidates and to refer them to surgical centers; reluctance of third party payers to provide coverage for the costly presurgical diagnostic testing and procedures; and further, a reluctance on the part of the patient to voluntarily elect removal of cortical tissue.

Vagal nerve stimulation for the treatment of some patients with epilepsy has proven to be safe and well tolerated. A device is implanted in the upper quadrant that can be programmed to directly stimulate the vagal nerve. Stimulation of this autonomic nerve has lead to a documented 25% reduction of seizure frequency in refractory patients. The device does not appear to have similar efficacy when implanted in a partial epilepsy patient population. The use of the surgically implanted device has recently only been approved in the U.S. (June, 1997) for patients over 12 years of age with known refractory partial epilepsy. Transient hoarseness is a frequent side-effect of this device as a result of over-stimulation of the vagal nerve.

III. Limitations of Current Therapies for Epilepsy

The limitations of current medical management of epilepsy are 1) partial response to therapy or refractory epilepsy, 2) undesired side effects, 3) continuing medical management of refractory or partial response in epilepsy patients, and 4) noncompliance.

Partial Response to Therapy and Refractory Epilepsy as a Therapeutic Limitation

Approximately 80% of patients with epilepsy are medically managed with current pharmacotherapy. In the remaining 20%, epileptic seizure frequency and severity are refractory to currently available medications. Medical personnel are left with attempting combination therapy of available anti-convulsive therapy. Standard regimens of multiple anticonvulsant therapy are fraught with greater tendency towards unwanted side effects. Interestingly, 20% of the primary generalized epilepsy patients and 35% of partial epilepsy patients are refractory. A poor response to anti-epileptic therapy may be result of many different therapeutic or diagnostic causes. Since the focus of therapeutic management of refractory epilepsy is combination antiepileptic drug therapy, the balance of beneficial therapy and the patient's intolerance of the adverse effects of the AEDs must be appropriately monitored.

Undesired Side Effects or Toxicities as a Therapeutic Limitation

All of the anti-epilepsy agents or compounds have undesired side effects. For example, nausea, dizziness, diplopia, ataxia, sedation, impaired mentation, hyperactivity, folic acid deficiency, leukopenia, elevated serum alkaline phosphatase levels, pruritis, blood dyscrasias, hirsutism, gingival hyperplasia, coarsening features, weight gain, and alopecia have been described for various anticonvulsant therapies.

Individuals with epilepsy have an increased rate of mortality as compared to the general population. Mortality is associated with treatment and with seizures and may include one or more of the following: trauma, burns, and drowning, habitual seizures with cardiopulmonary disease, severe aspiration, food bolus, and sudden unexplained death. Sudden unexplained death in epilepsy patients (SUDEP) has been reported as high as 1 in 270 patients that are refractory to antiepilepsy drugs, and is a statistic that does not include suicides.

Additional concern of combination therapy besides increased propensity to experience undesirable side effects is the effect of metabolic rates and blood levels of the combinations. There is ample literature on the effect one antiepileptic agent has on another, for example carbamazepine decreases the blood levels of clanzepam, ethosuximide, methsuximide, primidone, tiagabine, topiramate, and valproic acid while increasing phenobarbital blood levels. Clonazepam decreases the blood levels of carbamazepine while decreasing primidone blood levels.

Continuous Medical Management as a Therapeutic Limitation

Antiepileptic drug (AED) therapy of epilepsy requires continuous medical monitoring. Factors involving lifestyle may trigger seizures in a patient diagnosed with epilepsy who have seemingly medically managed disease. For example, emotional stress, sleep deprivation, menstrual cycle, flickering lights and other sensory stimuli, alcohol use or withdrawal, or comorbidities (i.e. infections) may exacerbate seizures.

Noncompliance as a Limitation of Current Therapies

Noncompliance or partial compliance is a major concern in both monotherapy or combination therapy. Many patients who are in what appears to be remission, tend to noncompliance of their prescribed therapy. Determining plasma levels of the drug or drugs can monitor compliance, but this places an added burden on the patient and family members. Noncompliance can result from additional factors: missed medication, failure to refill the medication, a complicated dosing regimen, problems with memory or vision, postictal confusion, denial of medical condition, fear of teratogenic effects of the drug or drugs during pregnancy, concerns about the effects (both short and long-term) of the medication, and inability to afford the medication.

Clearly, for some patients, refined therapeutic management of seizure frequency and severity would have benefits above and beyond the clinical setting. Without many therapeutic alternatives to refine combination antiepileptic agent regimens, epilepsy poses a continued impact on health-related quality of life for each patient.

IV. Impact of Pharmacogenomics on Drug Development for Epilepsy

Genetic mechanisms of epilepsy have recently been described. However, the clinical genetics of seizure disorders has been a relatively slowly progressing field. Molecular genetic approaches have been useful to identify genes or gene clusters involved in linkage analysis.

Genetic polymorphism analysis and effects of antiepileptic drug therapy was recently described for the cytochrome P450 2C9 and 2C19 genes and these variance differences on the metabolic rates of phenyloin. The polymorphisms considered in this study were the arg144cys and the ile359leu of the CYP2C9 gene and the *1, *2, and *3 polymorphisms of CYP2C19. In this study of 134 Japanese patients, the mean maximal metabolic rates of phenyloin were 42% lower in individuals having the ile359leu genotype. From this analysis, the authors conclude that patients with the ile359leu genotype may not tolerate higher daily doses of phenyloin and may require genetic identification prior to implementation of medical strategies.

The evidence that a variance in a gene involved in a pathway that affects antiepilepsy drug response, indicates and supports the expectation that there is a likelihood that other genes have similar qualities to various degrees. As drug research and development proceeds to identify more lead candidate therapeutic interventions for epilepsy, there is possible utility in stratifying patients based upon their genotype for these yet to be correlated variances. Further, as described in the Detailed Description, methods for the identification of candidate genes and gene pathways, stratification, clinical trial design, and implementation of genotyping for appropriate medical management of a given disease is easily translated for epilepsy. As described below in section V. below there are likely gene pathways as are those that are outlined in the gene pathway Table 1 and in the gene pathway and indication matrix Table 2.

Advantages of Pharmacogenomic Clinical Development of AntiEpileptic Drug Therapies

The advantages of a clinical research and drug development program that includes the use of polymorphic genotyping for the stratification of patients for the appropriate selection of candidate therapeutic intervention includes 1) identification of patients that may respond earlier to antiepileptic therapy, 2) identification of the primary gene and relevant polymorphic variance that directly affects efficacy, safety, or both, 3) identification of pathophysiologic relevant variance or variances and potential therapies affecting those allelic genotypes or haplotypes, and 4) identification of allelic variances or haplotypes in genes that indirectly affects efficacy, safety or both.

Based upon these advantages, designing and performing a clinical trial, either prospective or retrospective, which includes a genotype stratification arm will incorporate analysis of clinical outcomes and genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of epilepsy.

By identifying subsets of epilepsy patients that respond earlier to AED or agents, optimal candidate therapeutic interventions may reduce the period of time prior to relief of seizure frequency and severity. Appropriate genotyping and correlation to dosing regimen would be beneficial to the patient, caregivers, medical personnel, and the patient's loved ones.

Optimization of GABAergic or ion channel modulation mediated therapy of epilepsy further demonstrates the utility of selection of a potential epilepsy patient that has a predisposing genotype in which selective AED or agents are more effective and or are safer. In considering an optimization protocol, one could potentially predetermine variance or variances within the GABAergic receptor, ion channel or ion channel mediated mechanisms of neurotransmission, or GABAergic receptor mediated intracellular mechanism of action that is preeminently responsible for drug response. By embarking on the previously described gene pathway approach, it is technically feasible to determine the relevant genes within such a targeted drug development program for epilepsy.

A sample of therapies approved or in development for preventing or treating the progression of symptoms of epilepsy currently known in the art is shown in table 9. In this table, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

V. Mechanism of Action Hypotheses for Novel Therapies for Epilepsy: Utility of Genotyping

Further studies have demonstrated that there is a genetic component to epilepsy. These genetic factors may predispose by an individual to epilepsy by inheriting one or more of the following 1) low threshold for aberrant seizure activity; 2) traits that underlie certain specific primary epilepsy disorders; and 3) a disease of the CNS in which there are associated structural disturbances that produce seizures.

As described above there is an urgent need for the discovery and development of therapeutic alternatives for the medical management of epilepsy. Recent research and development programs have included the following exemplary hypothesis testing programs. In a first hypothesis, glutamate neurotransmitter pathway has been implicated in aberrant excitatory neurotransmission. Glutamate and aspartate are ligands for the N-methyl-D-aspartate receptors and ionophore receptors (AMPA and Glu 1-4). Research efforts have been dedicated to eliciting glutaminergic specific antagonists that may be productive inhibitors of aberrent exicitatory neural signals or may be effective to attentuate neural modulatory mechanisms that are defective in epileptogenic tissue.

Another hypothesis includes the glycinergic pathway. Because glycine is an additional excitatory neurotransmitter, efforts to identify glycinergic specific ligands that may be of therapeutic benefit to prevent, reduce, or ablate seizure activity in cortical tissue. A third hypothesis is the histamine receptor ligands and tachykinin receptor ligands may be useful for neuromodulation of excitatory neurotransmission.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with epilepsy based upon genotype. Current pathways that may have involvement in the therapeutic benefit of epilepsy include glutaminergic, serontonergic, dopaminergic, adrenergic, cholinergic, purinergic, GABAergic, glycinergic, taurine, oxytocin, vasopressin, calcium, potassium, or sodium channels, mitochondrial maintenance, protein maturation and degradation, and second messenger cascade gene pathways that are listed in Tables 1, 2, 3 and 4. One skilled in the art would be able to identify these pathway specific gene or genes that may be involved in the manifestation of epilepsy, are likely candidate targets for novel therapeutic approaches, or are involved in mediating patient population differences in drug response to therapies for epilepsy.

Based upon these varying hypotheses there are many products in development for epilepsy. Table 9 below lists current therapies that have not yet received U.S. marketing approval. The listed candidate therapeutic interventions response in patients with epilepsy may be affected by polymorphism in genes as described above.

### EXAMPLE 5

Migraine

I. Description of Migraine

Migraine is a neurologic disorder having an incidence of 0.5 in 1,000 in the U.S. population. It is a specific neurological syndrome that has multiple, complex manifestations. Migraine with auras, unilateral throbbing, and associated nausea is the basic clinical symptomatic presentation. The premonitory phase may be up to 24 hours and may be associated with auras or alterations of mood, appetite, visual, sensory, or motor functions. Migraine headache is a unilateral throbbing that is associated with photophobia, hypacusis, polyuria, and diarrhea.

There are many clinical subtypes of migraine. Broadly, these subtypes can be distinguished by the presence or absence of auras. Migraines without auras are defined as the classic type. Migraines with auras can be further classified as 1) migraine with typical auras, 2) migraine with prolonged auras, 3) familial hemiplegic migraine, 4) basilar migraine, 5) migraine without headache, and 6) migraine with acute-onset aura. Additional migraine types include ophthalmologic migraine and retinal migraine.

II. Current Therapies for Migraine

Migraine medical therapy depends on the acute or prophylactic nature of the therapy and whether the migraine is diagnosed as mild, moderate, or severe. Many patients will take a step approach to each separate migraine attack, starting with weakly acting agents and progressing to more potent drugs. For patients with severe migraine, therapy includes prophylactic management.

Therapy for Acute Migraine

Mild migraine is a headache that may accompanied by nausea, is unilaterally throbbing, and can be treated by nonprescription analgesics. Patients infrequently consult a neurologist for care of mild migraines because the level of impairment imparted by the headache portion is not debilitating and is relatively short lived. Mild migraine is thus treated with aspirin, acetominophen, ibuprophen, indomethacin, naproxen sulfate, and in some cases antiemetic drugs (diphenhyrdamine, prochlorperazine, promethazine, and metchlorpramide).

Moderate migraine is generally characterized by similar symptoms of mild migraine, however the frequency and or severity are increased. Patients with moderate migraine are generally not relieved with non-narcotic analgesics, and require medications that combine aspirin or acetominophen with a mild sedative or α and β adrenergic receptor mediated vasoconstriction.

Severe migraine is characterized by similar symptoms as mild and moderate migraine. However, the severity and frequency of headache is debilitating. Patients seek relief from the headache pain in the acute stage and frequently require prophylactic maintenance therapy. The drugs used for the therapy of acute migraine are members of the ergot alkaloid family or sumitriptan.

The ergot alkaloids are partial agonists and antagonists for a variety of receptor types; serotoninergic, adrenergic, dopaminergic, muscarinic, and GABAergic. Synthetic products with similar chemical structures to ergotamine predominantly are agonists at the serotonin subtype 1D or 1B. Both of these two subtypes act by inhibiting adenylyl cyclase activity in cortical neurons. Ergotamine is also a vasoconstrictor; this activity is thought to occur through activation of the α1 adrenergic receptor system. Ergotamine is metabolized by undefined pathways and metabolites are excreted primarily in the bile. The bioavailability of ergotamine is approximately 1% due the potent first pass effect after parenteral delivery of the drug and erratic absorption between individuals.

Sumitripan is another drug used for the acute, severe migraine attacks. Sumitripan is a serotonin 1B, 1D selective receptor agonist. Because these receptor subtypes are auto receptors, activation of 5HT1B and 5HT1D receptors can act by controlling the release of the serotonin and other neurotransmitter release. Sumitriptan may also be efficacious in the treatment of migraine because it may block proinflammatory receptors at the level of nerve terminal in the perivascular space.

Other drugs used as adjunct therapy for acute, severe migraine attacks are corticosteroids and opioid analgesics. Due to their addictive qualities, opioid or narcotic analgesics are limited to acute, infrequent attacks.

Prophylactic Therapy of Migraine

There are currently six classes of standard treatments for the prophylactic therapy of migraine. They are 1) tricyclic antidepressants (amytriptylline), 2) 5HT antagonists (methylsergide), 3) β adrenergic receptor antagonists (propanolol, timolol, atenolol, metropolol, nadolol), 4) monoamine oxidase inhibitors (depranil), 5) calcium channel blockers (verapamil, flunarizine), and 6) anticonvulsants (divalproex sodium, chlorpromazine). The criteria for the selection of prophylactic therapy are 1) patient has 6 or more headaches each month, 2) symptomatic medications are contraindicated or ineffective, 3) medication is necessary more than twice each week, and 4) there is an expressed need on the part of the patient to prevent infrequent attacks, e.g. hemiplegic migraine, those headaches producing profound disruption, or those associated with a risk of stroke. The ultimate choice of the prophylactic medication is based upon the measured effect on the type of migraine and the patient's willingness to withstand the associated side effects.

III. Limitations of Current Therapies for Migraine

The current therapy of migraine includes management of acute attacks of the mild, moderate and severe categories. Therapies of severe migraine further include prophylactic therapies. Regardless of the acute or prophylactic nature of the therapy, there are both efficacy and toxicity limitations in which migraine remains problematic for medical management.

Toxicity or Undesired Side Effects of Acute Migraine Therapy

Ergotamine and its derivatives are useful drugs for the management of acute severe migraine attacks, however there are side effects associated with administration of the drug. Ergotamine is an activator of the CNS emetic centers, and nausea and vomiting are a frequent side effect that can be confused with a manifestation of the migraine attack. Other undesirable side effects are weakness of the legs, muscle pains, numbness and tingling of toes, and transient tachy- or bradycardia.

A known side effect of sumitriptan is coronary vasospasm and it is thus contraindicated in patients with ischemic heart disease or Prinzmetal's angina.

Limitations of Prophylactic Migraine Therapy

Although prophylactic therapy for migraine can reduce the frequency and intensity of the migraine attack, there are patients that achieve dramatic improvement and there are those that achieve only a 50% reduction, indicating a limited efficacy and benefit for a significant population subgroup. In those patients, the severity and intensity must be significant to require daily prophylactic medication.

Of the six categories of prophylactic agents all have associated side effects that may or may not be tolerable to each individual patient. They are 1) tricyclic antidepressants: sedation, dry mouth, weight gain, tremor, cardiac arrythmias, aggravation of angle-closure glaucoma, and difficulty in urinating; 2) 5HT antagonist: weight gain, muscle cramps, vasoconstriction, and retroperitoneal pleuroperitoneal and subendocaridal fibrosis; 3) β adrenergic receptor antagonists: aggravation of asthma, bradycardia, hypotension, fatigue, depression, masking the symtpoms of diabetes mellitus; 4) monoamine oxidase inhibitors: orthostatic hypotension, insomnia, and nausea; 5) calcium channel blockers: are not frequently used, however are associated with constipationa and orthostatic hypotension; and 6) anticonvulsants: nausea, fatigue, weight gain, alopecia, tremor, liver dysfunction, and neural tube defects in developing embryos.

The least desired effect of prolonged prophylactic therapy of migraine is the associated increased frequency of headaches. Headaches, not of the migraine type, can occur daily and are related to rebound withdrawal from frequent use of the acute antimigraine medication. Patients experiencing this type of headache pattern are said to have transformed migraine and often experience episodic migraine attacks superimposed on their daily headaches. Ergotamines are frequently associated with chronic daily headaches, as are the triptans. Unfortunately, patients experiencing daily headaches are less likely to respond to acute therapy or any other preventative medications. Withdrawal of other migraine medications further render the patient more susceptible to daily headaches. Therefore, it is beneficial to prevent transformed migraine and chronic daily headaches. Drugs known to be associated with transformed migraine are generally limited to occasional use in patients that have greater than two migraines each month. It is additionally recommended for patients that experience more frequent headaches requiring over-the-counter or prescription medications be put on a rotating schedule.

IV. Impact of Pharmacogenomics on Drug Development for Migraine

As described above, there is evidence to suggest that there are efficacy and safety different responses to drug therapy within the migraine patient population. Although not all of these responses may be attributable to genotypic differences, it is expected that if stratification based upon genotype were performed, a reasonable correlation between drug response and genotype may become obvious. As described below, there are gene pathways that are involved with current drug therapy and those that may be potentially involved in the future. As described in the Detailed Description, methods for the identification of candidate genes and gene pathways, stratification, clinical trial design, and implementation of genotyping for appropriate medical management of a given disease is easily translated for migraine and patients diagnosed with migraine. As described below in section V. below there are likely gene pathways as are those that are outlined in the gene pathway Table 1 and matrix Table 2.

Advantages of Pharmacogenomic Clinical development of Migraine

The advantages of a clinical research and drug development program that includes the use of polymorphic genotyping for the stratification of patients for the appropriate selection of candidate therapeutic intervention includes 1) identification of patients that may respond earlier to antimigraine therapy, 2) identification of the primary gene and relevant polymorphic variance that directly affects efficacy, safety, or both of acute or prophylactic therapies, 3) identification of pathophysiologic relevant variance or variances and potential therapies affecting those allelic genotypes or haplotypes, and 4) identification of allelic variances or haplotypes in genes that indirectly affects efficacy, safety or both of acute or prophylactic therapies.

Based upon these advantages, designing and performing a clinical trial, either prospective or retrospective, which includes a genotype stratification arm will incorporate analysis of clinical outcomes and genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of migraine.

By identifying subsets of migraine patients that respond earlier to acute or prophylactic or agents, optimal candidate therapeutic interventions may reduce the period of time prior to relief of headache frequency and severity. Appropriate genotyping and correlation to dosing regimen would be beneficial to the patient, caregivers, medical personnel, and the patient's loved ones.

Optimization of serontonergic, nonsteroidal antiinflammatory, or cerebral vasoconstrictor mediated mechanism of therapy of migraine further demonstrates the utility of selection of a potential migraine patient that has a predisposing genotype in which selective antimigraine or agents may be more effective and or have an more desirable safety profile. In considering an optimization protocol, one could potentially predetermine variance or variances within the serotonergic receptor pathway, nonsteroidal antinflammatory pathway, or serotonergic receptor or nonsteroidal antiinflammatory mediated intracellular mechanism of action that is preeminently responsible for antimigraine drug response. By embarking on the previously described gene pathway approach, it is technically feasible to determine the relevant genes within such a targeted drug development program for migraine.

A sample of therapies approved or in development for preventing or treating the progression of migraine currently known in the art is shown in table 11. In this table, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

V. Description of Mechanism of Action Hypotheses for Future Migraine Drug Development

The pathogenesis of migraine includes the following theories: vascular, depression of cortical electrical activity, serotonergic abnormalities, alteration of neurotransmitter modulation, and modulation of neuroendocrine mechanisms. These are described briefly below.

The vascular theory of migraine posits that there is abnormal cerebral blood flow and it apparently plays a pivotal role in the clinical symptoms of migraine. Studies have shown that a decrease in cerebral blood flow during an aura and an increase in blood flow during headache occur in migraine patients. This theory is somewhat substantiated indirectly by the pharmacologic action of therapies for acute migraine and prophylaxis, as previously described.

There have been additional studies that point to a mechanism of spreading depression of cortical electrical activity and a concurrent alteration of blood flow. This theory suggests that focal reduction of electrical activity and concurrent increase in blood flow occurs focally and spreads across the hemisphere at a rate of 2-3 mm each minute. This spreading hypothesis has been refined to a description of migraine as an evolving process in the cerebral cortex that occurs secondarily to decreased cortical function, decreased coritcal metabolism, and or vasoconstriction of cortical arterioles.

Many studies have addressed the effect of serotonergic mechanism of the pathogenesis of migraine. These studies used the following premises: 1) there have been reports of decreased concentrations of serotonin in platelets and plasma, 2) increased levels of serotonin and serotonergic metabolites in urine, 3) lastly, migraine may be precipitated by abnormal release of biogenic amines, a theory borne out of the fact that reserpine and fenfluramine can precipitate a migraine attack.

Other theories propose that alteration s of neurotransmitter systems e.g. nitric oxide, glutamate, and opioid receptors may be part of the pathogenesis of migraine. Further, Some studies have included anatomical differences in the raphe system and within the cerebral vasculature as well as alterations of the autonomic nervous system.

Therapy of migraine is dependent on the appropriate diagnosis, as well as the type, frequency, and severity of the throbbing headache. Upon diagnosis, patient education to identify and avoid trigger factors is a critical first step in all patients.

Trigger factors may include but are not exclusive to alcohol (red wine), foods (chocolate, certain cheeses), irregular sleep patterns, and acute changes in stress levels. Triggers may also come from environmental factors, such as time-zone shifts, high altitudes, or barometric changes. In women, menstrual cycles may trigger a migraine attack. These trigger factors suggest that there are complicating factors to include in any pathophysiologic hypothesis of migraine, and that these hypotheses may include neuroendocrine, endocrine, and other metabolic factors.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with epilepsy based upon genotype. Current pathways that may have involvement in the therapeutic benefit of migraine include glutaminergic, serontonergic, dopaminergic, adrenergic, cholinergic, GABAergic, nitric oxide, peptide hormone processing, opiates, tachykinin, bradykinin, corticotropin releasing hormone, calcitonin/calcitonin gene related peptide, calcium channel, hemostasis, and second messenger cascade gene pathways that are listed in Tables 1, 2, 3 and 4. One skilled in the art would be able to identify these pathway specific gene or genes that may be involved in the manifestation of migraine, are likely candidate targets for novel therapeutic approaches, or are involved in mediating patient population differences in drug response to therapies for migraine.

Based upon these varying hypotheses as stated above, there are many products in development for migraine. Table 11 below lists current therapies that have not yet received U.S. marketing approval.

### EXAMPLE 6

Psychosis

I. Clinical Problem

Psychosis is a general term for major mental disorder characterized by loss of contact with reality, often manifested by disordered thought, delusions or hallucinations. Psychosis can be part of several distinct psychiatric diseases, including schizophrenia, manic-depressive disease, severe depression with psychotic features, organic psychotic disorders, as well as in alcohol or drug intoxication and acute idiopathic psychotic illnesses. The most common of these is schizophrenia. The antipsychotic drugs are also used to treat non-psychiatric conditions such as, for example, nausea and vomiting, movement disorders associated with neurodegenerative diseases such as Huntington's disease and Tourette's syndrome, pruritis and chronic hiccough. Example 6 focuses predominantly on schizophrenia, however similar analysis, in terms of the relevant pathways, genes, polymorphisms and analytical methods for establishing relationships between polymorphisms and drug responses, would obtain in all the other diseases treated with antipsychotic drugs.

Approximately 2 million Americans are affected by schizophrenia, at an estimated annual cost in excess of $32 billion dollars. Criteria for the diagnosis of schizophrenia and other psychoses, as well as diagnostic criteria for the other disorders treated with antipsychotics, are well established. (Diagnostic and Statistical Manual of Mental Disorders, 4th ed., American Psychiatric Association Press, Washington, D.C., 1994.)

II. Current Medical Management of Schizophrenia

Over 15 drugs are approved for treatment of psychosis in the US. They include the so-called conventional or typical antipsychotic drugs and the more recently introduced atypical antipsychotic drugs. The former class includes phenothiazines (e.g. chlorpromazine, the first antipsychotic to be widely used), thioxanthenes (e.g. thiothixene), butyrophenones (e.g. haloperidol, one of the most useful conventional antipsychotics) and other heterocyclic compounds. The atypical antipsychotics include compounds such as clozaril (the first, and best studied member of the class), risperidone, olanzapine, quetiapine, ziprasidone and iloperidone. Some drugs, such as loxapine, have pharmacology intermediate between that of the typical and atypical drugs.

The typical antipsychotics are believed to act predominantly by antagonizing dopamine receptors, particularly D2-dopamine receptors. These medications can be effective in reducing the positive symptoms of schizophrenia (hallucinations, delusions) but are generally not effective at alleviating the negative symptoms (withdrawal, flat affect, anhedonia, lack of will), nor do they generally result in improved cognitive function. In fact, negative symptoms and cognitive function may worsen on typical antipsychotics. Typical antipsychotics exhibit dose dependent efficacy, and the optimal dose for a given patient must be determined empirically by gradually increasing the dose until adequate control of symptoms is achieved (without unacceptable side effects—see below). A therapeutic dose is usually reached within 2-3 weeks of initiating therapy.

The atypical antipsychotic drugs have replaced the typical agents as front line therapy for schizophrenia and other psychoses because they have a beneficial impact on the negative symptoms as well as the positive symptoms of schizophrenia, and because, based on recent research, they may also improve cognitive function. The atypical drugs affect a number of neurotransmitter systems, with modulation of serotonergic neurotransmission—particularly 5HT2C receptor antagonism, a prominent effect in addition to modulation of dopaminergic function. The best studied of this class of drugs is clozapine, which binds dopamine receptors with low affinity, and also interacts with muscarinic, adrenergic, serotonergic, and histaminergic receptors. Table 15 depicts the relative receptor affinity (0-5 on a scale of 5, where 5 indicates a high affinity interaction) of a conventional drug (haloperidol) and an atypical drug (clozapine).

TABLE 15
Relative Receptor Affinities of Haloperidol and Clozapine

| | Neurotransmitter Receptor Subtype | | | | | | | |
|---|---|---|---|---|---|---|---|---|
| | D1 | D2 | 5HT_{2A} | 5HT_{1A} | α1 | α2 | H1 | M1 |
| Haloperidol | +3 | +4 | +1 | 0 | +2 | 0 | 0 | 0 |
| Clozapine | +2 | +2 | +1 | +3 | +3 | +3 | +4 | +5 |

The effectiveness of the atypical antipsychotic drugs has revealed the inadequacy of a simplistic dopamine excess hypothesis of schizophrenia. The clinical effects of the atypical antipsychotic drugs are likely to reflect the summation of a complex set of interactions with a variety of neurotransmitter receptors. Interpatient differences in the function, levels or anatomical distribution of these different receptors are likely to account for a substantial fraction of interpatient variation in response to atypical antipsychotic drugs. Further, the function, levels and anatomical distribution of receptors is largely under genetic control, as is the associated biosynthetic, catabolic, recycling and signal transduction machinery. An understanding of the specific genetic variants that have major effects on drug efficacy would allow a far more sophisticated selection of appropriate therapy and dose than is possible currently.

III. Limitations of Current Therapies

The chief limitations of antipsychotic medicines are (i) conventional and atypical neuroleptic agents do not reduce the signs and symptoms of schizophrenia in all patients (an estimated one third to one quarter of psychotic patients are resistant to therapy); (ii) a wide range of serious adverse effects. Further, it is impossible to predict the response of any given patient, particularly the mix of drug effects on positive symptoms, negative symptoms, cognitive deficits and side effects. As a result, selection of therapy is at present completely empirical. This approach is costly, as (i) multiple physician visits may be required before an optimal dose of an effective agent is attained; (ii) even after determining an effective drug regiment, the long term effects of therapy in specific patients generally remain unknown, particularly with respect to side effects; (iii) these problems result in low rates of compliance with therapy. Hence there is a need for tools that would allow the prospective identification of patients likely to be responsive to—and free from short or long term side effects from—a particular drug.

Efficacy Limitations

The dilemma confronting psychiatrists and other clinicians selecting therapy for psychotic patients has been described by Baldessarini in Goodman and Gilman's The Pharmacological Basis of Therapeutics (9th edition) as follows: “No one drug or combination of drugs has a selective effect on a particular symptom complex in groups of psychotic patients; although individual patients may appear to do better with one agent than another, this can be determined only by trial and errror”. Thus, a clinician selecting therapy for a newly diagnosed psychotic patient, generally selects a compound with which he is comfortable, based on past experience. If that agent is not effective, or is producing a side effect, then a second agent is selected, again, entirely based on the physicians clinical judgement, and so on. This approach to optimization of pharmacotherapy has both medical and economic drawbacks. From the medical point of view, it does not always result in the selection of optimal treatment, with the attendant drawbacks in patient compliance. From an economic viewpoint the number of physician visits required to reach an effective dose of an effective drug are greater than necessary, and some patients may require hospitalization during the period when various drug regimens are being tested. There are other costs of using less than optimal therapy: (i) a patient might experience an improvement in cognitive symptoms on an optimal drug that would allow performance of a regular job; suboptimal therapy, even while adequately controlling positive symptoms, might not be sufficient to enable job performance. (ii) An optimal drug would minimize side effects, and thereby reduce physician visits, while also resulting in greater compliance. (Noncompliance is likely to ultimately lead to more hospitalization.) Determination of an optimal dose of an antipsychotic is another challenging aspect of therapy with these agents. Baldessarini (Goodman and Gilman, 9th ed.) writes: “Optimal dosage of antipsychotic drugs requires individualization to determine doses that are effective, well-tolerated, and accepted by a patient. Careful observation of the patients changing response is the best guide to dosage.” As with selection of an optimal agent, discussed above, the determination of an optimal dose presently requires multiple physician visits. Clearly some fraction of interpatient variation in optimal dose level is likely due to genetic, and consequent biochemical differences between patients. Such differences may involve drug metabolizing enzymes or proteins that mediate pharmacodynamic effects. A list of such proteins is provided in Table 1. Many typical antipsychotic drugs are metabolised by cytochrome P450 enzymes, with consequent wide interpatient variation in pharmacokinetic parameters. Further, many antipsychotic drugs are converted to active metabolites which can have therapeutic effects or side effects. The metabolism of the tricyclic atypical drugs (clozapine, olanzapine, and quetiapine) occurs via N+-oxidation, N-glucuronidation, and phases 1 and 2 metabolism with final glucuronidation before renal excretion. The non-tricyclic atypical antipsychotic drugs (e.g. risperidone, sertindole and ziprasidone) have diverse chemical structures and there is less data on their metabolism, but it appears to include diverse phase 1 biotransformation reactions. As a rule, conventional antipsychotics are mainly effective against positive symptoms (hallucinations, delusions, illusions), while not significantly ameliorating negative symptoms (withdrawal and flat affect). They are also associated with a high incidence of adverse effects, particularly extrapyramidal symptoms (EPS) and tardive dyskinesia. Atypical antipsychotics constitute a significant improvement, in that they are at least as effective as conventional drugs against positive symptoms, they show at least some effectiveness against negative symptoms and, according to recent studies, they may also produce improvement in the cognitive deficits associated with schizophrenia (e.g. attention, executive function, short and long term memory), while causing substantially fewer extrapyramidal symptoms.

Toxicity Limitations

Unfortunately, conventional anti-psychotic drugs are uniformly associated with undesirable dose-dependent side effects. These include (but are not limited to) extrapyramidal effects, electrocardiogram abnormalities, sedation, weight gain, cognitive deficits, sexual or reproductive dysfunction, blood dyscrasias (particularly agranulocytosis associated with clozapine), neuroleptic malignant syndrome (parkinsonism with catatonia), jaundice, skin reactions, epithelial keratopathy and seizures. Skin reactions include uticaria and dermititis and are usually associated with phenothiazines. Epithelial keratopathy and corneal opacities are associated with chlorpromazine therapy. In extreme cases these effects impair vision, but they tend to spontaneously disappear upon discontinuation of chlorpromazine.

The extrapyramidal side effects of conventional neuroleptics include dystonia (facial grimacing, torticollis, oculgyric crisis), akathesia (feeling of distress or discomfort leading to restlessness or constant movement), and parkinsonian syndrome (rigidity and tremor at rest, flat facial expression).

Tardive dyskinesia is a common side effect of long term usage of conventional neuroleptic drugs. Tardive dyskinesia is a syndrome of abnormal involuntary repetitive, painless movements. These movements vary in intensity over time, dependent on the level of arousal or emotional distress. Typically there are quick choreiform (ticlike) movements of the face, eyelids (blinks or spasms), mouth (grimaces), tongue, extremities, or trunk. Increasing the dose of the conventional neuroleptic agent can reverse extrapyramidal effects short term, but at the cost of more severe dyskinesia long term. Not infrequently a clinician is compelled to change medication for a patient with adequately controlled schizophrenia because of dose related tardive dyskinesia or other extrapyramidal side effects.

Another important side effect of many antipsychotic drugs is QT wave prolongation, which has recently resulted in the withdrawal of an atypical antipsychotic compound. Cardiac conduction abnormalities associated with antipsychotic therapy have resulted in patient deaths, presumably as a consequence of ventricular tachycardias. The mechanism of the conduction abnormalities appears to involve drug binding to cardiac potassium channels and consequent interference with repolarization current. Sertindole, for example, is a new antipsychotic agent that binds with high affinity (3-14 nM, depending on conditions) to and antagonizes HERG, a cardiac potassium channel. The degree of interpatient variation in these effects is not well characterized. Genes likely to account for these differences encode potassium channels (which may also have some role in the central actions of these compounds), sodium channels and the genes associated with inherited forms of long QT wave syndrome (QT1, QT2, QT3, QT4, QT5 and QT6).

Yet another important side effect of antipsychotic drugs is weight gain which can lead to obesity.

IV. Impact of Genotyping on Drug Development for Schizophrenia

Most traditional neuroleptics have a narrow therapeutic-to-toxic index, and thus, the novel antipsychotics are the result of a search to substantially widen the distance between the dose that treats psychosis and the one that produces adverse effects. In vitro binding profiles have been created for the atypical antipsychotics that have been approved by the U.S. Food and Drug Administration (FDA)-clozapine, olanzapine, and risperidone and those that are under FDA review-quetiapine and sertindole. These profiles, which were compared with that of the typical neuroleptic haloperidol, provide guidance for predicting the adverse effects produced by these drugs. Most conventional antipsychotics have central nervous system effects, particularly extrapyramidal symptoms (EPS) and tardive dyskinesia, sedation, and dulling of cognition. Other adverse effects of the typical antipsychotics include the neuroleptic malignant syndrome, orthostatic hypotension, changes in liver function, anticholinergic and antiadrenergic side effects, sexual dysfunction, and weight gain. The newer agents have a lower incidence of EPS and tardive dyskinesia, while weight gain and changes in blood pressure and liver function tests are adverse effects that have been associated with the use of the newer agents. The favorable side effect profile of these new antipsychotics is likely to make patients more willing to continue treatment, and thus these agents represent a step forward in the treatment of patients with severe, chronic mental illness.

This paper reviews the current literature describing the metabolism of both multi-receptor clozapine analogue atypical antipsychotic drugs (clozapine, olanzapine, and quetiapine) and serotonin-dopamine antagonist atypical antipsychotic drugs (risperidone, sertindole and ziprasidone), to highlight the significance of those data in the context of clinical practice. The former group of atypical antipsychotic drugs shares a similar tricyclic structural nucleus and are metabolized through three major categorical metabolic pathways—N+-oxidation, N-glucuronidation, and phases 1 and 2 biotransformation with final glucuronidation before renal excretion.

There have been reports of polymorphisms in key genes that affect neuroleptic activity in schizophrenic patients. For example, within the dopamine D4 receptor subtype, there are known tandem repeats in exon 3. In a recent study, schizophrenic patients on maintenance doses of chlorpromazine were stratified into two groups, one having 2 tandem base pair repeats and the other having 4 tandem base pair repeats. Thirty-four percent of group one patients and 62% of group two patients had a favorable response to chlorpromazine therapy during acute stage treatments. The presence of homogeneous four 48 base pair repeats in both alleles in exon 3 of the dopamine D4 receptor subtype thus appears to be associated with beneficial chlorpromazine response.

Recently, a study of the serotonin receptor subtype 6, polymorphism (T267T vs. C267T) in a group of patients refractory to clozapine therapy was reported. In this study, it appeared that the T267T genotype patients were more likely to respond to continued therapy that those patients with C267T genotype patients.

A recent report documented a correlation of the serotonin 5HTC2 receptor subtype biallelic polymorphism and neuroleptic efficacy. A significant number of schizophrenic patients homozygous for the allele C2 who responded unsatisfactorily to antipsychotic medication as compared to control.

Three polymorphisms in the serotonergic receptors, i.e. 5HT2A (T102C); 5HT2C (cys23ser); and 5HT2A (his452tyr) have reports of positive or negative correlation with efficacy of antipsychotic therapies. This disparity in the literature will, in the future, be further examined in schizophrenic patient populations and correlation may be discovered.

The evidence that a variance in a gene involved in a pathway that affects neuroleptic drug response, indicates and supports the theory that there is a likelihood that other genes have similar qualities to various degrees. As drug research and development proceeds to identify more lead candidate therapeutic interventions for schizophrenia, there is possible utility in stratifying patients based upon their genotype for these yet to be correlated variances. Further, as described in the Detailed Description, methods for the identification of candidate genes and gene pathways, stratification, clinical trial design, and implementation of genotyping for appropriate medical management of a given disease is easily translated for schizophrenia. As described below in section V. below there are likely gene pathways as are those that are outlined in the gene pathway table 1 and matrix table 2.

Advantages of Pharmacogenomic Clinical Development of Neuroleptics

The advantages of a clinical research and drug development program that includes the use of genotyping for the stratification of patients for the appropriate selection of candidate therapeutic intervention includes 1) identification of patients that may respond earlier to neuroleptic therapy, 2) identification of polymorphic variances that directly affect efficacy, safety, or both, 3) identification of a pathophysiologically relevant variance or variances and potential therapies affecting those allelic genotypes or haplotypes.

Based upon these advantages, designing and performing a clinical trial, either prospective or retrospective, which includes a genotype stratification arm will incorporate analysis of clinical outcomes and genetic variation associated with those outcomes, and hypothesis testing of the statistically relevant correlation of the genotypic stratification and therapeutic benefits. If statistical relevance is detectable, these studies will be incorporated into regulatory filings. Ultimately, these clinical trial data will be considered during the approval for marketing process, as well as, incorporated into accepted medical management of schizophrenia.

By identifying subsets of schizophrenia patients that respond earlier to neuroleptic agents, optimal candidate therapeutic interventions may reduce the lag time (2 to 3 weeks) prior to relief of psychiatric symptoms. Appropriate genotyping and correlation to dosing regimen would be beneficial to the patient, caregivers, medical personnel, and the patient's loved ones.

As an example of identification of the primary gene and relevant polymorphic variance that directly affects efficacy, safety, or both one could select a gene pathway as described in the Detailed Description, and determine the effect of genetic polymorphism and therapy efficacy, safety, or both within that given pathway. Specifically, the optimization of dopaminergic antagonist therapy of schizophrenia by determining whether the schizophrenic patient has a predisposing genotype in which selective dopaminergic antagonists are more effective and or are more safe. In considering an optimization protocol, one could potentially predetermine the dopaminergic receptor or dopaminergic receptor mediated intracellular mechanism of action that is preeminently responsible for neuroleptic drug response. By embarking on the previously described gene pathway approach, it is technically feasible to determine the relevant genes within such a targeted drug development program for schizophrenia.

Identification of pathophysiologic relevant variance or variances and potential therapies affecting those allelic genotypes or haplotypes will speed the drug development. There is a need for therapies that are targeted to the disease and symptom management with limited or no undesirable side effects. Identification of a specific variance or variances within genes involved in the pathophysiologic manifestation of schizophrenia and specific genetic polymorphisms of these critical genes may assist the development of novel neuroleptic agents and the identification of those patients that may best benefit from therapy of these candidate therapeutic alternatives.

By identifying allelic variances or haplotypes in genes that indirectly affects efficacy, safety or both one could target specific secondary drug or agent therapeutic actions that affect the overall therapeutic action of conventional, atypical, or novel neuroleptic action.

A sample of therapies approved or in development for preventing or treating the progression of symptoms of schizophrenia currently known in the art is shown in Table 16. In this table, the candidate therapeutics were sorted and listed by mechanism of action. Further, the product name, the pharmacologic mechanism of action, chemical name (if specified), and the indication is listed as well.

Pharmacogenomics studies for these drugs, as well as other agents, drugs, compounds or candidate therapeutic interventions, could be performed by identifying genes that are involved in the function of a drug including, but not limited to is absorption, distribution metabolism, or elimination, the interaction of the drug with its target as well as potential alternative targets, the response of the cell to the binding of a drug to a target, the metabolism (including synthesis, biodistribution or elimination) of natural compounds which may alter the activity of the drug by complementary, competitive or allosteric mechanisms that potentiate or limit the effect of the drug, and genes involved in the etiology of the disease that alter its response to a particular class of therapeutic agents. It will be recognized to those skilled in the art that this broadly includes proteins involved in pharmacokinetics as well as genes involved in pharmacodynamics. This also includes genes that encode proteins homologous to the proteins believed to carry out the above functions, which are also worth evaluation as they may carry out similar functions. Together the foregoing proteins constitute the candidate genes for affecting response of a patient to the therapeutic intervention. Using the methods described above, variances in these genes can be identified, and research and clinical studies can be performed to establish an association between a drug response or toxicity and specific variances.

V. Mechanism of Action Hypotheses for Novel Therapies for Schizophrenia: Utility of Genotyping

The underlying etiology of schizophrenia is not established, however there is compelling evidence that modulation of several neurotransmitter systems has an impact on the disease. As discussed above, conventional anti-psychotic drugs, effective in the management of schizophrenia, are dopamine antagonists, specifically D2-receptor antagonists, which block dopaminergic neurotransmission in the forebrain. Additionally, drugs such as mescaline and amphetamines, which are known to stimulate dopaminergic pathways, have been shown to induce psychotic symptoms. Evidence of dysfunctional serotonergic neurotransmission in schizophrenia includes evidence of altered serotonin receptor density, altered serotonin metabolism, and the evidence that serotonin receptors appear to be important targets for the atypical neuroleptics.

Based on current knowledge, there are three hypotheses that underscore the utility of polymorphic genotype analysis within the schizophrenic population. In the first, it could be considered that endogenous dopamine levels and consequential dopaminergic tone varies among schizophrenic patients, affecting response to receptor antagonists. These genetic DNA variations may affect brain neurotransmitter modulation of dopaminergic transmission and dopaminergic receptor mediated intracellular mechanisms among schizophrenic patients. In the second hypothesis, genetic DNA variations may affect the level of expression and brain distribution of dopamine receptors, imparting a variation in drug response among schizophrenia patients. Further, consideration of other endogenous neurotransmitters, i.e. serotonin, levels and consequential endogenous neurotransmitter tone varies among schizophrenic patients, affecting response to neurotransmitter receptor ligands or neurotransmitter receptor mediated intracellular mechanisms.

Further, there may be genes within pathways that are either involved in metabolism of neurotransmitters or are involved in metabolism of various drugs or compounds. In Tables 1, 3 and 4, there are listings of candidate genes and specific single nucleotide polymorphisms that may be critical for the identification and stratification of a patient population diagnosed with epilepsy based upon genotype. Current pathways that may have involvement in the therapeutic benefit of schizophrenia include glutaminergic, serontonergic, dopaminergic, adrenergic, cholinergic, histaminergic, GABAergic, glycinergic, opiates, cholecystokinin, neurotensin, tachykinin, calcium channels, and second messenger cascade gene pathways that are listed in Tables 1, 3, and 4. One skilled in the art would be able to identify these pathway specific gene or genes that may be involved in the manifestation of schizophrenia, are likely candidate targets for novel therapeutic approaches, or are involved in mediating patient population differences in drug response to therapies for schizophrenia.

### EXAMPLE 7

Method for Producing cDNA

In order to identify sequence variances in a gene by laboratory methods it is in some instances useful to produce cDNA(s) from multiple human subjects. (In other instances it may be preferable to study genomic DNA.). Methods for producing cDNA are known to those skilled in the art, as are methods for amplifying and sequencing the cDNA or portions thereof. An example of a useful cDNA production protocol is provided below. As recognized by those skilled in the art, other specific protocols can also be used.

cDNA Production

** Make sure that all tubes and pipette tips are RNase-free. (Bake them overnight at 100° C. in a vaccum oven to make them RNase-free.)
1. Add the following to a RNase-free 0.2 ml micro-amp tube and mix gently:
   24 ul water (DEPC treated)
   12 ul RNA (1 ug/ul)
   12 ul random hexamers (50 ng/ul)
2. Heat the mixture to 70° C. for ten minutes.
3. Incubate on ice for 1 minute.
4. Add the following:
   16 ul 5× Synthesis Buffer
   8 ul 0.1 M DTT
   4 ul 10 mM dNTP mix (10 mM each dNTP)
   4 ul SuperScript RT II enzyme
   Pipette gently to mix.
5. Incubate at 42° C. for 50 minutes.
6. Heat to 70° C. for ten minutes to kill the enzyme, then place it on ice.
5. Incubate at 42° C. for 50 minutes.
6. Heat to 70° C. for ten minutes to kill the enzyme, then place it on ice.
7. Add 160 ul of water to the reaction so that the final volume is 240 ul.
8. Use PCR to check the quality of the cDNA. Use primer pairs that will give a 800 base pair long piece. See “PCR Optimization” for the PCR protocol.

The following chart shows the reagent amounts for a 20 ul reaction, a 80 ul reaction, and a batch of 39 (which makes enough mix for 36) reactions:

| | 20 ul × 1 | 80 ul × 1 | 80 ul × 39 | |
|---|---|---|---|---|
| | tube | tube | tubes | |
| Water | 6 ul | 24 ul | 936 | water |
| RNA | 3 ul | 12 ul | | RNA |
| Random hexamers | 3 ul | 12 ul | 468 | random |
| | | | | hexamers |
| synthesis buffer | 4 ul | 16 ul | 624 | synthesis buffer |
| 0.1 M DTT | 2 ul | 8 ul | 312 | 0.1 M DTT |
| 10 mM dNTP | 1 ul | 4 ul | 156 | 10 mM dNTP |
| SSRT | 1 ul | 4 ul | 156 | SSRT |

### EXAMPLE 8

Method for Detecting Variances by Single Strand Conformation Polymorphism (SSCP) Analysis

This example describes the SSCP technique for identification of sequence variances of genes. SSCP is usually paired with a DNA sequencing method, since the SSCP method does not provide the nucleotide identity of variances. One useful sequencing method, for example, is DNA cycle sequencing of ³²P labeled PCR products using the Femtomole DNA cycle sequencing kit from Promega (WI) and the instructions provided with the kit. Fragments are selected for DNA sequencing based on their behavior in the SSCP assay.

Single strand conformation polymorphism screening is a widely used technique for identifying an discriminating DNA fragments which differ from each other by as little as a single nucleotide. As originally developed by Orita et al. (Detection of polymorphisms of human DNA by gel electrophoresis as single-strand conformation polymorphisms. Proc Natl Acad Sci USA. 86(8):2766-70, 1989), the technique was used on genomic DNA, however the same group showed that the technique works very well on PCR amplified DNA as well. In the last 10 years the technique has been used in hundreds of published papers, and modifications of the technique have been described in dozens of papers. The enduring popularity of the technique is due to (1) a high degree of sensitivity to single base differences (>90%) (2) a high degree of selectivity, measured as a low frequency of false positives, and (3) technical ease. SSCP is almost always used together with DNA sequencing because SSCP does not directly provide the sequence basis of differential fragment mobility. The basic steps of the SSCP procedure are described below.

When the intent of SSCP screening is to identify a large number of gene variances it is useful to screen a relatively large number of individuals of different racial, ethnic and/or geographic origins. For example, 32 or 48 or 96 individuals is a convenient number to screen because gel electrophoresis apparatus are available with 96 wells (Applied Biosystems Division of Perkin Elmer Corporation), allowing 3×32, 2×48 or 96 samples to be loaded per gel.

The 32 (or more) individuals screened should be representative of most of the worlds major populations. For example, an equal distribution of Africans, Europeans and Asians constitutes a reasonable screening set. One useful source of cell lines from different populations is the Coriell Cell Repository (Camden, N.J.), which sells EBV immortalized lyphoblastoid cells obtained from several thousand subjects, and includes the racial/ethnic/geographic background of cell line donors in its catalog. Alternatively, a panel of cDNAs can be isolated from any specific target population.

SSCP can be used to analyze cDNAs or genomic DNAs. For many genes cDNA analysis is preferable because for many genes the full genomic sequence of the target gene is not available, however, this circumstance will change over the next few years. To produce cDNA requires RNA. Therefore each cell lines is grown to mass culture and RNA is isolated using an acid/phenol protocol, sold in kit form as Trizol by Life Technologies (Gaithersberg, Md.). The unfractionated RNA is used to produce cDNA by the action of a modified Maloney Murine Leukemia Virus Reverse Transcriptase, purchased in kit form from Life Technologies (Superscript II kit). The reverse transcriptase is primed with random hexamer primers to initiate cDNA synthesis along the whole length of the RNAs. This proved useful later in obtaining good PCR products from the 5′ ends of some genes. Alternatively, oligodT can be used to prime cDNA synthesis.

Material for SSCP analysis can be prepared by PCR amplification of the cDNA in the presence of one α³²P labeled dNTP (usually α³²P dCTP). Usually the concentration of nonradioactive dCTP is dropped from 200 uM (the standard concentration for each of the four dNTPs) to about 100 uM, and ³²P dCTP is added to a concentration of about 0.1-0.3 uM. This involves adding a 0.3-1 ul (3-10 uCi) of ³²P cCTP to a 10 ul PCR reaction. Radioactive nucleotides can be purchased from DuPont/New England Nuclear.

The customary practice is to amplify about 200 base pair PCR products for SSCP, however, an alternative approach is to amplify about 0.8-1.4 kb fragments and then use several cocktails of restriction endonucleases to digest those into smaller fragments of about 0.1-0.4 kb, aiming to have as many fragments as possible between 0.15 and 0.3 kb. The digestion strategy has the advantage that less PCR is required, reducing both time and costs. Also, several different restriction enzyme digests can be performed on each set of samples (for example 96 cDNAs), and then each of the digests can be run separately on SSCP gels. This redundant method (where each nucleotide is surveyed in three different fragments) reduces both the false negative and false positive rates. For example: a site of variance might lie within 2 bases of the end of a fragment in one digest, and as a result not affect the conformation of that strand; the same variance, in a second or third digest, would likely lie in a location more prone to affect strand folding, and therefore be detected by SSCP.

After digestion, the radiolabelled PCR products are diluted 1:5 by adding formamide load buffer (80% formamide, 1×SSCP gel buffer) and then denatured by heating to 90% C for 10 minutes, and then allowed to renature by quickly chilling on ice. This procedure (both the dilution and the quick chilling) promotes intra- (rather than inter-) strand association and secondary structure formation. The secondary structure of the single strands influences their mobility on nondenaturing gels, presumably by influencing the number of collisions between the molecule and the gel matrix (i.e., gel sieving). Even single base differences consistently produce changes in intrastrand folding sufficient to register as mobility differences on SSCP.

The single strands were then resolved on two gels, one a 5.5% acrylamide, 0.5×TBE gel, the other an 8% acrylamide, 10% glycerol, 1×TTE gel. (Other gel recipes are known to those skilled in the art.) The use of two gels provides a greater opportunity to recognize mobility differences. Both glycerol and acrylamide concentration have been shown to influence SSCP performance. By routinely analyzing three different digests under two gel conditions (effectively 6 conditions), and by looking at both strands under all 6 conditions, one can achieve a 12-fold sampling of each base pair of cDNA. However, if the goal is to rapidly survey many genes or cDNAs then a less redundant procedure would be optimal.

### EXAMPLE 9

Method for Detecting Variances by T4 Endonuclease VII (T4E7) Mismatch Cleavage Method

The enzyme T4 endonuclease VII is derived from the bacteriophage T4. T4 endonuclease VII is used by the bacteriophage to cleave branched DNA intermediates which form during replication so the DNA can be processed and packaged. T4 endonuclease can also recognize and cleave heteroduplex DNA containing single base mismatches as well as deletions and insertions. This activity of the T4 endonuclease VII enzyme can be exploited to detect sequence variances present in the general population.

The following are the major steps involved in identifying sequence variations in a candidate gene by T4 endonuclease VII mismatch cleavage:
1. Amplification by the polymerase chain reaction (PCR) of 400-600 bp regions of the candidate gene from a panel of DNA samples The DNA samples can either be cDNA or genomic DNA and will represent some cross section of the world population.
2. Mixing of a fluorescently labeled probe DNA with the sample DNA. Heating and cooling the mixtures causing heteroduplex formation between the probe DNA and the sample DNA.
3. Addition of T4 endonuclease VII to the heteroduplex DNA samples. T4 endonuclease will recognize and cleave at sequence variance mismatches formed in the heteroduplex DNA.
4. Electrophoresis of the cleaved fragments on an ABI sequencer to determine the site of cleavage.
5. Sequencing of a subset of PCR fragments identified by T4 endonuclease VI to contain variances to establish the specific base variation at that location.
   A more detailed description of the procedure is as follows:

A candidate gene sequence is downloaded from an appropriate database. Primers for PCR amplification are designed which will result in the target sequence being divided into amplification products of between 400 and 600 bp. There will be a minimum of a 50 bp of overlap not including the primer sequences between the 5′ and 3′ ends of adjacent fragments to ensure the detection of variances which are located close to one of the primers.

Optimal PCR conditions for each of the primer pairs is determined experimentally. Parameters including but not limited to annealing temperature, pH, MgCl₂ concentration, and KCl concentration will be varied until conditions for optimal PCR amplification are established. The PCR conditions derived for each primer pair is then used to amplify a panel of DNA samples (cDNA or genomic DNA) which is chosen to best represent the various ethnic backgrounds of the world population or some designated subset of that population.

One of the DNA samples is chosen to be used as a probe. The same PCR conditions used to amplify the panel are used to amplify the probe DNA. However, a flourescently labeled nucleotide is included in the deoxy-nucleotide mix so that a percentage of the incorporated nucleotides will be fluorescently labeled.

The labeled probe is mixed with the corresponding PCR products from each of the DNA samples and then heated and cooled rapidly. This allows the formation of heteroduplexes between the probe and the PCR fragments from each of the DNA samples. T4 endonuclease VII is added directly to these reactions and allowed to incubate for 30 min. at 37 C. 10 ul of the Formamide loading buffer is added directly to each of the samples and then denatured by heating and cooling. A portion of each of these samples is electrophoresed on an ABI 377 sequencer. If there is a sequence variance between the probe DNA and the sample DNA a mismatch will be present in the heteroduplex fragment formed. The enzyme T4 endonuclease VII will recognize the mismatch and cleave at the site of the mismatch. This will result in the appearance of two peaks corresponding to the two cleavage products when run on the ABI 377 sequencer.

Fragments identified as containing sequencing variances are subsequently sequenced using conventional methods to establish the exact location and sequence variance.

### EXAMPLE 10

Method for Detecting Variances by DNA Sequencing.

Sequencing by the Sanger dideoxy method or the Maxim Gilbert chemical cleavage method is widely used to determine the nucleotide sequence of genes. Presently, a worldwide effort is being put forward to sequence the entire human genome. The Human Genome Project as it is called has already resulted in the identification and sequencing of many new human genes. Sequencing can not only be used to identify new genes, but can also be used to identify variations between individuals in the sequence of those genes.

The following are the major steps involved in identifying sequence variations in a candidate gene by sequencing:
1. Amplification by the polymerase chain reaction (PCR) of 400-700 bp regions of the candidate gene from a panel of DNA samples The DNA samples can either be cDNA or genomic DNA and will represent some cross section of the world population.
2. Sequencing of the resulting PCR fragments using the Sanger dideoxy method. Sequencing reactions are performed using flourescently labeled dideoxy terminators and fragments are separated by electrophoresis on an ABI 377 sequencer or its equivalent.
3. Analysis of the resulting data from the ABI 377 sequencer using software programs designed to identify sequence variations between the different samples analyzed.
   A more detailed description of the procedure is as follows:

A candidate gene sequence is downloaded from an appropriate database. Primers for PCR amplification are designed which will result in the target sequence being divided into amplification products of between 400 and 700 bp. There will be a minimum of a 50 bp of overlap not including the primer sequences between the 5′ and 3′ ends of adjacent fragments to ensure the detection of variances which are located close to one of the primers.

Optimal PCR conditions for each of the primer pairs is determined experimentally. Parameters including but not limited to annealing temperature, pH, MgCl₂ concentration, and KCl concentration will be varied until conditions for optimal PCR amplification are established. The PCR conditions derived for each primer pair is then used to amplify a panel of DNA samples (cDNA or genomic DNA) which is chosen to best represent the various ethnic backgrounds of the world population or some designated subset of that population.

PCR reactions are purified using the QIAquick 8 PCR purification kit (Qiagen cat# 28142) to remove nucleotides, proteins and buffers. The PCR reactions are mixed with 5 volumes of Buffer PB and applied to the wells of the QIAquick strips. The liquid is pulled through the strips by applying a vacuum. The wells are then washed two times with 1 ml of buffer PE and allowed to dry for 5 minutes under vacuum. The PCR products are eluted from the strips using 60 ul of elution buffer.

The purified PCR fragments are sequenced in both directions using the Perkin Elmer ABI Prism™ Big Dye™ terminator Cycle Sequencing Ready Reaction Kit (Cat# 4303150). The following sequencing reaction is set up: 8.0 ul Terminator Ready Reaction Mix, 6.0 ul of purified PCR fragment, 20 picomoles of primer, deionized water to 20 ul. The reactions are run through the following cycles 25 times: 96° C. for 10 second, annealing temperature for that particular PCR product for 5 seconds, 60° C. for 4 minutes.

The above sequencing reactions are ethanol precipitated directly in the PCR plate, washed with 70% ethanol, and brought up in a volume of 6 ul of formamide dye. The reactions are heated to 90° C. for 2 minutes and then quickly cooled to 4° C. 1 ul of each sequencing reaction is then loaded and run on an ABI 377 sequencer.

The output for the ABI sequencer appears as a series of peaks where each of the different nucleotides, A, C, G, and T appear as a different color. The nucleotide at each position in the sequence is determined by the most prominent peak at each location. Comparison of each of the sequencing outputs for each sample can be examined using software programs to determine the presence of a variance in the sequence. One example of heterozygote detection using sequencing with dye labeled terminators is described by Kwok et. al. (Kwok, P.-Y.; Carlson, C.; Yager, T. D., Ankener, W., and D. A. Nickerson, Genomics 23, 138-144, 1994). The software compares each of the normalized peaks between all the samples base by base and looks for a 40% decrease in peak height and the concomitant appearance of a new peak underneath. Possible variances flagged by the software are further analyzed visually to confirm their validity.

### EXAMPLE 11

Hardy-Weinberg Equilibrium

Evolution is the process of change and diversification of organisms through time, and evolutionary change affects morphology, physiology and reproduction of organisms, including humans. These evolutionary changes are the result of changes in the underlying genetic or hereditary material. Evolutionary changes in a group of interbreeding individuals or Mendelian population, or simply populations, are described in terms of changes in the frequency of genotypes and their constituent alleles. Genotype frequencies for any given generation is the result of the mating among members (genotypes) of their previous generation. Thus, the expected proportion of genotypes from a random union of individuals in a given population is essential for describing the total genetic variation for a population of any species. For example, the expected number of genotypes that could form from the random union of two alleles, A and a, of a gene are AA, Aa and aa. The expected frequency of genotypes in a large, random mating population was discovered to remain constant from generation to generation; or achieve Hardy-Weinberg equilibrium, named after its discoverers. The expected genotypic frequencies of alleles A and a (AA, 2Aa, aa) are conventionally described in terms of p²+2pq+q² in which p and q are the allele frequencies of A and a. In this equation (p²+2pq+q²=1), p is defined as the frequency of one allele and q as the frequency of another allele for a trait controlled by a pair of alleles (A and a). In other words, p equals all of the alleles in individuals who are homozygous dominant (AA) and half of the alleles in individuals who are heterozygous (Aa) for this trait. In mathematical terms, this is
p=AA+½Aa
Likewise, q equals the other half of the alleles for the trait in the population, or
q=aa+½Aa
Because there are only two alleles in this case, the frequency of one plus the frequency of the other must equal 100%, which is to say
p+q=1
Alternatively,
p=1−q OR q=1−p
All possible combinations of two alleles can be expressed as:
(p+q)²=1
or more simply,
p²+2pq+q²=1
In this equation, if p is assumed to be dominant, then p² is the frequency of homozygous dominant (AA) individuals in a population, 2pq is the frequency of heterozygous (Aa) individuals, and q² is the frequency of homozygous recessive (aa) individuals.

From observations of phenotypes, it is usually only possible to know the frequency of homozygous dominant or recessive individuals, because both dominant and recessives will express the distinguishable traits. However, the Hardy-Weinberg equation allows us to determine the expected frequencies of all the genotypes, if only p or q is known. Knowing p and q, it is a simple matter to plug these values into the Hardy-Weinberg equation (p²+2pq+q²=1). This then provides the frequencies of all three genotypes for the selected trait within the population.

This illustration shows Hardy-Weinberg frequency distributions for the genotypes AA, Aa, and aa at all values for frequencies of the alleles, p and q. It should be noted that the proportion of heterozygotes increases as the values of p and q approach 0.5.

Linkage Disequilibirum

Linkage is the tendency of genes or DNA sequences (e.g. SNPs) to be inherited together as a consequence of their physical proximity on a single chromosome. The closer together the markers are, the lower the probability that they will be separated during DNA crossing over, and hence the greater the probability that they will be inherited together. Suppose a mutational event introduces a “new” allele in the close proximity of a gene or an allele. The new allele will tend to be inherited together with the alleles present on the “ancestral,” chromosome or haplotype. However, the resulting association, called linkage disequilibrium, will decline over time due to recombination. Linkage disequilibrium has been used to map disease genes. In general, both allele and haplotype frequencies differ among populations. Linkage disequilibrium is varied among the populations, being absent in some and highly significant in others.

Quantification of the Relative Risk of Observable Outcomes of a Pharmacogenetics Trial

Let PlaR be the placebo response rate (0% (PlaR (100%) and TntR be the treatment response rate (0% (TntR (100%) of a classical clinical trial. ObsRR is defined as the relative risk between TntR and PlaR:
ObsRR=TntR/PlaR.

Suppose that in the treatment group there is a polymorphism in relation to drug metabolism such as the treatment response rate is different for each genotypic subgroup of patients. Let q be the allele a frequency of a recessive biallelic locus (e.g. SNP) and p=1−q the allele A frequency. Following Hardy-Weinberg equilibrium, the relative frequency of homozygous and heterozygous patients are as follow:

| | | |
|---|---|---|
| AA: p2 | Aa: 2pq | aa: q2 |

with
(p2+2pq+q2)=1.
Let's define AAR, AaR, aaR as respectively the response rates of the AA, Aa and aa patients. We have the following relationship:
TntR=AAR*p2+AaR*2pq+aaR*q2.

Suppose that the aa genotypic group of patients has the lowest response rate, i.e. a response rate equal to the placebo response rate (which means that the polymorphism has no impact on natural disease evolution but only on drug action) and let's define ExpRR as the relative risk between AAR and aaR, as
ExpRR=AAR/aaR.
From the previous equations, we have the following relationships:
ObsRR (ExpRR (1/PlaR
TntR/PlaR=(AAR*p2+AaR*2pq+aaR*q2)/PlaR
The maximum of the expected relative risk, max(ExpRR), corresponding to the case of heterozygous patients having the same response rate as the placebo rate, is such that:
ObsRR=ExpRR*p2+2pq+q2 [symbol] ExpRR=(ObsRR−2pq−q2)/p2
The minimum of the expected relative risk, min(ExpRR), corresponding to the case of heterozygous patients having the same response rate as the homozygous non-affected patients, is such that:
ObsRR=ExpRR*(p2+2pq)+q2 [symbol] ExpRR=(ObsRR=q2)/(p2+2pq)

For example, if q=0.4, PlaR=40% and ObsRR=1.5 (i.e. TntR=60%), then 1.6 (ExpRR (2.4. This means that the best treatment response rate we can expect in a genotypic subgroup of patients in these conditions would be 95.6% instead of 60%.

This can also be expressed in terms of maximum potential gain between the observed difference in response rates (TntR−PlaR) without any pharmacogenetic hypothesis and the maximum expected difference in response rates (max(ExpRR)*PlaR−TntR) with a strong pharmacogenetic hypothesis:
(max(ExpRR)*PlaR−TntR)=[(ObsRR−2pq−q2)/p2]*PlaR−TntR [symbol]
(max(ExpRR)*PlaR−TntR)=[TntR−PlaR*(2pq+q2)−TntR*p2]/p2 [symbol]
(max(ExpRR)*PlaR−TntR)=[TntR*(1−p2)−PlaR*(2pq+q2)]/p2 [symbol]
(max(ExpRR)*PlaR−TntR)=[(1−p2)/p2]*(TntR−PlaR)
that is for the previous example,
(95.6%−60%)=[(1−0.62)/0.62]*(60%−40%)=35.6%

Suppose that, instead of one SNP, we have p loci of SNPs for one gene. This means that we have 2p possible haplotypes for this gene and (2p)(2p−1)/2 possible genotypes. And with 2 genes with p1 and p2 SNP loci, we have [(2p1)(2p−1)/2]*[(2p2)(2p2−1)/2] possibilities; and so on. Examining haplotypes instead of combinations of SNPs is especially useful when there is linkage disequilibrium enough to reduce the number of combinations to test, but not complete since in this latest case one SNP would be sufficient. Yet the problem of frequency above still remains with haplotypes instead of SNPs since the frequency of a haplotype cannot be higher than the highest SNP frequency involved.

Statistical Methods to be Used in Objective Analyses

The statistical significance of the differences between variance frequencies can be assessed by a Pearson chi-squared test of homogeneity of proportions with n−1 degrees of freedom. Then, in order to determine which variance(s) is(are) responsible for an eventual significance, we can consider each variance individually against the rest, up to n comparisons, each based on a 2×2 table. This should result in chi-squared tests that are individually valid, but taking the most significant of these tests is a form of multiple testing. A Bonferroni's adjustment for multiple testing will thus be made to the P-values, such as p*=1−(1−p)ⁿ.

The statistical significance of the difference between genotype frequencies associated to every variance can be assessed by a Pearson chi-squared test of homogeneity of proportions with 2 degrees of freedom, using the same Bonferroni's adjustment as above.

Testing for unequal haplotype frequencies between cases and controls can be considered in the same framework as testing for unequal variance frequencies since a single variance can be considered as a haplotype of a single locus. The relevant likelihood ratio test compares a model where two seqarate sets of haplotype frequencies apply to the cases and controls, to one where the entire sample is characterized by a single common set of haplotype frequencies. This can be performed by repeated use of a computer program (Terwilliger and Ott, 1994, Handbook of Human Linkage Analysis, Baltimore, John Hopkins University Press) to successively obtain the log-likelihood corresponding to the set of haplotype frequency estimates on the cases (lnL_{case}), on the controls (lnL_{control}), and on the overall (lnL_{combined}). The test statistic 2((lnL_{case})+(lnL_{control})−(lnL_{combined})) is then chi-squared with r−1 degrees of freedom (where r is the number of haplotypes).

To test for potentially confounding effects or effect-modifiers, such as sex, age, etc., logistic regression can be used with case-control status as the outcome variable, and genotypes and covariates (plus possible interactions) as predictor variables.

### EXAMPLE 12

Exemplary Pharmacogenetic Analysis Steps

In accordance with the discussion of distribution frequencies for variances, alleles, and haplotypes, variance detection, and correlation of variances or haplotypes with treatment response variability, the points below list major items which will typically be performed in an analysis of the pharmacogenetic determination of the effects of variances in the treatment of a disease and the selection/optimization of treatment.
1) List candidate gene/genes for a known genetic disease, and assign them to the respective metabolic pathways.
2) Determine their alleles, observed and expected frequencies, and their relative distributions among various ethnic groups, gender, both in the control and in the study (case) groups.
3) Measure the relevant clinical/phenotypic (biochemical/physiological) variables of the disease.
4) If the causal variance/allele in the candidate gene is unknown, then determine linkage disequilibria among variances of the candidate gene(s).
5) Divide the regions of the candidate genes into regions of high linkage disequilibrium and low disequilibrium.
6) Develop haplotypes among variances that show strong linkage disequilibrium using the computation methods.
7) Determine the presence of rare haplotypes experimentally. Confirm if the computationally determined rare haplotypes agree with the experimentally determined haplotypes.
8) If there is a disagreement between the experimentally determined haplotypes and the computationally derived haplotypes, drop the computationally derived rare haplotypes, construct cladograms from these haplotypes using the Templeton (1987) algorithm.
9) Note regions of high recombination. Divide regions of high recombination further to see patterns of linkage disequilibria.
10) Establish association between cladograms and clinical variables using the nested analysis of variance as presented by Templeton (1995), and assign causal variance to a specific haplotype.
11) For variances in the regions of high recombination, use permutation tests for establishing associations between variances and the phenotypic variables.
12) If two or more genes are found to affect a clinical variable determine the relative contribution of each of the genes or variances in relation to the clinical variable, using step-wise regression or discriminant function or principal component analysis.
13) Determine the relative magnitudes of the effects of any of the two variances on the clinical variable due to their genetic (additive, dominant or epistasis) interaction.
14) Using the frequency of an allele or haplotypes, as well as biochemical/clinical variables determined in the in vitro or in vivo studies, determine the effect of that gene or allele on the expression of the clinical variable, according to the measured genotype approach of Boerwinkle et al (Ann. Hum. Genet 1986).
15) Stratify ethnic/clinical populations based on the presence or absence of a given allele or a haplotype.
16) Optimize drug dosages based on the frequency of alleles and haplotypes as well as their effects using the measured genotype approach as a guide.

### EXAMPLE 13

Exemplary Pharmacogenetic Analysis Steps—Biological Function Analysis

In many cases when a gene which may affect drug action is found to exhibit variances in the gene, RNA, or protein sequence, it is preferable to perform biological experiments to determine the biological impact of the variances on the structure and function of the gene or its expressed product and on drug action. Such experiments may be performed in vitro or in vivo using methods known in the art.

The points below list major items which may typically be performed in an analysis of the effects of variances in the treatment of a disease and the selection/optimization of treatment using biological studies to determine the structure and function of variant forms of a gene or its expressed product.

1) List candidate gene/genes for a known genetic disease, and assign them to the respective metabolic pathways.

2) Identify variances in the gene sequence, the expressed mRNA sequence or expressed protein sequence.

3) Match the position of variances to regions of the gene, mRNA, or protein with known biological functions. For example, specific sequences in the promotor of a gene are known to be responsible for determining the level of expression of the gene; specific sequences in the mRNA are known to be involved in the processing of nuclear mRNA into cytoplasmic mRNA including splicing and polyadenylation; and certain sequences in proteins are known to direct the trafficking of proteins to specific locations within a cell and to constitute active sites of biological functions including the binding of proteins to other biological constituents or catalytic functions. Variances in sites such as these, and others known in the art, are candidates for biological effects on drug action.

4) Model the effect of the variance on mRNA or protein structure. Computational methods for predicting the structure of mRNA are known and can be used to assess whether a specific variance is likely to cause a substantial change in the structure of mRNA. Computational methods can also be used to predict the structure of peptide sequences enabling predictions to be made concerning the potential impact of the variance on protein function. Most useful are structures of proteins determined by X-ray diffraction, NMR or other methods known in the art which provide the atomic structure of the protein. Computational methods can be used to consider the effect of changing an amino acid within such a structure to determine whether such a change would disrupt the structure and/or function of the protein. Those skilled in the art will recognize that this analysis can be performed on crystal structures of the protein known to have a variance as well as homologous proteins expressed from different loci in the human genome, or homologous proteins from other species, or non-homologous but analogous proteins with similar functions from humans or other species.

5) Produce the gene, mRNA or protein in amounts sufficient to experimentally characterize the structure and function of the gene, mRNA or protein. It will be apparent to those skilled in the art that by comparing the activity of two genes or their products which differ by a single variance, the effect of the variance can be determined. Methods for producing genes or gene products which differ by one or more bases for the purpose of experimental analysis are known in the art.

6) Experimental methods known in the art can be used to determine whether a specific variance alters the transcription of a gene and translation into a gene product. This involves producing amounts of the gene by molecular cloning sufficient for in vitro or in vivo studies. Methods for producing genes and gene products are known in the art and include cloning of segments of genetic material in prokaryotes or eukaryotic hosts, run off transcription and cell-free translation assays that can be performed in cell free extracts, transfection of DNA into cultured cells, introduction of genes into live animals or embryos by direct injection or using vehicles for gene delivery including transfection mixtures or viral vectors.

7) Experimental methods known in the art can be used to determine whether a specific variance alters the ability of a gene to be transcribed into RNA. For example, run off transcription assays can be performed in vitro or expression can be characterized in transfected cells or transgenic animals.

8) Experimental methods known in the art can be used to determine whether a specific variance alters the processing, stability, or translation of RNA into protein. For example, reticulocyte lysate assays can be used to study the production of protein in cell free systems, transfection assays can be designed to study the production of protein in cultured cells, and the production of gene products can be measured in transgenic animals.

9) Experimental methods known in the art can be used to determine whether a specific variant alters the activity of an expressed protein product. For example, protein can be producted by reticulocyte lystae systems or by introducing the gene into prokaryotic organisms such as bacteria or lowre eukaryotic organisms such as yeast or fungus), or by introducing the gene into cultured cells or transgenic animals. Protein produced in such systems can be extracted or purified and subjected to bioassays known to those in the art as measures of the nction of that particular protein. Bioassays may involve, but are not limited to, binding, inhibition, or catalytic functions.

10) Those skilled in the art will recognize that it is sometimes preferred to perform the above experiments in the presence of a specific drug to determine whether the drug has differential effects on the activity being measured. Alternatively, studies may be performed in the presence of an analogue or metabolite of the drug.

11) Using methods described above, specific variances which alter the biological function of a gene or its gene product that could have an impact on drug action can be identified. Such variances are then studied in clinical trial populations to determine whether the presence or absence of a specific variance correlates with observed clinical outcomes such as efficacy or toxicity.

12) It will be further recognized that there may be more than one variance within a gene that is capable of altering the biological function of the gene or gene product. These variances may exhibit similar, synergistic effects, or may have opposite effects on gene function. In such cases, it is necessary to consider the haplotype of the gene, namely the combination of variances that are present within a single allele, to assess the composite function of the gene or gene product.

13) Perform clinical trials with stratification of patients based on presence or absence of a given variance, allele or haplotype of a gene. Establish associations between observed drug responses such as toxicity, efficacy, drug response, or dose toleration and the presence or absence of a specific variance, allele, or haplotype.

14) Optimize drug dosage or drug usage based on the presence of the variant.

### EXAMPLE 14

Stratification of Patients by Genotype in Prospective Clinical Trials.

In a prospective clinical trial, patients will be stratified by genotype to determine whether the observed outcomes are different in patients having different genotypes. A critical issue is the design of such trials to assure that a sufficient number of patients are studied to observe genetic effects.

The number of patients required to achieve statistical significance in a conventional clinical trial is calculated from:
N=2(z_{α}+z_{2β})²/(δ/σ)₂ (two tailed test) 1.1

From this equation it may be inferred that the size of a genetically defined subgroup Nᵢ required to achieve statistical significance for an observed outcome associated with variance or haplotype “i” can be calculated as:
Nᵢ=2(z_{α}+z_{2β})²(δᵢ/σᵢ)² 1.2

If Pᵢ is the prevalence of the genotype “i” in the population, the total number of patients that need to be incorporated in a clinical trial N_{g} to identify a population with haplotype “i” of size Nᵢ is given by:
N_{g}=Nᵢ/Pᵢ 1.3

It should be noted that N_{g} describes the total number of patients that need to be genotyped in order to identify a subset of Nᵢ patients with genotype “i”.

If genotyping is used as means for statistical stratification of patients, N_{g} represents the number of patients that would need to be enrolled in a trial to achieve statistical significance for subgroup “i”. If genotyping is used as a means for inclusion, it represents the number of patients that need to screened to identify a population of Nᵢ individuals for an appropriately powered clinical trial. Thus, N_{g} is a critical determinant of the scope of the clinical trial as well as Nᵢ.

A clinical trial can also be designed to test associations for multiple genetic subgroups “j” defined by a single allele in which case:
N_{g}=max(N_{gi}) for i=1 . . . j 1.4

If more than one subgroup is tested, but there is no overlap in the patients contained within the subgroups, these can be considered to be independent hypotheses and no multiple testing correction should be required. If consideration of more than one subgroup constitutes multiple testing, or if individual patients are included in multiple subgroups, then statistical corrections may required in the values of z_{α} or z_{2β} which would increase the number of patients required. It should be emphasized that a clinical trial of this nature may not provide statistically significant data concerning associations with any genotype other than “i”. The total number of patients that would be required in a clinical trial to test more than one genetically defined subgroup would be determined by the maximum value of N_{g} for any single subgroup.

The power of pharmacogenomics to improve the efficiency of clinical trials arises from the fact it is possible to have N_{g}<N. The goal of pharmacogenomic analysis is to identify a genetically define subgroup in which the magnitude of the clinical response is greater and the variability in response is reduced. These observations correspond to an increase in the magnitude of the (mean) observed response δ or a decrease the degree of variability σ. Since the value of Nᵢ calculated in equation 1.2 decreases non-linearly as the square of these changes, the total number of patients N_{g} can also decrease non-linearly, resulting in a clinical trial that requires fewer patients to achieve statistical significance. If δᵢ and σᵢ are not different than δ and σ, then N_{g} is greater than N as given by N_{g}=Nᵢ/Pᵢ. Values of δᵢ and σᵢ that give N_{g}<N can be calculated:
N_{g}<N if: Pᵢ>[(δ/σ)²]/[(δᵢ/σᵢ)²] 1.5

It is apparent from this analysis that N_{g} is not uniformly less than N, even with modest improvements in the values for δ₁ and σᵢ.

As with a conventional clinical trial, the incorporation of an appropriate control group in the study design is critical for achieving success. In the case of a prospective clinical trial, the control group commonly is selected on the basis of the same inclusion criteria as the treatment group, but is treated with placebo or a standard therapeutic regimen rather than the investigational drug. In the case of a study with subgroups that are defined by haplotype, the ideal control group for a treatment subgroup with hapotype “i” is a placebo-treated subgroup with haplotype “i”. This is often a critical control, since haplotypes which may be associated with the response to treatment may also affect the natural course of the disease.

A critical issue in considering control groups is that σ for the control group placebo treated population with haplotype “i” may not be equivalent to that of the control population. If so, 1.5 may overestimate the benefits of any reduction in σᵢ in the treatment response group if there is not also a reduction in as in σᵢ the control group.

If σ of the treatment and control groups are not equivalent, δ would be still calculated as the difference in the response of the two groups, but σ would be different in the two groups with values of σ₀ or σ₁ respectively. In this case, the number of patients in the genetically defined subgroup Nᵢ would be defined by:
Nᵢ=(σZ_{α}+σᵢZ_{β})²/δ² 2.1

The total number of patients that would need to be enrolled in such a trial would be the maximium of
N or N/Pi 2.2

It will be apparent that such an analysis remains sensitive to increases in δ, but is less sensitive to changes in σ which are not also reflected in the control group.

Certain analysis may be performed by comparing individuals with one haplotype against the entire normal population. Such an analysis may be used to establish the selectivity of the response associated with a specific haplotype. For example, it may be desirable to establish that the response or toxicity observed in a specific subgroup is greater than that associated observed with the entire population. It may also be of interest to compare the response to treatment between two different subgroups. If σ differs between the groups, then the estimate of the number of patients that need to be enrolled in the trial must be calculated using equations 2.1 with N being the maximum of Nᵢ/Pᵢ for the different subgroups.

Another issue in controls is the relative size of the treatment and control groups. In a prospectively designed clinical trial which selectively incorporates patients with haplotype “i” the number of patients in the control and treatment group will be essentially equivalent. If the control group is different, or if haplotypes are used for stratification but not inclusion, statistical corrections may need to be made for having populations of different size.

### EXAMPLE 15

Stratification of Patients by Phenotype.

The identification of genetic associations in Phase II or retrospective studies can be performed by stratifying patients by phenotype and analyzing the distribution of genotypes/haplotypes in the separate populations. A particularly important aspect of this analysis is that any gene may have only a partial effect on the observed outcome, meaning that there will be an association value (A) corresponding to the fraction of patients in a phenotypically-defined subgroup who exhibit that phenotype due to a specific genotype/phenotype.

It will be recognized to those skilled in the art that the fraction of individuals who exhibit a phenotype due to any specific allele will be less than 1 (i.e. A<1). This is true for several reasons. The observed phenotype may occur by random chance. The observed phenotype may be associated with environmental influences, or the observed phenotype may be due to different genetic effects in different individuals. Furthermore, the construction of haplotypes and analysis of recombination may not group all alleles with pheontypically-significant variances within a single haplotype or haplotype cluster. In this case, causative variances at a single locus may be associated with more than one haplotype or haplotype cluster and the association constant A for the locus would be A=A₁+A₂+ . . . +Aₙ<1. It is likely that many phenotypes will be associated with multiple alleles at a given locus, and it is particularly important that statistical methods be sufficiently robust to identify association with a locus even if Aᵢ is reduced by the presence of several causative alleles.

Statistical methods can be used to identify genetic effects on an observed outcome in patient groups stratified by phenotype, eg the presence or absence of the observed response. One such method entails determining the allele frequencies in two populations of patients stratified by an observed clinical outcome, for example efficacy or toxicity and performing a maximum likelihood analysis for the association between a given gene and the observed phenotype based on the allele frequencies and a range of values for A (the association constant between a specific allele and the observed outcome used to stratify patients).

This analysis is performed by comparing the observed gene frequencies in a patient population with an observed outcome to gene frequencies in a table in which the predicted frequencies of different alleles of the gene assuming different values of the association constant A for that allele. This table of predicted gene frequencies can be constructed by those skilled in the art based on the frequency of any specific allele in the normal population, the predicted inheritance of the effect (e.g. dominant or recessive) and the fraction of a subgroup with a specific outcome who would have that allele based on the association constant A.

For example, if a specific outcome was only observed in the presence of a specific allele of a gene, the expected frequency would be 1. If a specific outcome was never observed in the presence of a specific allele of a gene, the expected frequency would be 0. If there was no association between the allele and the observed outcome, the frequency of that allele among individuals with an observed outcome would be the same as in the general population. A statistical analysis can be performed to compare the observed allele frequencies with the predicted allele frequencies and determine the best fit or maximum likeihood of the association. For example, a chi square analysis will determine whether the observed outcome is statistically similar to predicted outcomes calculated for different modes of inheritance and different potential values of A. P values can then be calculated to determine the likelihood that any specific association is statistically significant. A curve can be calculated based on different values of A, and the maximal likelihood of an association determined from the peak of such a curve. Methods for chi square analysis are known to those in the art.

A multidimensional analysis can also be performed to determine whether an observed outcome is associated with more than one allele at a specific genetic locus. An example of this analysis considering the potential effects of two different alleles of a single gene is shown. It will be apparent to those skilled in the art that this analysis can be extended to n dimensions using computer programs.

This analysis can be used to determine the maximum likelihood that one or more alleles at a given locus are associated with a specific clinical outcome.

It will be apparent to those skilled in the art that critical issues in this analysis include the fidelity of the phenotypic association and identification of a control group. In particular, it may be useful to perform an identical analysis in patients receiving a placebo to eliminate other forms of bias which may contribute to statistical errors.

### Other Embodiments

The invention described herein provides a method for identifying patients with a risk of developing neurological disease or dysfunction by determining the patients allele status for a gene listed in Tables 1, 3, and 4 and providing a forecast of the patients ability to respond to or tolerate a given drug treatment. In particular, the invention provides a method for determining, based on the presence or absence of a polymorphism, a patient's likely response to drug therapies of neurological disease or dysfunction. Given the predictive value of the described polymorphisms a candidate polymorphism is likely to have a similar predictive value for other drugs acting through other pharmacological mechanisms. Thus, the methods of the invention may be used to determine a patient's response to other drugs including, without limitation, antihypertensives, anti-obesity, anti-hyperlipidemic, or anti-proliferative, antioxidants, or enhancers of terminal differentiation.

In addition, while determining the presence or absence of the candidate allele is a clear predictor determining the efficacy of a drug on a given patient, other allelic variants of reduced catalytic activity are envisioned as predicting drug efficacy using the methods described herein. In particular, the methods of the invention may be used to treat patients with any of the possible variances, e.g., as described in Table 3 of Stanton et al., U.S. application Ser. No. 09/300,747.

In addition, while the methods described herein are preferably used for the treatment of human patients, non-human animals (e.g., dogs, cats, sheep, cattle and other bovines, swine, and apes and other non-human primates) may also be treated using the methods of the invention.

It will be readily apparent to one skilled in the art that varying substitutions and modifications may be made to the invention disclosed herein without departing from the scope and spirit of the invention. For example, using other compounds, and/or methods of administration are all within the scope of the present invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

1. A method for selecting a treatment for a patient suffering from a neurological or psychiatric disease, disorder, or condition, comprising: determining whether cells of said patient contain at least one variance in a gene from Tables 1, 3, or 4, wherein the presence or the absence of said at least one variance in said gene is indicative of the effectiveness of said treatment for said disease, disorder, or condition.

2-22. (canceled)
